(19) United States
(12) Patent Application Publication
(10) Pub. No.: US 2022/0375543 A1
(43) Pub. Date: Nov. 24, 2022

(54) TECHNIQUES FOR SINGLE SAMPLE EXPRESSION PROJECTION TO AN EXPRESSION COHORT SEQUENCED WITH ANOTHER PROTOCOL

(71) Applicant: BostonGene Corporation, Waltham, MA (US)

(72) Inventors: Nikita Kotlov, Moscow (RU); Kirill Shaposhnikov, Kemerovo (RU); Maksim Chelushkin, Moscow (RU); Ilya Cheremushkin, Moscow (RU); Artur Baisangurov, Moscow (RU); Svetlana Podsvirova, Moscow (RU); Svetlana Khorkova, Moscow (RU); Dmitry Kravchenko, Moscow (RU); Cagdas Tazearslan, Waltham, MA (US); Alexander Bagaev, Moscow (RU); Ekaterina Postovalova, Moscow (RU)

(21) Appl. No.: 17/747,824

(22) Filed: May 18, 2022

Related U.S. Application Data
(60) Provisional application No. 63/190,171, filed on May 18, 2021.

(51) Int. Cl. G16B 25/10; C12Q 1/6886; G16B 40/20

(52) U.S. Cl. CPC G16B 25/10; C12Q 1/6886; G16B 40/20; C12Q 1/6869

(57) ABSTRACT

Aspects of the disclosure relate to methods for improving compatibility of nucleic acid sequencing data obtained using different techniques. The disclosure is based, in part, on methods for mapping expression levels for genes expressed in a biological sample and obtained from a subject using a first protocol to expression levels as would have been determined through a second protocol if the second protocol were used to process the biological sample instead of the first protocol.

### RELATED APPLICATIONS

This application claims the benefit under 35 U.S.C. § 119(e) of the filing date of U.S. provisional application Ser. No. 63/190,171, filed May 18, 2021, the entire contents of which are incorporated by reference herein.

### BACKGROUND

Gene expression profiling (GEP) is a powerful tool widely used in oncology research. GEP utilizes techniques such as NGS and microarrays to simultaneously evaluate expression levels of multiple genes. Each expression level measurement is typically evaluated against a cohort of samples sequenced using the same methodology to understand whether the expression level values of a sample are high or low.

### SUMMARY

Aspects of the disclosure relate to methods for improving compatibility of nucleic acid sequencing data obtained using different techniques. The disclosure is based, in part, on methods for mapping RNA expression levels for genes expressed in a biological sample and obtained from a subject using a first protocol to RNA expression levels as would have been determined through a second protocol if the second protocol were used to process the biological sample instead of the first protocol.

Accordingly, in some aspects, the disclosure provides a method for mapping RNA expression levels for genes expressed in a biological sample and obtained from a subject using a first protocol to RNA expression levels as would have been determined through a second protocol if the second protocol were used to process the biological sample instead of the first protocol, the method comprising using at least one computer hardware processor to perform: obtaining first RNA expression data for a set of genes expressed in the biological sample obtained from the subject, the first RNA expression data indicative of first RNA expression levels (e.g., comprising first RNA expression levels) of genes in the set of genes, the first RNA expression data previously determined by processing the biological sample using the first protocol; and mapping the first RNA expression levels of genes in the set of genes to second RNA expression levels of genes in the set of genes, the second RNA expression levels indicating RNA expression levels as would have been determined through the second protocol, the second protocol being different from the first protocol, if the second protocol were used to process the biological sample instead of the first protocol, the mapping comprising for a first gene in the set of genes: obtaining, from among the first RNA expression levels, a first set of RNA expression levels including a first RNA expression level for the first gene and zero, one, or multiple first RNA expression levels for zero, one, or multiple other genes in the set of genes associated with the first gene; obtaining a first transformation for estimating an RNA expression level for the first gene as would have been determined according to the second protocol from RNA expression levels of one or more genes as determined through the first protocol; and determining, for inclusion in the second RNA expression levels, a second RNA expression level for the first gene by applying the first transformation to the first set of RNA expression levels.

In some aspects, the disclosure provides a system, comprising at least one computer hardware processor; and at least one computer-readable storage medium storing processor-executable instructions that, when executed by the at least one computer hardware processor, cause the at least one computer hardware processor to perform a method for mapping RNA expression levels for genes expressed in a biological sample and obtained from a subject using a first protocol to RNA expression levels as would have been determined through a second protocol if the second protocol were used to process the biological sample instead of the first protocol, the method comprising using at least one computer hardware processor to perform: obtaining first RNA expression data for a set of genes expressed in the biological sample obtained from the subject, the first RNA expression data indicative of first RNA expression levels of genes in the set of genes, the first RNA expression data previously determined by processing the biological sample using the first protocol; and mapping the first RNA expression levels of genes in the set of genes to second RNA expression levels of genes in the set of genes, the second RNA expression levels indicating RNA expression levels as would have been determined through the second protocol, the second protocol being different from the first protocol, if the second protocol were used to process the biological sample instead of the first protocol, the mapping comprising for a first gene in the set of genes: obtaining, from among the first RNA expression levels, a first set of RNA expression levels including a first RNA expression level for the first gene and zero, one, or multiple first RNA expression levels for zero, one, or multiple other genes in the set of genes associated with the first gene; obtaining a first transformation for estimating an RNA expression level for the first gene as would have been determined according to the second protocol from RNA expression levels of one or more genes as determined through the first protocol; and determining, for inclusion in the second RNA expression levels, a second RNA expression level for the first gene by applying the first transformation to the first set of RNA expression levels.

In some embodiments, the processor-executable instructions, when executed by the at least one computer hardware processor, cause the at least one computer hardware processor to perform a method as described herein.

In some aspects, the disclosure provides at least one computer-readable storage medium storing processor-executable instructions that, when executed by at least one computer hardware processor, cause the at least one computer hardware processor to perform a method for mapping RNA expression levels for genes expressed in a biological sample and obtained from a subject using a first protocol to RNA expression levels as would have been determined through a second protocol if the second protocol were used to process the biological sample instead of the first protocol, the method comprising using at least one computer hardware processor to perform: obtaining first RNA expression data for a set of genes expressed in the biological sample obtained from the subject, the first RNA expression data indicative of first RNA expression levels of genes in the set of genes, the first RNA expression data previously determined by processing the biological sample using the first protocol; and mapping the first RNA expression levels of genes in the set of genes to second RNA expression levels of genes in the set of genes, the second RNA expression levels indicating RNA expression levels as would have been determined through the second protocol, the second protocol being different from the first protocol, if the second protocol were used to process the biological sample instead of the first protocol, the mapping comprising for a first gene in the set of genes: obtaining, from among the first RNA expression levels, a first set of RNA expression levels including a first RNA expression level for the first gene and zero, one, or multiple first RNA expression levels for zero, one, or multiple other genes in the set of genes associated with the first gene; obtaining a first transformation for estimating an RNA expression level for the first gene as would have been determined according to the second protocol from RNA expression levels of one or more genes as determined through the first protocol; and determining, for inclusion in the second RNA expression levels, a second RNA expression level for the first gene by applying the first transformation to the first set of RNA expression levels.

In some aspects, the method further comprises identifying a cohort, from among a plurality of cohorts, with which to associate the subject using the second RNA expression levels.

In some embodiments, the set of genes comprises a second gene and a second set of genes associated with the second gene; wherein the mapping comprises obtaining, from among the first RNA expression levels, a second set of RNA expression levels including a first RNA expression level for the second gene and RNA expression levels for genes in the second set of genes associated with the second gene; obtaining a second transformation for estimating, from RNA expression levels of one or more genes as determined through the first protocol, an RNA expression level for the second gene as would have been determined according to the second protocol, wherein the second transformation is different than the first transformation; and determining, for inclusion in the second RNA expression levels a second RNA expression level for the second gene by applying the second transformation to the second set of RNA expression levels.

In some embodiments, the set of genes comprises one or more additional genes, and a further set of genes associated with the one or more additional genes; wherein the mapping comprises obtaining, from among the first RNA expression levels, a set of RNA expression levels including RNA expression levels for each of at least some of the one or more additional genes and RNA expression levels for at least some of the genes of the further set of genes associated with the one or more additional genes; obtaining respective transformations for estimating RNA expression levels for each of the one or more additional genes as would have been determined according to the second protocol; and determining, for inclusion in the second RNA expression levels, second RNA expression levels for each of the at least some of the additional genes of the subset by applying the second transformation to the first set of RNA expression levels. In some embodiments, a set of RNA expression levels comprises respective RNA expression levels for the one or more additional genes and RNA expression levels for at least some of the genes of the further set of genes associated with the one or more additional genes.

In some embodiments, the method comprises, prior to the mapping, determining, for each gene of at least a subset of the set of genes, a respective transformation for estimating the RNA expression level for each gene of the subset as would have been determined according to the second protocol from RNA expression levels of one or more genes of the subset as determined through the first protocol.

In some embodiments, the transformation is a linear transformation, and wherein determining the first transformation is performed using a regularized linear regression technique using training data. In some embodiments, the transformation is a non-linear transformation, and the first transformation is performed using a non-linear regression technique using training data.

In some embodiments, the training data comprises a plurality of paired values of RNA expression levels for each of at least some of the set of genes, wherein each pair of values in the plurality of paired values comprises an RNA expression level as determined through applying the first protocol to a particular biological sample and another RNA expression level as determined through applying the second protocol to the particular biological sample.

In some embodiments, the obtaining the first set of expression levels consists of obtaining a first expression level for the first gene and zero other RNA expression levels. In some embodiments, the obtaining the first set of RNA expression levels comprises identifying one or multiple other genes associated with the first gene. In some embodiments, the identifying is performed using Pearson correlation.

In some embodiments, the multiple other genes in the set of genes comprises between 2 and 100 genes associated with the first gene.

In some embodiments, the biological sample comprises a blood sample or tissue sample.

In some embodiments, the tissue sample comprises tumor tissue.

In some embodiments, the subject is a mammal. In some embodiments, the subject is a human.

In some embodiments, first RNA expression data and the second RNA expression data comprise normalized RNA expression levels. In some embodiments, the normalized RNA expression levels are normalized to transcripts per million (TPM) units.

In embodiments, the first protocol and the second protocol each comprise one or more sample processing steps and a sequencing step, and the first protocol comprises a sample processing step and/or a sequencing step that does not form part of the second protocol. In some embodiments, the first protocol comprises preserving the biological sample by a formalin-fixation and paraffin-embedding (FFPE) technique. In some embodiments, the first protocol further comprises performing exome capture (EC) RNA sequencing on the FFPE preserved biological sample.

In some embodiments, the second protocol comprises preserving the biological sample by a freshly frozen (FF) technique. In some embodiments, the second protocol comprises performing poly-A RNA sequencing on the FF preserved biological sample.

In some embodiments, the method further comprises generating the first RNA expression data by applying the first protocol to the biological sample.

In some embodiments, the identifying the cohort comprises associating the second RNA expression levels to RNA expression levels of a particular cohort of the plurality of cohorts; and identifying the subject as a member of the particular cohort to which the second RNA expression levels are associated.

In some embodiments, the method further comprises selecting a cancer therapeutic for the subject using the second RNA expression levels. In some embodiments, selecting the cancer therapeutic comprises determining a plurality of gene group RNA expression levels using the second RNA expression levels, the plurality of gene group RNA expression levels comprising a gene group RNA expression level for each gene group in a set of gene groups, wherein the set of gene groups comprises at least one gene group associated with cancer malignancy, and at least one gene group associated with cancer microenvironment; and selecting a cancer therapeutic using the determined gene group RNA expression levels.

In some embodiments, the method further comprises administering the selected cancer therapeutic to the subject.

### BRIEF DESCRIPTION OF DRAWINGS

FIG. 1A shows a schematic indicating that the RNA expression data obtained from a single biological sample using a first protocol (e.g., Exome Capture (EC) RNA sequencing) is not comparable with reference RNA expression data obtained from samples obtained using a different protocol (e.g., polyA RNA sequencing).

FIG. 1B shows a schematic indicating that methods according to some embodiments of the technology as described herein (e.g., Single Sample Mapping) may be applied to RNA expression data obtained from a single biological sample using a first protocol (e.g., Exome Capture (EC) RNA sequencing) in order to make the RNA expression data of the biological sample comparable to reference RNA expression data obtained from samples obtained using a different protocol (e.g., polyA RNA sequencing).

FIG. 2A shows a schematic depicting a Single-Gene Linear Mapping technique according to some embodiments of the technology as described herein.

FIG. 2B shows a schematic depicting a Single-Gene General Mapping technique according to some embodiments of the technology as described herein.

FIG. 2C shows a schematic depicting a Multi-Gene Linear Mapping technique according to some embodiments of the technology as described herein.

FIG. 2D shows a schematic depicting a Multi-Gene General Mapping technique according to some embodiments of the technology as described herein.

FIG. 3 is a diagram depicting a flowchart of an illustrative process 300 for mapping RNA expression levels for genes expressed in a biological sample obtained from a subject using a first protocol to RNA expression levels as would have been determined through a second protocol if the second protocol were used to process the biological sample instead of the first protocol, according to some embodiments of the technology as described herein.

FIG. 4 is a diagram depicting a flowchart of an illustrative process for mapping first RNA expression levels obtained from a subject using a first protocol to second RNA expression levels as would have been determined through a second protocol if the second protocol were used to process the biological sample instead of the first protocol, according to some embodiments of the technology as described herein.

FIG. 5 shows number of sample pairs per diagnosis in the MET500 data set.

FIG. 6 shows a principal components analysis (PCA) projection of the expression of 320 paired RNA-seq samples per protocol in the MET500 cohort.

FIG. 7 shows expression (log 2+1) correlation of representative examples of cancer or immune system genes; Exome capture (EC) values are plotted on the x-axis, poly-A values are plotted on the y-axis.

FIG. 8 shows UMAP projections for effective correction of the batch effect retaining cancer-specific grouping, with predicted samples mixed with Poly-A samples.

FIG. 9 shows concordance correlation values in the Biologically Meaningful Genes (BMG) space before and after correction by methods according to some embodiments of the technology as described herein.

FIG. 10 shows microenvironment gene signature concordance correlation coefficient (CCC) values against paired Poly-A and EC samples before and after correction.

FIG. 11 shows difference in CCC values for each single sample gene set enrichment assay (ssGSEA) process. Correlation values before correction subtracted from correlation values after correction. Dotted line denotes a difference equal to zero.

FIG. 12 shows CCC values for representative deconvolution processes before and after the correction of expression values.

FIG. 13 shows PolyA- vs. EC-predicted CD4+ T cells RNA percentage (before renormalization using RNA per cell type coefficient) before correction (left) and after correction (right). The line represents y=x.

FIG. 14 shows Pearson correlation of expression values for CXCR6 vs. CCR5. Efficiency of expression correction for CXCR6 gene: Single Gene vs. Multi-Gene techniques (measured in CCC).

FIG. 15 shows CCC values in the BMG space before and after correction with two developed “Single Gene” and “Multi Gene” techniques, according to some embodiments of the technology as described herein.

FIG. 16 shows the amount of variance by each of 20 Principal Components (PCs) of merged poly-A and EC expression data.

FIGS. 17A-17C show performance of a PCA method on the training set, removing 1st and 2nd PCs.

FIGS. 17D-17F show performance of a PCA method on the training set, removing 3rd and 5th PCs.

FIGS. 18A-18C show performance of a PCA method on the holdout set, removing 1st and 2nd PCs.

FIGS. 18D-18F show performance of a PCA method on the holdout set, removing 3rd and 5th PCs.

FIG. 19 shows a schematic depicting a workflow for mutual nearest neighbors (MNN)-transformation-based analysis.

FIG. 20 shows representative data for PCA on holdout and MNN-transformed data indicating the batch effect on paired samples sequenced using poly-A RNA-seq vs EC. “Original” means holdout expression data before correction.

FIG. 21 shows concordance correlation values in the BMG space before and after correction using MNN compared to a Single Gene sample mapping method according to some embodiments of the technology as described herein.

FIG. 22 shows concordance correlation values in the BMG space before and after correction using ComBat compared to a Single Gene sample mapping method according to some embodiments of the technology as described herein.

FIG. 23 shows PCA on holdout data showing the batch effect after correction of EC-expressions by ComBat.

FIG. 24 shows representative data for performance of methods according to some embodiments of the technology as described herein vs. other batch correction methods in four predefined groups of genes. CCC values are divided into three intervals.

FIG. 25A shows PCA on training data indicating the batch effect on paired samples sequenced using poly-A RNA-seq vs EC. Upper plot colored by the protocol, and lower plot colored by sample type.

FIG. 25B shows PCA on training data indicating different sample types separately demonstrate existing batch effect between protocols.

FIG. 26 shows PCA on validation data before correction indicating a batch effect. The upper plot is shaded by the protocol, and the lower plot is shaded by sample origin.

FIG. 27 shows PCA on validation data after correction indicating no batch effect. The upper plot shaded by the protocol, the middle plot is shaded by sample origin, and the lower plot shaded by sample type. Points from the same samples are grouped together.

FIGS. 28A-28B show gene expression correlation between FF-Poly-A and FFPE-EC_V7 on the same samples. CCC values are shown in the captions.

FIGS. 29A-29B show representative data for intra-sample correlation after correction. Average mean inter-sample correlation is ˜0.95.

FIG. 30 shows CCC distributions of BMG before correction, after correction with a Single Gene-ElasticNetCV technique, and after correction with a Multi-GeneCV technique.

FIG. 31 shows performance of methods according to some embodiments of the technology as described herein on laboratory data. CCC values are divided into three intervals.

FIG. 32 shows an exemplary process 3200 for processing sequencing data to obtain RNA expression data from sequencing data.

FIG. 33 depicts an illustrative implementation of a computer system that may be used in connection with some embodiments of the technology described herein.

### DETAILED DESCRIPTION

Aspects of the disclosure relate to methods for improving compatibility of nucleic acid sequencing data obtained using different protocols, for example RNA sequencing data obtained from samples prepared according to different preservation, nucleic acid extraction, and/or nucleic acid sequencing techniques.

Significant variability in the absolute expression values of genes within a single biological sample can be caused by one or more differences in the protocols used to derive the absolute expression values (e.g., differences in preservation, extraction, and/or nucleic acid sequencing techniques). Even when using the same protocol, significant variability in the absolute expression values of genes can be observed between samples that have not been processed together or completely identically (e.g. using different batches of reagents, different operators, in different conditions, etc.). This variability may be referred to as a batch effect in that it impacts (effects) multiple samples that are processed (as a batch) using the same protocol.

There are conventional techniques for mitigating the impact of such batch effects on genomic data. However, such techniques are applicable only in the context of mitigating batch effects between samples across large cohorts. That is a significant problem because such techniques cannot be applied to correct for batch effects when comparing an individual sample to a reference cohort comprising multiple samples (the single-sample batch effect setting) and can only be used when comparing two cohorts each with numerous samples (the multi-cohort batch effect setting).

This limitation of conventional techniques for correcting for batch effects in gene expression levels (e.g., RNA expression levels) is especially problematic in current precision medicine applications. Many precision medicine applications involve identifying biomarkers from sequencing data obtained from a subject (e.g., a subject having, suspected of having, or at risk of having cancer), identifying a cohort for the subject by comparing the subject's biomarkers to that of others in each of multiple cohorts, and taking a diagnostic, prognostic and/or therapeutic action on the basis of the identified cohort. Frequently, the biomarkers used either are themselves gene expression levels (e.g., RNA expression levels) or are derived from gene expression levels (e.g., RNA expression levels). When biomarkers for the subject depend on gene expression levels (e.g., RNA expression levels) obtained using one protocol and biomarkers for subjects in studied cohorts depend on gene expression levels (e.g., RNA expression levels) obtained using a different protocol, batch effects may render comparison of biomarkers between subject and cohorts improper, incorrect and/or meaningless. Improper diagnostic, prognostic, and/or treatment action could flow from such a comparison.

The following is a concrete example of the situation. Biological samples are usually preserved and stored as fresh frozen (FF) samples or formalin-fixed paraffin-embedded (FFPE) samples. FF storage is uncommon in clinical practice because it requires the purchase and maintenance of costly freezers. Nucleic acids are typically better preserved in FF samples, enabling high-quality sequencing output. On the other hand, FFPE samples are often used for routine pathological examination and are the primary method for clinical sample storage. However, the fixation step of FFPE preservation induces changes to nucleic acids. For example, FFPE treatment physically cross-links the nucleic acids and proteins in a sample, and degrades long molecules into smaller fragments, creating challenges for downstream RNA extraction and sequencing. Additionally, while fresh frozen samples may typically be sequenced using any of several different nucleic acid sequencing techniques (e.g., polyA RNA sequencing, Exome capture RNA sequencing, etc.), samples prepared by FFPE are not suitable for PolyA sequencing techniques because RNAs from FFPE materials are often degraded to small sizes and may lack a polyA tail.

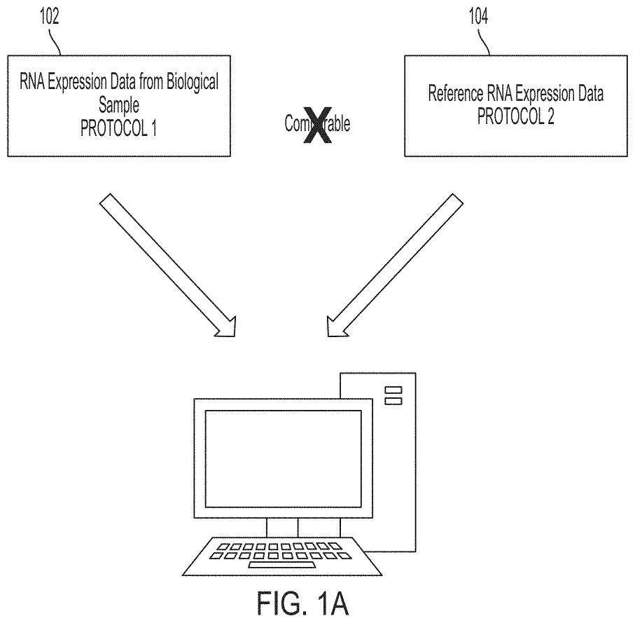

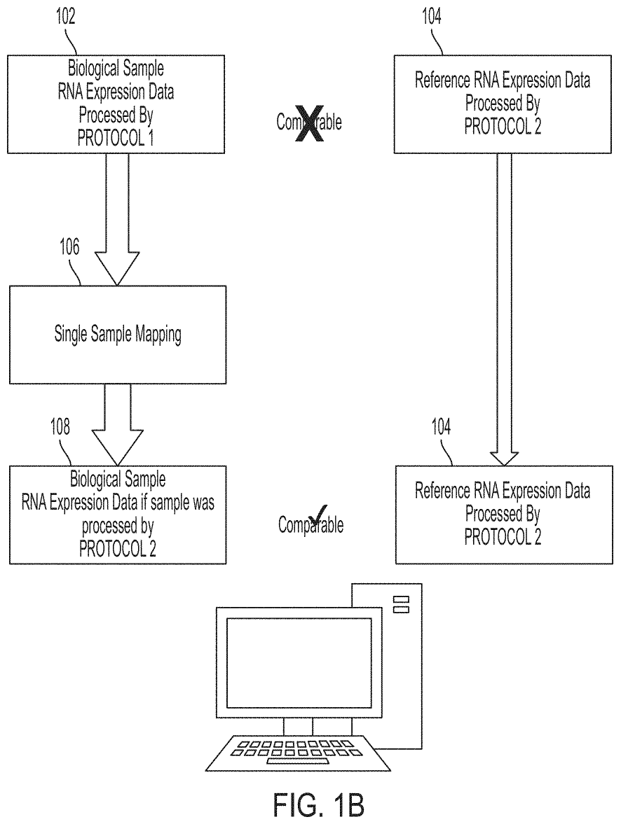

Continuing with this example, FIG. 1A illustrates the challenges to the technology of nucleic acid sequencing caused by the inapplicability of conventional techniques to address the batch effect problem in the single-sample setting. In FIG. 1A, expression data (e.g., RNA expression data) obtained from a single biological sample using a first protocol (e.g., FFPE preparation followed by Exome Capture (EC) RNA sequencing), 102, is not comparable with reference expression data (e.g., reference RNA expression data for a cohort of patients) obtained from samples obtained using a different protocol (e.g., FF preparation followed by polyA RNA sequencing), 104. For example, The Cancer Genome Atlas (TCGA) has established a database of well-annotated Poly-A RNA-sequenced samples from FF tissues for more than thirty cancer types, and represents a valuable resource of sequencing data that can potentially be utilized as a comparison gene expression profiling (GEP) cohort (e.g., FIG. 1A, 104). In contrast, samples obtained from cancer patients in the clinic almost exclusively comprise tissues preserved with the formalin-fixed paraffin-embedded (FFPE) tissue method (e.g., FIG. 1A, 102). Since these patient samples cannot be sequenced using Poly-A sequencing, GEP is performed using Exome Capture (EC) RNA-seq protocols. However, EC protocols often differ and are dependent on customized gene panels; therefore, patient samples and cohorts are often sequenced using different protocols and panels.

As described above, there is no available conventional technique to make gene expression data (e.g., RNA expression data) from single biological samples sequenced using Exome Capture techniques compatible, and therefore meaningfully comparable, with PolyA RNA-seq data. Thus, large cohorts of patient data obtained by polyA RNA-seq (e.g., TCGA data) of cancer research subjects may be of limited utility for a clinician needing to analyze expression data obtained from FFPE patient samples sequenced by EC. The lack of compatibility between sequencing data for FF-preserved samples and FFPE-preserved samples at the single sample level therefore has negative impacts on the quality of bioinformatic analysis of patient samples and the application of cancer research discoveries to clinical settings.

Accordingly, the inventors have developed statistical techniques for mapping RNA expression levels for genes expressed in a biological sample and obtained from a subject using a first protocol to RNA expression levels as would have been determined through a second protocol if the second protocol were used to process the biological sample instead of the first protocol.

In some embodiments, the mapping may be done on a gene-by-gene basis such that each particular gene is associated with a respective mapping that is used to estimate, from RNA expression levels of one or multiple genes as determined applying a first protocol to a biological sample, the RNA expression level of that particular gene as would have been determined had the biological sample been processed using the second protocol instead. In some embodiments, the mapping may be a linear mapping (e.g., a linear transformation) and its exact values may be estimated using linear regression techniques (e.g., linear regression, least absolute shrinkage, and selection operator (LASSO) regression, ridge regression, ElasticNet regression, or any other suitable regression or regularized regression technique) from training data, as described herein. Application of the statistical techniques developed by the inventors can be used to render the gene expression data (e.g., RNA expression data) of the biological sample compatible with gene expression data obtained by other sample preparation or sequencing techniques, allowing for direct single-sample comparisons.

In particular, the above described problem with respect to FIG. 1A may be addressed by the techniques developed by the inventors. As shown, in FIG. 1B, embodiments of the technology as described herein may be implemented as part of a software module (e.g., shown as “Single Sample Mapping” software module, 106, in FIG. 1B) that may be applied to RNA expression data obtained from a single biological sample using a first protocol (e.g., Exome Capture (EC) RNA sequencing), 102, in order to make the RNA expression data of the biological sample comparable (FIG. 1B, 108) to reference RNA expression data obtained from samples obtained using a different protocol (e.g., FIG. 1B, 104, such as TCGA data obtained by polyA RNA sequencing).

Accordingly, some embodiments provide for a computer-implemented method for identifying a (e.g., mammal, for example, human) subject as a member of a cohort, the method comprising: (A) obtaining first RNA expression data for a set of genes expressed in a biological sample (e.g., blood, tissue, tumor tissue) obtained from the subject, the first RNA expression data indicative of first RNA expression levels of genes in the set of genes, the first RNA expression data previously determined by processing the biological sample using a first protocol; (B) mapping the first RNA expression levels of genes in the set of genes to second RNA expression levels of genes in the set of genes, the second RNA expression levels indicating RNA expression levels as would have been determined through a second protocol different from the first protocol if the second protocol were used to process the biological sample instead of the first protocol, the mapping comprising for a first gene in the set of genes: (i) obtaining, from among the first RNA expression levels, a first set of RNA expression levels including a first RNA expression level for the first gene and zero, one, or multiple first RNA expression levels for zero, one, or multiple other genes, in the set of genes, which are associated with the first gene; (ii) obtaining a first transformation (e.g., a linear transformation) for estimating, from RNA expression levels of one or more genes as determined through the first protocol, an RNA expression level for the first gene as would have been determined according to the second protocol; and (iii) determining, for inclusion in the second RNA expression levels, a second RNA expression level for the first gene by applying the first transformation to the first set of RNA expression levels; and (C) identifying a cohort (e.g., a cohort of subjects, cohort of samples, etc.), from among a plurality of cohorts (e.g., a plurality of cohorts of subjects, plurality of cohorts of samples, etc.), with which to associate the subject using the second RNA expression levels.

Multiple genes may have their RNA expression levels mapped from “first protocol” values (measured in practice) to projected “second protocol values.” Thus, in some embodiments, the set of genes comprises a second gene and a second set of genes associated with the second gene, and the mapping comprises: (i) obtaining, from among the first RNA expression levels, a second set of RNA expression levels including a first RNA expression level for the second gene and RNA expression levels for genes in the second set of genes associated with the second gene; (ii) obtaining a second transformation for estimating, from RNA expression levels of one or more genes as determined through the first protocol, an RNA expression level for the second gene as would have been determined according to the second protocol, wherein the second transformation is different than the first transformation; and (iii) determining, for inclusion in the second RNA expression levels a second RNA expression level for the second gene by applying the second transformation to the second set of RNA expression levels.

More generally, in some embodiments, the set of genes comprises one or more additional genes, and a further set of genes associated with the one or more additional genes, and the mapping comprises: (i) obtaining, from among the first RNA expression levels, a set of RNA expression levels including RNA expression levels for each of at least some of the one or more additional genes and RNA expression levels for at least some of the genes of the further set of genes associated with the one or more additional genes; (ii) obtaining respective transformations for estimating RNA expression levels for each of the one or more additional genes as would have been determined according to the second protocol; and (iii) determining, for inclusion in the second RNA expression levels second RNA expression levels for each of the at least some of the additional genes of the subset by applying the second transformation to the first set of RNA expression levels.

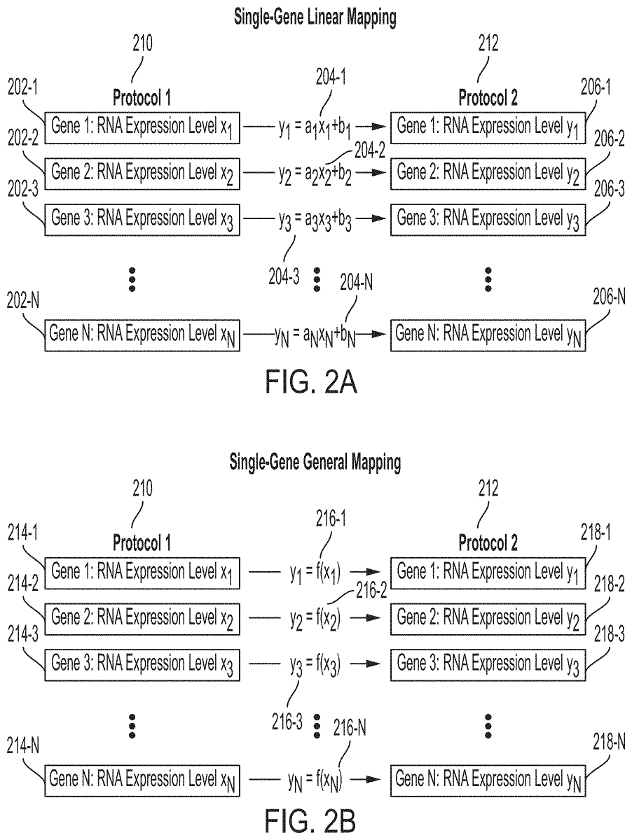

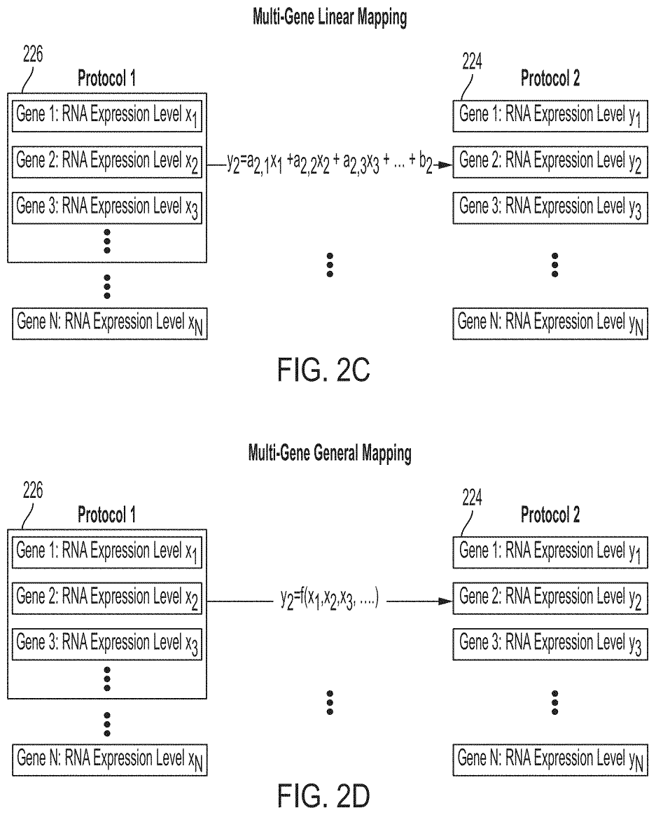

In some embodiments, the first transformation may map the expression value of a single gene as determined using the first protocol to an estimate of an RNA expression value for that single gene as would have resulted had the second protocol been applied to the same biological sample. Such a transformation may be termed a “one-gene-to-one-gene” or a “one-to-one” transformation. In some embodiments, such a transformation may be a linear transformation (e.g., as shown in FIG. 2A) or a any function f( ) that maps expression levels in a first protocol to expression levels in a second protocol, including, for example, a non-linear transformation (e.g., as shown in FIG. 2B). Examples of non-linear transformations that may be used include transformations implemented using a generalized linear model, polynomial regression, random forest regression, support vector machine (SVM) regression, neural networks, gradient boosting and/or any other suitable non-linear regression technique. In particular, FIG. 2A shows illustrative examples of one-to-one linear transformations, with a separate linear transformation used for each gene in a set of genes. For example, the RNA expression level of Gene 1, 202-1, according to Protocol 1, 210, is mapped using linear transformation 204-1, to obtain a Gene 1 second RNA expression level, 206-1, as would have resulted had Protocol 2, 212, been used. In another example, the RNA expression level of Gene 2, 202-2, according to Protocol 1, 210, is mapped using linear transformation 204-2, to obtain a Gene 2 second RNA expression level, 206-2, as would have resulted had Protocol 2, 212, been used. In another example, the RNA expression level of Gene 3, 202-3, according to Protocol 1, 210, is mapped using linear transformation 204-3, to obtain a Gene 3 second RNA expression level, 206-1, as would have resulted had Protocol 2, 212, been used. An RNA expression level of Gene N 202-N is mapped using linear transformation 204-N, to obtain a Gene N second RNA expression level, 206-N, as would have resulted had Protocol 2, 212, been used. Each such linear transformation may have been estimated using paired values of expression levels for the gene. The paired values of expression levels for each gene i are indicative of the expression levels of the gene when it has been sequenced by a first protocol, 210 (e.g., FFPE preparation followed by EC RNA-seq, “xᵢ”), and a second protocol, 212, (e.g., FF preparation followed by polyA RNA-seq, “yᵢ”). A linear transformation, 214, is then fit between the paired expression values to produce coefficients (e.g., aᵢ and bᵢ) that can be used to project gene expression level of the gene from the first protocol to the second protocol.

Other types of transformations (e.g., non-linear transformations) may be used as well, as shown in FIG. 2B, which illustrates that the linear transformations shown in FIG. 2A may be replaced with other types of transformations, as aspects of the technology described herein are not limited in this respect. As shown in FIG. 2B, the RNA expression levels may be mapped using any other suitable transformations fᵢ, rather than linear transformations as shown in FIG. 2A. As shown in FIG. 2B, the RNA expression level of Gene 1, 214-1, according to Protocol 1, 210, is mapped using function 216-1, to obtain a Gene 1 second RNA expression level, 218-1, as would have resulted had Protocol 2, 212, been used. In another example, the RNA expression level of Gene 2, 214-2, according to Protocol 1, 210, is mapped using function 216-2, to obtain a Gene 2 second RNA expression level, 218-2, as would have resulted had Protocol 2, 212, been used. In another example, the RNA expression level of Gene 3, 214-3, according to Protocol 1, 210, is mapped using function 216-3, to obtain a Gene 3 second RNA expression level, 218-3, as would have resulted had Protocol 2, 212, been used. An RNA expression level of Gene N, 214-N, is mapped using function 216-N, to obtain a Gene N second RNA expression level, 218-N, as would have resulted had Protocol 2, 212, been used.

In some embodiments, the first transformation may map the RNA expression values of multiple genes as determined using the first protocol to an estimate of an RNA expression value of one of the multiple genes as would have resulted had the second protocol been applied. Such a transformation may be termed a “many-gene-to-one-gene” or a “many-to-one” transformation. The second RNA expression level 224, under a second protocol, for a selected gene may be predicted from the RNA expression levels 226 for multiple genes obtained using a first protocol. The RNA expression levels 226 include an RNA expression level for the selected gene under the first protocol and one or more RNA expression levels (as determined by the first protocol) for one or more genes associated with the selected gene. In some embodiments, a separate linear transformation used to estimate a “second protocol” RNA expression value for each gene in the set of genes. Each such linear transformation may have been estimated using paired values of RNA expression levels for the genes. The estimation may have been performed in any suitable way including via linear regression or regularized linear regression (e.g., LASSO, ridge regression, ElasticNET). Other types of transformations (e.g., non-linear transformations) may be used as well, as shown in FIG. 2D, which illustrates that the linear transformations shown in FIG. 2C may be replaced with other types of transformations, as aspects of the technology described herein are not limited in this respect.

In some embodiments, the many-to-one transformations may improve the accuracy of the projection as compared to the single gene method using one-to-one transformations. That is because a many-to-one transformation may utilize a combination of paired values for 1) RNA expression levels of a gene of interest, and 2) RNA expression levels for genes associated with the gene of interest. In some embodiments, a gene of interest refers to a gene for which the transformation is being produced. In some embodiments, genes associated with the gene of interest are genes that have RNA expression levels correlated with the expression levels of the gene of interest (e.g. as determined by Pearson correlation).

Regardless of the type of transformation, in some embodiments, the transformation may be estimated from training data (using suitable estimation techniques, such as, linear or non-linear regression techniques). As may be appreciated from the foregoing, in some embodiments, the training data comprises a plurality of paired values of RNA expression levels for each at least some of the set of genes, wherein each pair of values in the plurality of paired values comprises an RNA expression level as determined through applying the first protocol to a particular biological sample and another RNA expression level as determined through applying the second protocol to the particular biological sample.

In some embodiments, obtaining the first set of RNA expression levels comprises identifying one or multiple other genes associated with the first gene. In some embodiments, the identifying may be performed using Pearson correlation and/or any other suitable correlation measure.

In some embodiments, the first and second protocols may be different protocols for obtaining sequencing data (e.g., RNA sequencing data). The difference may lie in the sample preservation, preparation, sequencing and/or any other aspect of processing a biological sample to obtain sequencing data. For example, the first protocol may comprise: (1) preserving the biological sample by a formalin-fixation and paraffin-embedding (FFPE) technique; and (2) performing exome capture (EC) RNA sequencing on the FFPE preserved biological sample. As another example, the second protocol may comprise: (1) preserving the biological sample by a freshly frozen (FF) technique; and (2) performing poly-A RNA sequencing on the FF preserved biological sample.

In some embodiments, identifying the cohort comprises: (1) associating the second RNA expression levels to RNA expression levels of a particular cohort of the plurality of cohorts; and (2) identifying the subject as a member of the particular cohort to which the second RNA expression levels are associated.

In some embodiments, the techniques further include selecting a cancer therapeutic for the subject using the second RNA expression levels and, optionally, administering the selected cancer therapeutic to the subject.

In some embodiments, the selecting a cancer therapeutic comprises: determining a plurality of gene group RNA expression levels using the second RNA expression levels, the plurality of gene group RNA expression levels comprising a gene group RNA expression level for each gene group in a set of gene groups, wherein the set of gene groups comprises at least one gene group associated with cancer malignancy, and at least one gene group associated with cancer microenvironment; and selecting a cancer therapeutic using the determined gene group expression levels.

Projecting RNA expression levels from a patient-derived sample sequenced by EC RNA-seq to expression levels if the sample had been prepared by polyA RNA-seq improves the compatibility of the patient expression data with currently-existing RNA expression data references, and allows comparison of RNA expression levels of a single sample with any other samples or cohorts of subjects, regardless of disease/non-disease state or the particular disease being investigated. Being able to directly compare RNA expression data from patient samples to RNA expression data of large clinical research reference datasets (e.g., cancer cohort expression data, such as TCGA data) will better enable researchers and physicians to associate patients with the cohorts and improve the quality and accuracy of downstream analysis of the patient expression data, for example in characterizing the tumor microenvironment (TME) of the patient and/or selecting cancer therapies for the patient.

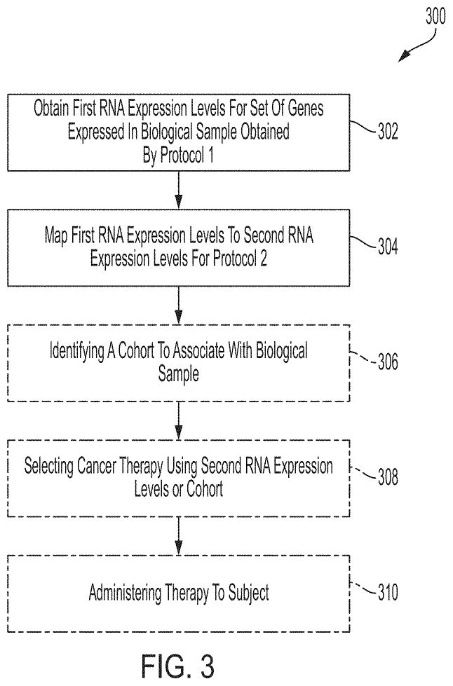

FIG. 3 is a flowchart of an illustrative process 300 for mapping RNA expression levels for genes expressed in a biological sample and obtained from a subject using a first protocol to RNA expression levels as would have been determined through a second protocol if the second protocol were used to process the biological sample instead of the first protocol, according to some embodiments of the technology as described herein.

Various (e.g., some or all) acts of process 300 may be implemented using any suitable computing device(s). For example, in some embodiments, one or more acts of the illustrative process 300 may be implemented in a clinical or laboratory setting. For example, one or more acts of the process 300 may be implemented on a computing device that is located within the clinical or laboratory setting. In some embodiments, the computing device may directly obtain expression data from a sequencing apparatus located within the clinical or laboratory setting. For example, a computing device included in the sequencing apparatus may directly obtain the RNA expression data from the sequencing apparatus. In some embodiments, the computing device may indirectly obtain RNA expression data from a sequencing apparatus that is located within or external to the clinical or laboratory setting. For example, a computing device that is located within the clinical or laboratory setting may obtain RNA expression data via a communication network, such as Internet or any other suitable network, as aspects of the technology described herein are not limited to any particular communication network.

Additionally or alternatively, one or more acts of the illustrative process 300 may be implemented in a setting that is remote from a clinical or laboratory setting. For example, the one or more acts of process 300 may be implemented on a computing device that is located externally from a clinical or laboratory setting. In this case, the computing device may indirectly obtain RNA expression data that is generated using a sequencing apparatus located within or external to a clinical or laboratory setting. For example, the RNA expression data may be provided to computing device via a communication network, such as Internet or any other suitable network.

It should be appreciated that, in some embodiments, not all acts of process 300, as illustrated in FIG. 3, may be implemented using one or more computing devices. For example, the act 308 of selecting a cancer therapy using the second expression levels or cohort associated with the subject may be implemented manually (e.g., by a clinician), automatically (e.g., by software identifying the cancer therapy), or in part manually and in part automatically (e.g., a clinician may select the cancer therapy or cohort for the subject using information generated by the software, for example, using the techniques described herein). In another example, the act 310 of administering a therapy to the subject may be implemented manually (e.g., by a clinician).

Process 300 begins at act 302 where first RNA expression data is obtained. The first RNA expression data may indicate (e.g., specify) first RNA expression levels for a set of genes expressed in a biological sample obtained from a subject by a first protocol are obtained. In some embodiments, the first RNA expression levels may have been previously determined (i.e., prior to start of process 300) by processing the biological sample using a first protocol. In other embodiments, the first protocol may be applied to the biological sample as part of act 302.

In some embodiments, the first protocol comprises: (1) preserving the biological sample using formalin-fixation and paraffin embedding (FFPE); and (2) sequencing the biological sample using an Exome Capture (EC) RNA sequencing technique to obtain the first RNA expression levels. This and other examples of first protocols are described herein including in the section called “Extraction of DNA and/or RNA” and “Obtaining RNA Expression Data.”

As described above, the first RNA expression data obtained at act 302 may indicate first RNA expression levels for a set of genes. Examples of RNA expression data, sources of RNA expression data, and formats of RNA expression data are described herein including in the section called “Obtaining RNA Expression Data.”

The set of genes expressed in the biological sample may comprise any suitable number of genes present (e.g., expressed) in the biological sample. In some embodiments, the set of genes comprises all of the genes present (e.g., expressed) in the biological sample. In some embodiments, the set of genes comprises less than all of the genes present (e.g., expressed) in the biological sample, for example a subset of genes. In some embodiments, the set of genes comprises between 10 and 25,000 genes. In some embodiments, the set of genes comprises between 10 and 1000, 500 and 5000, 2500 and 10000, 5000 and 15000, or 10000 and 25000 genes. In some embodiments, the set of genes comprises between 1000 and 2500 genes. In some embodiments, the set of genes comprises or consists of the genes set forth in Table 2 or Table 3. In some embodiments, the set of genes comprises or consists of at least 10%, at least 20%, at least 30%, at least 40%, at least 50%, at least 60%, at least 70%, at least 80%, at least 90%, at least 95%, at least 99%, or 100% of the genes set forth in Table 2 or Table 3.

As one illustrative example, in some embodiments, the first RNA expression data may comprise bulk sequencing data (e.g., bulk sequencing data obtained from a single biological sample). The bulk sequencing data may comprise at least 1 million reads, at least 5 million reads, at least 10 million reads, at least 20 million reads, at least 50 million reads, or at least 100 million reads. In some embodiments, the sequencing data comprises bulk RNA sequencing (RNA-seq) data, single cell RNA sequencing (scRNA-seq) data, or next generation sequencing (NGS) data. In some embodiments, the first RNA expression data comprises Exome Capture (EC) RNA sequencing data.

Next, process 300 proceeds to act 304, where the first RNA expression levels obtained at act 302 are mapped to second RNA expression levels for a second protocol different from the first protocol. For example, if the first protocol comprises obtaining RNA expression levels by EC RNA-seq, the second protocol may not involve obtaining EC RNA-seq expression levels and may, for example, involve obtaining polyA RNA-seq expression levels. Examples of second protocols are described herein including in the sections called “Extraction of DNA and/or RNA” and “Obtaining RNA Expression Data.”

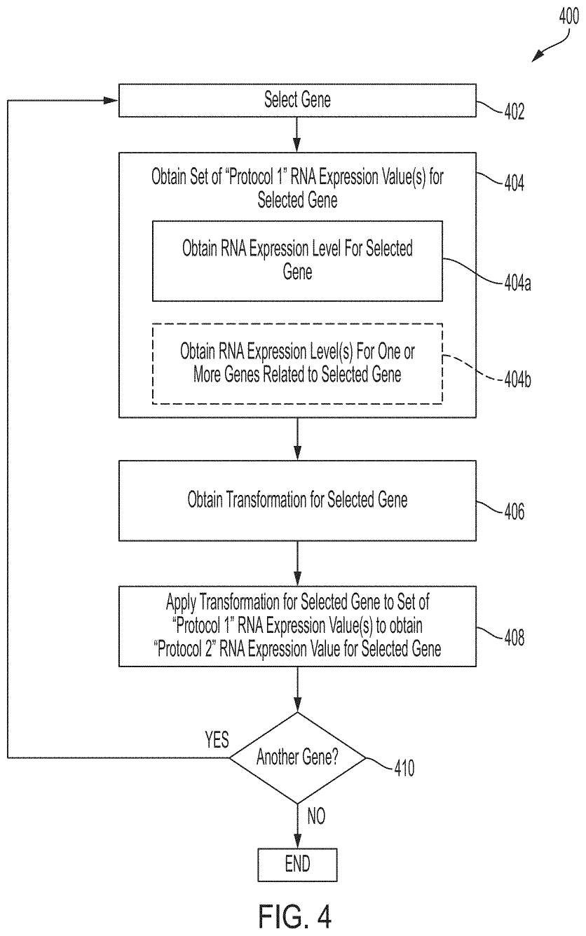

At act 304, the mapping may be performed in any suitable way described herein. For example, in some embodiments, the mapping may involve determining a projected RNA expression level for each gene in the set of genes and, for each such gene, a respective gene-specific transformation is used to determine the projected gene RNA expression level. For example, if the first RNA expression levels contain “N” expression levels for a set of N genes, the mapping performed at act 304 may involve projecting each of the “N” RNA expression levels using a respective transformation. As a result “N” different transformation may be used one for each of the N genes. Each such transformation may be a one-to-one transformation (see e.g., FIGS. 2A and 2B) or a many-to-one transformation (see e.g., FIGS. 2C and 2D). In some embodiments, each such transformation may be linear. In some embodiments, each such transformation is independently a linear or a non-linear transformation (e.g., a first linear transformation and a second non-linear transformation). In some embodiments, each such transformation may have been estimated (i.e., the parameters of the transformation were determined) from training data (comprising paired values as described herein) using any suitable estimation technique (e.g., linear regression or regularized linear regression, examples of which are provided herein). “Projected” RNA expression levels refers to estimated RNA expression levels for the genes in the set of genes expressed in a biological sample as would have been determined through the second protocol if the second protocol were used to process the biological sample instead of the first protocol. Aspects of the mapping performed at act 304 are described herein including with reference to FIG. 4.

In some embodiments, process 300 may complete after act 304 completes. In other embodiments, process 300 may continue and one or more of optional acts 306, 308 and 310 may be performed. For example, only act 306 may be performed, or only act 308 may be performed, or both acts 306 and 308 may be performed, or both acts 308 and 310 may be performed, or all three acts 306, 308, and 310 may be performed.

At act 306, the second RNA expression levels obtained as a result of the mapping performed at act 304 are used to identify a cohort with which to associate the subject from which the biological sample was obtained. Aspects of how identify a cohort using second RNA expression levels are described herein including in the section called “Post-Mapping Processing.”

At act 308, a cancer therapy may be selected using the second RNA expression levels, and at act 310, the selected therapy may be administered to the subject. Aspects of how acts 308 and 310 may be performed are described herein including in the sections called “Post-Mapping Processing” and “Anti-Cancer Therapies.”

FIG. 4 is a flowchart depicting an illustrative process 400 for mapping RNA expression levels obtained using a first protocol to RNA expression levels obtained using a second different protocol, in accordance with some embodiments of the technology described herein. Process 400 may be used to implement act 304 described with reference to process 300. Process 400 may be implemented using any computing device(s) as aspects of the technology described herein is not limited in this respect.

Process 400 begins at act 402, where a particular gene is selected from a set of genes. Examples of genes and sets of genes are provided herein.

Next, process 400 proceeds to act 404 where a set of RNA expression levels is obtained for the selected gene. The RNA expression levels may be those as determined by applying a first protocol (e.g., EC RNA-seq) to a biological sample obtained from a subject. As shown in FIG. 4, the set of RNA expression levels may include a single RNA expression level, which may be obtained at act 404a, and that single RNA expression level may be the RNA expression level for the gene selected at act 402. Optionally, the set of RNA expression levels may include one or more additional RNA expression levels, which may be obtained at act 404b, for one or more other genes that are associated with the gene selected at act 402.

In some embodiment, the one or multiple other genes may be any suitable number of genes. In some embodiments, the multiple genes comprises between 1 and 10, 5 and 20, 10 and 50, 25 and 100, 50 and 200, 125 and 500, 250 and 1000, or any other range within these ranges or more than 1000 genes. In some embodiments, the one or multiple RNA expression levels of the one or multiple other genes comprises between 1 and 10, 5 and 20, 10 and 50, 25 and 100, 50 and 200, 125 and 500, 250 and 1000, or any other range within these ranges or more than 1000 genes.

A gene that is “associated with” a selected gene is a gene that has an RNA expression level that correlates with the RNA expression level of the selected gene. Correlation of RNA expression levels may be measured by any suitable methods known. Examples of techniques used to identify associations between RNA expression levels include but are not limited to Pearson correlation. Accordingly, in some embodiments, for each particular gene, genes that are “associated with” the particular gene may be identified by Pearson correlation.

Next, process 400 proceeds to act 406, where a transformation for the selected gene is obtained. In some embodiments, the transformation has been previously determined (e.g., determined prior to the commencement of process 400). In some embodiments, the transformation may be a linear transformation although, in other embodiments, a non-linear transformation may be used.

In some embodiments, the transformation may have been previously determined from training data by using any suitable linear (or non-linear) regression technique. For example, linear regression (e.g., ordinary least squares (OLS)) or regularized linear regression (LASSO, ridge regression, ElasticNet or ElasticNetCV regression) may have been used. ElasticNet or ElasticNetCV regression is described by Zou and Hastie, 2005 “Regularization and variable selection via the elastic net.” Journal of the Royal Statistical Society. Series B, Statistical methodology 67 (2): 301-320, which is incorporated by reference herein in its entirety.

In some embodiments, the training data comprises paired values of RNA expression levels for selected genes of a set of RNA expression data. Each of the paired values of the RNA expression levels may include an RNA expression level as determined through applying the first protocol to a particular biological sample (e.g., a Protocol 1 RNA expression level) and another RNA expression level as determined through applying the second protocol to the particular biological sample (e.g., a Protocol 2 RNA expression level). The training data (for each gene) may comprise any suitable number of training values (e.g., at least 5, 10, 100, 1000, 5000, 10,000, between 5 and 1000, between 100 and 10,000 pairs of values, or any other suitable range within these ranges). The training data may comprise paired values of RNA expression levels for selected genes for a single sample (e.g., all paired values of RNA expression levels are obtained from a single biological sample) or RNA expression levels for selected genes in multiple biological samples (e.g., the paired RNA expression levels are obtained from a plurality of biological samples, such as 1, 2, 5, 10, 100, 500, 1000, 5000, or 10000 samples).

Next, process 400 proceeds to act 408, where the selected transformation at act 406 is applied to the set of RNA expression levels obtained at act 404 to obtain a projected “Protocol 2” RNA expression level for the selected gene. The projected “Protocol 2” RNA expression level for the selected gene is indicative of the RNA expression level of the selected gene in the biological sample, if the biological sample had been processed according to a second protocol rather than the first protocol.

Next, process 400 proceeds to act 410, which determines whether or not acts 404-408 will be repeated. If RNA expression levels of no other genes of the biological sample are to be mapped, process 400 terminates at act 410. If RNA expression levels of one or more additional genes are to be mapped, process 400 returns to act 402 to select another gene for mapping, and acts 404-410 are repeated.

The number of genes in a biological sample that have RNA expression levels mapped from Protocol 1 to Protocol 2 RNA expression levels may vary. In some embodiments, all genes of the biological sample are mapped using process 400. In some embodiments, less than all (e.g., a subset of genes) of the genes in the biological sample are mapped using process 410. That subset may have between 10 and 25,000 genes, between 10 and 1000, 500 and 5000, 2500 and 10000, 5000 and 15000, or 10000 and 25000 genes. In some embodiments, a subset of genes comprises between 1000 and 2500 genes. In some embodiments, a subset comprises or consists of the genes set forth in Table 2 or Table 3.

### Biological Sample

Aspects of the disclosure relate to methods for mapping RNA expression levels for genes expressed in a biological sample and obtained from a subject using a first protocol to RNA expression levels as would have been determined through a second protocol if the second protocol were used to process the biological sample instead of the first protocol.

In some embodiments, a subject is a mammal (e.g., a human, a mouse, a cat, a dog, a horse, a hamster, a cow, a pig, or other domesticated animal). In some embodiments, a subject is a human. In some embodiments, a subject is an adult human (e.g., of 18 years of age or older). In some embodiments, a subject is a child (e.g., less than 18 years of age). In some embodiments, a human subject is one who has or has been diagnosed with at least one form of cancer. In some embodiments, a cancer from which a subject suffers is a carcinoma, a sarcoma, a myeloma, a leukemia, a lymphoma, or a mixed type of cancer that comprises more than one of a carcinoma, a sarcoma, a myeloma, a leukemia, and a lymphoma. Carcinoma refers to a malignant neoplasm of epithelial origin or cancer of the internal or external lining of the body. Sarcoma refers to cancer that originates in supportive and connective tissues such as bones, tendons, cartilage, muscle, and fat. Myeloma is cancer that originates in the plasma cells of bone marrow. Leukemias (“liquid cancers” or “blood cancers”) are cancers of the bone marrow (the site of blood cell production). Lymphomas develop in the glands or nodes of the lymphatic system, a network of vessels, nodes, and organs (specifically the spleen, tonsils, and thymus) that purify bodily fluids and produce infection-fighting white blood cells, or lymphocytes. Non-limiting examples of a mixed type of cancer include adenosquamous carcinoma, mixed mesodermal tumor, carcinosarcoma, and teratocarcinoma. In some embodiments, a subject has a tumor. A tumor may be benign or malignant. In some embodiments, a cancer is any one of the following: skin cancer, lung cancer, breast cancer, prostate cancer, colon cancer, rectal cancer, cervical cancer, and cancer of the uterus. In some embodiments, a subject is at risk for developing cancer, e.g., because the subject has one or more genetic risk factors, or has been exposed to or is being exposed to one or more carcinogens (e.g., cigarette smoke, or chewing tobacco).

The disclosure is based, in part, on projecting RNA expression levels of genes in a biological sample prepared according to a first protocol to RNA expression levels of the genes in the biological sample if the sample had been prepared by a second protocol (e.g., a different protocol than the first protocol). As used herein, the term “protocol” refers to one or more techniques used to obtain, isolate, preserve, or process a biological sample obtained from a subject. Examples of techniques for obtaining tissue from a subject include but are not limited to fluid (e.g., blood, CSF, lymph node, etc.) collection, tissue biopsy, cell scraping, urine sample collection, fecal sample collection, saliva collection, etc. Examples of methods of preserving biological samples include but are not limited to fresh frozen preservation techniques and tissue fixation techniques (e.g., alcohol-fixation, formalin-fixation, paraffin-embedding, optimal cutting temperature (OCT) preservation, RNAlater® preservation, etc.). Examples of processing techniques include but are not limited to nucleic acid extraction, nucleic acid purification, and nucleic acid sequencing.

In some embodiments, RNA expression data is obtained from a biological sample prepared by a protocol comprising formalin-fixation and paraffin-embedding (FFPE). Examples of FFPE techniques include but are not limited to laser capture microdis section (LCM), microtome sectioning, and FFPE core isolation. Methods of FFPE preservation of tissue are well-known, for example as described by Amini et al., BMC Molecular Biology volume 18, Article number: 22 (2017). Typically, FFPE protocols comprise the following steps: tissue coring, tissue fixation, paraffin embedding, mounting, and storage. FFPE-preserved samples may be stored at room temperature or below room temperature, for example 4° C.

In some embodiments, a protocol comprising FFPE preservation further comprises nucleic acid extraction and/or nucleic acid purification. Examples of nucleic acid extraction and purification techniques are described herein in the section called “Extraction of DNA and/or RNA.” In some embodiments, a protocol comprising FFPE preservation further comprises nucleic acid sequencing. In some embodiments, the nucleic acid sequencing is Exome Capture (EC) RNA sequencing (RNA-seq). Methods of sequencing, including EC RNA-seq are described herein including in the section called “Obtaining Gene Expression Data.”

In some embodiments, RNA expression data is obtained from a biological sample prepared by a protocol comprising a fresh frozen preservation technique. Methods for preserving fresh frozen tissue generally comprise the following steps: tissue collection, snap freezing by immersion in liquid nitrogen, and storage at −80° C., for example as described by Mager et al. Standard operating procedure for the collection of fresh frozen tissue samples. Eur J Cancer 2007, 43(5):828-834.

In some embodiments, a protocol comprising FF preservation further comprises nucleic acid extraction and/or nucleic acid purification. Examples of nucleic acid extraction and purification techniques are described herein in the section called “Extraction of DNA and/or RNA.” In some embodiments, a protocol comprising FF preservation further comprises nucleic acid sequencing. In some embodiments, the nucleic acid sequencing is polyA RNA-seq. Methods of sequencing, including polyA RNA-seq are described herein including in the section called “Obtaining Gene Expression Data.”

The biological sample may be from any source in the subject's body including, but not limited to, any fluid such as blood (e.g., whole blood, blood serum, or blood plasma), lymph node, stomach, small intestine. Other source in the subject's body may be from saliva, tears, synovial fluid, cerebrospinal fluid, pleural fluid, pericardial fluid, ascitic fluid, and/or urine], hair, skin (including portions of the epidermis, dermis, and/or hypodermis), oropharynx, laryngopharynx, esophagus, bronchus, salivary gland, tongue, oral cavity, nasal cavity, vaginal cavity, anal cavity, bone, bone marrow, brain, thymus, spleen, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue). The biological sample may be any type of sample including, for example, a sample of a bodily fluid, one or more cells, one or more pieces of tissue(s) or organ(s).

In some embodiments, a tissue sample may be obtained from a subject using a surgical procedure, bone marrow biopsy, punch biopsy, endoscopic biopsy, or needle biopsy (e.g., a fine-needle aspiration, core needle biopsy, vacuum-assisted biopsy, or image-guided biopsy).

A sample of lymph node or blood, in some embodiments, refers to a sample comprising cells, e.g., cells from a blood sample or lymph node sample. In some embodiments, the sample comprises non-cancerous cells. In some embodiments, the sample comprises pre-cancerous cells. In some embodiments, the sample comprises cancerous cells. In some embodiments, the sample comprises blood cells. In some embodiments, the sample comprises lymph node cells. In some embodiments, the sample comprises lymph node cells and blood cells.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

In some embodiments, a sample of blood is collected to obtain the cell-free nucleic acid (e.g., cell-free DNA) in the blood.

In some embodiments, the sample may be from a cancerous tissue or an organ or a tissue or organ suspected of having one or more cancerous cells. In some embodiments, the sample may be from a healthy (e.g., non-cancerous) tissue or organ. In some embodiments, a sample from a subject (e.g., a biopsy from a subject) may include both healthy and cancerous cells and/or tissue. In certain embodiments, one sample will be taken from a subject for analysis. In some embodiments, more than one (e.g., 2, 3, 4, 5, 6, 7, 8, 9, 10, 11, 12, 13, 14, 15, 16, 17, 18, 19, 20, or more) samples may be taken from a subject for analysis. In some embodiments, one sample from a subject will be analyzed. In certain embodiments, more than one (e.g., 2, 3, 4, 5, 6, 7, 8, 9, 10, 11, 12, 13, 14, 15, 16, 17, 18, 19, 20, or more) samples may be analyzed. If more than one sample from a subject is analyzed, the samples may be procured at the same time (e.g., more than one sample may be taken in the same procedure), or the samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure). A second or subsequent sample may be taken or obtained from the same region (e.g., from the same tumor or area of tissue) or a different region (including, e.g., a different tumor). A second or subsequent sample may be taken or obtained from the subject after one or more treatments, and may be taken from the same region or a different region. As a non-limiting example, the second or subsequent sample may be useful in determining whether the cancer in each sample has different characteristics (e.g., in the case of samples taken from two physically separate tumors in a patient) or whether the cancer has responded to one or more treatments (e.g., in the case of two or more samples from the same tumor prior to and subsequent to a treatment).

Any of the biological samples described herein may be obtained from the subject using any known technique. See, for example, the following publications on collecting, processing, and storing biological samples, each of which is incorporated by reference herein in its entirety: Biospecimens and biorepositories: from afterthought to science by Vaught et al. (Cancer Epidemiol Biomarkers Prev. 2012 Feb.;21(2):253-5), and Biological sample collection, processing, storage and information management by Vaught and Henderson (IARC Sci Publ. 2011;(163):23-42).

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one form to another form such that the first form is no longer detected at the same level as before degradation.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris.Cl; 0.5 mM EDTA, pH 9.0)) and other coagulants, and Acids Citrate Dextronse (e.g., for blood specimens).

In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Extraction of DNA and/or RNA

In some embodiments of any one of the methods described herein, RNA is extracted from a biological sample to prevent it from being degraded and/or to prevent the inhibition of enzymes in downstream processing, e.g., the preparation of DNA (i.e., a cDNA library from RNA). In some embodiments, the term “extraction” in the context of obtaining RNA from a biological sample is used interchangeably with the term “isolation.”

Methods described herein involve extraction of RNA from a biological sample (e.g., a tumor sample or sample of blood). As described above, a biological sample may be comprised of more than one sample from one or more than one tissues (e.g., one or more than one different tumors). In some embodiments, RNA is extracted from a combined sample. In some embodiments, RNA is extracted from multiple biological samples from a subject, and then combined before further processing (e.g., storage, or DNA library preparation). In some embodiments, more than one sample of extracted RNA are combined with each other after retrieval from storage. In some embodiments, at least tumor is extracted from one or more tumor tissues. In some embodiments, at least tumor RNA is extracted from one or more tumor tissues. In some embodiments, at least normal RNA is extracted from one of more normal tissues. In some embodiments RNA is extracted from normal samples to serve as a control.

Methods for extracting RNA from biological samples are known, and reagents and kits for doing so are commercially available. Gomez-Acata et al. (Methods for extracting ′omes from microbialites, J Microbiol Methods. 2019 Mar. 12; 160:1-10) describes methods for extracting applied for RNA extraction from microbialites and describes their advantages and disadvantages and is incorporated herein by reference in its entirety. The methods described in Gòmez-Acata et al. are generally applicable for RNA extracted from tissue. Dowhan (Curr. Protoc. Essential Lab. Tech. 6:5.2.1-5.2.21) describes purification and concentration of RNA from aqueous solutions and is also incorporated by reference herein in its entirety. In some embodiments, RNA is extracted from a biological sample using a kit suitable for RNA-seq, for example by methods described in Cortes-Esteve et al. PLoS One. 2017; 12(1): e0170632.

In some embodiments, extracting RNA comprises lysing cells of a biological sample and isolating RNA from other cellular components. Examples of methods for lysing cells include, but are not limited to, mechanical lysis, liquid homogenization, sonication, freeze-thaw, chemical lysis, alkaline lysis, and manual grinding.

Methods for extracting RNA include, but are not limited to, solution phase extraction methods and solid-phase extraction methods. In some embodiments, a solution phase extraction method comprises an organic extraction method, e.g., a phenol chloroform extraction method. In some embodiments, a solution phase extraction method comprises a high salt concentration extraction method, e.g., guanidinium thiocyantate (GuTC) or guanidinium chloride (GuCl) extraction method. In some embodiments, a solution phase extraction method comprises an ethanol precipitation method. In some embodiments, a solution phase extraction method comprises an isopropanol precipitation method. In some embodiments, a solution phase extraction method comprises an ethidium bromide (EtBr)-Cesium Chloride (CsCl) gradient centrifugation method. In some embodiments, extracting DNA and/or RNA comprises a nonionic detergent extraction method, e.g., a cetyltrimethylammonium bromide (CTAB) extraction method.

In some embodiments, extracting RNA comprises a solid phase extraction method. Any solid phase that binds to RNA may be used for extracting RNA in methods and systems described herein. Examples of solid phases that bind RNA include, but are not limited to, silica matrices, ion exchange matrices, glass particles, magnetizable cellulose beads, polyamide matrices, and nitrocellulose membranes.

In some embodiments, a solid phase extraction method comprises a spin-column based extraction method. In some embodiments, a solid phase extraction method comprises a bead-based extraction method. In some embodiments, a solid phase extraction method comprises a cation exchange resin, e.g., a styrene divinylbenzene copolymer resin.

Systems and methods described herein encompass extracting RNA from a single biological sample or a plurality of biological samples. In some embodiments, extracting RNA comprises extracting RNA from a single sample. In some embodiments, extracting RNA comprises extracting RNA from a plurality of samples. In some embodiments, extracting RNA comprises extracting RNA from a first sample and a second sample. In some embodiments, extracting RNA comprises extracting RNA from one or more, two or more, three or more, four or more, five or more, six or more, seven or more, eight or more, nine or more, or ten or more samples.

Extracted RNA from a biological sample may be combined with extracted RNA from another biological sample. This may be accomplished by combining one or more biological samples and extracting nucleic acids or by combining nucleic acids extracted from one or more biological samples. In some embodiments, a first biological sample is combined with a second biological sample to form a combined sample and extracting RNA from the combined sample. In some embodiments, extracted RNA from a first biological sample may be combined with extracted DNA and/or RNA from a second biological sample.

Systems and methods described herein encompass extracting any type of RNA from a biological sample. In some embodiments, extracting RNA comprises extracting messenger RNA (mRNA). In some embodiments, extracting RNA comprises extracting precursor mRNA (pre-mRNA). In some embodiments, extracting RNA comprises extracting ribosomal RNA (rRNA). In some embodiments, extracting RNA comprises extracting transfer RNA (tRNA).

In some embodiments, a single kit is used to purity DNA and RNA from the same sample. A non-limiting example of kit for doing so is the Qiagen AllPrep DNA/RNA kit. In some embodiments, robotics is employed to carry out DNA and/or RNA extraction.

In some embodiments, before extracted RNA is processed further for RNA sequencing or whole exome sequencing (WES), the quality and/or quantity of RNA is checked. In some embodiments, a sample of extracted RNA is at least 1000-6000 ng in total mass. In some embodiments, a sample of extracted RNA is at least 100-60000 ng (e.g., 100-60000 ng, 500-30000 ng, 800-20000 ng, 1000-15000 ng, 1000-10000 ng, 1000-8000 ng, 1000-6000 ng, 10000-20000 ng, 20000-60000 ng) in total mass. In some embodiments, the acceptable total RNA amount for further sequencing is at least 100-1,000 ng (e.g., 100-1,000 ng, 500-1,000 ng, or 300-900 ng). In some embodiments, the target total RNA amount for further sequencing is more than 200-1,000 ng (e.g., 200-1,000 ng, 500-1,000 ng, or 300-1,000 ng). In some embodiments, the purity of a sample of extracted RNA is such that it corresponds to a ratio of absorbance at 260 nm to absorbance at 280 nm of at least 1 (e.g., at least 1, at least 1.2, at least 1.4, at least 1.6, at least 1.8, or at least 2). In some embodiments, the purity of a sample of extracted RNA is such that it corresponds to a ratio of absorbance at 260 nm to absorbance at 280 nm of at least 2. The ratio of absorbance at 260 nm and 280 nm is used to assess the purity of DNA and RNA. A ratio of ˜1.8 is generally accepted as “pure” for DNA; a ratio of ˜2.0 is generally accepted as “pure” for RNA. If the ratio is appreciably lower in either case, it may indicate the presence of protein, phenol or other contaminants that absorb strongly at or near 280 nm. Absorbances can be measured using a spectrophotometer.

In some embodiments, the purity or integrity of extracted RNA is such that it corresponds to a RNA integrity number (RIN) of at least 4 (e.g., at least 4, at least 5, at least 6, at least 7, at least 8, or at least 9). In some embodiments, the purity of extracted RNA is such that it corresponds to a RNA integrity number (RIN) of at least 7. RIN has been demonstrated to be robust and reproducible in studies comparing it to other RNA integrity calculation algorithms, cementing its position as a preferred method of determining the quality of RNA to be analyzed (Imbeaud et al., Towards standardization of RNA quality assessment using user-independent classifiers of microcapillary electrophoresis traces; Nucleic Acids Research. 33 (6): e56).

In some embodiments, a sample of extracted RNA has a target concentration of at least 2 ng/μl (e.g., 2 ng/μl, 4 ng/μl, 6 ng/μl). In some embodiments, a sample of extracted RNA has an acceptable concentration of at least 4 ng/μl (e.g., 4 ng/μl, 6 ng/μl, 10 ng/μl). In some embodiments, the concentration of the extracted DNA is performed by a fluorometer, for example for quantification of RNA (e.g., a Qubit fluorometer available from ThermoFisher Scientific, www.thermofisher.com).

In some embodiments, a sample of extracted RNA has a target concentration of at least 4 ng/μl (e.g., 4 ng/μl, 6 ng/μl, 8 ng/μl). In some embodiments, a sample of extracted RNA has an acceptable concentration of at least 1.5 ng/μl (e.g., 1.5 ng/μl, 3.5 ng/μl, 5.5 ng/μl). In some embodiments, the concentration of the extracted RNA is performed by Tapestation. In some embodiments, the acceptable RNA integrity number (RIN) is at least 5 (e.g., 5, 6, 7). In some embodiments, the target RNA integrity number (RIN) is at least 8 (e.g., 8, 9, 10). In some embodiments, the RIN is performed by Tapestation.

In some embodiments, the target purity of a sample of extracted RNA is such that it corresponds to a range of a ratio of absorbance at 260 nm to absorbance at 280 nm of at least 1.8-2 (e.g., at least 1.8-2, at least 1.8-1.9). In some embodiments, the purity of a sample of extracted RNA is such that it corresponds to a ratio of absorbance at 260 nm to absorbance at 280 nm of at least 1.8. In some embodiments, the acceptable purity of a sample of extracted RNA is such that it corresponds to a ratio of absorbance at 260 nm to absorbance at 280 nm of at least 1.5 (e.g., at least 1.5, at least 1.7, at least 2). In some embodiments, the target purity of a sample of extracted RNA is such that it corresponds to a range of a ratio of absorbance at 260 nm to absorbance at 230 nm of at least 2-2.2 (e.g., at least 2-2.2, at least 2-2.1). In some embodiments, the acceptable purity of a sample of extracted RNA is such that it corresponds to a ratio of absorbance at 260 nm to absorbance at 230 nm of at least 1.5 (e.g., at least 1.5, at least 1.7, at least 2). In some embodiments, the purity of a sample of extracted RNA as described herein is analyzed by a spectrophotometer, for example a small volume full-spectrum, UV-visible spectrophotometer (e.g., Nanodrop spectrophotometer available from ThermoFisher Scientific).

In some embodiments, the purity of a sample of extracted RNA as described herein can be analyzed by any other suitable technologies or tools. In some embodiments, a sample of extracted RNA or DNA is not processed further if it does not meet a particular quantity or purity standard as described above. In some embodiments, if a sample of extracted RNA does not meet a particular quantity or purity standard, it is combined with another sample.

### Obtaining RNA Expression Data

Aspects of the disclosure relate to methods of determining RNA expression levels of genes of a subject using sequencing data or RNA expression data obtained from a biological sample from the subject.

The sequencing data may be obtained from the biological sample using any suitable sequencing technique and/or apparatus. In some embodiments, the sequencing apparatus used to sequence the biological sample may be selected from any suitable sequencing apparatus known including, but not limited to, Illumina™, SOLid™, Ion Torrent™, PacBio™, a nanopore-based sequencing apparatus, a Sanger sequencing apparatus, or a 454TM sequencing apparatus. In some embodiments, the sequencing apparatus or technique used to sequence the biological sample is an Illumina sequencing (e.g., TrueSeq™, NovaSeq™, NextSeq™, HiSeq™, MiSeq™, or MiniSeg™) apparatus or technique. In some embodiments, the sequencing apparatus or technique used to sequence the biological sample is an Agilent sequencing apparatus or technique (e.g., SureSelect™) or a NimbleGen sequencing apparatus or technique, for example as described by Sulonen et al. Comparison of solution-based exome capture methods for next generation sequencing. Genome Biol 12, R94 (2011). doi.org/10.1186/gb-2011-12-9-r94.

In some embodiments, the term “RNA sequencing” can be used interchangeably with “RNA seq,” “RNA-seq,” or the variations thereof as known referring to any technologies, tools, or platforms that interrogate the transcriptome. It is noted that when “RNA sequencing,” “RNA seq,” “RNA-seq,” or the variations thereof is referred in the present disclosure, it does not refer to a specific technology or tool that is associated with a particular platform or company, unless indicated otherwise by way of non-limiting examples for demonstrating the processes or systems as described herein. In some embodiments, RNA sequencing can be conducted by using any suitable sequencing platforms and/or sequencing methods. Non-limiting examples of high-throughput sequencing platforms include mRNA-seq, total RNA-seq, targeted RNA-seq, single-cell RNA-Seq, RNA exome capture platform, or small RNA-seq (e.g., Illumina, www.illumina.com), SMRT (single molecule, real-time) sequencing (e.g., Pacific Biosciences), and RNA sequencing (e.g., ThermoFisher).

As described above, RNA sequencing can be targeted or untargeted. Targeted approaches include using sequence-specific probes or oligonucleotides to sequence one or more specific regions of the transcriptome. In some embodiments, targeted RNA sequencing includes methods such as mRNA enrichment (e.g., by polyA enrichment or rRNA depletion).

In some embodiments, RNA sequencing is whole transcriptome sequencing. Whole transcriptome sequencing comprises measurement of the complete complement of transcripts in a sample. In some embodiments, whole transcriptome sequencing is used to determine global expression levels of each transcript (e.g., both coding and non-coding), identify exons, introns and/or their junctions.

In some embodiments, RNA is sequenced directly without preparing cDNA from a sample of RNA. In some embodiments, direct RNA sequencing comprises single molecule RNA sequencing (DRS™)

In some embodiments, RNA sequencing is mRNA sequencing. In some embodiments, mRNA sequencing is the sequencing of only coding transcripts with the goal to exclude non-coding regions. In some embodiments, mRNA sequencing is independent of polyA enrichment. In some embodiments, mRNA sequencing depends on polyA enrichment.

In some embodiments, RNA is extracted from a biological sample, mRNA is enriched from the extracted RNA, cDNA libraries are constructed from the enriched mRNA. In some embodiments, single pieces (e.g., molecules) of cDNA from a cDNA library are attached to a solid matrix. In some embodiments, single pieces (e.g., molecules) of cDNA from a cDNA library are attached to a solid matrix by limited dilution. In some embodiments, cDNA pieces (e.g., molecules) attached to a matrix are then sequenced (e.g., using Pacbio or Pacifbio technology). In some embodiments, cDNA pieces (e.g., molecules) that are attached to a matrix are amplified and sequenced (e.g., using a specialized emulsion PCR (emPCR) in SOLiD, 454 Pyrosequencing, Ion Torrent, or a connector based on the bridging reaction (Illumina) platforms).

In some embodiments, cDNA transcripts can be sequenced in parallel, either by measuring the incorporation of fluorescent nucleotides (for example, Illumina), fluorescent short linkers (for example, SOLiD), by the release of the by-products derived from the incorporation of normal nucleotides (454), by measuring fluorescence emissions, or by measuring pH change (for example, Ion Torrent). In some embodiments, cDNA transcripts can be sequenced using any known sequencing platform. Jazayeri et al. (RNA-seq: a glance at technologies and methodologies; Acta biol. Colomb. vol. 20 no.2 Bogotà May/August 2015) provides a comparison of different RNA-seq platforms, and is incorporated herein by reference in its entirety, including RNA-seq technologies listed in Table 3 and Table 4. Mestan et al. (Genomic sequencing in clinical trials; Journal of Translational Medicine 2011, 9:222) provides a similar analysis for sequencing in clinical trials.

In some embodiments, RNA sequencing is stranded or strand-specific. cDNA synthesis from RNA results in loss of strandedness. In some embodiments, strandedness is preserved by chemically labeling either or both the RNA strand and the cDNA strand that is formed by reverse transcription or antisense transcription, or by using adapter-based techniques to distinguish the original RNA strand from the complementary DNA strand, as described above.

In some embodiments, nonstranded RNA sequencing is performed. In some embodiments, stranded RNA-seq is not preferred for clinical samples. In some embodiments, nonstranded RNA-seq is used to compare data obtained from a biological sample to RNA sequencing data in established data sets (e.g., The Cancer Genome Atlas (TCGA) and International Cancer Genome Consortium (ICGC)).

In some embodiments, RNA sequencing yields paired-end reads. Paired-end reads are reads of the same nucleic acid fragment and are reads that start from either end of the fragment. In some embodiments, RNA sequencing is performed with paired-end reads of at least 2×25 (2×25, 2×50, 2×75, 2×100, 2×125, 2×150, 2×175, 2×200, 2×225, 2×250, 2×275, 2×300, 2×325, or 2×350) paired-end reads. In some embodiments, RNA sequencing is performed with paired-end reads of at least 2×75 paired-end reads. RNA sequencing with 2×75 paired-end reads means that on average each read, which is paired-end, reads 75 base pairs. In some embodiments, RNA sequencing is performed with a total of at least 20 million (e.g., at least 20 million, at least 30 million, at least 40 million, at least 50 million, at least 60 million, at least 70 million at least 80 million, at least 90 million, at least 100 million, at least 120 million, at least 140 million, at least 150 million, at least 160 million, at least 180 million, at least 200 million, at least 250 million, at least 300 million, at least 350 million, or at least 400 million) paired-end reads. In some embodiments, RNA sequencing is performed with a total of at least 50 million paired-end reads. In some embodiments, RNA sequencing is performed with a total of at least 100 million paired-end reads.

In some embodiments, quality control is performed for RNA sequencing. In some embodiments, cluster density or cluster PF % is a parameter for determining the quality of the sample run. In some embodiments, the target range of cluster density or cluster PF % is at least 170-220 (e.g., 170-220, 190-220, 210-220). In some embodiments, the acceptable range of cluster density or cluster PF % is at least 280 (e.g., 280, 300, 450).

In some embodiments, %≥Q30 is a parameter for determining the quality of the sample run. In some embodiments, the target %≥Q30 is at least 85% (e.g., 85%, 90%, 95%). In some embodiments, the acceptable %≥Q30 is at least 75% (e.g., 75%, 85%, 95%).

In some embodiments, error rate % is a parameter for determining the quality of the sample run. In some embodiments, the target error rate % is less than 0.7% (e.g., 0.6%, 0.5%, 0.4%). In some embodiments, the acceptable error rate % is less than 1% (e.g., 0.9%, 0.8%, 0.7%).

After the sequencing data is obtained, it is processed in order to obtain the RNA expression data. RNA expression data may be acquired using any method known including, but not limited to: whole transcriptome sequencing, whole exome sequencing, total RNA sequencing, mRNA sequencing, targeted RNA sequencing, RNA exome capture sequencing, next generation sequencing, and/or deep RNA sequencing. In some embodiments, RNA expression data may be obtained using a microarray assay.

In some embodiments, the sequencing data is processed to produce RNA expression data. In some embodiments, RNA sequence data is processed by one or more bioinformatics methods or software tools, for example RNA sequence quantification tools (e.g., Kallisto) and genome annotation tools (e.g., Gencode v23), in order to produce expression data. The Kallisto software is described in Nicolas L Bray, Harold Pimentel, Pàll Melsted and Lior Pachter, Near-optimal probabilistic RNA-seq quantification, Nature Biotechnology 34, 525-527 (2016), doi:10.1038/nbt.3519, which is incorporated by reference in its entirety herein.

In some embodiments, microarray expression data is processed using a bioinformatics R package, such as “affy” or “limma,” in order to produce expression data. The “affy” software is described in Bioinformatics. 2004 Feb. 12;20(3):307-15. doi: 10.1093/bioinformatics/btg405. “affy—analysis of Affymetrix GeneChip data at the probe level” by Laurent Gautier 1, Leslie Cope, Benjamin M Bolstad, Rafael A Irizarry PMID: 14960456 DOI: 10.1093/bioinformatics/btg405, which is incorporated by reference herein in its entirety. The “limma” software is described in Ritchie M E, Phipson B, Wu D, Hu Y, Law C W, Shi W, Smyth G K “limma powers differential expression analyses for RNA-sequencing and microarray studies.” Nucleic Acids Res. 2015 Apr. 20;43(7):e47. 20. https://doi.org/10.1093/nar/gkv007 PMID: 25605792, PMCID: PMC4402510, which is incorporated by reference herein its entirety.

In some embodiments, sequencing data and/or RNA expression data comprises more than 5 kilobases (kb). In some embodiments, the size of the obtained RNA data is at least 10 kb. In some embodiments, the size of the obtained RNA sequencing data is at least 100 kb. In some embodiments, the size of the obtained RNA sequencing data is at least 500 kb. In some embodiments, the size of the obtained RNA sequencing data is at least 1 megabase (Mb). In some embodiments, the size of the obtained RNA sequencing data is at least 10 Mb. In some embodiments, the size of the obtained RNA sequencing data is at least 100 Mb. In some embodiments, the size of the obtained RNA sequencing data is at least 500 Mb. In some embodiments, the size of the obtained RNA sequencing data is at least 1 gigabase (Gb). In some embodiments, the size of the obtained RNA sequencing data is at least 10 Gb. In some embodiments, the size of the obtained RNA sequencing data is at least 100 Gb. In some embodiments, the size of the obtained RNA sequencing data is at least 500 Gb.

In some embodiments, the expression data is acquired through bulk RNA sequencing. Bulk RNA sequencing may include obtaining RNA expression levels for each gene across RNA extracted from a large population of input cells (e.g., a mixture of different cell types.) In some embodiments, the expression data is acquired through single cell sequencing (e.g., scRNA-seq). Single cell sequencing may include sequencing individual cells.

In some embodiments, bulk sequencing data comprises at least 1 million reads, at least 5 million reads, at least 10 million reads, at least 20 million reads, at least 50 million reads, or at least 100 million reads. In some embodiments, bulk sequencing data comprises between 1 million reads and 5 million reads, 3 million reads and 10 million reads, 5 million reads and 20 million reads, 10 million reads and 50 million reads, 30 million reads and 100 million reads, or 1 million reads and 100 million reads (or any number of reads including, and between).

In some embodiments, the expression data comprises next-generation sequencing (NGS) data.

RNA expression data (e.g., indicating RNA expression levels) for a plurality of genes may be used for any of the methods or compositions described herein. The number of genes which may be examined may be up to and inclusive of all the genes of the subject. In some embodiments, RNA expression levels may be determined for all of the genes of a subject. As a non-limiting example, four or more, five or more, six or more, seven or more, eight or more, nine or more, ten or more, eleven or more, twelve or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 35 or more, 40 or more, 50 or more, 60 or more, 70 or more, 80 or more, 90 or more, 100 or more, 125 or more, 150 or more, 175 or more, 200 or more, 225 or more, 250 or more, 275 or more, or 300 or more genes may be used for any evaluation described herein. As another set of non-limiting examples, the RNA expression data may include RNA expression data for at least 5, at least 10, at least 15, at least 20, at least 25, at least 35, at least 50, at least 75, at least 100 genes, at least 500, at least 1000, or at least 1500 genes selected from Table 2 or Table 3. In some embodiments, RNA expression data is obtained by accessing the RNA expression data from at least one computer storage medium on which the RNA expression data is stored. Additionally or alternatively, in some embodiments, RNA expression data may be received from one or more sources via a communication network of any suitable type. For example, in some embodiment, the RNA expression data may be received from a server (e.g., a SFTP server, or Illumina BaseSpace).

The RNA expression data obtained may be in any suitable format, as aspects of the technology described herein are not limited in this respect. For example, in some embodiments, the RNA expression data may be obtained in a text-based file (e.g., in a FASTQ, FASTA, BAM, or SAM format). In some embodiments, a file in which sequencing data is stored may contains quality scores of the sequencing data. In some embodiments, a file in which sequencing data is stored may contain sequence identifier information.

RNA expression data, in some embodiments, includes RNA expression levels. RNA expression levels may be detected by detecting a product of gene expression such as mRNA and/or protein. In some embodiments, RNA expression levels are determined by detecting a level of a mRNA in a sample. As used herein, the terms “determining” or “detecting” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

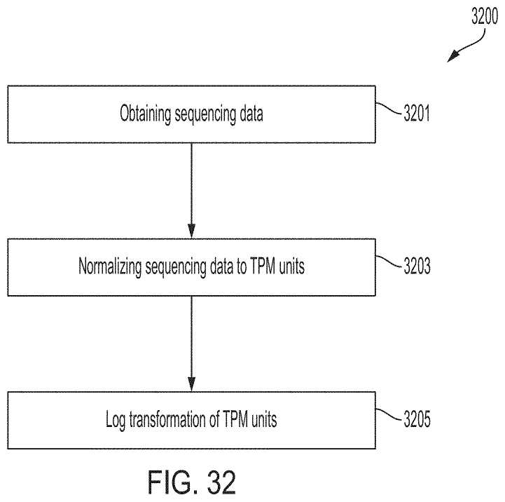

FIG. 32 shows an exemplary process 3200 for processing sequencing data to obtain RNA expression data from sequencing data. Process 3200 may be performed by any suitable computing device or devices, as aspects of the technology described herein are not limited in this respect. For example, process 3200 may be performed by a computing device part of a sequencing apparatus. In other embodiments, process 3200 may be performed by one or more computing devices external to the sequencing apparatus.

Process 3200 begins at act 3201, where sequencing data is obtained from a biological sample obtained from a subject. The sequencing data is obtained by any suitable method, for example, using any of the methods described herein including in the Section titled “Biological Samples.”

In some embodiments, the sequencing data obtained at act 3201 comprises RNA-seq data. In some embodiments, the biological sample comprises blood or tissue. In some embodiments, the biological sample comprises one or more tumor cells.

Next, process 3200 proceeds to act 3203 where the sequencing data obtained at act 3201 is normalized to transcripts per kilobase million (TPM) units. The normalization may be performed using any suitable software and in any suitable way. For example, in some embodiments, TPM normalization may be performed according to the techniques described in Wagner et al. (Theory Biosci. (2012) 131:281-285), which is incorporated by reference herein in its entirety. In some embodiments, the TPM normalization may be performed using a software package, such as, for example, the gcrma package. Aspects of the gcrma package are described in Wu J, Gentry RIwcfJMJ (2021). “gcrma: Background Adjustment Using Sequence Information. R package version 2.66.0.,” which is incorporated by reference in its entirety herein. In some embodiments, RNA expression level in TPM units for a particular gene may be calculated according to the following formula:

$$A \cdot \frac{1}{\sum ( A )} \cdot {10}^{6} \\ Where A = \frac{total reads mapped to gene \cdot {10}^{3}}{gene length in bp}$$

Next, process 3200 proceeds to act 3205, where the RNA expression levels in TPM units (as determined at act 3203) may be log transformed. Process 3200 is illustrative and there are variations. For example, in some embodiments, one or both of acts 3203 and 3205 may be omitted. Thus, in some embodiments, the RNA expression levels may not be normalized to transcripts per million units and may, instead, be converted to another type of unit (e.g., reads per kilobase million (RPKM) or fragments per kilobase million (FPKM) or any other suitable unit). Additionally or alternatively, in some embodiments, the log transformation may be omitted. Instead, no transformation may be applied in some embodiments, or one or more other transformations may be applied in lieu of the log transformation.

RNA expression data obtained by process 3200 can include the sequence data generated by a sequencing protocol (e.g., the series of nucleotides in a nucleic acid molecule identified by next-generation sequencing, sanger sequencing, etc.) as well as information contained therein (e.g., information indicative of source, tissue type, etc.) which may also be considered information that can be inferred or determined from the sequence data. In some embodiments, expression data obtained by process 3200 can include information included in a FASTA file, a description and/or quality scores included in a FASTQ file, an aligned position included in a BAM file, and/or any other suitable information obtained from any suitable file.

### Post-Mapping Processing

The second expression levels of genes of a biological sample may be used as inputs for any suitable downstream technique of processing expression data. Examples of downstream processing techniques include but are not limited to applying quality control techniques to the second expression levels, associating the biological sample to a cohort using the second expression levels, determining a tumor microenvironment of a subject using the second expression levels, performing cellular deconvolution using the expression levels, and selecting a therapeutic agent for the subject using the expression levels.

In some embodiments, the second expression levels of genes of the biological sample are used as input for applying one or more quality control techniques to the expression levels. Methods of applying quality control techniques to expression levels are known, for example as described in International Application Number PCT/IB2020/000928, filed Jul. 3, 2020, published as International Publication WO2021/028726 on Feb. 18, 2021, the entire contents of which are incorporated by reference herein.

In some embodiments, the second expression levels of genes of the biological sample are used as input for associating the biological sample to a cohort. Methods of associating the biological sample to a cohort are known, for example as described in International Application Number PCT/US2018/037008, filed Jun. 12, 2018, published as International Publication WO2018/231762 on Dec. 20, 2018, the entire contents of which are incorporated by reference herein.

In some embodiments, the second expression levels of genes of the biological sample are used as input for determining a tumor microenvironment of a subject. Methods of determining a tumor microenvironment of a subject are known, for example as described in International Application Number PCT/US2018/037017, filed Jun. 12, 2018, published as International Publication WO2018/231771 on Dec. 20, 2018, the entire contents of which are incorporated by reference herein.

In some embodiments, the second expression levels of genes of the biological sample are used as input for performing cellular deconvolution. Methods of performing cellular deconvolution are known, for example as described in International Application Number PCT/US2021/022155, filed Mar. 12, 2021, published as International Publication WO2021/183917 on Sep. 16, 2021, the entire contents of which are incorporated by reference herein.

In some embodiments, the second expression levels of genes of the biological sample are used as input for selecting a therapeutic agent for the subject. Methods of selecting a therapeutic agent for a subject are known, for example as described in International Application Number International Application Number PCT/US2018/037008, filed Jun. 12, 2018, published as International Publication WO2018/231762 on Dec. 20, 2018, the entire contents of which are incorporated by reference herein.

### Anti-Cancer Therapies

Aspects of the disclosure relate to methods of treating a subject having (or suspected or at risk of having) cancer by administering to the subject a cancer therapeutic selected using the second expression levels obtained by methods as described herein. In some embodiments, the methods comprise administering one or more (e.g., 1, 2, 3, 4, 5, or more) therapeutic agents to the subject. In some embodiments, the therapeutic agent (or agents) administered to the subject are selected from small molecules, peptides, nucleic acids, radioisotopes, cells (e.g., CAR T-cells, etc.), and combinations thereof. Examples of therapeutic agents include chemotherapies (e.g., cytotoxic agents, etc.), immunotherapies (e.g., immune checkpoint inhibitors, such as PD-1 inhibitors, PD-L1 inhibitors, etc.), antibodies (e.g., anti-HER2 antibodies), cellular therapies (e.g. CAR T-cell therapies), gene silencing therapies (e.g., interfering RNAs, CRISPR, etc.), antibody-drug conjugates (ADCs), and combinations thereof. In some embodiments, a subject is administered an effective amount of a therapeutic agent. “An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons, or for virtually any other reasons.

Empirical considerations, such as the half-life of a therapeutic compound, generally contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally (but not necessarily) based on treatment, and/or suppression, and/or amelioration, and/or delay of a cancer. Alternatively, sustained continuous release formulations of an anti-cancer therapeutic agent may be appropriate. Various formulations and devices for achieving sustained release are known.

In some embodiments, dosages for an anti-cancer therapeutic agent as described herein may be determined empirically in individuals who have been administered one or more doses of the anti-cancer therapeutic agent. Individuals may be administered incremental dosages of the anti-cancer therapeutic agent. To assess efficacy of an administered anti-cancer therapeutic agent, one or more aspects of a cancer (e.g., tumor microenvironment, tumor formation, tumor growth, or TME types, etc.) may be analyzed.

Generally, for administration of any of the anti-cancer antibodies described herein, an initial candidate dosage may be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression or amelioration of symptoms occurs or until sufficient therapeutic levels are achieved to alleviate a cancer, or one or more symptoms thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner (e.g., a medical doctor) wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy may be monitored by conventional techniques and assays and/or by monitoring GC TME types as described herein. The dosing regimen (including the therapeutic used) may vary over time.

When the anti-cancer therapeutic agent is not an antibody, it may be administered at the rate of about 0.1 to 300 mg/kg of the weight of the patient divided into one to three doses, or as disclosed herein. In some embodiments, for an adult patient of normal weight, doses ranging from about 0.3 to 5.00 mg/kg may be administered. The particular dosage regimen, e.g., dose, timing, and/or repetition, will depend on the particular subject and that individual's medical history, as well as the properties of the individual agents (such as the half-life of the agent, and other considerations well known).

For the purpose of the present disclosure, the appropriate dosage of an anti-cancer therapeutic agent will depend on the specific anti-cancer therapeutic agent(s) (or compositions thereof) employed, the type and severity of cancer, whether the anti-cancer therapeutic agent is administered for preventive or therapeutic purposes, previous therapy, the patient's clinical history and response to the anti-cancer therapeutic agent, and the discretion of the attending physician. Typically, the clinician will administer an anti-cancer therapeutic agent, such as an antibody, until a dosage is reached that achieves the desired result.

Administration of an anti-cancer therapeutic agent can be continuous or intermittent, depending, for example, upon the recipient's physiological condition, whether the purpose of the administration is therapeutic or prophylactic, and other factors known to skilled practitioners. The administration of an anti-cancer therapeutic agent (e.g., an anti-cancer antibody) may be essentially continuous over a preselected period of time or may be in a series of spaced dose, e.g., either before, during, or after developing cancer.

As used herein, the term “treating” refers to the application or administration of a composition including one or more active agents to a subject, who has a cancer, a symptom of a cancer, or a predisposition toward a cancer, with the purpose to cure, heal, alleviate, relieve, alter, remedy, ameliorate, improve, or affect the cancer or one or more symptoms of cancer, or the predisposition toward cancer.

Alleviating cancer includes delaying the development or progression of the disease, or reducing disease severity. Alleviating the disease does not necessarily require curative results. As used therein, “delaying” the development of a disease (e.g., a cancer) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detected and assessed using clinical techniques known. Alternatively, or in addition to the clinical techniques known, development of the disease may be detectable and assessed based on other criteria. However, development also refers to progression that may be undetectable. For purpose of this disclosure, development or progression refers to the biological course of the symptoms. “Development” includes occurrence, recurrence, and onset. As used herein “onset” or “occurrence” of a cancer includes initial onset and/or recurrence.

Examples of the antibody anti-cancer agents include, but are not limited to, alemtuzumab (Campath), trastuzumab (Herceptin), Ibritumomab tiuxetan (Zevalin), Brentuximab vedotin (Adcetris), Ado-trastuzumab emtansine (Kadcyla), blinatumomab (Blincyto), Bevacizumab (Avastin), Cetuximab (Erbitux), ipilimumab (Yervoy), nivolumab (Opdivo), pembrolizumab (Keytruda), atezolizumab (Tecentriq), avelumab (Bavencio), durvalumab (Imfinzi), and panitumumab (Vectibix).

Examples of an immunotherapy include, but are not limited to, a PD-1 inhibitor or a PD-L1 inhibitor, a CTLA-4 inhibitor, adoptive cell transfer, therapeutic cancer vaccines, oncolytic virus therapy, T-cell therapy, and immune checkpoint inhibitors.

Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

Examples of a surgical therapy include, but are not limited to, a curative surgery (e.g., tumor removal surgery), a preventive surgery, a laparoscopic surgery, and a laser surgery.

Examples of the chemotherapeutic agents include, but are not limited to, R-CHOP, Carboplatin or Cisplatin, Docetaxel, Gemcitabine, Nab-Paclitaxel, Paclitaxel, Pemetrexed, and Vinorelbine. Additional examples of chemotherapy include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin hydrochloride, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives or derivatives thereof); Purine antagonists (Thioguanine, Fludarabine, Cladribine, 6-Mercaptopurine, Pentostatin, clofarabine, and relatives or derivatives thereof) and Pyrimidine antagonists (Cytarabine, Floxuridine, Azacitidine, Tegafur, Carmofur, Capacitabine, Gemcitabine, hydroxyurea, 5-Fluorouracil (5FU), and relatives or derivatives thereof); Alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives or derivatives thereof); nitrosoureas (e.g., Carmustine, Lomustine, Semustine, Fotemustine, Nimustine, Ranimustine, Streptozocin, and relatives or derivatives thereof); Triazenes (e.g., Dacarbazine, Altretamine, Temozolomide, and relatives or derivatives thereof); Alkyl sulphonates (e.g., Busulfan, Mannosulfan, Treosulfan, and relatives or derivatives thereof); Procarbazine; Mitobronitol, and Aziridines (e.g., Carboquone, Triaziquone, ThioTEPA, triethylenemalamine, and relatives or derivatives thereof); Antibiotics, such as Hydroxyurea, Anthracyclines (e.g., doxorubicin agent, daunorubicin, epirubicin and relatives or derivatives thereof); Anthracenediones (e.g., Mitoxantrone and relatives or derivatives thereof); Streptomyces family antibiotics (e.g., Bleomycin, Mitomycin C, Actinomycin, and Plicamycin); and ultraviolet light.

### Computer Implementation

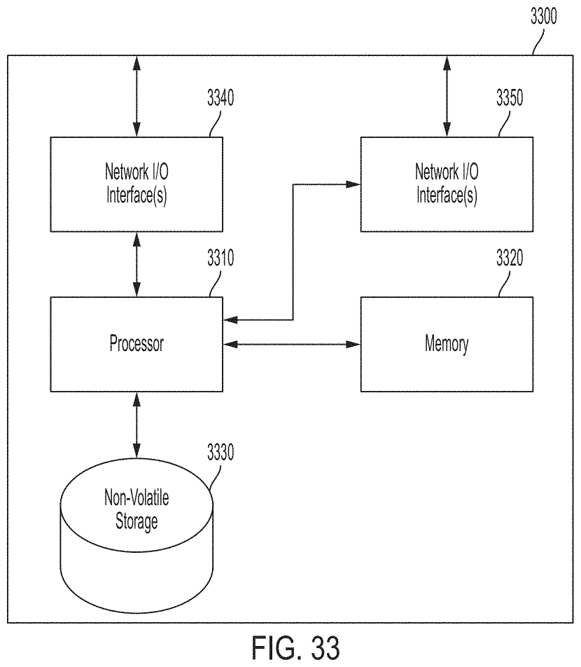

An illustrative implementation of a computer system 3300 that may be used in connection with any of the embodiments of the technology described herein (e.g., such as the method of FIG. 3) is shown in FIG. 33. The computer system 3300 includes one or more processors 3310 and one or more articles of manufacture that comprise non-transitory computer-readable storage media (e.g., memory 3320 and one or more non-volatile storage media 3330).

The processor 3310 may control writing data to and reading data from the memory 3320 and the non-volatile storage device 3330 in any suitable manner, as the aspects of the technology described herein are not limited to any particular techniques for writing or reading data. To perform any of the functionality described herein, the processor 3310 may execute one or more processor-executable instructions stored in one or more non-transitory computer-readable storage media (e.g., the memory 3320), which may serve as non-transitory computer-readable storage media storing processor-executable instructions for execution by the processor 3310.

Computing device 3300 may also include a network input/output (I/O) interface 3340 via which the computing device may communicate with other computing devices (e.g., over a network), and may also include one or more user I/O interfaces 3350, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

### EXAMPLES

### Example 1: Batch Effects

RNA-seq quantitatively measures gene expression across the whole genome, and higher expression values correspond to more abundant mRNAs in a sample. This linearity is the main property of any RNA quantification assay and the cause of high (>80%) intra-sample correlation across different platforms. This cross platform agreement of expression levels has been previously shown for qPCR/TaqMan, DNA microarrays (e.g. HuGene2) and different RNA-seq modifications (SOLID) through comparison of log ratios between expression levels of selected genes from the same samples. In the comparisons, the majority of gene-points (ratios) were proportional and followed y=a*x line, where the coefficient “a” depends on the pair of compared platforms. Almost all individual RNA expression assessment platforms (e.g., SOLID, ribo-Zero, EC, Nugen) correlated with the qPCR assessments, thereby supporting the idea of gene expression linear comparability across different methods, including poly-A and EC sequencing. Notably, linearity was more evident for protein-coding gene selection.

Absolute expression values of genes profiled with the same protocol differ depending on the tissue preservation method (in microarrays and total RNA-seq). Furthermore, the absolute values vary if samples were sequenced by alternative protocols, a problem known as a batch effect. Normalization, the adjustment of global properties of measurements for individual samples, does not eliminate batch effects. Additionally, the direct cause of batch effects are technical differences; therefore, the removal of these technical differences does not affect the biological variability. Gene expression in a specimen assessed using different GEP protocols (e.g., Poly-A RNA-seq, EC RNA-seq, microarray) will differ due to the batch effect; however, the relative expression values for all genes in comparison to each other will remain generally similar (i.e., high intra-sample multi-gene Pearson correlation within RNA-seq and high Spearman correlation across any platform). Although the absolute values produced by alternative protocols may substantially vary, most genes linearly correlate across different protocols.

Previously described batch effect correction algorithms have been developed to neutralize the batch effect between samples across large cohorts. However, these techniques are generally not suitable for batch correction of expression data obtained from an individual sample.

### Example 2: Single Sample Mapping

### Gene Selection

This example describes linear models that can be applied that map expression data of a single biological sample sequenced using a first protocol (e.g., FFPE tissue sequenced by EC RNA-seq) to reference expression data (e.g., expression data for a cohort of patients) obtained from biological samples sequenced using a different protocol than the first protocol (e.g., FF tissue sequenced by PolyA RNA-seq). Performance of the algorithms described herein was improved by training with paired samples sequenced using the two different protocols, enabling the data from the two protocols to be analyzed in combination.

Briefly, RNA transcripts per million (TPM) normalization was performed within the set of transcripts (gene isoforms) selected according to their biological types using the GENCODE v23 transcriptome annotation or their biological family. For TPM normalization, all transcripts of non-coding biological types were excluded, as previously performed in The Cancer Genome Atlas (TGCA) mRNA Analysis Pipeline for FPKM. Histone-coding and mitochondrial gene transcripts were also excluded due to uneven enrichment with different RNA extraction methods, e.g., PolyA vs Total RNA. The resulting set of genes which were retained for TPM normalization and expression quantification contained 20,062 genes, with a set of 1,899 genes that are cancer-specific, immune-related, and clinically and scientifically relevant for cancer (i.e., clinical biomarkers and genes that may be utilized for further processing, for example single sample gene set enrichment analysis (ssGSEA) and cell deconvolution techniques) chosen as the most relevant targets for the projection from one protocol to another.

Mapping of some genes from one protocol to another could be affected by technical or biological issues. For example, some genes may not intersect with probes utilized for EC and other genes may have transcripts with low annotation or reference sequence quality (e.g., low transcript support level, partially unknown coding sequences, and others). There are families of genes that are lost during Poly-A sequencing protocol in contrast to total RNA or EC protocols, which can be explained by specific polyadenylation (e.g., ubiquitin specific peptidase 17 like family, speedy/RINGO cell cycle regulator family, taste 2 receptor family, and some olfactory receptors). Also, the expression of TCR- and BCR-coding genes annotated in the transcriptome as corresponding to the V, D or J regions cannot be properly quantified without specific alignment tools such as MiXCR. Additionally, for more than 4,000 genes, direct measurements of Poly-A lengths in HeLa cell line cells were obtained. Genes that had Poly-A length less than the mean and differed more than one standard deviation from the mean were considered as having short Poly-A tails. 190 genes with the aforementioned issues were included into a target gene set of 1,899 genes alongside 271 additional genes (300 in total), which often have low expressions around noise levels measured across Poly-A or EC (e.g., Agilent SureSelect V7+UTR) protocols both or separately.

Overall four groups of genes (listed below in Table 1) were obtained for further analysis. Tables 2 and 3 provide examples of genes in the BMG and BMGEP groups described in Table 1.

TABLE 1

| Group Name | Description | Number of Genes |
|---|---|---|
| AG | All Genes (AG) present in | 20062 |
| | the BostonGene product | |
| AGEP | All Genes, Excluding 6624 | 13438 |
| | aforementioned | |
| | “Problematic” genes (AGEP) | |
| BMG | Biologically Meaningful | 1899 |
| | Genes | |
| BMGEP | Biologically Meaningful | 1438 |
| | Genes Excluding the | |
| | “Problematic” genes | |

### Single Gene Mapping

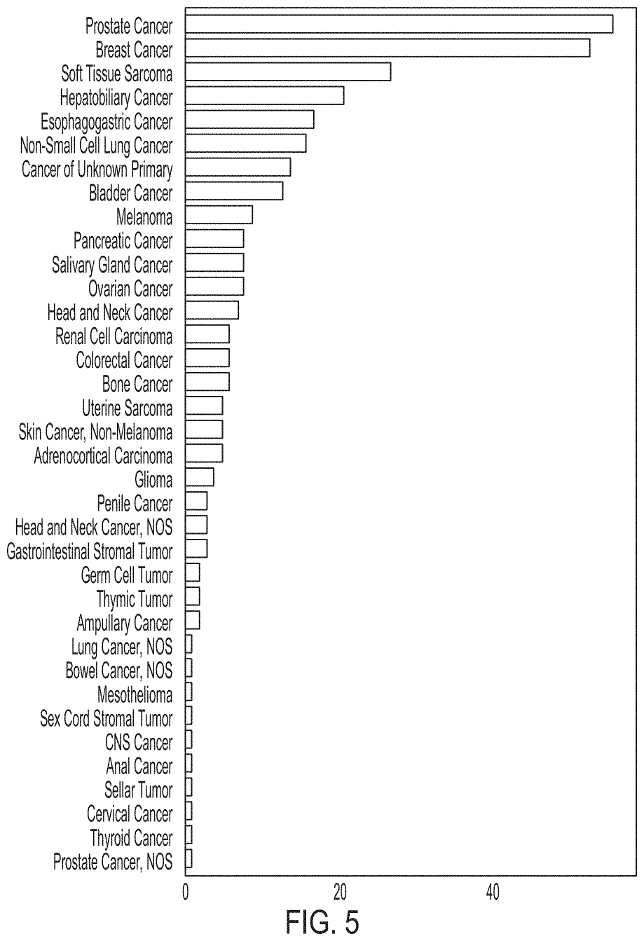

To investigate the possibility of creating a batch correction algorithm using paired samples sequenced with poly-A or EC RNA-seq, a publicly available cohort, MET500, containing paired samples of diverse cancer types was acquired (FIG. 5). Overall, 320 paired samples sequenced with both Poly-A RNA-seq and Agilent Sureselect V4 EC protocols from the same samples were included.

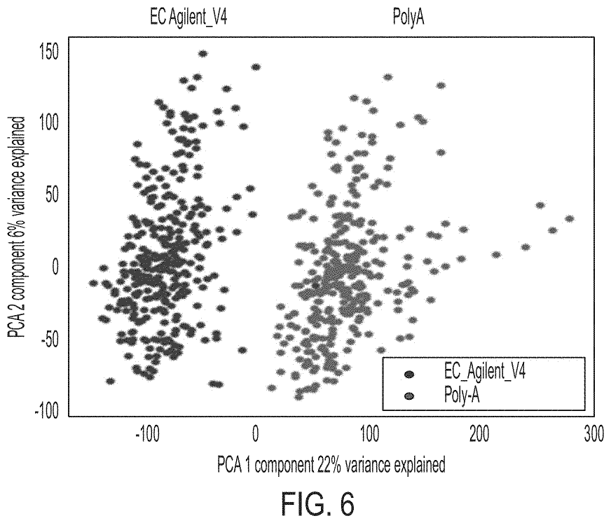

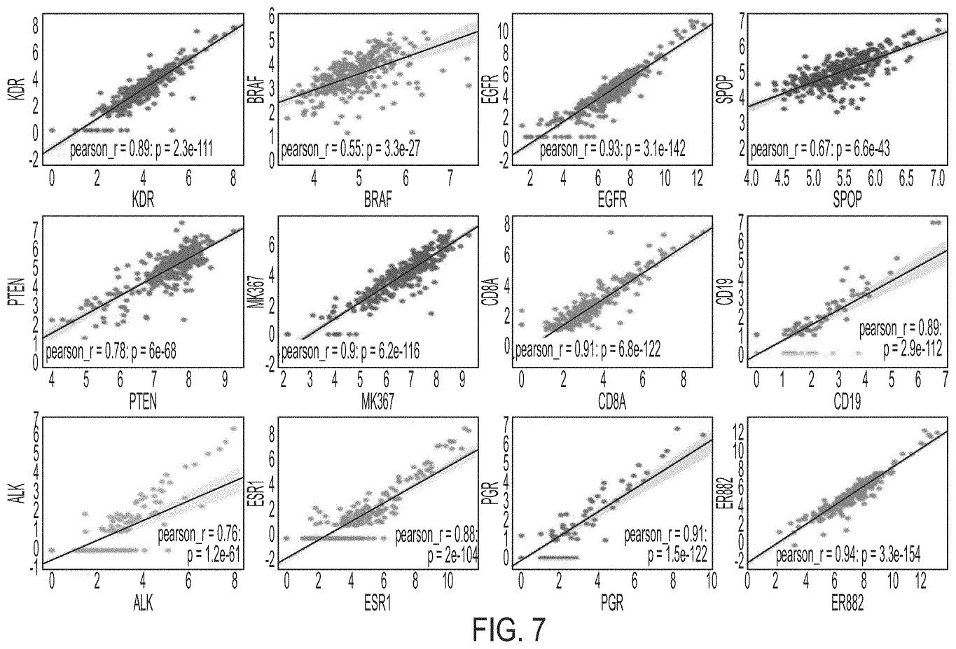

For the MET500 cohort, PCA demonstrates a clear separation between expression data produced by different protocols (FIG. 6). Absolute values differed for the majority of genes; however, high Pearson correlation values were observed between protocols for many of them (representative examples, FIG. 7).

Overall, 297 out of 320 samples passed the implemented quality control steps. 92 pairs of samples were selected as a holdout set to perform validation comparisons and the remaining set of samples was used to train single-gene models (e.g., Single-Gene Mapping, as shown in FIGS. 2A-2B). A brief description of the single-gene models is provided below:

Given p predictors, the linear regression model predicts the response y by

y=w₀+w₁x₁+ . . . +wₚxₚ.

A model fitting procedure produces a vector of coefficients w. For example, the ordinary least squares (OLS) estimates are obtained by minimizing the residual sum of squares. However, OLS often performs poorly in both prediction and interpretation. Penalization techniques are utilized to improve OLS. The lasso and the ridge regressions are penalized least squares methods imposing an 11- and 12-penalties on the regression coefficients, respectively.

In the case of expression data projection from one sequencing protocol to another, y is the projected expression and x is a vector of predictors. Concerning the aforementioned cross platform agreement of expression levels, when the majority of gene-points (ratios) follow linear dependence between different platforms, the linear regression model with an equation y=w₀+w₁x₁ could be useful, where x₁ is the target gene expression in EC and y is its projection to poly-A.

A machine learning tool named ElasticNet was used. This tool is based on regularization of linear regression coefficients by adjusting both 11- and 12-penalties through minimizing the following equation:

$${min}_{w} \frac{1}{2 {n}_{samples}} {‖ Xw - y ‖}_{2}^{2} + α ρ {‖ w ‖}_{1} + \frac{α ( 1 - ρ )}{2} {‖ w ‖}_{2}^{2} ,$$

where α is a constant which multiplies 11- and 12-penalties; p is an 11-ratio ranging from 0 to 1, where value equal to 1 means using Lasso penalty only.

In some embodiments, a version of ElasticNet named ElasticNetCV was used. This model provides an internal cross-validation estimator which can be utilized for searching of specified model parameters (i.e. a and 11-ratio) with more computing power efficiency compared to the canonical estimators.

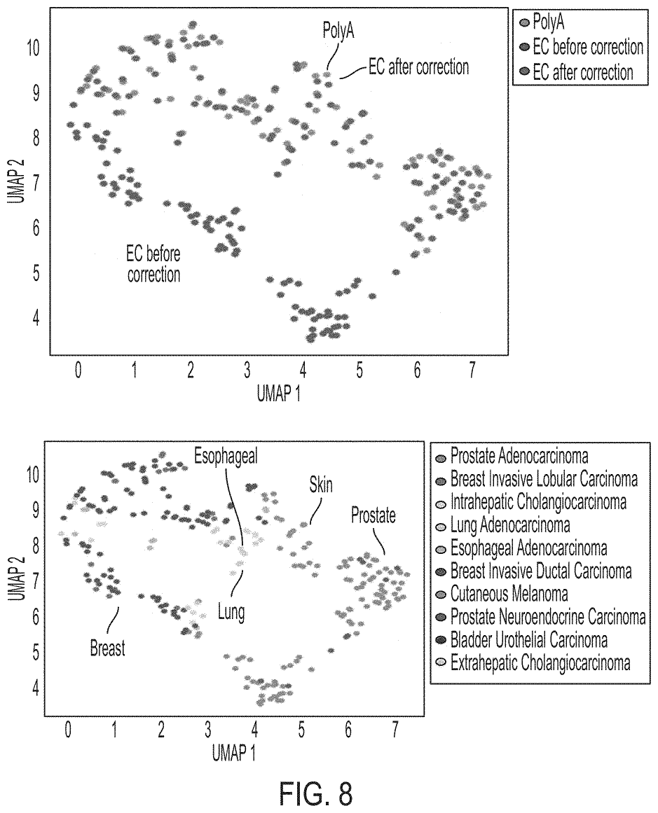

The ElasticNetCV regression models were utilized to automatically adjust parameters, and the concordance correlation coefficient (CCC) was used to measure whether the algorithm accurately overcame the batch effects between the two different technologies. Next, the linear models (also referred to as “transformations”) were applied to “correct” (e.g., map) expression values in the holdout set. The UMAP projection performed on the All Gene (AG) group showed that this algorithm effectively overcame the overall batch effects while maintaining a unique tissue gene expression pattern (FIG. 8).

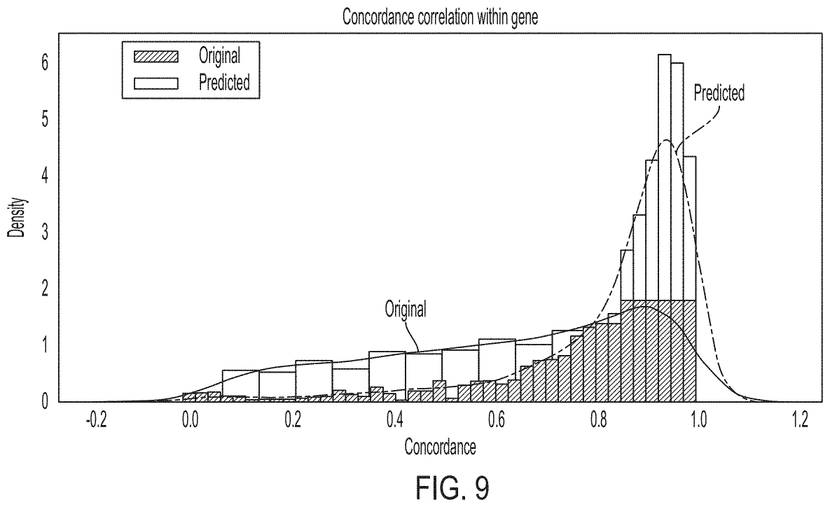

Next, correction performance of the algorithm across the Biologically Meaningful Genes (BMG) group. The CCC values for more than 1518 genes were above 0.75, demonstrating robust performance of the developed single-gene model (FIG. 9). Thus, using this type of the model, the cohort can be combined. Moreover, an individual sample can be mapped from one protocol to an expression distribution of another protocol by applying the correction.

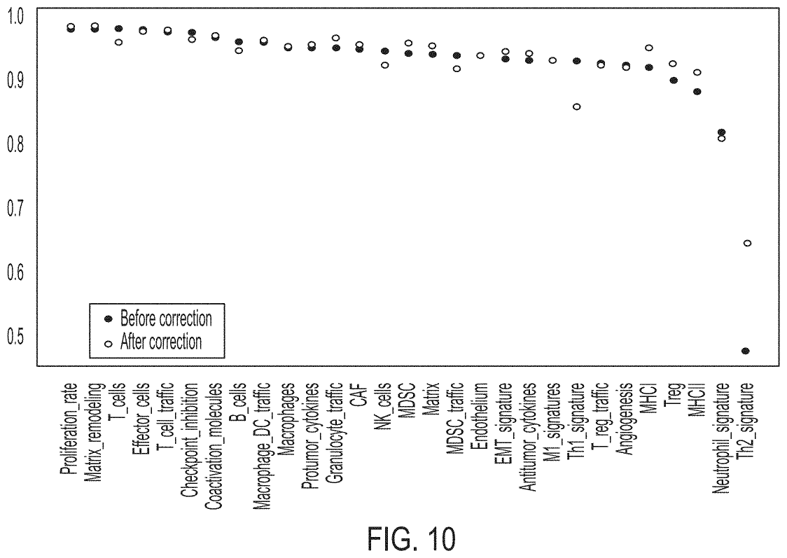

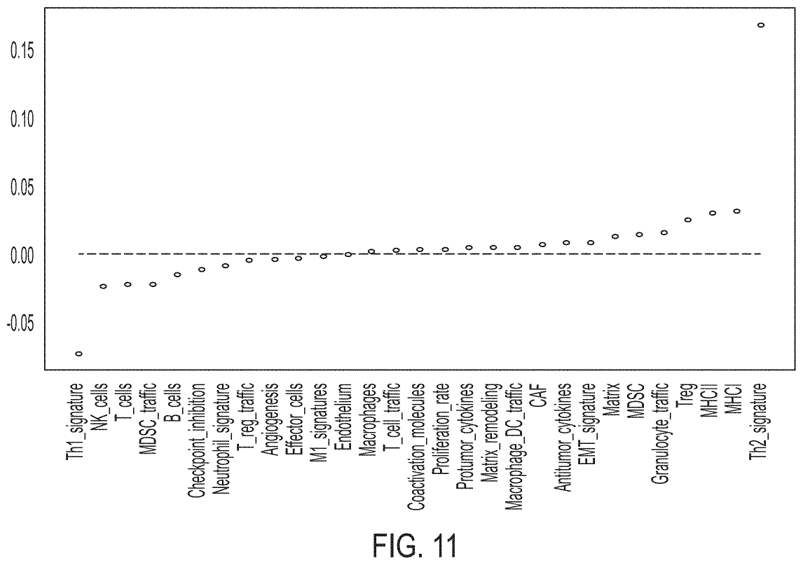

Next, reproducibility of gene signatures after correction was investigated. First, the values for representative gene signatures (e.g., as described by U.S. Patent Publication No. 2020-0273543, entitled “SYSTEMS AND METHODS FOR GENERATING, VISUALIZING AND CLASSIFYING MOLECULAR FUNCTIONAL PROFILES”, the entire contents of which are incorporated by reference herein) were calculated using ssGSEA. The initial and corrected values across paired Poly-A and EC samples were compared using CCC (PolyA vs. EC—Before correction and PolyA vs. EC—After correction). The CCC values for the majority of gene signatures before correction were above 90% and slightly increased after correction (FIG. 10 and FIG. 11).

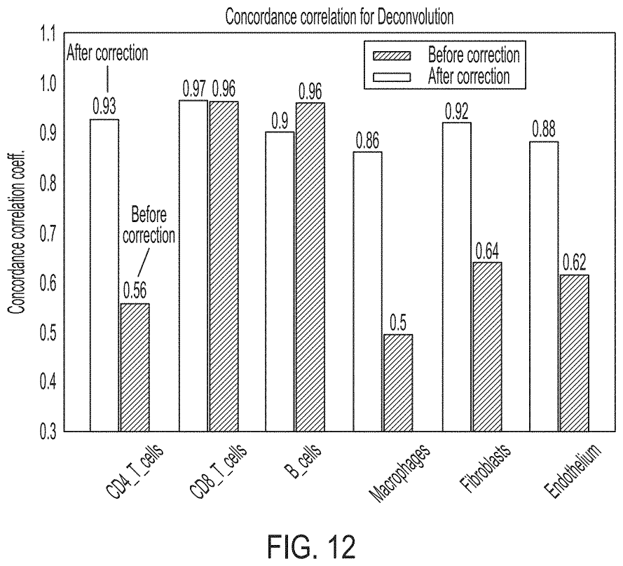

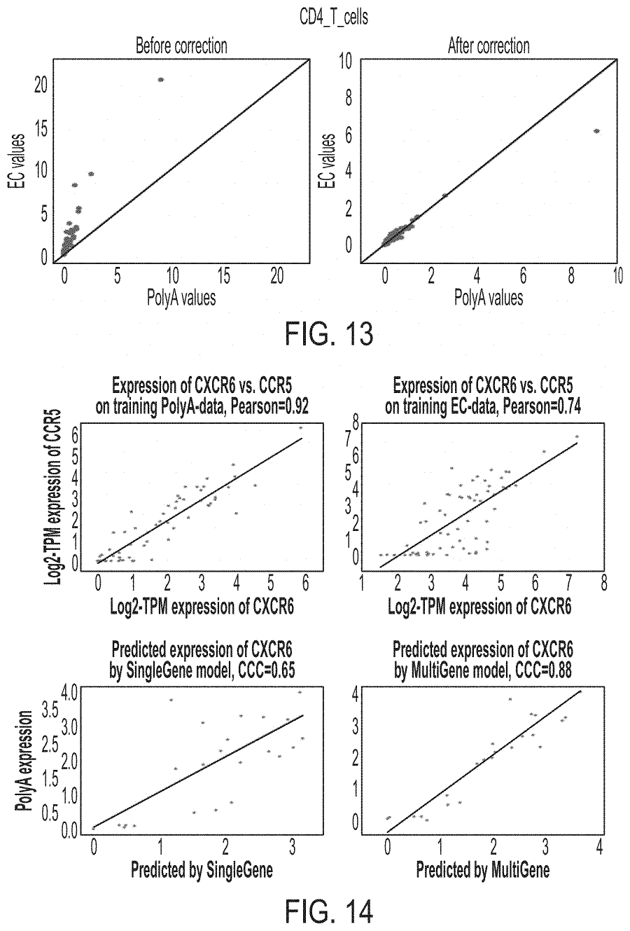

Next, comparisons were performed for Kassandra deconvolution (e.g., as described in U.S. patent application Ser. No. 17/200,492, filed on Mar. 12, 2021, and titled “SYSTEMS AND METHODS FOR DECONVOLUTION OF EXPRESSION DATA,” which is incorporated by reference in its entirety herein). CCC values were greatly improved across all major cell types (FIG. 12), showing the best results within CD4 T-Cells (FIG. 13).

### Multi-Gene Mapping

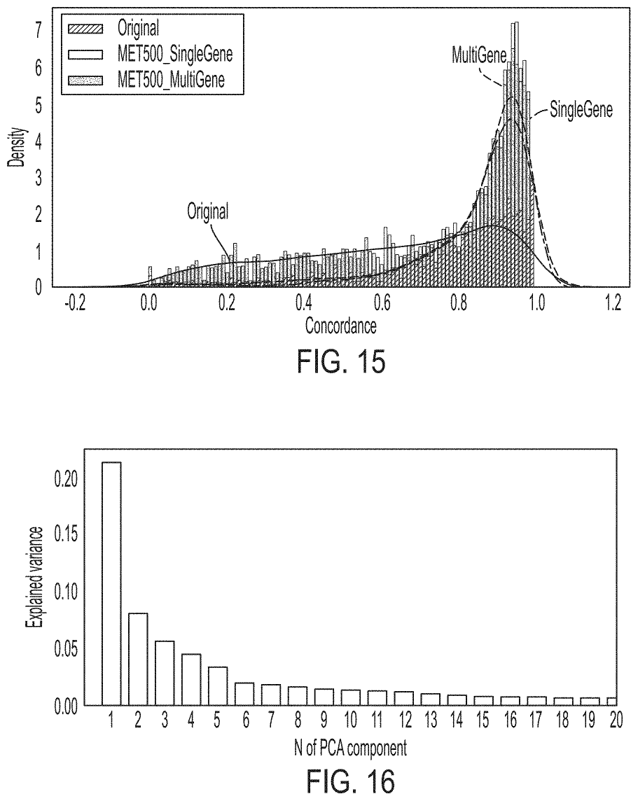

To develop a multi-gene model (e.g., Multi-Gene Mapping, as shown in FIGS. 2C-2D), Pearson correlations were calculated within the BMG group on TCGA expression-data, including different cancer types. FIG. 14 demonstrates a representative example of highly correlated genes with Pearson correlation values above 0.7 for both poly-A and EC samples. After that for each gene of interest, up to 50 most correlated genes were selected (e.g., by Pearson correlation of RNA expression levels), which then were used to build a Multi-Gene linear model. Briefly, the genes of interest and their correlated genes were used to train multi-gene models. ElasticNetCV regression models were utilized to automatically adjust parameters, and the concordance correlation coefficient (CCC) was used to measure whether the algorithm accurately overcame the batch effects between the two different technologies. Next, the transformations were applied to “correct” (e.g., map) expression values in the holdout set. It was observed that CCC values were higher within individual genes analyzed (FIG. 14, second row) and improved gene-wise CCC was observed across the BMG (FIG. 15) compared to the Single-Gene Mapping technique.

### Example 3: Comparison with Cohort-Based Corrections

To assess the effectiveness of the developed algorithms, they were compared against existing batch correction techniques: PCA-based correction, MNN-based correction, and ComBat. A comparative analysis is given below.

### PCA

Principal components analysis (PCA) was performed by removing one or more of the most important principal components (PC) and then reversing transformed data to original space (FIG. 16). Specifically, PCs can be obtained using the matrix with eigenvectors:

PC=VX.

where PC is the principal components, X is the original expression data (poly-A and EC merged to one data frame), and V is the matrix with eigenvectors.

Thus, reversed data after removing some of PCs can be achieved by solving the following equation:

V^{T}·PC=X.

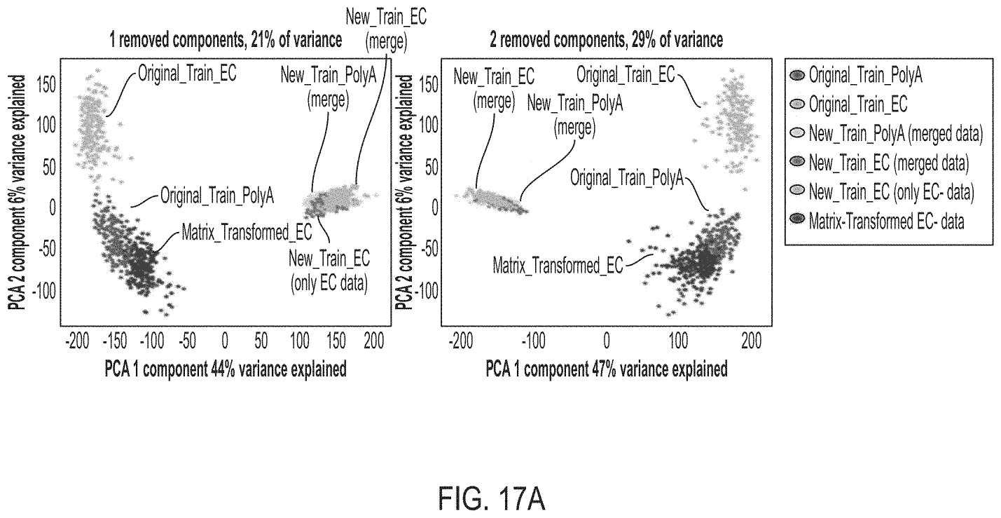

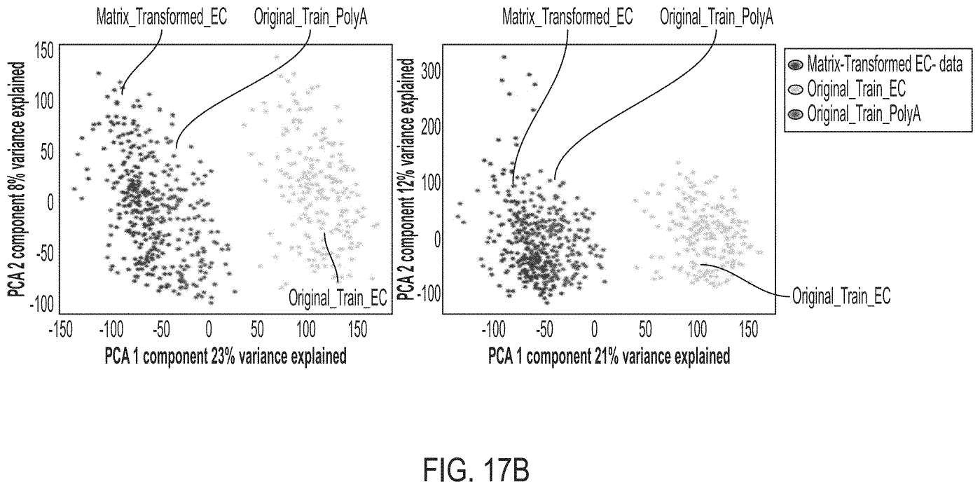

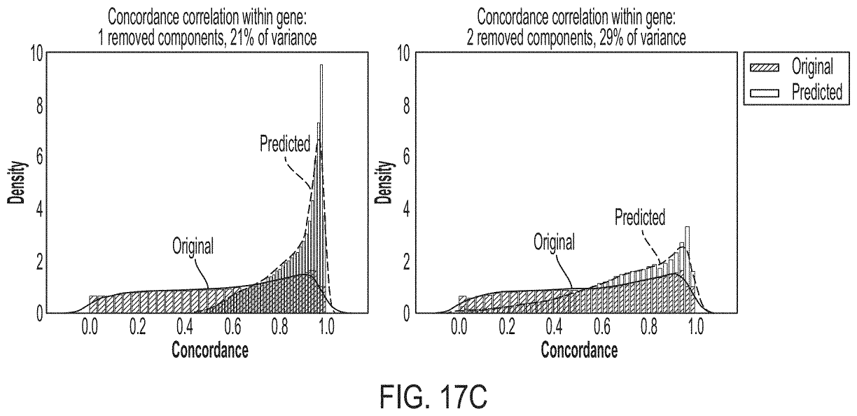

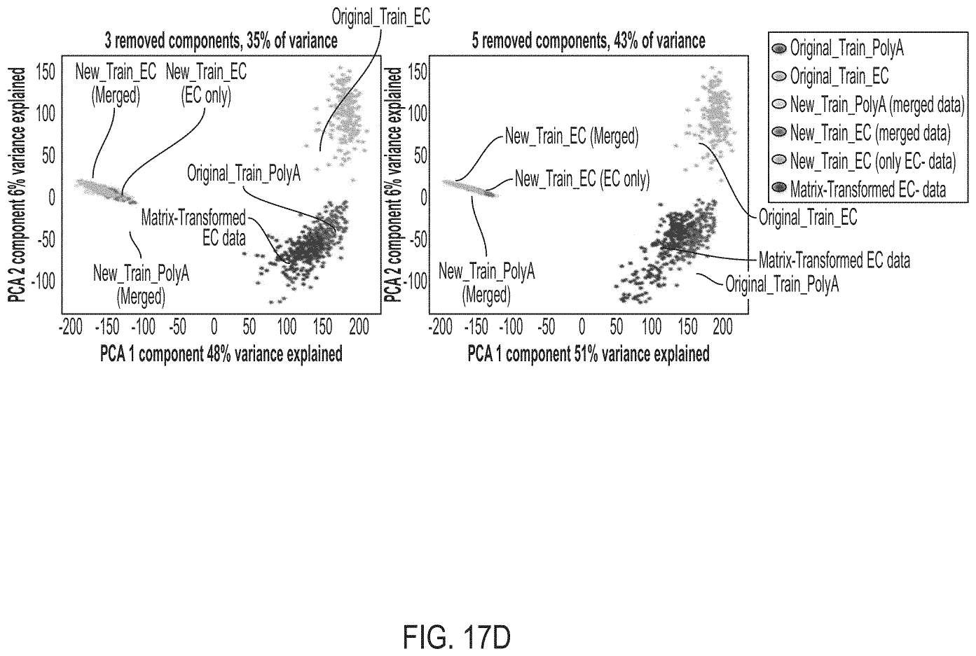

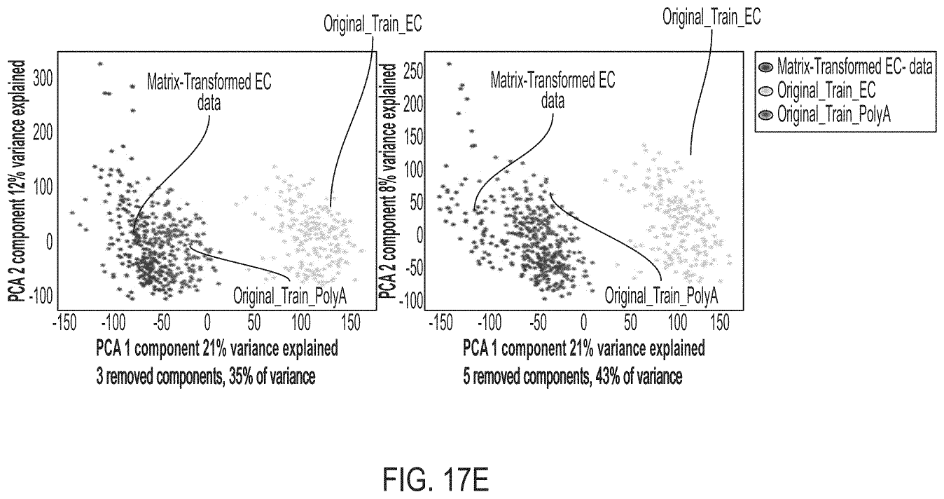

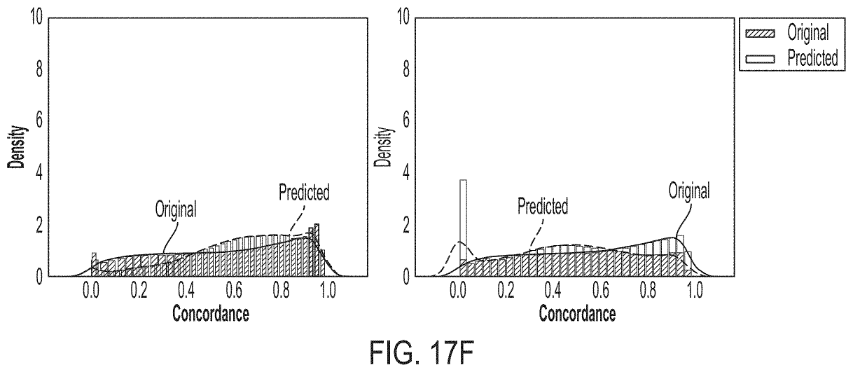

The results on the training data indicated that with increasing numbers of removed PCs there is a decrease in biological diversity of the expression data (FIGS. 17A-17F: 1st row). Also, upon removal of PCs, the EC and PolyA cohorts are merging together and projecting at the same space, but still remain not comparable with both original EC and PolyA expressions. Thus, it was attempted to identify a matrix, multiplication by which would lead transformed EC-expressions to the space of original PolyA-expressions (FIGS. 17A-17F: 2nd row). The results showed an improvement in gene-wise-performed CCC in case of removing the 1st PC (FIGS. 17A-17F: 3rd row).

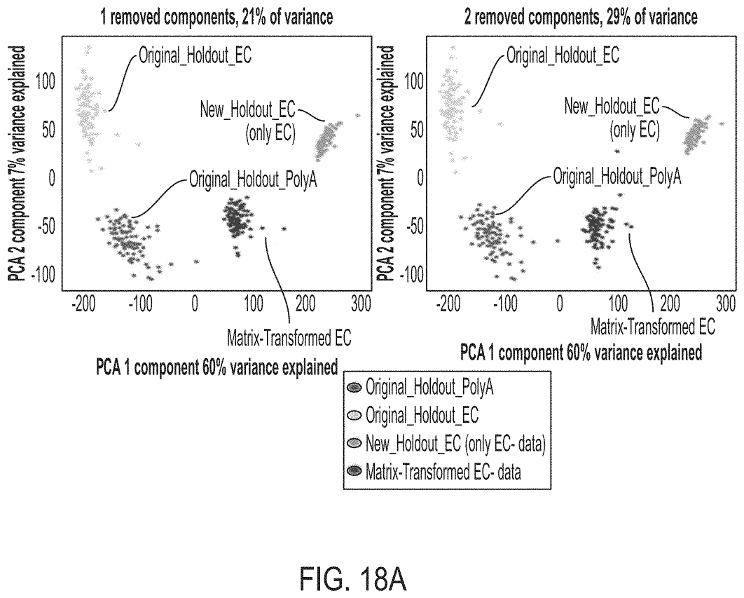

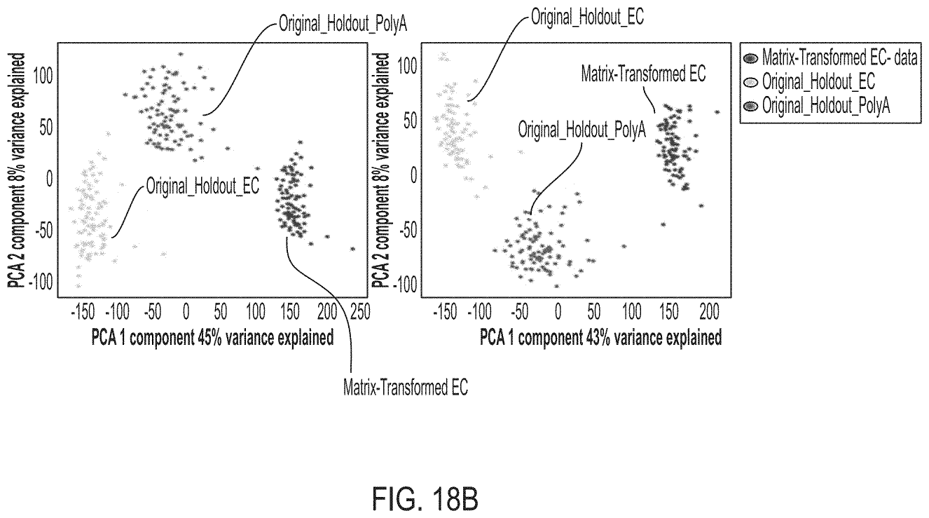

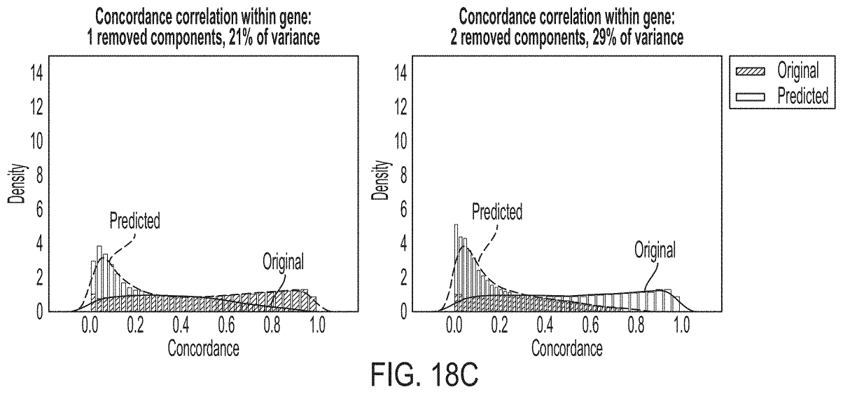

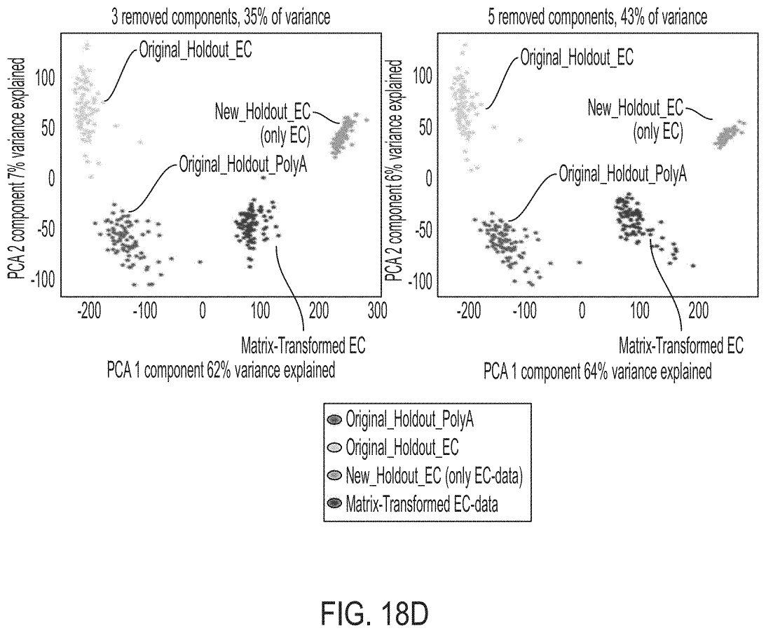

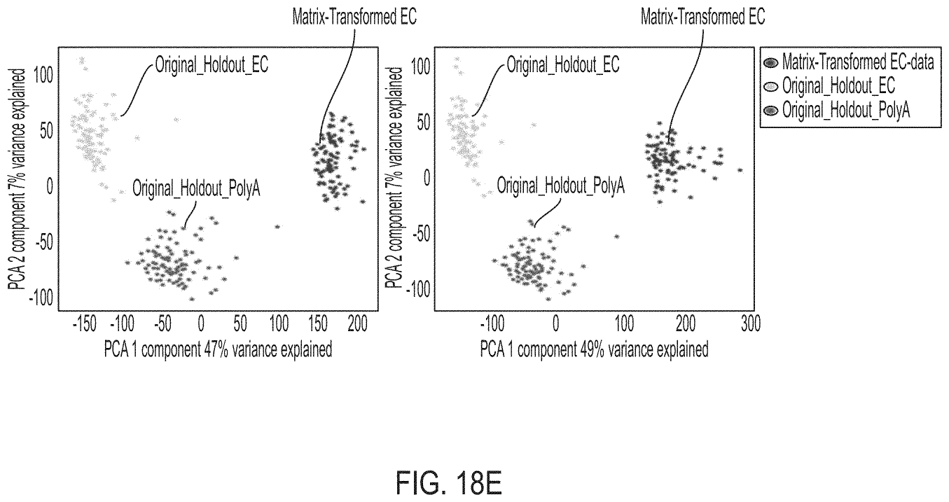

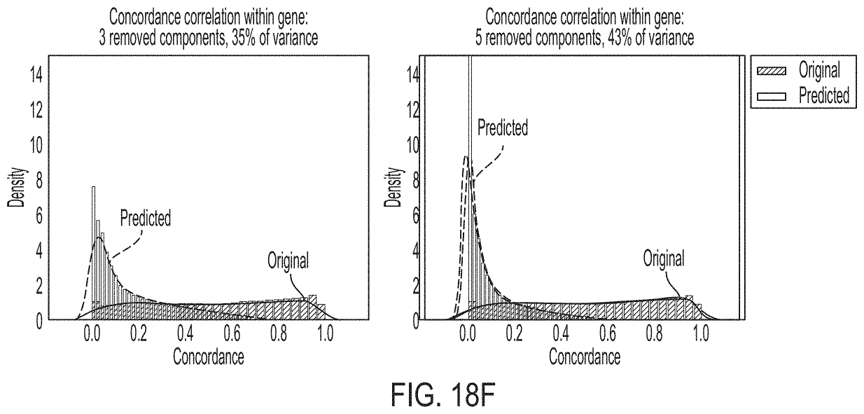

After that, the same PCs and the same multiplication matrix, which was obtained from the training samples, was used to perform transformation of the holdout samples (FIGS. 18A-18F). The results showed a decrease in gene-wise-CCC of transformed data compared to the original expressions. Thus, train-precalculated PCs and transition-matrices could not be used to transfer expression values from EC to poly-A for newly arrived samples.

### MNN-Based Correction

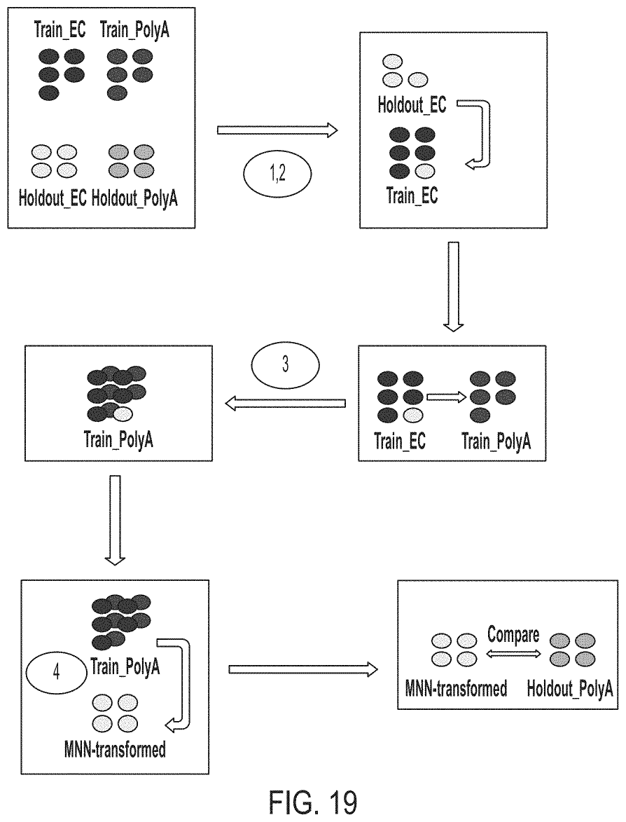

Next, a method based on detection of mutual nearest neighbors (MNN) was compared to the Single Sample Mapping techniques. In this approach, MNN pairs represent shared population structure and can be used to estimate batch-corrected values. To implement this method, each sample from the holdout-EC set were taken separately (one by one) and added to the training-EC set, and then the new set was fit with a training-polyA set. This way of utilizing MNN can be described by the following steps illustrated in FIG. 19:

1) take one sample from the holdout-EC set

2) add this sample to the train-EC set, which results in a “dummy set”

3) fit the “dummy set” with train-polyA expressions

4) select only the holdout-sample from transformed expressions and add it to the set of “MNN-transformed” samples.

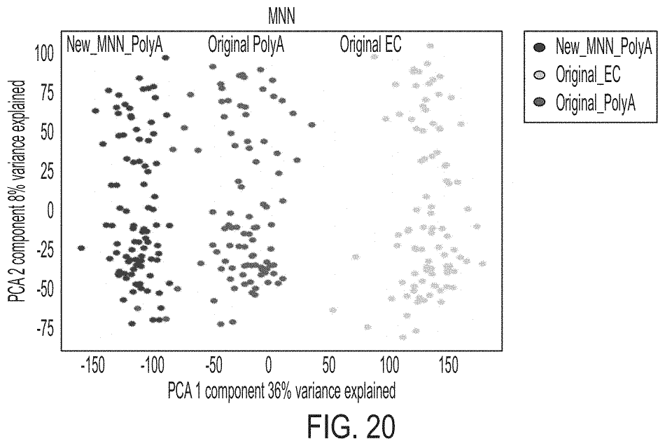

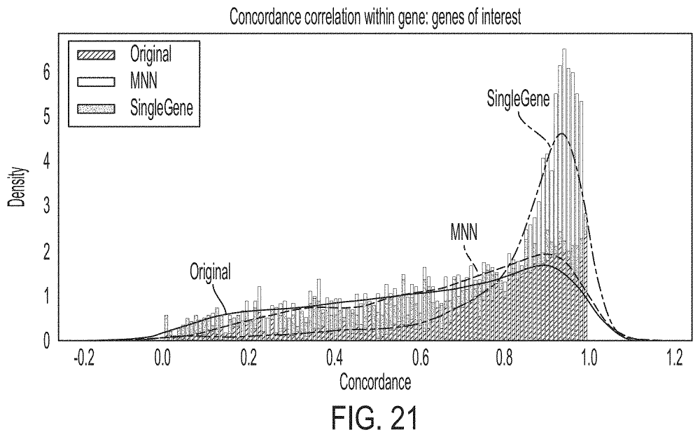

Then, the full “MNN-transformed” set of samples was compared with the holdout-polyA cohort. PCA projection showed that the transformed dataset did not perfectly fit the polyA expressions (FIG. 20). In terms of CCC values, MNN-based batch correction also could not overcome the performance of the Single Sample Mapping techniques (FIG. 21).

### COMBAT Correction

Finally, the Single Sample Mapping techniques were compared with another well-known batch correction tool—ComBat. ComBat was not able to be used “out of the box” in a technique for pretraining a model and then utilizing it for newly appeared single samples. Therefore, the same strategy as applied for MNN-algorithm was attempted—adding holdout-EC samples one by one to training-EC expressions and then merging this new data frame with training-polyA expressions (FIG. 19).

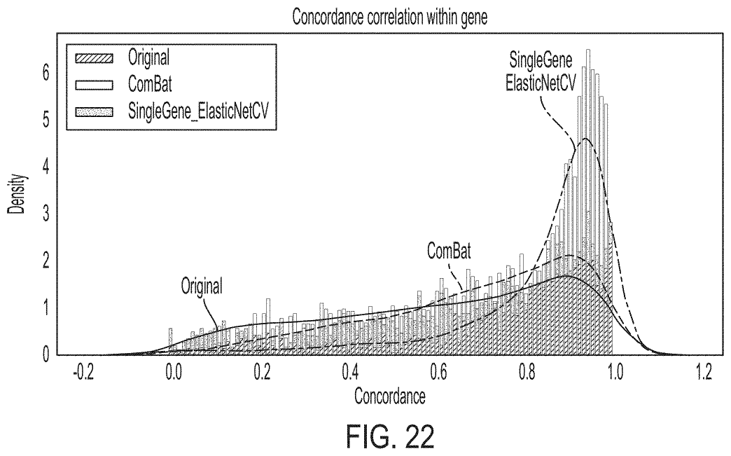

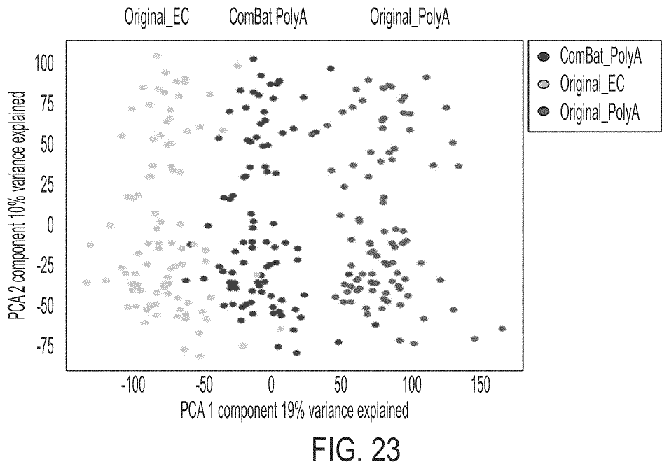

Performance of both methods was evaluated by calculating CCC for the expression values before and after correction. The Single Sample Mapping technique showed significantly higher CCC values and outperformed ComBat in this test (FIG. 22). Also, PCA demonstrated that ComBat's transformed-expressions were projected onto a different space compared to both EC and polyA holdout data (FIG. 23).

### Model Comparison

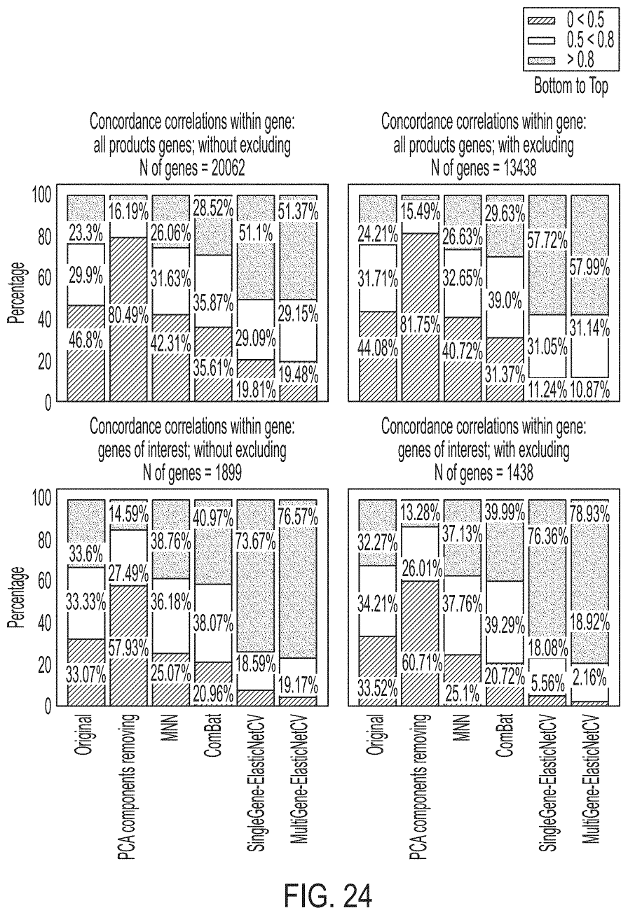

Different methods described in the previous sections were used to unify EC and poly-A expressions across four predefined groups of genes (e.g., Table 1) and compared their gene-wise-CCC values calculated on the holdout set of MET500 samples (FIG. 24). Single- and multi-gene linear models showed greater performance (more than 75% of genes with CCC>0.8) compared to the original data and other methods. Therefore, these 2 models were selected for further evaluation on laboratory data.

### Example 4: Single Sample Mapping and Cohort Identification

Models were created for the Agilent SureSelect V7+UTR protocol. In total, 88 pairs of samples from the same piece of tissue underwent different sample processing and sequencing procedures. FF samples were sequenced using Poly-A protocol, whereas in-house-prepared FFPE samples were sequenced using EC protocol Agilent V7+UTR. Overall, 64 of the paired samples were used for training of ElasticNetCV linear models (one for each gene), and the remaining 24 samples were used for the holdout dataset.

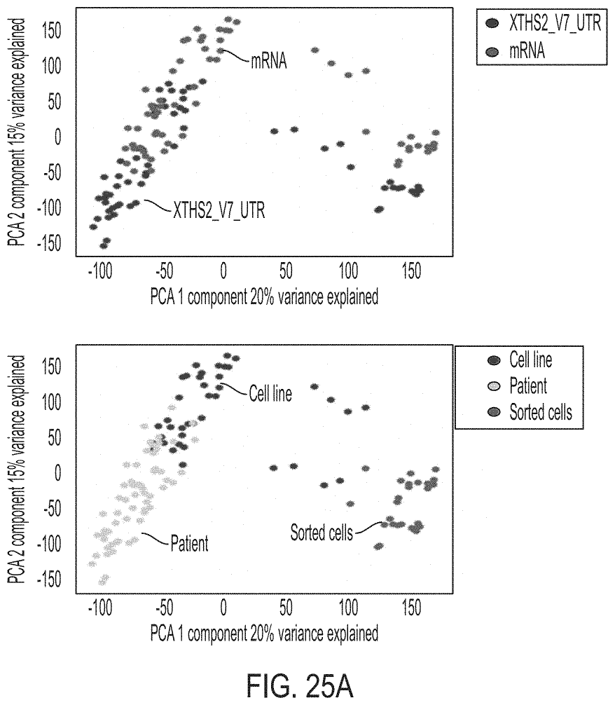

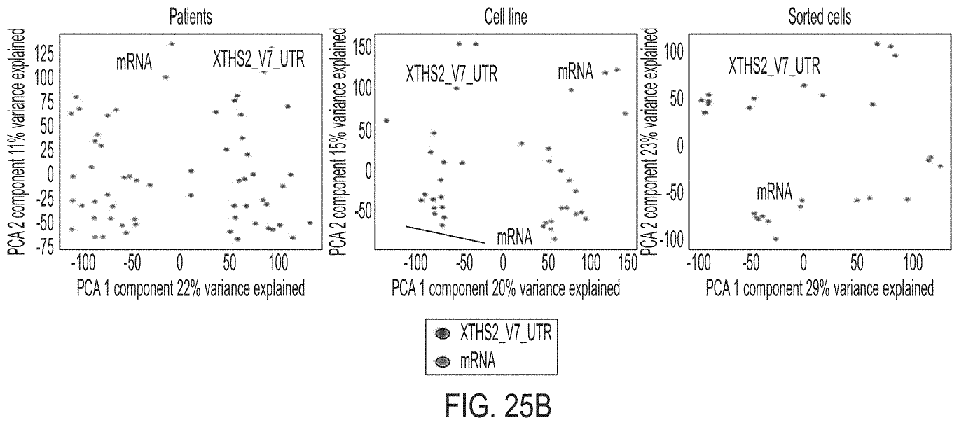

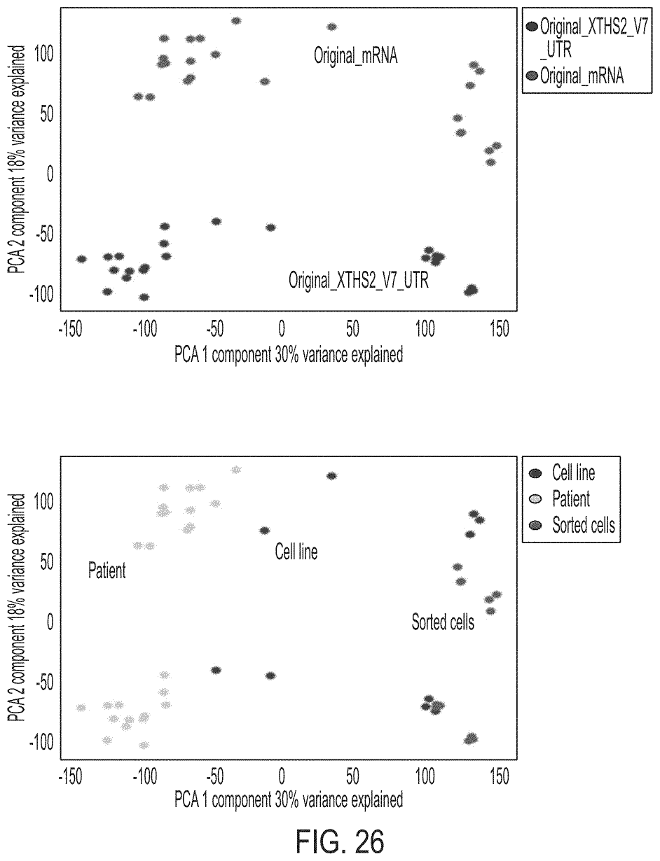

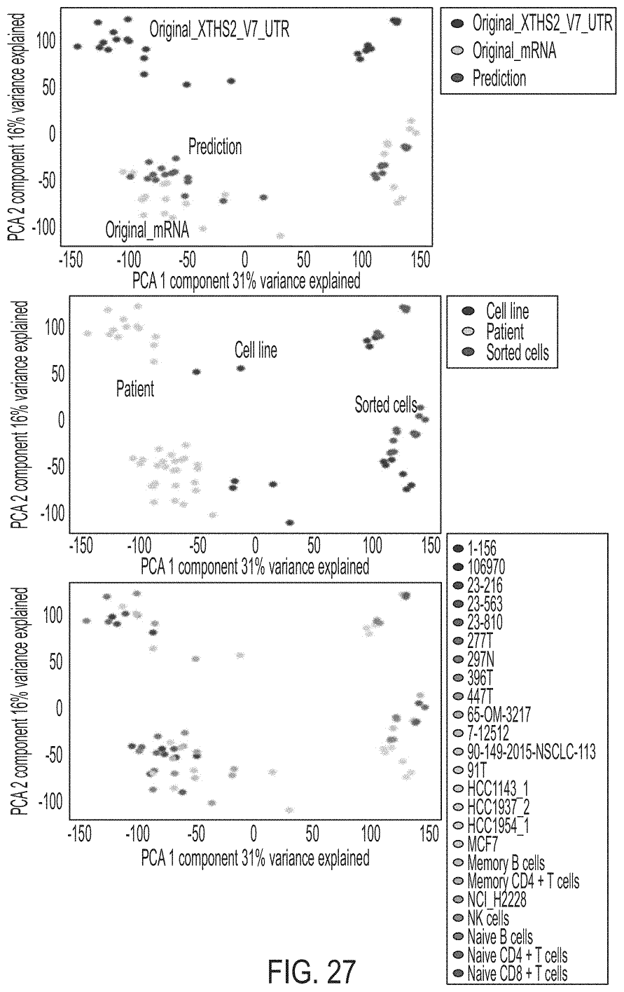

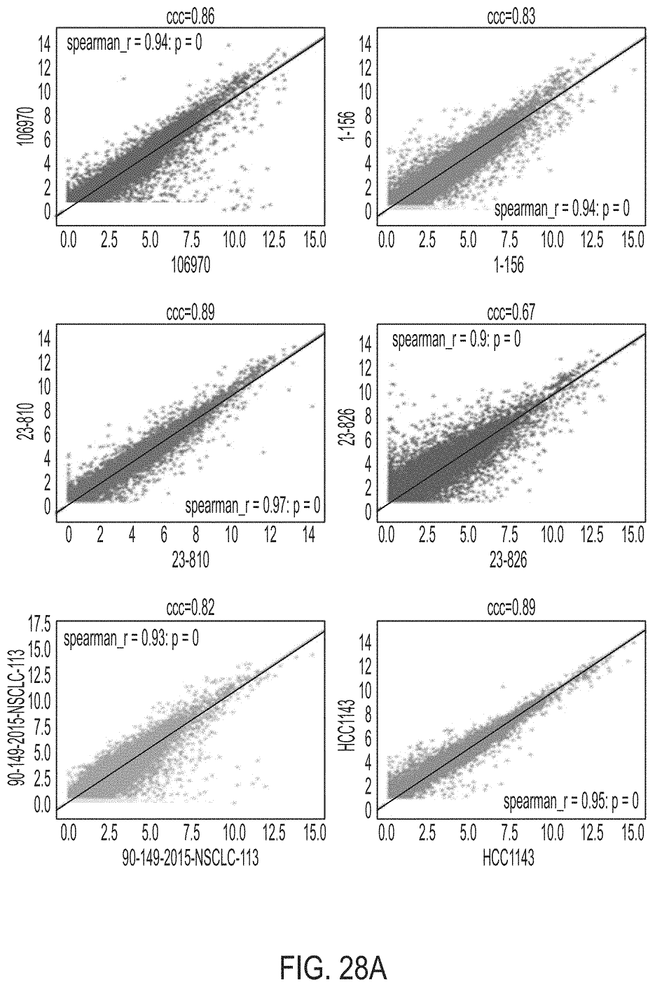

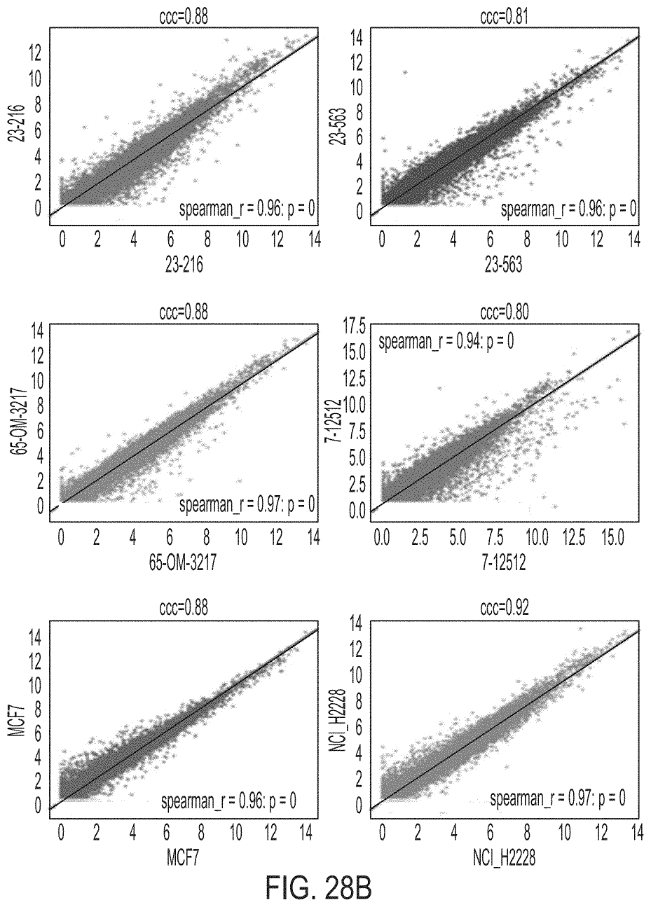

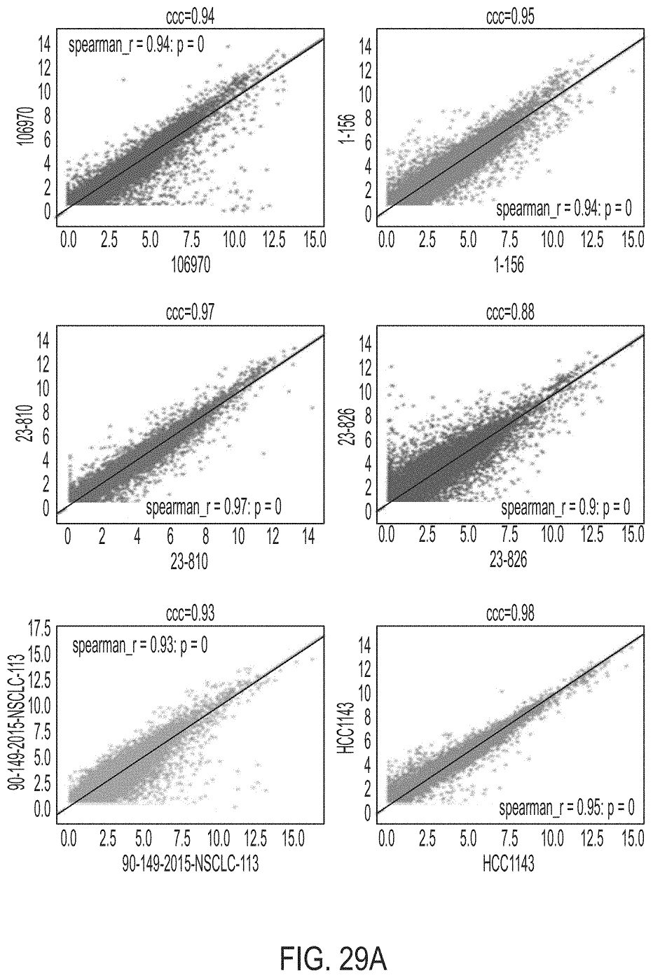

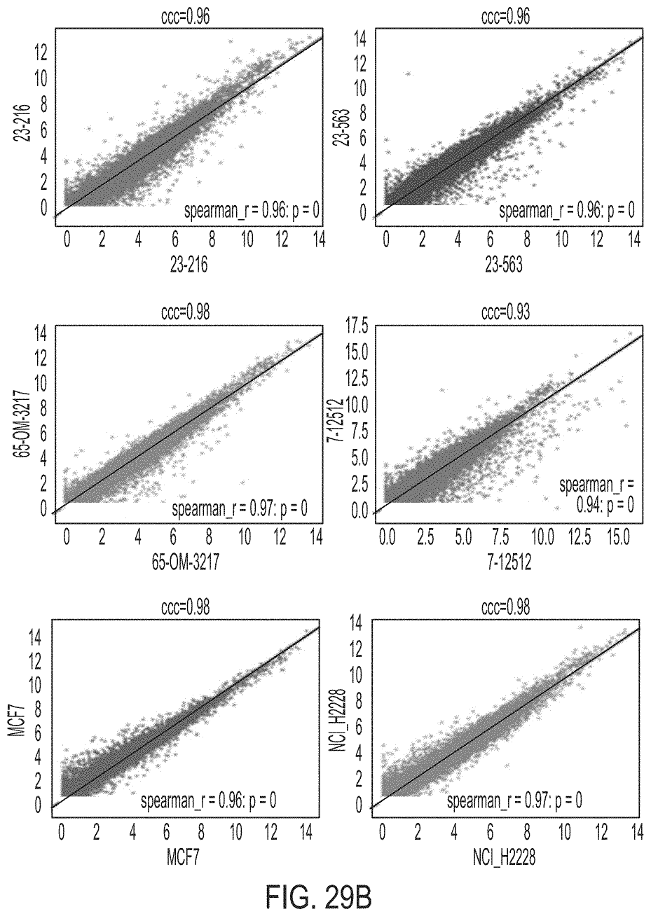

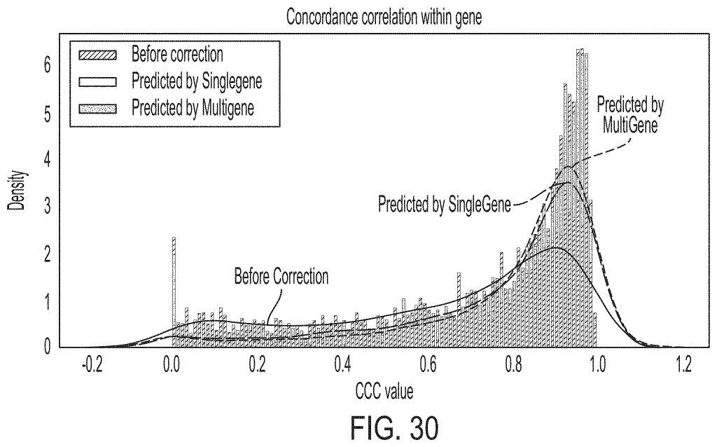

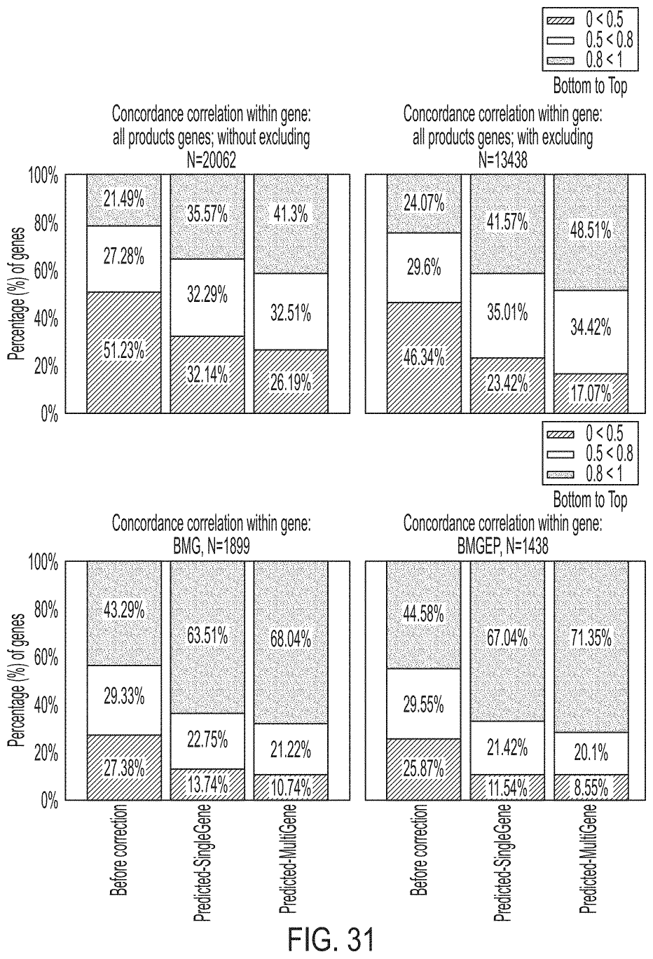

According to the PCA projections, the batch effect significantly decreased when these models were applied so that pairs of Poly-A and “corrected” EC samples began grouping together (FIGS. 25-27). Also, intra-sample correlation dramatically increased (average ˜85% to average 95%) (FIGS. 28A-28B and FIGS. 29A-29B). Focusing on the BMG group, 1,416 of 1,900 genes had CCC above 0.75 (1,292 genes>0.8) after correction and 1,695 had CCC above 0.50 (FIG. 30 and FIG. 31).

Kassandra deconvolution was also performed and the CCC values in major cell types for both predicted and validation-polyA expression sets were calculated. FIG. 32 demonstrates a slight decrease in all cases except the “Fibroblasts” group, where CCC values significantly increased after correction.

TABLE 2
Examples of genes in the Biologically Meaningful Group (BMG).
BMG

| | |
|---|---|
| A1BG | NM_130786 |
| ABCA2 | XM_047422921; NM_001606; XM_006716996; NM_212533 |
| ABCA3 | NM_001089 |
| ABCB1 | NM_001348946; NM_001348944; NM_000927; NM_001348945 |
| ABCB4 | XM_011516308; XR_007060049; XM_011516309; NM_018849; |
| | XR_007060045; XR_007060046; XR_007060053; XM_047420477; |
| | XR_007060051; XR_007060055; XR_007060047; XM_047420476; |
| | XR_007060048; XM_047420475; NM_000443; NM_018850; |
| | XR_001744810; XR_007060050; XR_007060054; XR_007060052 |
| ABCB5 | NM_001163941; NM_001163993; NM_178559; NM_001163942 |
| ABCC1 | XM_047434140; XM_047434149; XM_047434132; XM_047434131; |
| | XM_047434143; XM_047434145; XM_047434154; NM_019898; |
| | NM_019902; XM_047434133; XM_047434141; XM_047434144; |
| | NM_019899; NM_019901; XM_011522497; XM_047434142; NM_004996; |
| | XM_047434148; NM_019862; NM_019900; XM_047434134; |
| | XM_047434136; XM_047434137; XM_047434147; XM_047434152; |
| | XM_017023237; XM_047434135; XM_047434138; XM_047434146; |
| | XM_047434151; XM_047434153 |
| ABCC10 | XM_011514974; XM_047419500; XM_047419494; XM_047419496; |
| | NM_001350518; NR_146762; XM_011514986; XM_047419501; |
| | XM_047419502; NM_033450; XM_011514985; XM_047419495; |
| | XR_007059383; NM_001198934; XM_047419497; XM_047419499; |
| | XR_007059384; XM_047419498 |
| ABCC11 | XM_017023802; NM_001370496; NM_032583; XM_017023798; |
| | XM_017023797; XM_017023800; XM_017023799; XR_007064925; |
| | XM_017023801; NM_001370497; XM_011523398; NM_145186; |
| | XM_024450475; XM_047434818; NM_033151 |
| ABCC2 | XM_047424598; XM_011539291; XM_017015675; XM_006717631; |
| | XR_945604; NM_000392; XM_006717630 |
| ABCC3 | NM_001144070; NM_003786; NM_020037; NM_020038 |
| ABCC4 | NM_001301829; XM_047430035; NM_005845; NM_001301830; |
| | NM_001105515; XM_047430034; XM_017020320 |
| ABCC5 | XM_017005493; XM_011512314; XM_047447099; NM_001320032; |
| | NM_005688; XM_005247059; XM_011512315; XM_047447098; |
| | XM_047447100; NM_001023587; NR_135125 |
| ABCG2 | NM_001348989; NM_001348985; NM_001348988; XM_017008852; |
| | NM_001348987; NM_001257386; XM_011532420; NM_001348986; |
| | NM_004827 |
| ABL1 | NM_005157; NM_007313 |
| ACACA | XM_006721853; XM_047435889; NM_198834; XM_005257267; |
| | XM_047435878; XM_047435885; XM_047435890; XM_047435894; |
| | NM_198837; NM_000664; XM_047435881; NM_198839; XM_011524701; |
| | XM_047435879; XM_047435882; XM_047435886; XM_047435891; |
| | XM_047435892; XM_047435893; NM_198838; XM_011524703; |
| | XM_011524704; XM_047435888; NM_198836; NM_198835; |
| | XM_047435884; XM_047435880; XM_047435883 |
| ACACB | XM_011538265; XM_047428764; NM_001093; XM_011538264; |
| | XM_047428768; XR_007063072; XM_006719367; XM_011538263; |
| | XM_017019252; XM_047428762; XM_047428765; XM_047428766; |
| | XM_047428767; XM_047428763 |
| ACAP1 | NM_014716; XM_047437152; XM_047437151; XM_047437150 |
| ACP5 | XM_047438944; NM_001111035; NM_001322023; NM_001611; |
| | NM_001111034; NM_001111036; XM_047438945; XM_005259938; |
| | XM_011528069 |
| ACRBP | NM_032489 |
| ACSM2A | XM_017022925; XM_017022923; NM_001010845; NM_001308169; |
| | NM_001308954; XM_047433592; XM_017022926; XM_017022924; |
| | XM_047433591; NM_001308172; XR_001751833 |
| ACSM2B | XM_017023205; XR_001751899; NM_001105069; XM_011545824; |
| | NM_182617; XM_047434056 |
| ACSM5 | NM_001324371; NM_001324372; NM_001324373; NM_017888 |
| ACTA2 | NM_001141945; NM_001320855; NM_001613 |
| ACVR1B | XM_047429857; NM_004302; XM_047429856; XM_011538967; |
| | XM_017020201; NM_020327; NM_020328; XM_011538966; |
| | XM_047429858 |
| ACVR1C | NM_001111032; NM_001111033; NM_001111031; NM_145259 |
| ACVR2A | NM_001278580; NM_001616; XM_047446292; NM_001278579 |
| ACVR2B | XM_017007515; XM_005265583; NM_001106; XM_017007516; |
| | XM_017007514 |
| ADAM10 | NM_001110; NM_001320570 |
| ADAM15 | NM_003815; NM_207191; NR_048577; NR_048578; NM_207197; |
| | NM_001261464; NM_207196; NM_207195; NR_048579; NM_001261465; |
| | NM_001261466; NM_207194 |
| ADAM17 | XM_047445610; XM_047445612; NM_001382778; NM_003183; |
| | NM_001382777; NM_021832; XM_047445611 |
| ADAM28 | XM_047421270; XM_005273382; XM_047421272; NM_021777; |
| | XM_006716273; XM_017012975; XM_047421273; XM_011544367; |
| | NM_001304351; XM_005273380; XM_011544368; XM_011544369; |
| | XM_011544371; NM_014265; XM_006716274; XM_017012974; |
| | NR_130709; XM_047421271; XM_047421274; NR_130710 |
| ADAMTS2 | XM_047417895; NM_021599; NM_014244; XM_047417896 |
| ADAMTS4 | NM_001320336; NM_005099; XM_047434904 |
| ADAMTS5 | XM_047440680; NM_007038 |
| ADAP2 | XM_024450834; NM_001346714; NR_144488; XM_024450832; |
| | XM_024450833; NM_001346712; XM_024450835; NM_001346716; |
| | NM_018404 |
| ADGRE1 | NM_001974; NM_001256254; XM_011527794; NM_001256252; |
| | NM_001256253; NM_001256255 |
| ADGRE2 | XM_047438731; XM_011527955; XM_047438726; NM_001271052; |
| | NM_152916; XM_011527953; XM_011527954; XM_047438720; |
| | XM_047438727; NM_152918; XM_017026727; XM_047438721; |
| | XM_047438733; XM_047438736; XM_011527952; XM_011527948; |
| | XM_011527951; XM_017026726; XM_047438722; XM_047438724; |
| | NM_152919; XM_011527949; XM_047438723; XM_047438725; |
| | XM_047438729; XM_047438730; XM_047438735; XM_047438732; |
| | NM_013447; NM_152917; NM_152920; XM_047438728; XM_047438734; |
| | NM_152921 |
| ADGRE3 | NM_032571; NM_152939; XR_001753772; XM_011528374; |
| | XM_047439546; NM_001289158; NM_001289159 |
| ADGRG3 | XM_047433782; XM_011522954; XM_047433781; XM_047433783; |
| | XM_005255842; XM_011522953; XM_047433780; XM_006721170; |
| | NM_001308360; NM_170776 |
| ADH1A | NM_000667 |
| ADH4 | NM_000670; NM_001306172; NM_001306171 |
| ADH6 | NR_132990; NM_001102470; NM_000672 |
| ADIPOQ | NM_004797; NM_001177800 |
| ADORA1 | NM_001048230; XM_047446499; NM_000674; NM_001365065; |
| | NM_001365066 |
| ADORA2A | NM_000675; NR_103544; NM_001278498; NM_001278499; |
| | NM_001278500; NR_103543; NM_001278497 |
| ADORA2B | XM_017024197; XM_011523661; XM_047435375; NM_000676; |
| | XM_047435374; XM_011523659; XM_047435373 |
| ADORA3 | NM_001302679; NM_000677; NM_001302678 |
| AES | NM_198970; XM_006722664; NM_001130; NM_198969 |
| AFF2 | NM_001170628; NM_001169125; NM_001169123; NM_002025; |
| | NM_001169122; NM_001169124 |
| AFM | NM_001133; XM_017007843; XM_017007844; XM_017007842 |
| AGR2 | XM_005249581; NM_006408 |
| AGXT | NM_000030 |
| AHSG | NM_001354571; NM_001354572; NM_001622; NM_001354573 |
| AIF1 | NM_001318970; NM_032955; NM_004847; NM_001623; XM_005248870 |
| AIFM1 | NM_001130846; NM_145812; NM_145813; NM_001130847; NM_004208; |
| | NR_132647 |
| AJUBA | NM_032876; NM_198086; NM_001289097 |
| AKR1C4 | NM_001818 |
| AKT1 | NM_001014432; XM_047431069; XM_047431075; NM_005163; |
| | XM_047431071; NM_001382430; XM_047431072; NM_001382433; |
| | NM_001014431; NM_001382431; XM_047431073; XM_047431074; |
| | XM_047431070; NM_001382432 |
| AKT1S1 | NM_001278159; NM_001278160; NM_001098633; NM_001098632; |
| | NM_032375 |
| AKT2 | XM_011526616; XM_047438397; NM_001626; XM_047438398; |
| | XM_047438403; XM_011526619; XM_047438399; XM_047438401; |
| | NM_001243027; XM_011526618; NM_001243028; NM_001330511; |
| | XM_011526614; XM_047438400; XM_047438402; XM_011526615 |
| AKT3 | XM_047420641; XM_011544014; XM_047415643; XM_047422391; |
| | XM_047424332; NM_001370074; NM_005465; XM_047417359; |
| | NM_001206729; XM_024446892; NM_181690; XM_024447938; |
| | XM_047419186 |
| ALB | NM_000477 |
| ALDH1A1 | NM_000689 |
| ALDH1L1 | NR_072979; NM_001270365; XM_017005614; XM_024453325; |
| | NM_012190; NM_144776; NM_001270364; XM_006713481; |
| | XM_011512355; XM_017005613 |
| ALDOB | NM_000035 |
| ALK | NM_004304; NM_001353765; XR_001738688 |
| ALKBH2 | NM_001205180; NM_001205179; NM_001001655; XM_005253836; |
| | XM_047428309; NM_001145374; XM_005253835; NM_001145375 |
| ALKBH3 | NM_139178 |
| AMBP | NM_001633 |
| AMER1 | NM_152424 |
| AMHR2 | XM_011538179; XM_011538184; XM_017019179; NM_020547; |
| | XM_011538178; XM_011538176; XM_047428700; XM_011538181; |
| | XM_011538185; NM_001164691; XM_011538174; XM_011538183; |
| | XM_011538186; NM_001164690; XM_011538173; XM_011538180; |
| | XM_024448938 |
| ANGPT1 | NM_001314051; NM_001199859; NM_001146; XM_047421699; |
| | NM_139290 |
| ANGPT2 | NM_001118888; NM_001386335; NM_001386337; NM_001118887; |
| | NM_001147; NM_001386336 |
| ANGPTL3 | NM_014495 |
| ANKRD55 | XM_047417710; NM_001039935; NM_024669 |
| ANXA1 | XM_017014657; NM_000700 |
| ANXA2R | NM_001014279; NM_001382352 |
| AOAH | NM_001177507; XM_011515335; XM_011515341; XM_011515336; |
| | XM_011515340; XM_011515342; XM_017012105; XM_011515333; |
| | XM_011515334; XM_047420297; NM_001177506; NM_001637; |
| | XM_011515338; XM_011515339; XM_017012104; XM_017012102 |
| APAF1 | XM_047428759; NM_013229; XM_047428758; NM_181868; NM_181869; |
| | XM_047428760; XM_017019250; NM_001160; NM_181861 |
| APC | NM_001354895; NM_001354896; NM_001354906; NM_001354899; |
| | NM_001354901; NM_000038; NM_001354902; NM_001354903; |
| | NM_001127510; NM_001354898; NM_001354900; NM_001354905; |
| | NM_001127511; NM_001354897; NM_001354904 |
| APCS | NM_001639 |
| APEX1 | NM_080648; NM_001244249; NM_001641; NM_080649 |
| APEX2 | NM_001271748; NM_014481 |
| APH1A | NR_045035; NM_016022; NR_045033; NM_001077628; NM_001243771; |
| | NM_001243772; XM_047422066; NR_045034 |
| APLN | NM_017413 |
| APOA1 | XM_047426866; NM_001318018; NM_001318017; NM_000039; |
| | NM_001318021 |
| APOA2 | NM_001643 |
| APOA5 | NM_001371904; NM_001166598; NM_052968 |
| APOB | NM_000384 |
| APOBEC3A | NM_145699.4; NM_001270406.2 |
| APOBEC3D | NM_145699; NM_001270406 |
| APOBEC3G | NM_001349436; NM_001349437; NR_146179; NM_021822; NM_001349438 |
| APOC2 | NM_000483 |
| APOC3 | NM_000040 |
| APOC4 | NM_001646 |
| APOC4- | NR_037932.1 |
| APOC2 | |
| APOH | NM_000042 |
| APOM | XM_006715150; NM_019101; NM_001256169; NR_045828 |
| AQP1 | NM_001185061; NM_198098; NM_000385; NM_001329872; |
| | NM_001185062; NM_001185060 |
| AR | NM_001348064; NM_001011645; NM_001348063; NM_001348061; |
| | NM_000044 |
| ARAF | NM_001256196; NM_001256197; NM_001654 |
| ARAP1 | NM_001369489; NM_015242; NM_001135190; NR_161388; NM_001040118 |
| AREG | NM_001657 |
| ARG1 | NM_001369020; NM_000045; NM_001244438; NR_160934 |
| ARHGAP15 | NM_018460; XM_011511482; XM_024453000; XM_017004500; |
| | XM_011511483; XM_047445110; XM_047445112; XR_007078554; |
| | XM_011511484; XM_047445109; XM_047445111; XM_047445114; |
| | XM_047445113 |
| ARHGAP24 | NM_001025616; XM_047416235; NM_001042669; NM_001346093; |
| | XM_011532300; XM_024454238; NM_001287805; NM_031305 |
| ARHGAP30 | NM_001025598; NM_001287602; XM_005245070; NM_001287600; |
| | NM_181720; XM_011509391; XM_047417140; XM_005245073 |
| ARHGAP9 | XM_011538656; XM_011538659; XM_047429340; XM_047429337; |
| | XM_047429339; NM_001367422; NM_001367424; XM_047429334; |
| | NM_001367423; NM_001367425; NM_001367426; NM_001319851; |
| | XM_047429329; XM_047429332; XM_047429333; NM_001319852; |
| | XM_005269083; NM_001080157; NM_001319850; XM_047429330; |
| | XM_047429336; XM_047429335; NM_001080156; XM_047429331; |
| | XM_047429338; NM_032496 |
| ARHGDIB | NM_001175; NR_135637; NM_001321423; NM_001321421; |
| | NM_001321420; NM_001321422 |
| ARID1A | NM_018450; NM_006015; NM_139135 |
| ARNTL | NM_001297719; NM_001351819; NR_147787; NR_147790; |
| | XM_024448522; NM_001351808; NM_001351810; NM_001351813; |
| | NM_001351824; NR_147785; XM_047426958; NM_001351804; |
| | NM_001351805; NM_001351807; NM_001351811; NM_001351814; |
| | NM_001351817; NM_001351818; XM_011520107; NM_001351821; |
| | XM_017017739; XM_047426952; XM_047426953; XM_047426954; |
| | NM_001030272; NM_001297722; NM_001297724; NM_001351823; |
| | NM_001030273; NM_001351806; NM_001351809; NM_001351812; |
| | NM_001351815; NM_001351816; NM_001351822; NR_147786; |
| | XM_011520105; XM_011520109; XM_017017738; XM_047426955; |
| | XM_047426956; XM_047426957; NM_001351820; NR_147788; |
| | XM_017017741; NM_001178; NR_147789; NR_147791 |
| ARRDC1 | XM_006717320; XM_047424069; NM_001317968; NM_152285; |
| | XM_006717322; XM_006717321; XM_005266119; XM_017015288 |
| ASGR1 | XM_011523861; NM_001197216; NM_001671 |
| ASGR2 | XM_006721524; XM_017024651; XM_024450755; NM_080913; |
| | XM_024450757; NM_001201352; NM_080912; XM_011523863; |
| | NM_080914; XM_006721526; XM_011523862; XM_011523864; |
| | NM_001181; XM_024450756; XM_047436089; XM_047436090; |
| | XM_047436091 |
| ASPDH | XM_047439063; XM_047439061; XM_047439062; NM_001114598; |
| | NM_001024656; XM_024451585 |
| ATF3 | XM_047421211; NM_001206488; NM_001674; NM_001206484; |
| | NM_004024; XM_005273146; NM_001040619; NM_001206486; |
| | NM_001030287; XM_011509579; NM_001206485 |
| ATG12 | NM_004707; NM_001277783; NR_033362; NR_073603; NR_033363; |
| | NR_073604 |
| ATG13 | NM_001346319; NM_001346328; NM_001346352; NM_001346312; |
| | NM_001346317; NM_001346323; NM_001346325; NM_001346329; |
| | NM_001346335; NM_001346336; NM_001346338; NM_001346357; |
| | NM_001346360; NM_001346359; NR_144424; NM_001346314; |
| | NM_001346318; NM_001346333; NM_001346340; NM_001346351; |
| | NM_001346353; NM_001205119; NM_001346315; NM_001346322; |
| | NM_001346326; NM_001346346; NR_144422; NM_001205122; |
| | NM_001346311; NM_001346320; NM_001346321; NM_001346330; |
| | NM_001346350; NM_001346358; NM_001142673; NM_001205121; |
| | NM_001346324; NM_001346334; NM_001346349; NM_001346356; |
| | NM_014741; NR_144423; NM_001205120; NM_001346313; |
| | NM_001346327; NM_001346342; NM_001346344; NM_001346354; |
| | NM_001346316; NM_001346332; NM_001346331; NM_001346337; |
| | NM_001346348; NM_001346355 |
| ATG14 | NM_014924; XM_011536563 |
| ATG4B | XM_047443739; XM_047443738; XM_047443742; XM_047443741; |
| | XM_005246992; NM_178326; XM_047443740; NM_013325 |
| ATG5 | NM_001286108; NM_004849; NM_001286107; XM_047419574; |
| | NR_104403; NM_001286106; XM_024446590; XM_047419573; |
| | NM_001286111; NR_104402 |
| ATG7 | XM_024453312; XM_047447296; XM_047447309; NM_001349238; |
| | XM_006712931; XM_047447294; XM_047447298; XM_047447302; |
| | NM_001349234; XM_017005543; XM_017005550; XM_047447293; |
| | XM_047447307; NM_001349233; XR_007095622; XR_007095623; |
| | XM_047447304; XM_047447306; NM_001349232; XM_047447299; |
| | XM_047447308; NM_001349237; NM_006395; XM_017005542; |
| | XM_011533277; XM_047447297; XM_047447301; XM_047447305; |
| | NM_001349236; XM_047447300; NM_001349235; XM_006712932; |
| | XM_017005548; XM_017005551; XM_047447295; XM_047447303; |
| | NM_001136031; NM_001144912 |
| ATG9A | NM_001077198; NR_104255; NM_024085 |
| ATG9B | NM_001317056; XM_011516065; XR_002956421; NR_073169; NR_133652; |
| | XR_007060009 |
| ATM | XM_011542844; XM_047426976; XM_047426978; NM_001351834; |
| | XM_011542840; XM_011542842; XM_047426975; NM_138293; |
| | XM_005271562; XM_006718843; XM_047426979; NM_000051; |
| | NM_001351835; XM_006718845; XM_047426981; NM_001351836; |
| | XM_011542843; XM_017017790; XM_047426977; NM_138292 |
| ATP7A | NM_000052; NR_104109; NM_001282224 |
| ATP7B | XM_047430388; XM_005266424; XM_047430393; NM_000053; |
| | XM_006719837; XM_047430386; XM_047430385; XM_047430392; |
| | NM_001330578; XM_005266423; XM_011535117; XM_005266431; |
| | XM_047430389; XM_047430391; NM_001005918; NM_001330579; |
| | XM_005266430; XM_017020627; XM_047430387; XM_047430390; |
| | NM_001243182 |
| ATR | XM_047448362; XM_011512925; NM_001354579; XM_011512924; |
| | XM_047448361; XM_047448363; NM_001184; XM_047448364; |
| | XM_047448360 |
| ATRIP | NM_001271023; NM_032166; NM_130384; NM_001271022 |
| ATRX | XM_005262153; XM_017029601; XM_005262154; XM_006724668; |
| | XM_005262156; NM_138271; XM_047442191; NM_138270; |
| | XM_005262157; XM_017029604; XM_006724666; NM_000489 |
| AURKA | XM_047440428; XM_047440427; NM_001323304; NM_001323303; |
| | NM_198435; NM_198437; NM_198433; NM_198434; NM_198436; |
| | XM_017028034; XM_017028035; NM_001323305; NM_003600 |
| AURKB | NM_001313950; NM_001313953; XM_017025311; XM_047437050; |
| | NM_001313952; NM_004217; NM_001313954; NR_132730; NR_132731; |
| | NM_001284526; XM_011524072; XM_047437051; NM_001256834; |
| | NM_001313951; NM_001313955 |
| AXIN1 | XM_011522682; XM_011522686; XM_017023747; XM_047434731; |
| | NM_003502; NM_181050; XM_017023745; NR_134879; XR_001751996; |
| | XM_011522683; XM_017023746; XM_047434732; XM_017023748 |
| AXIN2 | XM_011525319; XM_047436873; XM_011525320; NM_001363813; |
| | XM_017025192; XM_047436871; XM_047436872; XM_017025193; |
| | XM_011525321; XM_047436870; XM_047436874; NM_004655 |
| AXL | NM_001699; NM_021913; NM_001278599 |
| B2M | XM_005254549; NM_004048 |
| B3GAT1 | XM_017017551; XM_024448439; XM_047426772; XM_047426773; |
| | NM_001367973; NM_018644; XM_011542753; XM_047426771; |
| | NM_054025 |
| BAAT | NM_001127610; NM_001701; NM_001374715 |
| BACH1 | NM_206866; NM_001011545; NR_027655; NM_001186 |
| BACH2 | NM_001170794; NM_021813 |
| BAK1 | XM_011514779; NM_001188; XM_047419194; XM_047419196; |
| | XM_011514780 |
| BANK1 | NM_001127507; NM_001083907; NM_017935 |
| BAP1 | XM_011534149; XM_011534150; XM_017007303; XM_011534151; |
| | XM_047449044; XM_011534152; NM_004656 |
| BATF | NM_006399 |
| BATF3 | XR_001737289; NM_018664 |
| BAX | NM_001291429; NM_138763; NM_001291428; NM_001291430; |
| | NR_027882; XM_047439168; NM_004324; NM_001291431; NM_138761; |
| | NM_138764; NM_138762 |
| BBC3 | XM_006723141; XM_011526722; NM_001127241; NM_001127242; |
| | NM_001127240; NM_014417; XM_047438606 |
| BCHE | NR_137636; NM_000055; NR_137635 |
| BCL10 | NM_001320715; NM_003921; XM_011542398; XM_011542399; |
| | XM_011542397 |
| BCL11A | XM_047444756; NM_001405721; NM_001405728; XM_047444761; |
| | NM_001365609; NM_001405718; NM_001405724; NM_022893; |
| | NM_138553; XM_017004335; XM_024452962; NM_001405709; |
| | NM_001405714; NM_001405715; NM_001405722; NM_001405726; |
| | NM_001405735; NM_001405736; NR_175964; NM_001405723; |
| | XM_024452963; XM_047444760; NM_001405708; NM_001405711; |
| | XM_017004333; XM_047444757; XM_047444758; XM_047444759; |
| | NM_001405712; NM_001405719; NM_001405732; NM_138559; |
| | XM_017004336; XM_011532910; NM_001405713; NM_001405731; |
| | NM_001405733; NM_018014; NM_001363864; NM_001405717; |
| | NM_001405720; NM_001405730; NM_001405734; NM_001405710; |
| | NM_001405716; NM_001405725; NM_001405727; NM_001405729 |
| BCL2 | XM_047437734; NM_000657; XM_011526135; XM_017025917; |
| | NM_000633; XM_047437733 |
| BCL2A1 | NM_001114735; NM_004049 |
| BCL2L1 | XM_047440353; NM_001317919; NM_001322240; NM_001322242; |
| | XM_011528964; XM_047440351; NM_001191; NM_001317920; |
| | NR_134257; XM_017027993; NM_001317921; NM_138578; |
| | XM_047440352; NM_001322239 |
| BCL2L11 | NM_001204107; NM_138621; NM_138622; NM_207003; XR_007068189; |
| | XM_005263553; XM_047442104; NM_138624; NM_138625; NM_138626; |
| | XM_005263559; XM_011510461; NM_138627; XR_007068187; |
| | XM_005263552; XM_005263557; XM_017003101; XM_047442101; |
| | NM_006538; NM_207002; XM_047442102; NM_001204108; |
| | NM_001204113; NM_138623; XM_011510464; XM_047442099; |
| | NM_001204109; NM_001204112; XM_005263555; NM_001204106; |
| | NM_001204110; NM_001204111; XM_005263556 |
| BCL2L2 | NM_004050; NM_001199839 |
| BCL6 | NM_001130845; XM_011513062; NM_001706; XM_047448655; |
| | NM_001134738; NM_138931; XM_005247694 |
| BCR | NM_004327; NM_021574 |
| BECN1 | XM_017025263; XM_005257760; NM_001314000; XM_017025264; |
| | NM_001313998; NM_001313999; NM_003766; XM_005257759 |
| BHMT | NM_001713 |
| BHMT2 | NM_017614; NM_001178005 |
| BID | NM_001244567; NM_001196; NM_001244569; NM_197967; |
| | NM_001244572; NM_197966; NM_001244570 |
| BIRC2 | NM_001256166; NM_001256163; NM_001166 |
| BIRC3 | NM_001165; NM_182962 |
| BIRC5 | NM_001168; NM_001012270; NM_001012271 |
| BLK | XM_047422081; NM_001330465; XM_011543829; XM_011543824; |
| | XM_011543827; XM_047422083; XM_047422084; XM_011543828; |
| | XM_047422082; NM_001715; XM_011543825 |
| BLM | NM_001287247; NM_001287248; XM_006720632; XM_047432934; |
| | XM_011521882; NM_000057; NM_001287246 |
| BLNK | NR_047682; NM_001114094; NM_001258441; NM_013314; NR_047681; |
| | NM_001258440; NM_001258442; NR_047680; NR_047683 |
| BMI1 | NM_005180.9 |
| BMP10 | NM_014482 |
| BMP15 | NM_005448 |
| BMP2 | NM_001200 |
| BMP3 | NM_001201; XM_006714291 |
| BMP4 | NM_001347915; NM_001347916; NM_130851; NM_001347917; |
| | NM_130850; NM_001202; NM_001347914; NM_001347912; |
| | NM_001347913 |
| BMP5 | XM_011514817; NM_001329756; XM_024446524; NM_001329754; |
| | NM_021073 |
| BMP6 | NM_001718 |
| BMP7 | NM_001719 |
| BMP8B | XM_024449299; XR_946748; XR_946749; XM_017002155; XR_946750; |
| | XM_011542025; XM_005271149; XM_017002156; NM_001720; |
| | XM_011542024; XM_011542022 |
| BMPR1A | XM_011540104; XM_047425676; NM_004329; XM_047425680; |
| | XM_047425678; XM_047425679; XM_011540103; XM_047425677 |
| BMPR1B | XM_017008558; XM_047416095; NM_001203; NM_001256793; |
| | XM_011532201; XM_047416093; NM_001256794; NM_001256792; |
| | XM_017008559; XM_047416091; XM_017008560; XM_047416094 |
| BMPR2 | NM_001204; NM_033346; XM_011511687 |
| BNIP3 | NM_004052 |
| BRAF | XM_047420766; XM_047420768; NM_001374244; NM_001374258; |
| | NM_001378471; NM_001378473; NR_148928; XM_047420767; |
| | XM_047420769; XM_047420770; NM_001378467; NM_001378468; |
| | XM_017012559; NM_001378470; NM_001378472; NM_001378475; |
| | NM_001354609; NM_001378469; NM_001378474; NM_004333 |
| BRCA1 | NM_007299; NM_007303; NM_007294; NM_007306; NM_007298; |
| | NM_007295; NM_007301; NM_007300; NR_027676; NM_007305; |
| | NM_007296; NM_007297; NM_007302 |
| BRCA2 | NM_000059 |
| BRD4 | XM_047438540; XM_047438541; XM_047438542; XM_047438543; |
| | NM_001379292; XM_047438544; NM_001379291; NM_014299; |
| | NM_058243; NM_001330384; XR_007066712 |
| BRD7 | XM_017023180; NM_001173984; XM_011523047; NM_013263; |
| | XM_011523049; XM_011523046; XM_017023179; XM_047434006; |
| | XM_047434007; XM_047434008; XM_011523048; XM_011523050 |
| BRMS1 | XM_024448425; XM_024448426; NM_015399; NM_001024958; |
| | NM_001024957 |
| BTG1 | NM_001731 |
| BTG2 | NM_006763 |
| BTG3 | XM_011529441; NM_006806; XM_047440672; XM_047440670; |
| | XM_047440671; NM_001130914; XM_047440673 |
| BTK | NM_001287344; NM_001287345; NM_000061 |
| BTLA | NM_001085357; NM_181780; XM_011512447; XM_017005748; |
| | XM_047447496 |
| BUB1 | NM_004336; NM_001278617; XM_047445616; NM_001278616 |
| C10orf54 | NM_022153 |
| C15orf48 | NM_032413; NM_197955 |
| C1orf54 | XM_047430455; NM_001301042; NM_001301039; XM_047430461; |
| | NM_024579; XM_047430446; NM_001301040; NM_001301041 |
| C1QA | NM_001347465; NM_001347466; NM_015991 |
| C1QC | NM_001114101; NM_001347619; NM_001347620; NM_172369 |
| C3AR1 | NM_004054; NM_001326475; NM_001326477 |
| C4BPB | XM_047429635; NM_000716; NM_001017367; XM_005273254; |
| | NM_001017366; XM_005273255; XM_024449464; NM_001017365; |
| | NM_001017364 |
| C5AR1 | XM_047439300; NM_001736 |
| C8A | NM_000562; XM_011542079; XM_017002234 |
| C8B | NM_000066; NM_001278544; XM_047429957; NM_001278543 |
| C8G | XM_047423843; NM_000606 |
| C9 | NM_001737 |
| CA9 | XM_047423849; NM_001216; XM_047423850 |
| CADM1 | XM_047426693; NM_001098517; XM_017017457; XM_047426690; |
| | XM_047426694; NM_001301043; NM_001301045; XM_047426692; |
| | XM_047426695; NM_001301044; XM_005271494; NM_014333; |
| | XM_047426691 |
| CADM3 | NM_001346510; XM_024448760; NM_021189; NM_001127173 |
| CALR | NM_004343 |
| CAMK4 | NM_001744; NM_001323376; XR_948303; NM_001323375; |
| | XM_047417783; NM_001323374; NM_001323377 |
| CANX | XM_011534665; XM_047417792; NM_001746; NM_001363998; |
| | NR_157048; NM_001024649; NM_001363997; NM_001364000; |
| | NM_001363996; NM_001363993; NM_001363994; NM_001364001; |
| | NM_001363995; NM_001363999 |
| CARD11 | NM_032415; NM_00 1324281 |
| CASP3 | XM_047416237; NM_032991; NM_001354783; XM_047416239; |
| | NM_001354779; NM_001354780; NM_004346; XM_047416236; |
| | NM_001354782; NM_001354784; XM_047416238; NM_001354777; |
| | NM_001354781 |
| CASP8 | NM_001080124; NM_001080125; NM_001400651; NM_001400653; |
| | NM_001400669; NM_033355; NR_174599; NM_033358; NM_001400642; |
| | NM_001400665; NM_001400751; NR_174581; NR_174588; NR_174593; |
| | NR_174602; NM_001400663; NM_001400666; NM_001400667; |
| | NM_001400676; NM_001400680; NR_174584; NR_174589; NR_174600; |
| | NM_033357; XM_011511969; NM_001372051; NM_001400645; |
| | NM_001400660; NM_001400662; NM_001400671; NR_174592; |
| | NR_174594; XM_005246893; XM_047445959; XM_047445961; |
| | NM_001400654; NM_001400658; NM_001400659; NM_001400661; |
| | NM_001400668; NM_001400670; NM_001400672; NR_174565; |
| | NR_174583; NR_174585; NR_174586; NR_174598; NM_001228; |
| | NM_001400648; NM_001400655; NM_001400664; NM_001400674; |
| | NM_001400679; NM_033356; NR_174564; NR_174590; NR_174601; |
| | NM_001400673; NM_001400675; NM_001400677; NM_001400678; |
| | NM_001400750; NR_111983; NR_174591; NR_174595; XM_047445960; |
| | NM_001400656; NM_001400657; NR_174582; NR_174596; XR_007082538; |
| | XR_007082539 |
| CASP9 | XM_005246014; XM_011542273; NM_001229; NM_001278054; |
| | NR_102732; NR_102733; XM_047432034; NM_032996; XR_007064158 |
| CAV1 | NM_001753; NM_001172895; NM_001172897; NM_001172896 |
| CBFB | NM_001755; NM_001368707; NM_001368709; NM_022845; |
| | NM_001368708; NM_001368710 |
| CBL | NM_005188 |
| CBLB | XM_017007396; XM_047449114; XM_047449117; NM_001321798; |
| | NM_001321806; NM_001321813; NR_135811; XM_017007397; |
| | NM_001321791; NM_001321795; NR_135806; XM_011513257; |
| | NM_001321789; NM_001321808; XM_017007395; XM_047449112; |
| | XM_047449113; NM_001321788; NM_001321799; NM_001321807; |
| | XR_007095761; XR_007095762; XM_011513259; NM_001321790; |
| | NM_001321797; NM_001321820; NM_170662; NR_135807; NR_135812; |
| | NM_004351; XM_047449115; NM_001321811; NR_135809; NR_135810; |
| | XM_017007400; XM_047449116; XM_047449118; NM_001321794; |
| | NM_001321796; NM_001321816; NR_135808; XM_017007398; |
| | NM_001321786; NM_001321793; NM_001321822 |
| CBX4 | NM_003655; XM_011525399 |
| CCDC69 | NM_015621 |
| CCL1 | NM_002981 |
| CCL11 | NM_002986 |
| CCL13 | NM_005408 |
| CCL15 | NM_004167; NM_032965; NM_032964 |
| CCL17 | XM_047434448; XM_011523256; NM_002987; XM_017023530 |
| CCL18 | NM_002988 |
| CCL19 | NM_006274 |
| CCL2 | NM_002982 |
| CCL20 | NM_001130046; NM_004591 |
| CCL21 | NM_002989 |
| CCL22 | XM_047434450; XM_047434449; NM_002990 |
| CCL24 | XM_011516460; NM_002991; NM_001371193 |
| CCL26 | NM_006072; NM_001371936; NM_001371938 |
| CCL28 | NM_001301874; NM_001301873; NM_019846; XR_007058611; |
| | XR_007058613; XR_925633; NM_148672; NM_001301875; XR_007058610; |
| | XR_007058612; XR_427660; XR_241706 |
| CCL3 | NR_168496; NR_168495; NM_002983; NR_168494 |
| CCL3L3 | NM_001001437.4 |
| CCL4 | NM_002984.4 |
| CCL4L2 | NM_001291475.2; NM_001291468.2; NM_001291469.2; NM_001291470.2; |
| | NM_001291471.2; NM_001291472.2; NM_001291473.2; NM_001291474.2; |
| | NR_111970.2 |
| CCL5 | NM_001278736; NM_002985 |
| CCL7 | NM_006273 |
| CCL8 | NM_005623 |
| CCNB1 | NM_001354844; NM_031966; NM_001354845 |
| CCND1 | NM_053056 |
| CCND2 | NM_001759 |
| CCND3 | XM_047419491; NM_001287434; NM_001136017; NM_001760; |
| | NM_001136125; NM_001136126; XM_011514971; NM_001287427 |
| CCNE1 | XM_011527440; NM_001238; NM_001322259; NM_001322261; |
| | XM_047439606; NM_001322262; NM_057182 |
| CCNE2 | XM_047422411; XM_017013958; NM_057749; XM_011517366; |
| | XM_017013959; NM_004702; NM_057735 |
| CCNH | NR_157070; NM_001363539; NR_157071; NM_001239; NM_001364076; |
| | XM_047417863; NM_001199189; NR_157068; NR_157069; NM_001364075 |
| CCR1 | NM_001295 |
| CCR10 | NM_001296 |
| CCR2 | NM_001123041; NM_001123396 |
| CCR3 | NM_001164680; NM_001837; NM_178328; NM_178329; XM_017005685; |
| | XM_006712960 |
| CCR4 | XM_017005687; NM_005508 |
| CCR5 | NM_001100168; NM_001394783; NM_000579 |
| CCR6 | NM_001394582; NM_031409; NM_004367 |
| CCR7 | NM_001301716; NM_001301717; NM_001838; NM_001301718; |
| | NM_001301714 |
| CCR8 | NM_005201 |
| CD101 | XM_047434715; NM_004258; NM_001256109; NM_001256106; |
| | NM_001256111; XM_047434718 |
| CD14 | NM_001040021; NM_000591; NM_001174105; NM_001174104 |
| CD160 | NM_007053; XM_005272929; XM_011509104; NR_103845 |
| CD163 | XM_047429895; XM_024449278; NM_203416; NM_001370145; |
| | NM_001370146; NM_004244; NR_163255 |
| CD177 | XM_017027021; XM_017027022; NM_020406 |
| CD19 | NM_001178098; NM_001385732; NM_001770; XR_950871; NR_169755; |
| | XM_011545981 |
| CD1A | NM_001320652; NM_001763; XM_024450738 |
| CD1C | XM_005245579; NM_001765 |
| CD1D | NM_001371762; XM_047433945; NM_001371761; NM_001319145; |
| | NM_001371763; NM_001766 |
| CD1E | NM_001185113; XM_011510133; NM_001185115; XM_047434054; |
| | NM_001042583; NM_001185108; NM_001185110; NM_001185114; |
| | XM_047434055; NM_001042584; NM_001185107; NM_001185112; |
| | NM_030893; XM_047434050; XM_047434051; NM_001042585; |
| | NM_001042586; NM_001042587 |
| CD2 | NM_001328609; NM_001767 |
| CD200 | NM_001318830; NR_158642; NM_001004197; NM_001365853; |
| | NM_001365855; NM_001318826; NM_001365852; NM_001004196; |
| | NM_001318828; NM_001365851; NM_005944; NM_001365854 |
| CD207 | XM_011532876; XM_011532875; NM_015717 |
| CD209 | NR_026692; NM_001144895; NM_001144894; NM_001144893; |
| | NM_021155; NM_001144896; NM_001144897; NM_001144899 |
| CD22 | NM_001185100; NM_001185099; NM_024916; NM_001185101; |
| | NM_001771; NM_001278417 |
| CD226 | NM_006566; XM_047437274; NM_001303619; XM_047437275; |
| | XM_047437276; XM_006722374; XM_005266642; XM_047437277; |
| | NM_001303618 |
| CD24 | NM_001291739; NR_117090; NR_117089; NM_001291738; |
| | NM_001291737; NM_013230; NM_001359084 |
| CD244 | NM_001166663; XM_047422535; XM_011509622; NM_016382; |
| | NM_001166664; XM_011509623; XM_011509621 |
| CD247 | NM_001378516; NM_198053; XM_011510144; XM_011510145; |
| | NM_000734; NM_001378515 |
| CD248 | NM_020404 |
| CD27 | NM_001242; XM_011521042; XM_017020234; XM_047429900 |
| CD274 | XM_047423262; NM_001314029; NM_001267706; NR_052005; NM_014143 |
| CD276 | XM_005254700; XM_047433148; XM_047433147; NM_025240; |
| | XM_017022638; NM_001329628; XM_011522095; NM_001024736; |
| | NM_001329629 |
| CD28 | NM_006139; NM_001243078; NM_001243077; XM_011512194 |
| CD300A | XM_005256991; NM_001330457; NM_001330456; XM_005256990; |
| | NM_007261; NM_001256841 |
| CD300C | NM_006678; XM_017024033; XM_047435157 |
| CD300E | NM_181449 |
| CD300LB | NM_174892; XM_005257027 |
| CD302 | NM_014880; NM_001198764; NM_001198763 |
| CD33 | NM_001082618; XM_017027509; XM_047439730; XM_017027510; |
| | NM_001772; XM_011527532; XM_017027508; XM_047439729; |
| | XM_011527531; XM_047439728; XM_047439731; NM_001177608; |
| | XM_047439732; XR_007067046 |
| CD36 | XM_024447002; NM_000072; NM_001289909; NM_001371081; |
| | NR_110501; NM_001001548; NM_001127443; XM_005250715; |
| | NM_001371074; XM_047421045; NM_001001547; XM_047421042; |
| | XM_047421043; XM_047421044; XM_047421048; XM_047421049; |
| | NM_001371075; XM_047421046; XM_047421047; NM_001127444; |
| | NM_001371077; NM_001371078; NM_001371079; NM_001371080; |
| | XM_024447003; XM_047421041; NM_001289908; NM_001289911 |
| CD37 | XM_005259435; XM_011527542; NM_001774; XM_011527543; |
| | NM_001040031 |
| CD38 | NM_001775; NR_132660 |
| CD3D | NM_001040651; NM_000732 |
| CD3E | NM_000733 |
| CD3G | XM_005271724; XM_006718941; NM_000073 |
| CD4 | NM_001195014; NM_001382707; NM_001382714; NM_001195016; |
| | NM_000616; NR_036545; NM_001195015; NM_001382705; |
| | NM_001195017; NM_001382706 |
| CD40 | NM_001302753; NM_001322422; NM_152854; NM_001322421; |
| | NM_001362758; NM_001250; XM_047440601; NR_136327; |
| | XM_011529109; XM_005260619; XM_017028135; XM_017028136; |
| | NR_126502 |
| CD40LG | NM_000074 |
| CD46 | XM_047420901; XM_011509563; NM_172351; NM_172359; NM_172352; |
| | NM_172361; NM_002389; NM_172355; NM_172358; XM_047420909; |
| | NM_153826; NM_172353; XM_047420888; XM_047420894; NM_172356; |
| | NM_172354; NM_172360; NM_172350; NM_172357 |
| CD48 | XM_017002867; XM_047435011; NM_001778; XM_005245625; |
| | NM_001256030 |
| CD5 | NM_014207; NM_001346456 |
| CD53 | NM_000560; NM_001040033; XM_047435014; XM_047435015; |
| | NM_001320638; XM_047435013 |
| CD55 | NM_001300904; NM_001114543; NM_001114544; NM_001114752; |
| | NM_001300902; NM_001300903; XR_007095644; NM_000574; NR_125349 |
| CD58 | NM_001779; NM_001144822; NR_026665 |
| CD59 | NM_000611; NM_203330; NM_001127223; NM_203331; NM_001127227; |
| | NM_001127225; NM_001127226; NM_203329 |
| CD6 | XM_047427875; XM_047427876; XM_047427879; XM_011545360; |
| | XM_047427878; XM_047427881; NM_001254750; NM_001254751; |
| | NM_006725; NR_045638; XM_006718738; XM_006718739; |
| | XM_047427877; XM_006718740; XM_011545362; XM_047427874; |
| | XM_047427880 |
| CD63 | NM_001267698; NM_001257389; NM_001257400; XM_024449283; |
| | NM_001780; NM_001257401; NM_001257391; NM_001257390; |
| | NM_001257392; NM_001040034 |
| CD68 | NM_001251; NM_001040059 |
| CD69 | NR_026672; NR_026671; NM_001781 |
| CD7 | NM_006137 |
| CD70 | NM_001252; NM_001330332 |
| CD72 | XM_047424157; XM_006716893; XM_047424154; NM_001782; |
| | XM_047424155; XM_047424156 |
| CD74 | NM_001364083; NM_001364084; NR_157074; NM_001025159; |
| | NM_001025158; NM_004355 |
| CD79A | NM_021601; NM_001783 |
| CD79B | NM_001039933; NM_021602; NM_000626; NM_001329050 |
| CD80 | NM_005191 |
| CD81 | XM_047427932; XM_047427931; XM_047427933; XM_047427934; |
| | NM_001297649; NM_004356 |
| CD82 | XM_011520067; XM_047426901; XM_047426904; XM_047426900; |
| | NM_002231; XM_047426903; NM_001024844 |
| CD83 | NM_001040280; NM_001251901; NM_004233 |
| CD84 | NM_003874; XM_011510095; NM_001184882; NM_001330742; |
| | NM_001184881; NM_001184879; XR_921991 |
| CD86 | NM_001206924; NM_006889; NM_176892; NM_001206925; NM_175862 |
| CD8A | NM_001145873; NM_001382698; NM_001768; NR_168478; NR_168479; |
| | NM_171827; NR_168480; NR_168481; NR_027353 |
| CD8B | NM_172102; NM_172100; NM_001178100; NM_004931; NM_172101; |
| | NM_172213; NM_172099; XM_011533164 |
| CD9 | NM_001769; NM_001330312; XM_005253814 |
| CD96 | XR_007093316; NR_134917; XR_007093335; XR_241462; XM_006713470; |
| | NM_005816; XR_924090; XM_006713469; NM_198196; XR_007093273; |
| | XR_007093326; XR_007093307; XM_047447184; NM_001318889; |
| | XR_001739977; XR_007093366 |
| CDC25A | XM_047449366; NM_001789; XM_047449364; XM_047449367; |
| | XM_047449363; XM_047449365; NM_201567 |
| CDC6 | XM_047437207; XM_011525542; XM_011525541; NM_001254 |
| CDC73 | NM_024529; XM_006711537 |
| CDCA7 | NM_031942; NM_145810; XM_047445957 |
| CDH1 | NM_001317186; NM_004360; NM_001317185; NM_001317184 |
| CDH10 | XR_001753699; XM_047438909; XM_017026863; NM_001136197; |
| | XM_005259573; XM_047438908; NM_016263; NM_001136198 |
| CDH11 | NM_001317222; NM_001190450; NM_001317224; NM_001362460; |
| | XM_011513923; NM_006727 |
| CDH12 | NM_001308392; NM_001797; XM_047433486; NM_001330576; |
| | NM_033664 |
| CDH13 | NM_001317227; NM_001364105; NM_001364106; NM_001364104; |
| | NM_004061; NM_001317228; NM_001364109; NM_001364107; |
| | NM_001364108; XM_047416602 |
| CDH15 | XM_017022849; NM_001220488; XM_017022848; NM_001220492; |
| | NM_001220491; NM_001257; NM_001220489; XM_011522804; |
| | NM_001220490 |
| CDH16 | XM_047433490; NM_001204746; XM_011522807; NM_004062; |
| | XM_005255770; NM_001204744; NM_001204745 |
| CDH17 | NM_004063; XM_011516790; NM_001144663 |
| CDH18 | XM_011513930; XM_017008924; XM_017008926; XM_017008927; |
| | NM_001349556; NM_001349559; XM_017008930; NM_001349560; |
| | XM_017008929; NM_001349563; XM_017008928; NM_001167667; |
| | NM_004934; XM_006714435; XM_005248228; NM_001291956; |
| | NM_001349562; NM_001291957; NM_001349558; NM_001349561 |
| CDH19 | XM_011525932; XM_011525931; NM_021153; NR_073130; |
| | XM_047437485; NM_001271028; XM_047437484 |
| CDH2 | XM_011525788; NM_001308176; XM_017025514; NM_001792 |
| CDH20 | XR_001753187; NM_031891; XR_001753186; XM_024451165 |
| CDH22 | XM_024451966; XM_047440374; XM_047440373; XM_011528994; |
| | XM_024451967; NM_021248 |
| CDH23 | NM_001171932; NM_001171936; NM_001171930; NM_001171933; |
| | NM_001171935; NM_001171934; NM_022124; NM_052836; |
| | NM_001171931 |
| CDH3 | NM_001793; XM_011522800; XM_047433450; NM_001317195; |
| | NM_001317196 |
| CDH4 | NM_001252339; XM_047439813; NM_001794; XM_047439812; |
| | NM_001252338 |
| CDH5 | XM_047433469; XM_047433470; NM_001114117; NM_001795; |
| | XM_047433471; XM_011522801 |
| CDH6 | NM_004932; NM_001362435; XM_011513921; XM_047416591 |
| CDH7 | NM_001317214; NM_004361; NM_001362438; NM_033646 |
| CDH8 | XM_005255760; XM_047433482; XM_047433484; XM_047433483; |
| | NM_001796 |
| CDH9 | NM_016279 |
| CDK1 | NM_001320918; NM_033379; NM_001170406; NM_001786; |
| | NM_001130829; XM_005270303; NM_001170407 |
| CDK2 | NM_001290230; XM_011537732; NM_052827; NM_001798 |
| CDK4 | NM_000075; NM_052984 |
| CDK6 | XM_047419716; NM_001145306; NM_001259 |
| CDK7 | NM_001324074; NM_001324072; NM_001324078; XM_011543094; |
| | NM_001324069; NM_001324070; NM_001324075; NM_001324077; |
| | NM_001324071; XM_047416609; NM_001799; NR_136690 |
| CDK8 | NM_001346501; XM_011534865; NM_001260; XM_047430033; |
| | NM_001318368 |
| CDKN1A | NM_001220778; NM_001374510; NM_078467; NR_164655; |
| | NM_001291549; NM_001374511; NM_001374509; NR_164656; |
| | NM_000389; NM_001220777; NM_001374512; NM_001374513 |
| CDKN1B | NM_004064 |
| CDKN2A | XM_011517676; XM_011517675; NM_001363763; NM_001195132; |
| | XM_047422597; NM_058195; XM_047422596; XM_047422598; |
| | NM_000077; NM_058196; NM_058197 |
| CDKN2B | NM_078487; NM_004936 |
| CDKN2C | NM_001262; NM_078626 |
| CDKN3 | NM_001330173; NM_005192; NM_001130851 |
| CDO1 | NM_001323565; NR_136619; NM_001323567; NM_001801; NR_136618; |
| | XM_047416629; NR_136620; XM_047416628; NM_001323566; NR_136621 |
| CDT1 | NM_030928 |
| CDX2 | XM_011534876; NM_001354700; XM_011534879; XM_011534875; |
| | XM_011534878; NM_001265 |
| CEACAM1 | NM_001024912; NM_001184813; NM_001205344; XM_011527206; |
| | NM_001712; NM_001184815; NM_001184816 |
| CEACAM5 | XM_011526322; XM_017026146; NM_001291484; NM_004363; |
| | XM_017026145; NM_001308398 |
| CEACAM8 | XM_011526340; XM_011526342; XM_047438091; NM_001816; |
| | XR_007066548; XR_007066546; XM_017026195; XM_011526341; |
| | XM_017026197 |
| CECR1 | XM_047441407; NM_001282228; NM_017424; XM_047441406; |
| | NM_001282225; NM_001282227; NM_177405; XM_011546133; |
| | NM_001282226; NM_001282229 |
| CELF2 | XM_047424490; XM_047424491; XM_047424492; XM_047424494; |
| | XM_047424505; NM_001326330; NM_001326334; NM_001326336; |
| | NM_001326338; NM_001326342; NM_001326346; XM_047424482; |
| | XM_047424486; XM_047424508; NM_001326317; NM_001326318; |
| | NM_001326323; NM_001326327; NM_001326349; NM_001394513; |
| | XM_047424489; NM_001025077; NM_001326320; NM_001326321; |
| | NM_001326326; NM_006561; XM_047424488; XM_047424496; |
| | XM_047424503; XM_047424504; XM_047424510; NM_001025076; |
| | NM_001326319; NM_001326328; NM_001326339; NM_001326343; |
| | NM_001326344; XM_047424484; XM_047424487; XM_047424500; |
| | XM_047424501; XM_047424502; NM_001083591; NM_001326324; |
| | NM_001326325; NM_001326335; NM_001326341; NM_001394518; |
| | XM_024447776; XM_047424495; XM_047424499; XM_047424506; |
| | XM_047424507; NM_001326329; NM_001326347; NM_001326348; |
| | NM_001394502; NM_001394517; XM_047424483; XM_047424485; |
| | XM_047424498; NM_001326331; NM_001326333; NM_001394519; |
| | XM_047424493; XM_047424509; NM_001326332; NM_001326337; |
| | NM_001326340; NM_001326345 |
| CEP55 | XM_017016373; XM_011539920; NM_001127182; NM_018131; |
| | XM_017016372; XM_011539919; XM_047425416; XM_011539918 |
| CETN2 | NM_004344 |
| CETN3 | NM_004365; NM_001297765; NM_001297768 |
| CETP | XM_006721124; NM_000078; NM_001286085 |
| CFC1 | NM_001270421; NM_001270420; XM_011511486; NM_032545 |
| CFD | NM_001317335; NM_001928 |
| CFH | NM_001014975; XM_017001108; XR_007059267; NM_000186; |
| | XM_047418835 |
| CFHR2 | XM_011509460; XM_011509458; XM_011509459; NM_005666; |
| | XM_017001109; XM_005245113; NM_001312672 |
| CFHR5 | NM_030787; XM_011510020 |
| CFI | NM_001375281; NM_000204; NM_001375282; NR_164673; |
| | XM_006714210; NM_001375280; NM_001318057; NM_001375279; |
| | XM_011531920; NM_001375284; NR_164671; XM_047415654; |
| | NM_001331035; NM_001375278; NM_001375283; NR_164672; |
| | XM_047415653 |
| CFL1 | NM_005507 |
| CFLAR | NM_003879; NR_147251; XR_007083740; XR_007083755; XM_047446185; |
| | NM_001351593; NR_147244; NR_147253; XR_007083702; NM_001202517; |
| | NM_001351590; NM_001351592; NR_147243; NR_147248; NR_147252; |
| | NR_147255; NM_001202515; NM_001202516; NR_147245; NR_147246; |
| | NR_147250; XM_047446197; NM_001127184; NM_001308042; |
| | NR_147241; XR_001739013; XR_007083715; NM_001308043; |
| | NM_001351594; NR_147242; NR_147249; XR_007083735; XR_007083742; |
| | NM_001202518; NM_001351591; XR_007083723; XM_047446191; |
| | NM_001127183; NM_001202519; NR_147247; XR_007083684; |
| | XR_007083713; XR_007083745 |
| CFP | NM_001145252; NM_002621 |
| CHD1L | NM_001256336; NM_001348459; NM_001348463; NM_001348465; |
| | NR_145681; NR_145684; NR_145685; NR_145695; XM_047435001; |
| | NM_001348453; NM_001348457; NM_001348462; NM_004284; |
| | NM_001348451; NR_046070; NR_145687; NM_001256338; |
| | NM_001348464; NR_145682; NR_145688; NR_145694; NR_145683; |
| | NR_145686; NR_145689; XM_024451051; NM_001348455; |
| | NM_001348458; NM_001348460; NM_024568; NR_145692; NR_145693; |
| | NM_001348454; NM_001348456; NM_001348466; NR_145690; |
| | NR_145691; NM_001256337; NM_001348452; NM_001348461 |
| CHEK1 | NM_001114121; NM_001114122; XR_007062447; XM_047426313; |
| | NM_001244846; NR_045205; XM_047426311; NM_001330427; |
| | XM_024448337; XM_047426312; NM_001274; XM_011542560; |
| | NM_001330428; NR_045204 |
| CHEK2 | XM_006724114; XM_011529845; XM_024452148; XM_047441105; |
| | XM_047441106; NM_001349956; XM_006724116; XR_007067954; |
| | XM_017028560; XM_047441104; NM_001257387; NM_007194; |
| | XM_011529842; XM_047441108; NM_145862; XM_011529839; |
| | XM_011529844; XM_024452149; XM_047441107; XR_937806; |
| | XR_937807; XM_011529840; NM_001005735; XR_007067955 |
| CHGA | NM_001275; NM_001301690; XM_011536370 |
| CHST15 | XM_047425327; XM_047425325; XM_047425332; XM_005269892; |
| | XM_047425328; XM_047425329; XM_047425334; NM_014863; |
| | XM_006717891; XM_017016321; NM_001270764; XM_017016319; |
| | XM_047425330; XM_047425331; XM_017016320; XM_047425324; |
| | NM_001270765; XM_011539857; XM_005269893; XM_047425326; |
| | NM_015892; XM_047425333 |
| CHUK | XM_047424543; XM_047424542; XM_047424540; NM_001278; |
| | NM_001320928; XM_017015612; XM_047424541 |
| CIDEA | NM_001279; NR_134607; NM_001318383 |
| CIITA | XM_047434115; NM_001379332; XR_007064880; XM_006720880; |
| | XM_011522491; XM_047434119; NM_001379334; XM_047434118; |
| | XM_047434120; XM_047434123; NM_001379333; XM_011522486; |
| | NM_000246; NM_001286402; XM_047434122; XM_047434126; |
| | XR_001751904; XR_007064879; XM_047434117; XM_047434114; |
| | XM_047434125; NM_001286403; NM_001379331; XM_011522485; |
| | XM_047434127; XM_047434128; NR_104444; XM_011522484; |
| | XM_011522490; XM_047434116; XM_047434124; NM_001379330 |
| CITED1 | XM_047442125; XM_047442126; NM_001144887; XM_047442128; |
| | NM_001144886; XM_047442127; NM_001144885; NM_004143 |
| CITED2 | NM_001168389; NM_006079; NM_001168388 |
| CLCA2 | NM_006536; XM_011542448 |
| CLDN1 | NM_021101 |
| CLDN10 | NM_006984; XM_047430765; NM_182848; NM_001160100 |
| CLDN11 | NM_005602; NM_001185056 |
| CLDN12 | NM_001185072; NM_012129; NM_001185073 |
| CLDN14 | XM_047440736; NM_144492; XM_047440735; NM_001146077; |
| | NM_001146079; NM_001146078; NM_012130 |
| CLDN15 | NM_014343; NM_138429; NM_001185080 |
| CLDN16 | NM_006580; XM_047447333; NM_001378492; NM_001378493 |
| CLDN17 | NM_012131 |
| CLDN18 | NM_001002026; NM_016369 |
| CLDN19 | NM_001123395; NM_001185117; NM_148960 |
| CLDN2 | NM_001171095; NM_020384; NM_001171092 |
| CLDN20 | NM_001001346 |
| CLDN22 | NM_001111319 |
| CLDN23 | NM_194284 |
| CLDN3 | NM_001306 |
| CLDN4 | NM_001305 |
| CLDN5 | NM_001363066; NM_001363067; NM_001130861; NM_003277 |
| CLDN6 | NM_021195 |
| CLDN7 | NM_001307; NM_001185022; NM_001185023 |
| CLDN8 | NM_199328; NM_012132 |
| CLDN9 | NM_020982 |
| CLDND2 | XM_047438137; NM_152353; XM_047438138; XM_047438140; |
| | XM_047438139; XM_011526428 |
| CLEC10A | NM_001330070; NM_182906; NM_006344; XM_011523613; |
| | XM_011523615 |
| CLEC14A | NM_175060 |
| CLEC17A | XM_017026788; XM_017026785; NM_207390; NR_109785; |
| | XM_017026787; NR_109784; XM_017026793; XM_047438796; |
| | XM_017026786; XM_017026791; XM_017026792; XM_017026794; |
| | NM_001204118 |
| CLEC2D | NM_001197318; NM_001004420; NM_013269; NR_036693; |
| | NM_001197319; NM_001004419; NM_001197317 |
| CLEC4C | NM_130441; XM_024448873; NM_203503; NM_001371391; |
| | NM_001371390 |
| CLEC5A | XR_007059995; XM_011515995; NM_013252; NM_001301167 |
| CLEC7A | NM_022570; NM_197948; NM_197951; NM_197953; XM_047429359; |
| | XM_047429360; NM_197947; NM_197954; NM_197952; NM_197950; |
| | NR_125336; XM_024449132; NM_197949; XM_006719135; XM_024449133 |
| CLEC9A | NM_207345 |
| CLIC1 | NM_001288; NM_001287593; NM_001287594 |
| CLK2 | NM_001294339; XM_047444414; NM_001294338; XM_047444422; |
| | XM_047444417; NM_003993; NM_001363704; XM_011509143; |
| | NM_001291; XM_047444409 |
| CMA1 | NM_001836; NM_001308083 |
| CMKLR1 | NM_001142343; XM_047428313; NM_001142345; NM_001142344; |
| | NM_004072 |
| CNN2 | NM_004368; NM_001303501; NM_001303499; NM_201277 |
| CNR1 | NM_001160259; NM_001370546; XM_011535425; XM_047418173; |
| | NM_001365870; NM_001370547; NM_001365872; NM_016083; |
| | XM_047418172; NM_001160226; NM_001160258; NM_001370545; |
| | NM_001365874; NM_033181; NM_001160260; XM_047418171; |
| | NM_001365869 |
| CNTN6 | NM_001289081; NM_001349352; NM_001349356; XM_017006174; |
| | NM_001349361; XM_011533591; XM_047447972; NM_001349358; |
| | NM_014461; NM_001289080; NM_001349353; NM_001349359; |
| | XM_011533590; XM_047447974; NM_001349350; NM_001349357; |
| | NM_001349354; NM_001349351; NM_001349355; NM_001349360; |
| | XM_017006172; XM_017006177; NM_001349362 |
| COL11A1 | XM_017000337; XM_017000335; XM_017000336; NR_134980; |
| | NM_080629; XR_007085257; XM_017000334; NM_001190709; |
| | NM_001854; NM_080630 |
| COL16A1 | XM_005270481; XM_017000338; XM_047446431; XM_011540729; |
| | XM_011540728; XM_011540722; XM_011540723; XM_011540727; |
| | XM_017000339; XM_011540730; XM_047446432; XM_047446439; |
| | XM_047446440; XR_001736983; XM_047446435; XM_011540724; |
| | XM_017000340; XM_017000341; NM_001856 |
| COL1A1 | XM_005257058; XM_005257059; XM_011524341; NM_000088 |
| COL1A2 | NM_000089 |
| COL3A1 | NM_000090; NM_001376916 |
| COL4A1 | NM_001845; NM_001303110 |
| COL5A1 | NM_000093; XM_017014266; NM_001278074 |
| COL6A1 | NM_001848 |
| COL6A2 | NM_001849; NM_058175; NM_058174 |
| COL6A3 | NM_057164; NM_057167; NM_057166; NM_004369; NM_057165 |
| CORO1A | NM_007074; NM_001193333 |
| CPA3 | NM_001870 |
| CPB2 | XM_017020393; NM_016413; NM_001872; NM_001278541 |
| CPNE5 | NM_001314019; NM_020939; XM_047419193; NM_001376893; |
| | XM_005249247; XM_011514769; NR_164866; XR_007059285; |
| | XM_047419191; NM_001376888; NM_001376890; NM_001376894; |
| | NM_001314017; NM_001376892; XM_011514773; XM_047419192; |
| | NM_001314020; XM_017011139; NM_001376891; XM_011514770; |
| | XM_011514768; XM_011514771; XM_011514772; XM_047419190; |
| | NM_001314018; NM_001376889; NM_001376895 |
| CR1 | NM_001381851; NM_000651; NM_000573 |
| CR2 | NM_001877; NM_001006658 |
| CRB1 | XM_017000852; XM_047416574; XM_047416575; NR_047563; |
| | NM_012076; XM_047416572; NR_047564; XM_011509367; |
| | XM_011509369; XM_047416573; NM_001257966; NM_201253; |
| | NM_001257965; XM_011509365; NM_001193640 |
| CRB2 | XM_047423244; XM_011518556; NR_104603; XM_011518558; |
| | XM_005251934; XM_011518557; NM_173689; XM_047423245 |
| CRB3 | NM_174881; NM_174882; NM_139161 |
| CRBN | XM_005265202; XM_011533791; NM_001173482; NM_016302; |
| | XM_047448254; XM_047448253 |
| CREBBP | XM_017022944; XM_005255125; XM_047433625; XM_005255124; |
| | XM_011522381; XM_047433624; XM_006720848; NM_001079846; |
| | XM_011522382; NM_004380 |
| CRTAM | XM_011542900; NM_019604; NM_001304782 |
| CSF1 | NM_000757; NM_172210; XM_017000369; XM_047446752; NM_172211; |
| | NM_172212 |
| CSF1R | NM_001375320; NM_005211; NR_164679; NM_001349736; |
| | NM_001288705; NM_001375321; NR_109969 |
| CSF2 | NM_000758 |
| CSF2RA | XM_047441847; XM_047441851; XM_047442715; NM_001379164; |
| | NM_001379166; XM_047441849; XM_047442710; XM_047442713; |
| | NM_001161530; NM_001379165; NM_006140; NM_172247; NM_172248; |
| | XM_011545627; XM_047441850; NM_001161531; NM_001161532; |
| | NM_001379168; NM_172245; XM_047441846; NM_001161529; |
| | NM_001379158; NM_001379161; NM_001379162; NM_172246; |
| | XM_011545620; XM_011546167; XM_047442711; XM_047442712; |
| | XM_047442716; NM_001379155; NM_001379163; NM_172249; |
| | XM_047441845; XM_047441848; NM_001379160; NM_001379167; |
| | NR_027760; XM_011545628; XM_011546174; XM_047441852; |
| | XM_047442718; NM_001379153; XM_011546175; XM_047441853; |
| | XM_047442714; XM_047442717; NM_001379154; NM_001379156; |
| | NM_001379159; NM_001379169 |
| CSF2RB | XM_011529904; XM_005261340; XM_047441149; XM_011529903; |
| | XM_047441150; XM_047441148; NM_000395 |
| CSF3 | NR_168489; NR_168491; NM_000759; NM_001178147; NM_172219; |
| | NM_172220; NR_168490; NR_033662 |
| CSF3R | NM_000760; XM_005270493; NM_156039; XM_011540749; NM_156038; |
| | NM_172313; XM_047446753 |
| CSMD3 | XM_017013010; XM_017013009; NM_001363185; XM_047421314; |
| | XM_011516815; NM_052900; XM_017013008; NM_198123; |
| | XM_011516816; NM_198124 |
| CSNK1D | XM_047435379; NM_139062; NM_001893; NR_110578; XR_007065265; |
| | XM_047435381; XM_047435380; NM_001363749; XM_005256336 |
| CSNK1E | NM_152221.3; NM_001894.5 |
| CSPG4 | XM_047432196; NM_001897 |
| CTBP2 | XM_005269567; XM_047424673; XM_011539355; XM_011539358; |
| | XM_047424666; XM_047424669; XM_047424676; NM_001321013; |
| | NM_022802; XM_047424667; XM_047424670; XM_047424671; |
| | NM_001363508; XM_024447830; XM_047424672; XM_047424675; |
| | XM_047424677; XM_047424678; NM_001290215; XM_047424680; |
| | NM_001083914; NM_001290214; NM_001329; XM_047424664; |
| | XM_047424679; NM_001321012; NM_001321014; XM_047424665; |
| | XM_047424668; XM_047424674 |
| CTLA4 | NM_001037631; NM_005214 |
| CTNNA1 | NM_001323982; NM_001323989; NM_001290312; NM_001323988; |
| | NM_001323990; NM_001323992; NM_001323998; NM_001324002; |
| | NM_001324008; NM_001324012; NM_001290310; NM_001323985; |
| | NM_001323987; NM_001323991; NM_001323995; NM_001323997; |
| | NM_001323999; NM_001324003; NM_001323984; NM_001323996; |
| | NM_001324005; NM_001324013; NM_001903; NM_001290309; |
| | NM_001323983; NM_001323986; NM_001324006; NM_001323993; |
| | NM_001290307; NM_001323994; NM_001324001; NM_001324004; |
| | NM_001324007; NM_001324010; NM_001324011; NM_001324000; |
| | NM_001324009 |
| CTNNA2 | XM_011532557; XM_011532556; XM_024452715; NM_001282598; |
| | NM_001282600; NM_001282599; NM_004389; XM_047443447; |
| | NM_001282597; NM_001399737; XM_017003403; XM_017003405; |
| | NM_001164883; XM_024452714; NM_001320810 |
| CTNNA3 | XM_017016155; XM_017016158; NM_001291133; NM_013266; |
| | XM_017016152; XM_017016156; XM_047425124; NM_001127384; |
| | XM_017016151; XM_017016157 |
| CTNNB1 | NM_001330729; NM_001904; XM_047447478; XM_047447482; |
| | XM_047447483; XM_024453356; XM_017005738; XM_047447480; |
| | XM_047447481; XM_047447477; XM_006712985; NM_001098209; |
| | XM_047447479; NM_001098210 |
| CTNND1 | NM_001206890; NM_001085458; NM_001085461; NM_001085463; |
| | NM_001085465; NM_001085467; NM_001085469; NM_001206891; |
| | NM_001331; NM_001206883; NM_001206885; NM_001206886; |
| | NM_001206887; NM_001206889; NM_001085460; NM_001085464; |
| | NM_001085468; NM_001206884; NM_001206888; NM_001085459; |
| | NM_001085462; NM_001085466 |
| CTNND2 | NM_001332; XM_005248251; XM_017009072; XM_047416777; |
| | NM_001288715; NM_001288716; NM_001288717; NM_001364128; |
| | XM_011513967; XM_017009074; XM_005248253; NR_109988 |
| CTSG | NM_001911; XM_011536499 |
| CTSS | NM_004079; NM_001199739 |
| CTSW | NM_001335 |
| CUL1 | NM_001370663; NM_001370664; NM_003592; NM_001370661; |
| | NM_001370662; NM_001370660 |
| CUL3 | NM_001257198; NM_003590; XM_011511996; XM_006712800; |
| | XM_011511995; NM_001257197; XM_047446024 |
| CUL4A | NM_001354941; NM_003589; NM_001354942; NM_001278514; |
| | NM_001278513; NM_001354938; NM_001354939; NM_001354940; |
| | NM_001008895; NM_001354943; NM_001354944 |
| CUL4B | NM_001330624; NM_001079872; NM_001369145; NM_003588 |
| CX3CL1 | NM_001304392; NM_002996 |
| CX3CR1 | NM_001171174; NM_001337; XM_047447538; NM_001171171; |
| | NM_001171172 |
| CXADR | NM_001207066; NM_001338; XR_001754814; XM_011529478; |
| | XM_011529479; XM_011529477; NM_001207065; XM_011529476; |
| | NM_001207063; NM_001207064 |
| CXCL1 | NM_001511; NR_046035 |
| CXCL10 | NM_001565; NR_168520 |
| CXCL11 | NM_001302123; NM_005409 |
| CXCL12 | NM_000609; NM_001277990; NM_199168; NM_001178134; |
| | NM_001033886 |
| CXCL13 | NM_001371558; NM_006419 |
| CXCL16 | NM_001100812; NM_001386809; NM_022059 |
| CXCL2 | NM_002089 |
| CXCL3 | NM_002090 |
| CXCL5 | NM_002994 |
| CXCL6 | NM_002993 |
| CXCL8 | NM_000584; NM_001354840 |
| CXCL9 | NM_002416 |
| CXCR1 | NM_000634 |
| CXCR2 | XM_047444190; XM_047444188; NM_001557; NM_001168298; |
| | XM_005246530; XM_047444189; XM_017003991; XM_047444191; |
| | XM_047444187 |
| CXCR3 | XM_017029435; XM_017029436; XM_047442010; NM_001504; |
| | NM_001142797; XM_005262256; XM_005262257 |
| CXCR4 | NM_001348059; NM_001348060; XM_047445802; NM_001348056; |
| | NM_003467; NM_001008540 |
| CXCR5 | NM_032966; NM_001716 |
| CXCR6 | NM_001386435; NM_001386436; NM_006564; NM_001386437 |
| CYBB | XM_047441855; NM_000397 |
| CYFIP2 | XM_047417100; XM_047417101; XM_047417102; XM_011534516; |
| | NM_001291721; NM_001037332; NM_001037333; NM_001291722; |
| | NM_014376 |
| CYP24A1 | XM_047439936; NM_001128915; XM_017027692; NM_000782; |
| | XM_047439937; XM_017027691; XM_017027693; XM_047439938; |
| | XM_005260304 |
| CYP2A6 | NM_000762 |
| CYP2C8 | NM_001198854; NM_001198855; NM_030878; NM_000770; |
| | NM_001198853 |
| CYP2C9 | NM_000771 |
| CYP2D6 | NM_000106; NM_001025161 |
| CYP2E1 | NM_000773 |
| CYP3A4 | NM_001202855; NM_017460; NM_001202856; NM_001202857 |
| CYP3A5 | NM_001291830; NM_001190484; NR_033807; NR_033812; |
| | NM_001291829; NM_000777; NR_033810; NR_033811 |
| CYP4A11 | NR_134994; NR_134991; XM_017000465; XM_005270539; NM_000778; |
| | NM_001319155; NR_134992; NR_134993; NR_134989; NR_134990; |
| | NM_001363587; NR_134988 |
| CYP8B1 | NM_004391 |
| CYTH4 | NM_013385; NM_001318024 |
| CYTIP | NM_004288; XM_017005386 |
| DCK | XM_047449689; NM_000788 |
| DCLRE1C | NM_001033858; NM_022487; NR_110297; XM_011519621; |
| | XM_047425651; NR_146962; XM_047425649; NM_001033855; |
| | NM_001289076; NM_001350965; NR_146961; XM_047425652; |
| | XM_047425648; NM_001289078; NM_001350967; NM_001033857; |
| | NM_001350966; XM_011519620; XM_047425650; NM_001289077; |
| | NM_001289079; NR_146960; XR_930515 |
| DCN | NM_133503; NM_133504; NM_133505; NM_001920; NM_133506; |
| | NM_133507 |
| DDB1 | NM_001923 |
| DDB2 | NR_174611; NM_001300734; NM_001399874; XM_047426487; |
| | NM_000107; NM_001399875; NM_001399876; NM_001399878; NR_174610 |
| DDR1 | XM_047419318; XM_047419327; NM_001387898; NM_013993; |
| | NM_001387895; NM_001387896; NM_001387906; NM_001387917; |
| | NM_013994; XM_024446540; XM_047419330; NM_001202521; |
| | NM_001297652; NM_001297654; NM_001387892; NM_001387893; |
| | NM_001387899; NM_001387914; NM_001387915; XM_017011268; |
| | XM_047419326; XM_047419331; XM_047419334; NM_001202523; |
| | NM_001387911; NM_001387912; NM_001387918; XM_047419333; |
| | NM_001202522; NM_001387894; NM_001387900; NM_001387905; |
| | NM_001387908; NM_001387913; XM_011514887; XM_047419324; |
| | XM_047419329; XM_047419332; NM_001387902; NM_001387904; |
| | XM_011514884; XM_024446541; XM_047419320; XM_047419322; |
| | XM_047419323; XM_047419325; NM_001387897; NM_001387901; |
| | NM_001387907; NM_001387909; NM_001387910; XM_047419319; |
| | XM_047419321; XM_047419328; NM_001297653; NM_001387903; |
| | NM_001387916; NM_001954 |
| DDR2 | XM_047421565; NM_001014796; XM_011509587; XM_011509588; |
| | NM_001354982; NM_006182; XM_047421554; NM_001354983 |
| DDX3X | NM_001356; NR_126094; NM_001193417; NM_024005; NM_001363819; |
| | NR_126093; NM_001193416; XM_011543892 |
| DDX6 | NM_004397; XM_005271417; XM_011542645; XM_047426488; |
| | XM_047426490; XM_017017251; XM_024448377; XM_047426489; |
| | NM_001257191; XM_011542644 |
| DEF6 | NM_022047; XM_047418838 |
| DEFB1 | NM_005218 |
| DEK | NM_003472; NM_001134709; XM_024446544; XM_047419335 |
| DENND1C | XM_047439458; XM_047439459; XM_047439460; XM_024451727; |
| | NM_001290331; XM_006722906; XM_011528318; XM_006722905; |
| | NM_024898 |
| DEPTOR | NM_001283012; NM_022783 |
| DERL3 | XM_047441595; XM_047441594; NM_001002862; XM_017029078; |
| | XM_047441597; NM_001135751; NM_198440; XM_047441596; |
| | NM_001363072; XM_011530505 |
| DES | NM_001927; NM_001382708; NM_001382710; NM_001382713; |
| | NM_001382709; NM_001382711; NM_001382712 |
| DGKZ | NM_003646; NM_201533; NM_001199267; NM_001199266; |
| | NM_001199268; NM_201532; NM_001105540 |
| DIABLO | NM_019887; NM_138929; NM_001278304; NM_001278303; |
| | NM_001278302; NM_138930; NM_001278342; NM_001371333 |
| DIRAS3 | NM_004675 |
| DIXDC1 | XM_017018466; XM_017018467; NM_001278542; NM_033425; |
| | XM_047427787; NM_001037954; XM_024448743; XM_024448742 |
| DKK1 | NM_012242 |
| DKK2 | NM_014421 |
| DKK3 | XM_047426775; NM_001018057; NM_013253; XM_017017555; |
| | XM_047426774; XM_006718178; NM_001330220; NM_015881 |
| DKK4 | NM_014420; XM_017013316; XM_011544488 |
| DLC1 | NM_001316668; NM_182643; XM_005273374; NM_001348081; |
| | NM_001348083; NM_001348084; NM_001164271; NM_006094; |
| | NM_024767; NM_001348082 |
| DLL1 | NM_005618; XM_005266934 |
| DLL4 | NM_019074 |
| DNA2 | XM_006717680; NM_001080449; XM_017015799; XM_011539417; |
| | NR_102264 |
| DNER | XM_005246950; NM_139072 |
| DOCK2 | XM_011534450; XM_011534451; NM_004946; XM_017009190; |
| | XM_011534448; NR_156756; XM_005265830; XM_011534449 |
| DPYD | XM_006710397; XM_017000507; XM_047448077; NM_000110; |
| | NM_001160301; XM_047448076; XR_001737014; XM_005270562 |
| DPYS | XM_011516903; XM_047421418; XM_047421416; NM_001385; |
| | XM_005250818; XM_047421415; XM_047421419; XM_006716518; |
| | XM_017013167 |
| DSC1 | NM_004948; NM_024421 |
| DSC2 | NM_004949; XM_005258206; NM_024422 |
| DSC3 | NM_001941; NM_024423 |
| DSG1 | NM_001942 |
| DSG2 | XM_047437315; NM_001943 |
| DSG3 | XM_011525850; NM_001944 |
| DSG4 | NM_001134453; NM_177986 |
| DSP | NM_001008844; NM_004415; NM_001319034 |
| DUSP22 | XM_017011062; NM_020185; XM_011514757; NR_104473; |
| | NM_001286555; NR_104474; NR_104475 |
| DUSP6 | NM_022652; NM_001946 |
| DUX4 | NM_001205218; NR_038191; NM_001363820; NR_137167; |
| | NM_001278056; NM_001293798; NM_001306068 |
| DVL1 | XM_005244733; XM_047448090; NM_004421; NM_182779; |
| | XM_005244732; NM_001330311; NM_181870 |
| DVL2 | NM_004422; XM_047435519; XM_005256502; XM_047435518; |
| | XM_047435520; XM_047435522 |
| DVL3 | NM_004423 |
| DYRK2 | XM_017020032; NM_003583; NM_006482 |
| E2F1 | XM_047439961; NM_005225 |
| E2F3 | NM_001949; XM_011514324; XM_047418265; XM_005248866; |
| | XM_017010330; XM_011514328; XM_005248865; XM_047418266; |
| | NM_001243076 |
| EAF2 | XM_047448577; XM_005247618; XM_017006861; XM_017006863; |
| | XM_047448576; NM_001320041; NM_018456 |
| EBF1 | XM_024454390; XM_024454391; XM_047416890; NM_001324101; |
| | XM_017009194; XM_017009199; XM_047416892; NM_001364156; |
| | NM_001364158; NM_001324103; NM_001324109; NM_001324111; |
| | NM_001364157; NM_001364159; XM_017009200; XM_047416889; |
| | XM_047416891; NM_001324106; NM_001324107; XM_017009197; |
| | XM_047416888; NM_001364155; NM_024007; NM_182708; |
| | XM_017009202; NM_001290360; XM_017009203; NM_001324108 |
| ECSCR | NM_001077693; NM_001293739; NR_121659 |
| ECT2 | XM_047447608; XM_047447628; NM_001349101; NM_018098; |
| | XM_006713524; XM_047447610; XM_047447613; XM_047447607; |
| | XM_047447620; XM_047447623; XM_047447630; XM_047447636; |
| | XM_047447637; NM_001349094; NM_001349100; XM_047447616; |
| | XM_047447618; XM_047447625; XM_047447629; XM_047447635; |
| | XM_047447638; NM_001258316; NM_001349097; XM_047447611; |
| | XM_047447634; XM_047447639; NM_001258315; NM_001349096; |
| | NM_001349099; NM_001349104; XM_047447614; XM_047447617; |
| | XM_047447631; XM_047447633; NM_001349102; NM_001349103; |
| | XM_047447609; XM_047447615; XM_047447619; XM_047447621; |
| | XM_047447622; XM_047447624; XM_047447626; NM_001349098; |
| | XM_047447612; XM_047447627; XM_047447632; NM_001349095 |
| EDNRB | NM_001122659; NM_003991; NM_001201397; NM_000115; NR_047024 |
| EEF1A2 | NM_001958 |
| EFNB1 | NM_004429 |
| EGF | XM_005262796; XM_011531707; XM_017007848; XM_017007850; |
| | XM_047449723; NM_001178131; XM_047449725; XM_017007847; |
| | XM_017007855; XM_047449726; XM_047449727; XM_047449729; |
| | XM_017007854; NM_001963; XR_001741156; XM_017007845; |
| | XM_017007849; XM_047449728; NM_001178130; XM_017007846; |
| | XM_017007853; NM_001357021; XM_017007851; XM_047449724; |
| | XM_047449730 |
| EGFR | XM_047419953; NM_001346899; NM_201282; XM_047419952; |
| | NM_201284; NM_001346898; NM_001346900; NM_001346897; |
| | NM_201283; NM_001346941; NM_005228 |
| EGR1 | NM_001964 |
| EHF | XM_047426755; XM_047426760; NM_001378041; NM_001378045; |
| | NM_001378046; NM_001378053; XM_047426757; XM_047426762; |
| | NM_001378048; NM_001378050; NR_165391; XM_024448434; |
| | NM_001378047; NM_001378056; NR_165390; NM_001378052; |
| | XM_047426759; XM_047426761; NM_001378044; NM_012153; |
| | XM_005252861; XM_047426756; NM_001206615; NM_001378051; |
| | XM_047426754; XM_047426753; XM_047426758; NM_001378042; |
| | NM_001378043; NM_001378049; NM_001378055; NM_001206616; |
| | NM_001378054 |
| EIF1AY | NM_001278612; NM_004681 |
| EIF4E | NM_001130678; NM_001968; NM_001130679; NM_001331017 |
| EIF4EBP1 | NM_004095 |
| ELANE | NM_001972 |
| ELF1 | XM_047430122; NM_001370330; NM_001370331; XM_047430123; |
| | XM_047430124; NM_001145353; XM_047430120; NM_001370332; |
| | XM_047430121; XM_047430118; NM_001370329; NM_172373; |
| | XM_047430119 |
| ELMO1 | XM_011515654; XM_047421091; XM_005249919; XM_047421086; |
| | XM_047421090; NM_001206480; NM_130442; XM_006715805; |
| | NM_001039459; XM_047421087; NR_038120; XM_017012839; |
| | XM_024447008; XM_047421088; NM_001206482; NM_014800; |
| | XM_047421089 |
| ELN | XM_011515869; XM_011515873; XM_017011814; XM_047419961; |
| | XM_047419973; XM_047419978; XM_005250187; XM_011515871; |
| | XM_011515872; XM_047419958; XM_047419962; XM_047419963; |
| | XM_047419965; NM_001278914; XM_005250188; XM_011515874; |
| | XM_047419957; XM_047419964; XM_047419966; XM_047419974; |
| | XM_047419979; NM_000501; NM_001278912; NM_001278939; |
| | XM_011515877; XM_047419960; XM_047419967; XM_047419971; |
| | XM_047419977; NM_001081753; XM_011515876; XM_047419955; |
| | XM_047419970; XM_047419975; NM_001081754; NM_001278917; |
| | XM_017011813; XM_047419956; XM_047419954; XM_047419968; |
| | XM_047419969; XM_047419972; XM_047419980; NM_001278915; |
| | NM_001278918; XM_011515868; XM_011515870; XM_047419981; |
| | NM_001081755; NM_001278916; XM_011515875; XM_047419959; |
| | XM_047419976; NM_001081752; NM_001278913 |
| EMCN | NM_001159694; XM_017008290; NM_016242; XM_011532024 |
| EMEI | XM_005257081; XM_017024236; XM_047435472; NM_001166131; |
| | XM_047435471; NM_152463; XR_007065270; XM_047435470 |
| EMILIN2 | NM_032048; XM_047437887; XM_047437886; XM_047437884; |
| | XM_047437885 |
| ENG | NM_000118; NM_001114753; NM_001278138 |
| ENO1 | NM_001201483; NM_001353346; NM_001428 |
| ENPP3 | NR_133007; NM_005021; XM_017010932; XM_011535897 |
| ENTPD1 | XM_017016963; NM_001164179; NM_001164181; XM_011540374; |
| | XM_047426024; NM_001164183; XM_011540371; NM_001164178; |
| | XM_011540372; XM_011540376; XM_047426027; XM_047426029; |
| | NM_001312654; XM_047426025; XM_047426026; XM_047426028; |
| | NM_001164182; NM_001776; XM_017016958; XM_017016964; |
| | XM_011540370; XM_011540373; XM_047426023; NM_001098175; |
| | NM_001320916 |
| EOMES | NM_001278182; XM_005265510; NM_005442; NM_001278183 |
| EP300 | NM_001362843; NM_001429 |
| EPAS1 | NM_001430; XM_011532698 |
| EPHA1 | NM_005232 |
| EPHA10 | XM_047418403; XM_017001080; XR_001737123; XR_946610; |
| | XM_017001081; XM_047418391; NM_001099439; NM_001004338; |
| | XR_001737124; XR_001737125; NM_173641; XR_001737127; XR_946613; |
| | XR_001737126 |
| EPHA2 | XM_017000537; XM_047448267; XM_047448259; NM_001329090; |
| | XM_047448272; NM_004431 |
| EPHA3 | NM_005233; XM_005264716; NM_182644; XM_005264715; |
| | XM_047447673 |
| EPHA4 | NM_001304537; NM_001363748; NM_004438; NM_001304536 |
| EPHA5 | XM_005265653; XM_047449763; XM_017007880; XM_017007881; |
| | NM_001318761; NM_001281765; XM_011531735; NM_001281767; |
| | NM_004439; XM_047449762; XM_017007878; NM_001281766; |
| | NM_182472 |
| EPHA6 | XM_017006210; XM_017006212; XM_017006213; XM_017006219; |
| | XM_017006218; XM_047448008; NM_001278300; XM_017006211; |
| | XM_017006214; XM_017006217; NM_001080448; XR_001740110; |
| | XM_006713592; XM_011512706; XM_047448009; NM_173655; |
| | XR_924126; XM_011512707; XM_011512705; NM_001278301; |
| | XM_017006215; XM_017006216 |
| EPHA7 | NM_001288630; XM_047418274; NM_001376467; XM_017010366; |
| | NM_001288629; NM_001376466; NM_001376471; NM_004440; |
| | NM_001376465; NM_001376470; NR_164810; NM_001376468; |
| | NM_001376469 |
| EPHA8 | XR_946576; NM_020526; XM_011540970; XM_011540975; |
| | XM_011540973; XM_011540969; XM_011540972; NM_001006943 |
| EPHB1 | NM_004441 |
| EPHB2 | XM_006710441; XM_047449104; NM_001309192; NM_004442; |
| | NM_001309193; NM_017449; XM_006710442 |
| EPHB3 | NM_004443 |
| EPHB4 | XM_017011816; NM_004444 |
| EPHB6 | XM_024446675; XM_047419983; NM_001280795; XM_047419984; |
| | XM_011515879; NM_001280794; NR_104001; XM_011515881; |
| | XM_011515880; XM_011515882; NM_004445 |
| EPOR | NR_033663; NM_000121 |
| EPX | NM_000502 |
| ERBB2 | NM_001005862; NM_001382784; NM_001382785; NM_001382788; |
| | NM_001382792; NM_001382793; NM_001382803; XM_047435590; |
| | NM_001289937; NM_001382786; NM_001382800; NM_001382802; |
| | NM_001382806; NM_001382782; NM_001382789; NM_001382795; |
| | NM_001289936; NM_001382797; NM_001382805; NM_004448; |
| | NR_110535; NM_001289938; NM_001382791; NM_001382801; |
| | NM_001382783; NM_001382790; NM_001382794; NM_001382798; |
| | NM_001382799; NM_001382787; NM_001382796; NM_001382804 |
| ERBB3 | XM_047428500; NM_001005915; XM_047428501; NM_001982 |
| ERBB4 | XM_005246376; XM_017003577; XM_017003578; XM_005246377; |
| | NM_001042599; XM_017003581; XM_006712364; XM_017003582; |
| | XM_017003579; XM_017003580; NM_005235 |
| ERCC1 | NM_001369419; NM_001369409; NM_001166049; NM_001369412; |
| | NM_001369417; NM_202001; NM_001369415; NM_001369418; |
| | NM_001369408; NM_001369410; NM_001369411; NM_001369413; |
| | NM_001369414; NM_001369416; NM_001983 |
| ERCC2 | XM_047438393; XR_007066680; NM_000400; NM_001130867; |
| | XR_001753633; XM_011526611 |
| ERCC3 | NM_001303416; NM_000122; XM_011510794; XM_011510795; |
| | NM_001303418 |
| ERCC5 | NM_000123 |
| ERCC6 | NM_000124; NM_001277058; NM_001277059; NM_001346440 |
| ERCC8 | NM_000082; NM_001007234; NM_001007233; NM_001290285 |
| EREG | NM_001432 |
| ERG | NM_001243429; NM_001136155; NR_111949; NM_182918; |
| | NM_001243428; NM_001243433; NM_004449; NM_001291391; |
| | NM_001331025; NM_001136154; NM_001243432 |
| ERRFI1 | XM_047422701; XM_005263477; XM_047422698; NM_018948 |
| ESAM | NM_138961 |
| ESCO2 | NM_001017420; XR_949378; XR_007060703; XM_011544422; |
| | XM_011544421 |
| ESM1 | NM_001135604; NM_007036 |
| ESR1 | XM_011535545; XM_017010378; XM_047418293; NM_001385568; |
| | XM_017010381; XM_047418294; XM_047418297; NM_001122741; |
| | NM_001328100; NM_001385570; XM_047418291; XM_017010383; |
| | XM_047418289; XM_047418299; NM_001385572; XM_011535547; |
| | XM_011535549; XM_017010377; XM_047418290; NM_001385571; |
| | XM_017010380; XM_047418298; NM_000125; NM_001122740; |
| | NM_001122742; NM_001291230; NM_001291241; XM_011535543; |
| | XM_017010379; XM_047418296; XM_047418292; XM_047418295; |
| | NM_0013 85569 |
| ESRP1 | XM_005250991; NM_001122827; NM_017697; NM_001122826; |
| | NM_001034915; XM_047421916; NM_001122825 |
| ETS1 | XM_047426526; NM_001143820; NM_005238; XM_011542650; |
| | XM_017017314; XM_047426527; XM_047426525; NM_001162422; |
| | NM_001330451; XM_017017315 |
| EVI2B | NM_006495 |
| EVL | XM_011536828; XR_007064013; XR_007064014; XM_047431463; |
| | NM_016337; XM_047431460; NM_001330221; XM_047431462; |
| | XM_047431464; XM_005267749; XM_047431465 |
| EZH2 | XM_011515890; XM_047419992; XM_047420004; XM_005249964; |
| | XM_011515889; XM_011515895; XM_047419994; NM_001203248; |
| | XM_011515883; XM_005249963; XM_017011817; XM_017011820; |
| | XM_047419993; XM_047419995; XM_047420000; NM_152998; |
| | XM_011515899; XM_047419990; XM_047419991; XM_047419997; |
| | XM_047419998; NM_001203249; XM_011515896; XM_047419996; |
| | XM_011515893; XM_005249962; XM_011515885; XM_017011819; |
| | XM_047419999; XM_047420001; XM_047420002; XM_047420005; |
| | XM_047420008; XM_047420009; NM_004456; XM_011515887; |
| | XM_011515892; XM_011515894; XM_011515901; XM_047419989; |
| | XM_047420006; XM_047420007; NM_001203247 |
| F11 | XM_047449812; XM_047449817; XM_047449816; XM_005262823; |
| | XM_005262821; XM_005262822; XM_047449814; NM_019559; |
| | XM_047449811; XM_047449815; NM_000128; NM_001354804; |
| | XM_017007886; XM_047449813; XM_017007885; XM_006714137; |
| | XM_017007884 |
| F12 | XM_011534462; NM_000505 |
| F2 | NM_000506; NM_001311257 |
| F2R | NM_001311313; NM_001992 |
| F3 | NM_001178096; NM_001993 |
| F7 | XM_011537476; XM_047430125; XM_011537475; NM_001267554; |
| | XM_011537474; NR_051961; XM_006719963; NM_019616; NM_000131 |
| F9 | NM_001313913; XM_005262397; NM_000133 |
| FABP4 | NM_001442 |
| FADD | NM_003824 |
| FAM129C | XM_011527789; XM_011527781; XM_017026454; NM_001321828; |
| | XM_017026453; XM_011527786; XM_047438389; NM_001098524; |
| | XM_047438388; NM_173544; XM_017026457; XM_047438390; |
| | NM_001321826; XM_011527787; NM_001321827; NM_001363609 |
| FAM49B | NM_001353249; NM_001353252; NM_001353260; NM_001353261; |
| | NM_001353282; NM_001353305; NM_001353243; NM_001353242; |
| | NM_001353253; NM_001353263; NM_001353266; NM_001353277; |
| | NM_001353279; NM_001353289; NM_001353296; NM_001353300; |
| | NM_001353302; NM_001353310; NM_001353311; NM_016623; |
| | XM_011517121; XM_024447180; NM_001330612; NM_001353256; |
| | NM_001353259; NM_001353270; NM_001353281; NM_001353284; |
| | NM_001353287; NM_001353290; XM_047421859; NM_001353245; |
| | NM_001353247; NM_001353267; NM_001353269; NM_001353283; |
| | NM_001353285; NM_001353286; NM_001353292; NM_001353304; |
| | NM_001353313; NM_001353314; NM_001353316; XM_047421856; |
| | NM_001353250; NM_001353254; NM_001353264; NM_001353268; |
| | NM_001353276; NM_001353280; NM_001353299; NM_001353306; |
| | NM_001353312; NM_001353244; NM_001353246; NM_001353251; |
| | NM_001353258; NM_001353265; NM_001353273; NM_001353288; |
| | NM_001353301; NM_001353307; XM_047421855; NM_001256763; |
| | NM_001353248; NM_001353255; NM_001353262; NM_001353271; |
| | NM_001353272; NM_001353278; NM_001353298; NM_001353308; |
| | NM_001353315; XM_047421857; NM_001353257; NM_001353274; |
| | NM_001353275; NM_001353291; NM_001353293; NM_001353294; |
| | NM_001353295; NM_001353297; NM_001353303; NM_001353309 |
| FAM78A | NM_001400581; NM_001400583; NM_001400588; XM_011518568; |
| | NM_001400584; NM_001400585; NM_001400593; NM_001400591; |
| | XM_047423250; NM_001400589; NM_001400590; NM_001400592; |
| | NM_001400595; NM_033387; NM_001400582; NM_001400586; |
| | NM_001400594; NM_001399459; NM_001400587 |
| FANCA | NM_000135; NM_001018112; NM_001286167; NM_001351830 |
| FAP | XM_011510797; NM_004460; XM_011510796; XM_017003585; |
| | XR_001738668; XR_922891; NM_001291807 |
| FAS | XM_047425180; NR_028033; XM_011539766; XM_011539765; |
| | NR_028034; NR_135314; NR_135315; NM_152877; XM_011539764; |
| | XM_047425179; NM_152873; NM_152876; XM_006717819; |
| | NM_001320619; NR_028035; XM_047425178; NM_152871; NM_152874; |
| | NM_152872; NR_028036; NM_152875; XM_011539767; NM_000043; |
| | NR_135313 |
| FASLG | NM_001302746; NM_000639 |
| FBLN1 | NM_006485; NM_006486; NM_001996; NM_006487 |
| FBLN2 | NM_001004019; NM_001998; NM_001165035 |
| FBN1 | NM_000138 |
| FBXW7 | XM_011532087; XM_047415900; NM_001257069; XM_011532084; |
| | XM_011532085; XM_047415899; XM_047415901; NM_001013415; |
| | XM_024454126; XM_047415898; XM_047415897; XM_047415902; |
| | XM_011532086; XM_024454123; NM_001349798; NM_018315; |
| | NM_033632 |
| FCER1A | NM_001387282; NM_001387281; NM_001387280; NM_002001 |
| FCER1G | NM_004106 |
| FCER2 | NM_002002; NM_001220500; XM_005272462; NM_001207019 |
| FCGR1A | NM_001378804; NM_001378805; NM_001378807; NM_001378810; |
| | NR_166122; NR_166123; NM_001378809; NM_001378811; |
| | NM_001378808; NR_166121; NM_000566; NM_001378806 |
| FCGR1B | NR_045213.2; NR_164758.1; NR_164759.1; NR_164760.1 |
| FCGR2A | NM_001136219; NM_021642; XM_024454040; XM_017000664; |
| | XM_017000665; XM_017000663; XM_017000666; XM_047449441; |
| | XM_011509290; XM_011509291; NM_001375296; NM_001375297 |
| FCGR2B | NM_001002273; NM_001386004; NR_169827; XM_024454047; |
| | NM_001002275; NM_001386006; NM_001190828; NM_004001; |
| | NM_001386002; NM_001002274; NM_001386001; NM_001386005; |
| | NM_001386000; NM_001386003; NM_001394477 |
| FCGR3A | χM_047449443; NM_001127595; NM_001329122; XM_047449444; |
| | NM_001127596; NM_001127592; NM_000569; NM_001386450; |
| | NM_001127593; NM_001329120 |
| FCGR3B | NM_001271036; NM_001271037; NM_000570; NM_001244753; |
| | NM_001271035 |
| FCMR | XM_047434335; NM_001193338; NM_005449; NM_001142473; |
| | XM_047434334; XM_047434331; NM_001142472; XM_005273351 |
| FCN1 | NM_002003 |
| FCRL1 | XM_005244869; XM_047444001; XM_011509125; XM_011509136; |
| | NM_001159397; XM_011509128; XM_011509133; XM_011509134; |
| | NM_001159398; XM_005244866; XM_005244867; XM_011509127; |
| | NM_052938; XR_921738; XM_011509135; XM_011509126; |
| | XM_011509129; XM_011509130 |
| FCRL2 | XR_001737404; XM_011509974; XM_017002318; XM_017002319; |
| | XM_047430273; XM_006711535; XM_011509976; NM_001159488; |
| | NM_138738; XM_017002316; XM_017002317; NR_125358; NM_138739; |
| | XM_011509975; XM_047430270; XM_047430275; NM_030764; |
| | XR_001737403 |
| FCRL3 | NR_135216; NR_135217; XM_006711145; NM_001320333; NM_052939; |
| | NR_135214; NR_135215; NM_001024667 |
| FCRL5 | XM_011510032; XM_047431422; XM_011510030; XM_011510033; |
| | XM_011510031; NM_031281; NM_001195388 |
| FCRL6 | XM_011509480; XM_047419607; NM_001004310; XM_011509481; |
| | XM_047419606; NM_001284217; XM_005245128; XM_005245129; |
| | XM_005245131 |
| FCRLA | NM_001184867; NM_001184870; NM_001184866; NM_032738; |
| | XM_006711581; XM_011510065; NM_001184873; NM_001184871; |
| | NM_001184872; NM_001366195; NM_001366196 |
| FEN1 | NM_004111 |
| FERMT3 | NM_001382362; NM_001382363; NM_001382364; NM_001382448; |
| | NM_031471; XM_047427676; NM_001382361; NM_178443 |
| FFAR2 | XM_047438699; NM_005306; NM_001370087; XM_017026711; |
| | XM_047438700 |
| FGA | NM_000508; NM_021871 |
| FGF2 | NM_001361665; NM_002006 |
| FGFBP2 | NM_031950 |
| FGFR1 | XM_024447097; XM_047421569; XM_047421570; NM_001174065; |
| | NM_001354370; NM_023111; XM_006716310; XM_006716303; |
| | XM_006716304; XM_011544445; XM_011544449; XM_017013221; |
| | XM_017013225; NM_001354368; NM_001354369; NM_015850; |
| | NM_023106; XM_006716307; XM_011544444; XM_047421571; |
| | XM_047421572; NM_001354367; NM_023105; XM_006716311; |
| | XM_011544446; XM_011544452; XM_017013219; XM_017013226; |
| | XM_047421573; XM_047421574; NM_023107; NM_023109; |
| | XM_011544447; XM_011544451; NM_023110; XM_006716312; |
| | XM_011544450; XM_017013220; XM_017013227; XM_017013231; |
| | NM_001174067; NM_032191; XM_006716314; XM_011544448; |
| | XM_047421575; NM_001174063; NM_001174064; NM_001174066; |
| | XM_047421576; NM_023108 |
| FGFR2 | XM_017015924; NM_001144919; XM_006717708; XM_017015925; |
| | NM_001144915; NM_001144917; NM_022975; NM_023028; |
| | XM_024447890; NM_000141; NM_001144913; NM_001320654; |
| | NM_022970; NR_073009; NM_022971; NM_022973; NM_023030; |
| | XM_006717710; XM_024447887; XM_024447888; NM_001320658; |
| | NM_022976; XM_017015920; NM_001144918; NM_022974; NM_023031; |
| | XM_024447889; XM_024447891; NM_023029; XM_017015921; |
| | NM_001144914; NM_001144916; NM_022972 |
| FGFR3 | XM_047449823; XM_047449824; XM_006713869; XM_006713873; |
| | NM_022965; XM_006713868; NM_001354810; XM_011513422; |
| | XM_047449821; XM_047449822; NM_000142; XM_011513420; |
| | XM_047449820; XM_006713870; XM_006713871; NM_001163213; |
| | NM_001354809; NR_148971 |
| FGFR4 | NM_213647; NM_022963; NM_002011; NM_001291980; NM_001354984 |
| FGG | NM_000509; NM_021870 |
| FGL1 | NM_201552; XM_047421577; NM_201553; NM_004467; NM_147203 |
| FGL2 | NM_006682 |
| FGR | NM_005248; NM_001042729; NM_001042747 |
| FHIT | NM_001320900; NM_002012; NR_148922; NR_135491; NM_001354589; |
| | NM_001354590; NM_001320901; NR_148921; NM_001320899; |
| | NM_001166243 |
| FKBP11 | XM_047428939; NM_001143782; NM_016594; XM_047428940; |
| | NM_001143781 |
| FKBP15 | XM_006717018; NM_015258; XM_006717019 |
| FLT1 | NM_001160030; NM_001159920; XM_011535014; XM_017020485; |
| | NM_001160031; NM_002019 |
| FLT3 | XM_011535015; NM_004119; XM_011535018; XM_017020488; |
| | NR_130706; XM_017020486; XM_047430216 |
| FLT3LG | XM_047438520; XM_047438522; NM_001204503; NM_001278637; |
| | NM_001278638; XM_011526678; XM_047438521; XM_011526677; |
| | XM_047438523; XM_006723116; XM_011526675; XM_011526680; |
| | XM_047438519; XM_011526676; NM_001204502; NM_001459 |
| FLT4 | XM_011534478; XM_047417002; NM_001354989; XM_047417003; |
| | XM_017009266; XM_017009268; NM_002020; NM_182925; |
| | XM_017009263; XM_011534484 |
| FMNL1 | XM_006722064; XM_047436644; NM_005892; XM_006722062; |
| | XM_006722069; XM_047436641; XM_006722070; XM_047436637; |
| | XM_047436642; XM_047436643; XM_011525179; XM_047436640; |
| | XM_006722065; XM_047436639; XM_011525180; XM_047436638; |
| | XM_006722063; XM_047436646; XM_006722066; XM_047436645 |
| FMO3 | XM_047416207; NM_001002294; NM_006894; NM_001319173; |
| | NM_001319174 |
| FN1 | NM_001306129; NM_001365519; NM_212474; NM_001306132; |
| | NM_001365517; NM_001365522; NM_001306131; NM_001365521; |
| | NM_212476; NM_212478; NM_212475; NM_001365523; NM_001365524; |
| | NM_002026; NM_001365520; NM_212482; NM_001365518; NM_054034; |
| | NM_001306130 |
| FNBP1 | XM_005251830; XM_005251834; XM_011518400; XM_005251820; |
| | XM_005251825; XM_011518401; XM_011518402; XM_047423011; |
| | XM_047423013; XM_047423017; XM_005251815; XM_005251823; |
| | XM_005251824; XM_005251827; XM_011518399; XM_047423015; |
| | XM_047423022; NM_001363755; XM_005251821; XM_005251832; |
| | XM_006717016; XM_047423012; XM_047423016; NM_015033; |
| | XM_005251826; XM_017014488; XM_047423020; XM_005251831; |
| | XM_005251833; XM_005251828; XM_047423018; XM_047423021; |
| | XM_005251822; XM_017014492; XM_047423014; XM_047423019 |
| FOLH1 | XM_017017432; XM_047426680; XM_047426681; NM_001193471; |
| | NM_001351236; XM_047426679; XM_011519958; XM_047426678; |
| | XM_047426682; NM_001193472; NM_001014986; XM_017017433; |
| | XM_017017434; NM_001193473; NM_004476 |
| FOLR1 | NM_000802; NM_016729; NM_016730; NM_016725; NM_016724 |
| FOLR2 | NM_001113535; XM_005273856; XM_047426683; NM_000803; |
| | NM_001113534; NM_001113536 |
| FOS | NM_005252 |
| FOXC2 | NM_005251 |
| FOXL1 | NM_005250 |
| FOXM1 | XM_011520932; XM_011520934; XM_047428547; XM_047428548; |
| | NM_001243088; XM_011520930; XM_011520933; XM_011520935; |
| | XR_931507; XM_047428549; XM_047428551; NM_202003; |
| | XM_047428546; NM_202002; XM_005253676; XM_011520931; |
| | XM_047428550; NM_001243089; NM_021953 |
| FOXO1 | XM_047430204; XM_011535008; XM_011535010; NM_002015 |
| FOXP3 | NM_001114377; NM_014009 |
| FOXQ1 | NM_033260 |
| FPR1 | NM_002029; NM_001193306 |
| FPR2 | NM_001005738; NM_001462; XM_006723120 |
| FPR3 | NM_002030; XM_011526687 |
| FRAT1 | NM_181355; NM_005479 |
| FRAT2 | NM_012083 |
| FSCN1 | NM_003088 |
| FTCD | NM_006657; XM_011529440; NM_206965; NM_001320412; |
| | XM_006723962; XM_011529436; XM_047440669 |
| FXYD1 | NM_001278718; XM_047438938; NM_001278717; NM_021902; |
| | XM_017026875; NM_005031; XM_017026874; XM_017026876 |
| FZD1 | NM_003505 |
| FZD10 | NM_007197 |
| FZD2 | NM_001466 |
| FZD3 | XM_047422239; NM_145866; XM_047422238; XM_047422240; |
| | XM_017013842; NM_017412; XM_017013841; XM_017013844; |
| | XM_017013843 |
| FZD4 | NM_012193 |
| FZD5 | NM_030804; NM_003468 |
| FZD6 | NM_003506; NM_001164615; NM_001164616; NM_001317796; NR_133921 |
| FZD7 | NM_003507 |
| FZD8 | NM_031866 |
| FZD9 | NM_003508 |
| G6PC | NM_000151; NM_001270397 |
| GABARAP | NM_007278 |
| GABARAPL1 | NM_031412; NM_001363598 |
| GABARAPL2 | NM_007285 |
| GADD45G | XM_011518163; NM_006705 |
| GAS6 | NM_001143945; NM_001143946; NM_000820 |
| GATA1 | NM_002049 |
| GATA3 | XM_005252443; XM_047425044; NM_002051; XM_005252442; |
| | XM_047425045; NM_001002295 |
| GCKR | XM_017003797; XM_011532763; XM_017003796; XR_001738699; |
| | NM_001486 |
| GEN1 | XM_011532821; XM_047444156; XM_006712005; XM_047444147; |
| | NM_182625; XM_011532822; XM_047444155; XM_047444158; |
| | XM_047444159; XM_005262613; NM_001130009; XM_011532820; |
| | XM_047444157 |
| GHR | NM_001242401; NM_001242460; NM_001242462; NM_001242402; |
| | NM_001242406; NM_001242399; NM_001242403; NM_001242461; |
| | NM_001242405; NM_000163; NM_001242400; NM_001242404 |
| GINS2 | NM_016095 |
| GJA1 | NM_000165 |
| GJA10 | NM_032602 |
| GJA3 | NM_021954; XM_011535048 |
| GJA4 | XM_017001043; XM_005270750; NM_002060 |
| GJA5 | NM_005266; NM_181703 |
| GJA8 | XM_011509417; NM_005267 |
| GJA9 | NM_030772 |
| GJB1 | NM_000166; XM_011530907; NM_001097642 |
| GJB2 | XM_011535049; NM_004004 |
| GJB3 | NM_024009; NM_001005752 |
| GJB4 | XM_011540679; NM_153212 |
| GJB5 | XM_005270751; NM_005268 |
| GJB6 | NM_001110219; NM_001110221; NM_001370090; XM_047430057; |
| | NM_001370091; NM_001110220; NM_001370092; XM_047430056; |
| | NM_006783 |
| GJB7 | NM_198568 |
| GJC1 | XM_047435078; XM_047435079; NM_005497; XM_024450526; |
| | XR_007065255; XM_047435080; XM_024450527; XR_934346; |
| | XM_047435077; NM_001080383 |
| GJC2 | NM_020435 |
| GJC3 | XM_047420329; NM_181538 |
| GJD2 | NM_153368 |
| GJD3 | NM_152219 |
| GJD4 | NM_153368.3 |
| GJE1 | NM_001358410.2 |
| GLCCI1 | NM_138426 |
| GLI1 | NM_001160045; NM_005269; XM_011538190; NM_001167609; |
| | XM_011538189 |
| GLI2 | XM_011510969; XM_017003818; XM_011510971; XM_047443947; |
| | NM_030380; NM_030379; NM_001374354; NM_005270; NM_030381; |
| | NM_001371271; NM_001374353 |
| GLI3 | XM_047420208; XM_047420207; NM_000168; XM_011515274; |
| | XM_017011997; XM_047420209; XM_047420206; XM_047420205 |
| GLT1D1 | NR_159493; NM_144669; XM_047428371; XM_047428372; |
| | XM_047428373; XR_001748588; XM_011537957; NM_001366886; |
| | NM_001366887; NM_001366888; NM_001366889; NR_133646 |
| GLYAT | NM_201648; XM_017017087; NM_005838 |
| GLYATL1 | XM_011545355; NM_001354699; NM_001389712; NM_001389717; |
| | NM_001389714; NM_001389716; XM_011545356; NM_001220494; |
| | NM_001220496; NM_001389713; XM_011545357; NM_001389715; |
| | XM_011545354; NM_001389711; NM_001389718; NM_080661; |
| | XM_011545358; XM_011545359 |
| GLYCTK | XM_047447465; XM_047447467; NR_026699; XM_017005730; |
| | NR_026700; XM_024453352; XM_047447466; NM_001144951; |
| | NR_026701; XM_024453351; NM_145262; NR_026702 |
| GNA13 | NM_006572; NM_001282425 |
| GNLY | XM_005264085; NM_001302758; XM_047442947; NM_006433; |
| | XM_005264084; NM_012483 |
| GNRHR | NM_001012763; NM_000406 |
| GPC3 | NM_004484; XM_017029413; NM_001164618; NM_001164617; |
| | NM_001164619 |
| GPC5 | XM_017020437; XM_047430153; XM_017020435; XM_017020436; |
| | XM_011521055; XM_011521054; XM_011521057; XM_011521058; |
| | XM_011521060; XM_011521056; XM_011521059; NM_004466 |
| GPNMB | XM_047419776; XM_005249578; NM_001005340; XM_017011678; |
| | NM_002510 |
| GPR174 | XM_047442580; NM_032553; XM_047442579 |
| GPR18 | NM_001098200; NM_005292 |
| GPR34 | NM_005300; NM_001033513; XM_005272597; NM_001033514; |
| | NM_001097579 |
| GPR35 | NM_001195382; NM_001195381; NM_001394730; NM_005301 |
| GPT | NM_005309; NM_001382664; NR_168476; NM_001382665; NR_168477 |
| GRAMD1A | XM_011527153; XM_047439133; XM_047439134; XM_017027035; |
| | XM_011527155; XM_024451622; NM_001320035; NM_020895; |
| | XM_011527149; NM_001136199; NM_001320036; XM_017027034; |
| | NM_001320034 |
| GRAP2 | NM_001291825; NM_001291826; XM_047441608; NM_001291824; |
| | XM_047441607; NM_004810; XR_007067996; NM_001291828; |
| | XR_007067995 |
| GRB2 | NM_203506; NM_002086 |
| GRPR | NM_005314 |
| GSK3B | NM_001354596; NM_002093; XM_006713610; NM_001146156 |
| GSTP1 | NM_000852 |
| GTF2H1 | XM_006718208; NM_001142307; XM_024448457; NM_005316; |
| | XM_024448458 |
| GTF2H2 | NM_001395393; XM_047417130; NM_001364567; NM_001395387; |
| | NM_001395390; NM_001395391; NM_001395394; NM_001395397; |
| | NM_001395396; NM_001364568; NM_001364571; NM_001364573; |
| | NM_001395395; NM_001395399; NM_001515; NM_001395388; |
| | NM_001364569; NM_001395389; NM_001364570; NM_001395392; |
| | XM_047417131; XM_047417132; NM_001364572; NM_001395398 |
| GTF2H3 | NM_001271866; NM_001516; XM_017019228; NM_001271867; |
| | NM_001271868 |
| GTF2H4 | NM_001517 |
| GTF2H5 | NM_207118 |
| GZMA | NM_006144 |
| GZMB | NM_001346011; NM_004131; NR_144343 |
| GZMH | NM_001270781; NM_001270780; NM_033423 |
| GZMK | NM_002104 |
| GZMM | NM_001258351; NM_005317 |
| HAL | NM_001258333; XM_011538249; NM_001258334; XM_017019246; |
| | NM_002108 |
| HAMP | NM_021175 |
| HAO1 | NM_017545 |
| HAO2 | XM_024447486; XM_024447484; XM_011541561; XM_024447495; |
| | NM_001377472; XM_047422154; XM_024447492; NM_001005783; |
| | NM_016527; XM_024447485 |
| HAVCR2 | NM_032782 |
| HCK | NM_001172132; NM_001172133; NM_001172130; NM_002110; |
| | NM_001172131; NM_001172129 |
| HCLS1 | NM_005335; NM_001292041 |
| HDAC1 | XM_011541309; NM_004964 |
| HDAC2 | NR_033441; XM_047418692; NR_073443; NM_001527 |
| HES1 | NM_005524 |
| HES2 | NM_019089 |
| HES3 | NM_001024598 |
| HES4 | XM_005244771; XM_047426314; NM_021170; NM_001142467; |
| | XM_047426320 |
| HES5 | XM_005244751; NM_001010926 |
| HEY1 | NM_001040708; NM_001282851; NM_012258 |
| HEY2 | NM_012259; XM_017010627; XM_017010628 |
| HEYL | NM_014571; XM_005270745 |
| HFE2 | NM_213652; NM_145277; NM_213653; NM_202004; NM_001379352; |
| | NM_001316767 |
| HGF | NM_000601; NM_001010933; XM_047420293; NM_001010934; |
| | NM_001010931; NM_001010932 |
| HGFAC | NM_001297439; XM_047450155; NM_001528 |
| HHIP | XM_005263178; NM_022475; XM_006714288 |
| HIC1 | NM_006497; NM_001098202 |
| HIF1A | NM_001530; NM_181054; NM_001243084 |
| HIPK2 | XM_011516081; NM_001113239; XM_011516078; XM_047420260; |
| | XM_047420261; XM_047420262; XM_047420265; NM_022740; |
| | XM_011516080; XM_011516077; XM_011516079; XM_047420263; |
| | XM_047420264 |
| HK3 | XM_047417134; XM_011534540; NM_002115; XR_941102 |
| HLA-A | XM_041680767; NM_001242758; XM_041680768; NM_002116 |
| HLA-B | NM_005514 |
| HLA-C | NM_002117; NM_001243042 |
| HLA-DMA | NM_006120 |
| HLA-DMB | NM_002118 |
| HLA-DOB | NM_002120 |
| HLA-DPA1 | NM_001405020; NM_001242525; NM_033554; XM_047418717; |
| | NM_001242524 |
| HLA-DPB1 | NM_002121 |
| HLA-DQA1 | NM_002122; XM_006715079 |
| HLA-DQB1 | NM_001243962; NM_001243961; NM_002123 |
| HLA-DRA | NM_019111 |
| HLA-DRB1 | XM_024452553; NM_001359194; XM_047444767; XM_047444769; |
| | NM_001243965; NM_002124; XM_047444770; NM_001359193; |
| | XM_047443024; XM_047444768 |
| HMGB1 | XM_047430284; NM_001370340; NM_002128; NM_001313892; |
| | NM_001313893; NM_001370339; NM_001363661; NM_001370341; |
| | XM_024449341; XM_047430285 |
| HMHA1 | XM_011527858; XM_047438547; NM_001282334; NM_001321232; |
| | XM_047438548; NM_001282335; NM_012292; XM_047438549; |
| | NM_001258328; XM_047438545; XM_047438546 |
| HOXA1 | NM_005522; NM_153620 |
| HOXB7 | NM_004502 |
| HOXB9 | NM_024017 |
| HOXC10 | NM_017409 |
| HP | NM_001126102; NM_005143; NM_001318138 |
| HPD | NM_001171993; NM_002150 |
| HPR | NM_001384360; XM_024450251; NM_020995 |
| HPSE | NM_001199830; NM_001098540; NM_006665; NM_001166498 |
| HPX | NM_000613 |
| HRAS | NM_005343; NM_001318054; NM_001130442; NM_176795 |
| HRG | NM_000412; XM_005247415 |
| HRK | NM_003806; NR_073189 |
| HRSP12 | NM_005836 |
| HSD17B13 | NM_001136230; NM_178135 |
| HSPA1A | NM_005345.6 |
| HSPA5 | NM_005347 |
| HSPB1 | NM_001540 |
| HSPH1 | XM_005266236; XM_017020364; XM_047430058; XM_011534888; |
| | NM_006644; XM_017020363; XM_017020362; XM_011534887; |
| | NM_001286504; NM_001286503; NM_001286505; NM_001349704 |
| HUS1 | NM_004507; NR_037917; NM_001363683 |
| ICAM1 | NM_000201 |
| ICAM3 | NM_001395374; NM_001395376; NM_001320605; NM_001320606; |
| | NM_002162; NM_001395375; NM_001320608 |
| ICOS | XR_007073112; XM_047444022; NM_012092 |
| ICOSLG | NM_001395918; XM_011529514; NM_001283051; NM_001283050; |
| | NM_001283052; XM_047440732; NM_015259; XM_047440731; |
| | NM_001365759; XM_011529516; XM_047440729; XM_047440730 |
| IDO1 | NM_002164 |
| IFI30 | NM_006332 |
| IFITM2 | NM_006435 |
| IFNA2 | NM_000605 |
| IFNAR1 | NM_000629; NM_001384499; NM_001384502; NM_001384498; |
| | NM_001384503; NM_001384501; NM_001384504; NM_001384500 |
| IFNAR2 | NM_001289126; NM_001385054; NM_001289128; NM_207584; |
| | NM_001385055; NM_000874; NM_001289125; NM_207585 |
| IFNB1 | NM_002176 |
| IFNG | NM_000619 |
| IFNGR1 | NM_001363526; XM_011535794; XM_047418726; XM_011535793; |
| | NM_000416; XM_006715470; NM_001363527 |
| IFNGR2 | NM_001329128; NM_005534 |
| IFNLR1 | NM_173064; XM_006710394; XM_017000479; NM_173065; NM_170743 |
| IGBP1 | XM_047442084; NM_001370193; NM_001551; XM_047442083; |
| | NM_001370194; NM_001370192 |
| IGF1 | XM_017019261; XM_017019262; XM_017019263; XM_017019259; |
| | NM_001111284; NM_001111285; NM_001111283; NM_000618 |
| IGF1R | XM_047432444; XM_011521517; NM_000875; XM_011521516; |
| | XM_017022137; XM_047432442; NM_152452; XM_047432443; |
| | XM_047432445; NM_001291858 |
| IGF2 | NM_001291862; NM_001291861; NM_000612; NM_001007139; |
| | NM_001127598 |
| IGF2BP3 | XM_011515092; NM_006547; XM_011515089; XM_011515090; |
| | XM_047419782; XM_047419784; XM_011515093 |
| IGFBP2 | NM_001313990; NM_000597; NM_001313992; NM_001313993 |
| IGFLR1 | NM_024660; NR_144338; NR_144340; NM_001346003; NM_001346004; |
| | NR_144341; NR_144342; NR_144339; NM_001346005; NM_001346006 |
| IGHG1 | NG_001019.6 |
| IGHG3 | NG_001019.6 |
| IGHM | NG_001019.6 |
| IGKC | NG_000834.1 |
| IGLL5 | NM_001178126; NM_001256296 |
| IGSF6 | NM_005849 |
| IKBKB | NM_001190720; NR_033818; XM_047421757; NM_001242778; |
| | NR_040009; NM_001190722; XM_047421764; XM_047421760; |
| | XM_047421758; XM_011544520; XM_047421759; XM_047421761; |
| | XM_047421763; NM_001556; NR_033819; XM_011544517; |
| | XM_047421762; NM_001190721 |
| IKBKG | NM_001099856; NM_003639; NM_001099857; NM_001377315; |
| | NM_001377312; NM_001145255; NM_001321397; NM_001321396; |
| | NM_001377313; NR_165197; NM_001377314 |
| IKZF1 | XM_011515064; XM_011515071; XM_011515073; XM_017011668; |
| | XM_047419729; XM_047419732; XM_047419733; XM_047419741; |
| | NM_001220767; NM_001291841; NM_001291842; NM_001220775; |
| | XM_011515061; XM_011515063; XM_011515065; XM_011515072; |
| | XM_011515078; XM_047419723; XM_047419730; XM_047419736; |
| | XM_047419742; XM_047419749; NM_001291837; NM_001291846; |
| | NM_001220774; XM_011515062; XM_011515066; XM_047419726; |
| | XM_047419739; XM_047419740; XM_047419743; XM_047419746; |
| | XM_047419747; NM_006060; NM_001220772; XM_047419748; |
| | NM_001220768; NM_001220771; NM_001291843; NM_001291845; |
| | XM_011515060; XM_011515067; XM_047419731; XM_047419738; |
| | NM_001291838; NM_001291840; NM_001220769; XM_011515058; |
| | XM_011515059; XM_011515070; XM_047419728; XM_047419734; |
| | XM_047419735; XM_047419745; NM_001220765; NM_001220770; |
| | NM_001291839; NM_001291844; XM_011515077; XM_047419724; |
| | XM_047419744; XM_047419750; NM_001220773; XM_011515074; |
| | XM_047419725; XM_047419727; NM_001291847; NM_001220766; |
| | NM_001220776 |
| IKZF2 | XM_011510804; XM_011510815; NM_001371277; XM_011510803; |
| | XM_011510808; XM_011510816; NM_001371275; XM_005246386; |
| | XM_011510807; XM_011510810; XM_047443723; XM_011510811; |
| | NM_001371276; XM_011510809; XM_047443722; XM_047443724; |
| | NM_001079526; NM_001371274; NM_001387220; NM_016260; |
| | XM_011510817; XM_011510818; XM_011510805; XM_011510812; |
| | XM_011510819; XM_047443721; XM_047443725; XM_047443727; |
| | XM_011510802; XM_047443726 |
| IKZF3 | NM_001257411; NM_001284516; NM_001257414; NM_001284515; |
| | NM_183230; NM_183231; NM_183232; NM_001257412; NM_001257413; |
| | NM_001257408; NM_001257409; NM_001284514; NM_183228; |
| | XM_047435625; NM_001257410; NM_012481; NM_183229 |
| IKZF4 | XM_017019810; XM_047429342; XM_047429345; XM_047429347; |
| | NM_001351089; NM_022465; XM_047429341; XM_047429346; |
| | XM_047429349; XM_047429351; NM_001351090; NM_001351091; |
| | XM_011538664; XM_011538669; XM_047429350; XM_047429352; |
| | XM_047429353; XM_017019806; XM_047429348; XM_005269089; |
| | XM_047429344; NM_001351092; XM_017019812 |
| IL10 | NM_000572; NR_168467; NR_168466; NM_001382624 |
| IL10RA | XM_047426883; NM_001558; XM_047426884; XM_047426882; NR_026691 |
| IL10RB | NR_175973; NM_001405849; NM_001405850; NM_000628 |
| IL11RA | NR_052010; NM_004512; NM_147162; NM_001142784 |
| IL12A | NM_000882; NM_001354583; NM_001354582; NM_001397992 |
| IL12B | NM_002187 |
| IL12RB2 | NR_047584; XM_011541384; XM_047419669; XM_047419670; |
| | XM_005270827; XM_006710617; NM_001374259; XM_011541383; |
| | XM_047419667; NM_001258215; NM_001258216; XM_047419665; |
| | XM_047419666; XM_047419668; NM_001258214; NM_001319233; |
| | XM_005270828; XM_017001203; NM_001559; NR_047583 |
| IL13 | NM_001354991; NM_001354992; NM_002188; NM_001354993 |
| IL13RA1 | XM_047442096; NM_001560 |
| IL13RA2 | NM_000640 |
| IL15RA | XM_011519471; XM_047425181; NM_002189; NR_046362; |
| | XM_011519463; XM_011519465; XM_011519468; XM_011519469; |
| | XM_017016197; XM_047425182; XM_047425184; XM_011519467; |
| | XM_017016198; XM_017016199; NM_001351095; XM_011519462; |
| | XM_011519464; XM_011519466; XM_047425185; XM_047425190; |
| | XM_011519475; XM_017016195; XM_047425187; XM_047425189; |
| | XM_047425191; NM_001243539; NM_001351096; NM_001351097; |
| | NM_172200; XM_011519470; XM_047425186; NM_001256765; |
| | XM_011519461; XM_011519474; XM_011519477; XM_047425183; |
| | XM_011519472; XM_011519476; XM_047425188 |
| IL16 | XM_047432448; NM_004513; NM_172217; XM_047432451; |
| | XM_047432458; NM_001172128; NM_001352684; NR_148035; |
| | XM_047432450; XM_047432457; NM_001352686; XM_047432452; |
| | NM_001352685; XM_047432447; XM_047432454; XM_047432449; |
| | XM_047432453; XM_047432455; XM_047432456 |
| IL17A | NM_002190 |
| IL17F | XM_011514276; NM_052872; NM_172343 |
| IL18RAP | NM_001393489; XM_047446162; XM_011512088; XM_024453197; |
| | NM_001393487; NM_001393486; XR_007083519; XM_024453199; |
| | XM_024453201; NM_003853; XM_024453198; XM_047446163; |
| | NM_001393488 |
| ILIA | NM_001371554; NM_000575 |
| IL1B | NM_000576; XM_047444175 |
| IL1RN | NM_001318914; XM_047444186; XM_047444184; XM_011511121; |
| | XM_047444185; NM_001379360; NM_173841; NM_173843; NM_173842; |
| | NM_000577 |
| IL2 | NM_000586 |
| IL21 | NM_021803; NM_001207006 |
| IL21R | XM_017023257; NM_181078; XM_047434180; NM_021798; NM_181079; |
| | XM_011545857 |
| IL22 | NM_020525 |
| IL23A | NM_016584 |
| IL23R | XM_011540791; XM_047447227; NM_144701; XM_011540790 |
| IL26 | NM_018402 |
| IL27 | NM_145659 |
| IL2RA | NM_001308243; NM_001308242; NM_000417 |
| IL2RB | NM_000878; NM_001346223; NM_001346222 |
| IL2RG | XM_047442089; NM_000206 |
| IL33 | XM_047424061; XM_047424063; NM_001314046; NM_001314048; |
| | XM_047424062; NM_001314047; NM_001353802; NM_033439; |
| | XM_047424064; NM_001199641; NM_001314045; XM_047424060; |
| | NM_001199640; XM_017015285; NM_001314044 |
| IL3RA | XM_047442090; XM_047442730; XM_005274431; XM_017030043; |
| | NM_002183; XM_005274781; NM_001267713; XM_005274432; |
| | XM_017029491; XM_005274780; XM_047442731; XM_047442091 |
| IL4 | NM_000589; NM_001354990; NM_172348 |
| IL4I1 | NM_001385639; NM_172374; NM_152899; NR_047577; NM_001258018; |
| | NM_001258017 |
| IL4R | XM_047434066; NM_001257406; NM_001257407; NM_001008699; |
| | XM_011545827; XM_011545834; XM_011545825; XM_011545833; |
| | XM_017023211; XM_005255308; XM_011545828; XM_047434068; |
| | NM_000418; XM_011545826; NM_001257997; XM_047434067 |
| IL5 | XM_047417148; XM_005271988; XM_011543373; NM_000879 |
| IL5RA | NM_175726; NM_175724; NM_175727; XM_011533677; NM_001243099; |
| | XM_011533678; NM_175725; NM_175728; NM_000564 |
| IL6 | NM_001318095; NM_000600; NM_001371096; XM_005249745 |
| IL6R | XM_047419649; XM_047419654; NM_001382771; XM_005245139; |
| | NM_001206866; NM_001382770; NM_181359; XM_047419650; |
| | XM_047419656; NM_001382773; XM_047419657; NM_000565; |
| | NM_001382769; NM_001382774; NM_001382772; XM_017001199; |
| | XM_047419648; XM_047419655 |
| IL6ST | NM_175767; NM_001364279; NR_157112; NM_001364276; |
| | NM_001364275; NM_001364277; NM_002184; NM_001190981; |
| | NM_001364278; NR_120480 |
| IL7R | XM_047417149; XM_005248299; XM_047417150; NR_120485; NM_002185 |
| IL9 | NM_000590 |
| IL9R | XM_011545650; XM_047442734; XM_017030051; XM_047442095; |
| | XM_011531155; XM_017029502; XM_017030055; NM_176786; |
| | XM_011545645; XM_017029495; XM_047442092; XM_047442732; |
| | XM_011531152; XM_011531157; XM_017030044; XM_047442094; |
| | XM_047442733; NM_002186; XM_011531151; XM_011545646; |
| | XM_011545652; XM_017029506; XM_047442093; XM_047442735 |
| ING4 | XM_047428932; NM_001127585; NM_016162; NM_198287; |
| | XM_011520965; NM_001127584; NM_001127586; NM_001127582; |
| | NM_001127583; XM_011520964; XM_047428931 |
| INHBA | NM_002192; XM_047420335; XM_017012174 |
| INHBB | NM_002193 |
| INPP4B | XM_047416356; XM_047416357; XM_047416365; NM_001385334; |
| | NM_001385339; NM_001385341; NM_001385348; NM_001385460; |
| | NM_001385461; NM_003866; NR_169615; XM_017008798; |
| | XM_024454274; NM_001385357; NM_001385362; NM_001385381; |
| | NM_001385452; NR_169614; XR_007057981; XM_047416352; |
| | XM_047416367; NM_001101669; NM_001385344; NM_001385379; |
| | NM_001385457; XM_047416354; XM_047416358; XM_047416361; |
| | NM_001385337; NM_001385342; NM_001385351; NM_001385458; |
| | NR_169619; NR_169623; NR_169624; XM_024454273; XM_011532391; |
| | XM_047416363; XM_047416366; NM_001385336; XM_017008797; |
| | XM_047416353; XM_047416359; XM_047416368; NM_001331040; |
| | NM_001385382; NM_001385383; NM_001385450; NM_001385454; |
| | NM_001385455; NR_169599; NR_169617; NR_169618; XM_047416362; |
| | NM_001385335; NM_001385340; NM_001385347; NM_001385459; |
| | XM_047416360; NM_001385338; NM_001385343; NM_001385350; |
| | NM_001385380; NR_169616 |
| INPP5D | XM_047444219; NM_005541; NM_001017915; XM_047444220 |
| INPPL1 | XM_047426890; XM_005273979; XM_047426889; XM_047426892; |
| | XM_047426888; XM_047426893; XM_047426887; NM_001567; |
| | XM_011544999; XM_047426891 |
| INSIG2 | NM_016133; NM_001321333; NM_001321330; XM_047444640; |
| | NM_001321331; NM_001321332; NM_001321329 |
| INSR | NM_001079817; NM_000208; XM_011527989; XM_011527988 |
| INSRR | NM_014215 |
| IRAK1 | XM_005274668; XM_047442097; NM_001025242; XM_047442098; |
| | NM_001025243; NM_001569 |
| IRAK2 | NM_001570 |
| IRF1 | XM_047417153; NR_149068; NM_001354924; NR_149069; |
| | XM_011543379; XM_047417154; NM_002198; NM_001354925 |
| IRF4 | NM_002460.4; NM_001195286.2; NR_046000.3 |
| IRF5 | XM_047420336; NM_001242452; XM_006715974; NM_032643; |
| | NM_001364314; XM_011516160; XM_011516158; XM_047420340; |
| | NM_001347928; XM_047420337; NM_001098629; XM_011516159; |
| | XM_047420338; NM_001098627; NM_001098630; XM_047420339 |
| IRF9 | NM_006084; NM_001385402; NM_001385400; NM_001385401 |
| ITGA1 | NM_181501 |
| ITGA10 | XM_017002623; XM_047432904; XM_047432906; XM_017002626; |
| | XM_047432895; XM_047432909; XM_047432910; NM_001303041; |
| | XR_007064482; XM_047432915; NM_001303040; XM_017002622; |
| | XM_017002625; NM_003637; XM_047432924; XR_001737504; |
| | XM_017002624; XM_011510083; XM_011510084; XM_017002627 |
| ITGA11 | XM_011521363; NM_012211; XM_005254228; NM_001004439 |
| ITGA2 | NR_073103; NR_073104; NR_073105; NR_073106; NR_073107; |
| | NM_002203 |
| ITGA2B | NM_000419; XM_011524749; XM_011524750 |
| ITGA3 | XM_005257308; NM_002204; NM_005501; XM_047435922 |
| ITGA4 | NM_001316312; NM_000885 |
| ITGA5 | NM_002205; XM_024448970 |
| ITGA6 | XM_047444221; XM_017004006; NM_000210; NM_001079818; |
| | NM_001394928; NM_001365530; NM_001316306; XM_017004005; |
| | NM_001365529; XM_047444222 |
| ITGA7 | XM_005268844; XM_005268848; XM_047428792; XM_047428799; |
| | XM_005268839; XM_017019265; XM_047428794; NM_002206; |
| | XM_047428793; NM_001374465; XM_047428798; XM_047428791; |
| | XM_005268840; XM_005268841; XM_047428795; XM_047428797; |
| | NM_001367993; NM_001367994; XM_047428796; NM_001144996; |
| | NM_001144997 |
| ITGA8 | NM_003638; XM_011519752; NM_001291494 |
| ITGA9 | NM_002207 |
| ITGAD | XM_047434071; NM_005353; XM_011545836; XM_011545838; |
| | XM_011545839; XM_011545844; XM_011545835; XM_011545837; |
| | XM_011545842; XR_950791; XM_011545845; XM_011545846; |
| | XM_011545847; XM_017023215; NM_001318185; XM_011545841; |
| | XM_011545843; XM_011545848 |
| ITGAE | XM_047435924; XM_011523825; XM_047435923; XM_011523828; |
| | XM_024450740; XM_017024586; NM_002208; XM_011523827 |
| ITGAL | XM_005255313; XM_006721044; NM_001114380; XR_950794; |
| | XM_047434073; XM_047434072; NM_002209 |
| ITGAM | XR_950796; NM_000632; XM_011545850; XM_011545851; |
| | XM_017023216; NM_001145808; XM_006721045; XR_007064878 |
| ITGAV | XM_047444225; NM_001144999; NM_002210; NM_001145000 |
| ITGAX | NM_001286375; XM_024450263; XM_011545852; XM_011545854; |
| | XM_047434075; NM_000887; XM_047434074 |
| ITGB1 | NM_033666; NM_033669; NM_002211; NM_033667; NM_033668; |
| | NM_133376 |
| ITGB2 | XM_047440763; NM_000211; NM_001303238; XM_006724001; |
| | NM_001127491 |
| ITGB3 | NM_000212 |
| ITGB4 | XM_047435927; XM_005257311; XM_006721866; XM_006721870; |
| | NM_000213; NM_001005619; NM_001005731; XM_005257309; |
| | XM_011524752; XM_006721867; XM_011524751; XM_047435929; |
| | NM_001321123; XM_047435926; XM_047435928; XM_006721868 |
| ITGB5 | NM_001354766; XM_047448087; XM_005247436; XM_006713630; |
| | XM_047448089; NM_002213; XM_017006353; NM_001354765; |
| | XM_047448088; NM_001354764 |
| ITGB6 | NM_001282354; NM_001282353; NM_000888; NM_001282389; |
| | NM_001282390; NM_001282355; NM_001282388 |
| ITGB7 | XM_005268851; XM_005268852; NM_000889; NR_104181; XM_047428800 |
| ITGB8 | XM_011515393; XM_047420343; NM_002214; XM_047420341; |
| | XM_047420344; XM_011515394; XM_017012182; XM_017012178; |
| | XM_017012183; XM_047420345; XM_017012179; XM_017012180; |
| | XM_047420342 |
| ITIH1 | NM_001166436; NM_002215; NM_001166434; NM_001166435 |
| ITIH2 | NM_002216 |
| ITIH3 | NM_001392021; NM_001392022; NM_002217; NM_001392024; |
| | NM_001392023; NM_001392025; NM_001392026; NM_001392019; |
| | NM_001392020; NM_001392027 |
| ITIH4 | NM_001166449; NM_002218 |
| ITIH5 | NM_001001851; NM_032817; XM_011519713; NM_030569; |
| | XM_011519714 |
| ITK | NM_005546 |
| ITM2A | NM_004867; NM_001171581 |
| ITPKB | NM_002221; NM_001388404; XM_017001211 |
| ITPR1 | NM_002222; NM_001099952; NM_001378452; NM_001168272 |
| ITPR2 | XM_047428801; XM_017019266; XR_001748687; XM_017019269; |
| | NM_002223; XR_001748686 |
| ITPR3 | XM_047418731; XM_017010832; XM_047418734; XM_011514577; |
| | XM_047418733; NM_002224; XM_047418732 |
| JAG1 | NM_000214 |
| JAG2 | NM_145159; XM_047431352; XM_047431354; NM_002226; |
| | XM_047431353 |
| JAK1 | NM_001321856; XM_047419677; NM_001321857; NM_001321853; |
| | NM_001321854; NM_001321855; XM_047419674; XM_047419675; |
| | NM_002227; XM_047419676; NM_001321852; NM_001320923 |
| JAK2 | NM_001322195; NM_001322196; NM_001322194; NM_001322198; |
| | NM_004972; NM_001322199; NM_001322204; NR_169763; NR_169764 |
| JAK3 | NM_000215; XR_007066796; XM_011527991; XM_047438786 |
| JAM2 | NM_001270408; NM_021219; NR_072999; NM_001270407 |
| JAM3 | NM_032801; NM_001205329 |
| JUP | XM_047435934; XM_047435935; XM_047435940; XM_017024590; |
| | XM_006721875; XM_047435942; NM_001352773; NM_001352776; |
| | XM_047435938; NM_001352777; NM_021991; XM_047435939; |
| | NM_001352775; NM_002230; XM_006721874; XM_011524758; |
| | XM_047435937; XM_047435941; NM_001352774 |
| KAT2B | NM_003884; XM_005265528; XM_047449147 |
| KAT5 | XM_047426253; NM_182710; XM_047426252; NM_001206833; |
| | NM_006388; NM_182709; XM_006718421 |
| KCNA3 | NM_002232; NR_109846; NR_109845 |
| KCNAB2 | XM_047432867; XM_047432870; XM_047432872; NM_003636; |
| | XM_047432878; XM_011542322; NM_001199860; NM_001199863; |
| | XM_017002620; XM_005263514; XM_011542321; NM_001199861; |
| | NM_001199862; XM_047432866; NM_172130 |
| KCNH2 | XM_017012195; XM_017012196; NM_000238; XM_011516185; |
| | NM_172056; XM_047420349; XM_047420348; NM_001204798; |
| | NM_172057 |
| KCNJ15 | NM_170736; NM_170737; NM_001276438; NM_001276439; NM_002243; |
| | NM_001276435; NM_001276436; NM_001276437 |
| KDM3A | XM_047445104; XM_047445105; NM_001146688; XM_006712051; |
| | XM_047445101; XM_047445102; NM_018433; XM_047445103 |
| KDM4A | NM_014663 |
| KDM5A | NM_005056; NM_001042603 |
| KDM8 | XM_047434657; XM_047434655; XM_047434654; NM_024773; |
| | XM_047434658; NM_001145348; XM_017023676; XM_047434656 |
| KDR | NM_002253 |
| KEAP1 | NM_203500; XM_005260174; XM_011528452; XM_005260173; |
| | NM_012289; XM_047439788 |
| KIF14 | XM_011510235; XM_047436199; NM_001305792; XM_047436181; |
| | XM_011510232; XM_017003006; XM_017003007; XM_011510231; |
| | XM_011510233; XM_047436195; XM_047436197; XM_047436184; |
| | XM_047436198; NM_014875; XM_047436190 |
| KIF20A | NM_005733 |
| KIR2DL1 | NM_014218 |
| KIR2DL2 | NM_014219 |
| KIR2DL3 | NM_015868; NM_014511 |
| KIR2DL4 | NM_001080770; NM_001080772; NM_002255; NM_001258383 |
| KIR2DS2 | NM_001291696; NM_001291700; NM_012312; NM_001291695; |
| | XM_017030275; NM_001291701 |
| KIR3DL1 | XM_017030274; NM_001322168; NM_013289 |
| KIR3DL2 | XM_047438795; NM_006737; NM_001242867 |
| KISS1 | NM_002256 |
| KIT | NM_001385284; NM_000222; NM_001385286; NM_001093772; |
| | NM_001385288; NM_001385292; NM_001385290; NM_001385285 |
| KITLG | NM_003994; NM_000899 |
| KLF4 | NM_004235; NM_001314052 |
| KLF6 | NM_001160125; NM_001008490; NM_001160124; NM_001300; NR_027653 |
| KLF8 | XM_047441795; XM_047441799; NM_001324105; XM_047441797; |
| | XM_006724575; NM_001324102; XM_047441800; NM_001159296; |
| | NR_136704; XM_047441796; NM_001324100; NM_001324099; |
| | NM_001324104; NR_136705; XM_024452332; XM_047441798; |
| | XM_017029250; NM_007250 |
| KLK10 | XM_047439102; XM_006723289; XM_005259061; NM_002776; |
| | NM_145888; NM_001077500; XM_017026993; XM_006723287; |
| | XM_005259062 |
| KLK3 | NM_001030050; NM_001030047; NM_145864; NM_001030049; |
| | NM_001030048; NM_001648 |
| KLKB1 | NM_001318396; XM_017008182; XM_017008184; NM_001318394; |
| | XM_011531930; XM_047415661; XM_017008181; NM_000892; |
| | XM_017008183 |
| KLRB1 | NM_002258 |
| KLRC1 | NM_002259; NM_007328; NM_001304448; NM_213657; NM_213658 |
| KLRC2 | NM_002260 |
| KLRC3 | NM_002261; NM_007333 |
| KLRC4 | NM_013431 |
| KLRD1 | XM_011520651; XM_047428824; XM_047428821; NM_001351062; |
| | XM_047428823; NR_147039; XM_047428825; NM_001114396; |
| | NM_001351060; NM_002262; NR_147038; NR_147040; XM_024448974; |
| | XM_047428822; NM_001351063; NM_007334 |
| KLRF1 | NM_001291822; XM_017019415; XM_047428956; NM_001366534; |
| | NR_120305; NM_001291823; NM_016523; NR_159359; NR_159360; |
| | NR_159361 |
| KLRG1 | XM_017018682; XM_017018684; XM_047428074; NR_137426; |
| | NM_001329102; NR_137427; NM_001329103; NM_001329099; |
| | NM_001329101; NM_005810; NR_137428; XM_017018685; XM_047428075 |
| KLRK1 | NM_007360.4 |
| KMT2A | XM_011542831; XM_006718839; XM_047426963; XM_011542829; |
| | XM_011542830; XM_047426964; NM_005933; NM_024891; |
| | NM_001197104; XM_011542833 |
| KMT2C | NM_021230; NM_170606 |
| KMT2D | NM_003482 |
| KNG1 | NM_000893; NM_001102416; NM_001166451 |
| KRAS | XM_047428826; NM_001369786; NM_033360; NM_004985; |
| | NM_001369787 |
| KRT19 | NM_002276 |
| LACC1 | XM_011534935; XM_047430105; NM_001350643; NM_001350641; |
| | XM_024449319; XM_047430101; XM_047430109; NM_001350647; |
| | XM_047430103; XM_047430102; XM_047430104; XM_047430106; |
| | NM_001350639; NM_001128303; NM_001350646; NM_001350645; |
| | NM_153218; XM_047430100; XM_047430099; XM_047430108; |
| | NM_001350642; NM_001350644; NM_001350648; XM_047430107; |
| | NM_001350638; NM_001350640 |
| LAG3 | NM_002286; XM_047428839; XM_011520956 |
| LAIR1 | NM_001289025; NM_002287; XM_017026803; XM_047438810; |
| | NM_001289023; NM_001289026; NM_001289027; NM_021706; |
| | XM_047438811; NR_110280; XM_047438812; NM_021708; NR_110279 |
| LAMA2 | NM_001079823; NM_000426 |
| LAMA3 | XM_011525981; XM_017025743; XM_047437504; NM_001127717; |
| | NM_000227; XM_011525978; XM_011525979; XM_047437503; |
| | NM_198129; XM_011525980; XM_017025744; XM_047437506; |
| | XM_011525982; XM_047437505; NM_001302996; NR_130106; |
| | NM_001127718 |
| LAMA4 | XM_047418771; NM_001105206; XM_005266984; NM_002290; |
| | XM_047418769; NM_001105207; NM_001105208; NM_001105209; |
| | XM_047418770; XM_017010854; XM_005266983 |
| LAMB1 | XM_047420360; XM_047420359; NM_002291 |
| LAMB3 | XM_005273124; XM_047420351; NM_001127641; XM_017001272; |
| | NM_000228; NM_001017402 |
| LAMC2 | NM_005562; NM_018891; XM_047420361; XM_047420358; |
| | XM_017001273 |
| LAMP3 | XM_011512688; XM_047447967; XM_005247360; NM_014398 |
| LAPTM5 | XM_011542098; NM_006762 |
| LAT | NM_001014988; NM_001014989; NM_014387; NM_001014987 |
| LATS1 | XM_017011477; XM_017011479; NM_001270519; XM_011536252; |
| | NM_001350340; NR_073033; NM_001350339; XM_017011474; |
| | XM_047419521; NM_004690; XR_007059386; XM_047419518; |
| | XM_047419519; XM_047419522; XM_006715603; XM_047419517; |
| | NM_001350392; XM_024446583 |
| LATS2 | XM_017020541; XM_017020542; XM_011535042; XM_047430267; |
| | NM_014572; XM_005266342; XM_047430266 |
| LAX1 | NM_001282878; NM_001136190; NM_017773; XM_006711397 |
| LCAT | NM_000229 |
| LCK | XM_047420403; XM_011541453; XM_024447046; XM_047420399; |
| | NM_001330468; XM_024447047; NM_005356; NM_001042771 |
| LCP1 | XM_047430303; XM_047430305; NM_002298; XM_047430304; |
| | XM_005266374 |
| LDHA | NR_028500; NM_001165414; NM_001165415; NM_001165416; |
| | NM_005566; NM_001135239 |
| LEF1 | XM_005263047; XR_007057926; XR_007057925; XM_005263046; |
| | XM_006714233; XM_005263048; NM_001130713; NM_001130714; |
| | XR_007057927; NM_001166119; NM_016269 |
| LEP | XM_005250340; NM_000230 |
| LGALS1 | NM_002305 |
| LGALS3 | XM_047431367; XM_047431368; NM_001357678; NR_003225; |
| | NM_002306; XM_047431369; NM_001177388 |
| LGALS7 | NM_002307.4 |
| LGALS9 | XM_011524796; NM_001330163; NR_024043; XM_006721893; |
| | XM_006721895; NM_002308; XM_006721892; NM_009587 |
| LGR4 | NM_001346432; NM_018490 |
| LGR5 | XM_047429801; NR_110596; XM_047429800; NM_001277227; |
| | NM_001277226; NM_003667 |
| LGR6 | XM_011509842; XM_047426930; XM_005245404; XM_011509843; |
| | XM_047426929; XM_047426931; NM_021636; XM_047426932; |
| | XM_011509844; XM_011509840; XM_011509838; XM_017001997; |
| | NM_001017403; XM_011509841; XM_017001996; XM_047426928; |
| | NM_001017404; XM_011509839; XM_011509846 |
| LHX6 | XM_047423223; NM_001348190; NM_199160; XM_005251916; |
| | XM_047423224; NM_014368; NM_001242334; NM_001242333; |
| | NM_001242335 |
| LIFR | XM_017009463; XM_047417172; NM_001127671; NM_001364298; |
| | NM_002310; XM_011514042; NM_001364297 |
| LIG4 | NM_001352598; NM_001379095; NM_002312; NM_001352601; |
| | NM_206937; NM_001352602; NM_001352603; NM_001352600; |
| | NM_001330595; NM_001352604; NM_001098268; NM_001352599 |
| LILRA3 | NM_006865; NM_001172654 |
| LILRA4 | NM_012276 |
| LILRA5 | NM_181985; NM_021250; NM_181879; NM_181986 |
| LILRA6 | XR_001756516; XR_007068733; NR_104098; XM_047442915; NM_024318; |
| | XR_007068732; NM_001360167; XM_011547130; XM_047442916 |
| LILRB1 | XM_017026192; NM_001081637; NM_001081639; NM_001278399; |
| | XM_047438080; XM_047438084; XM_047438085; NM_001081638; |
| | NM_006669; XM_047438081; NM_001278398; NM_001388358; |
| | XM_047438083; NM_001388355; NM_001388357; NR_103518; |
| | XM_047438082; XM_047438086; NM_001388356; XM_047438089; |
| | XM_047438087; XM_047438088 |
| LILRB2 | NM_001278403; NM_001278406; NM_005874; NM_001080978; |
| | NM_001278404; NM_001278405; NR_103521 |
| LILRB3 | NM_006864.4; NM_001081450.3; NM_001320960.2; NR_135493.2; |
| | NR_135494.2; NR_135495.2; NR_135496.2 |
| LILRB4 | NM_001278429; NM_001394939; NM_001394934; NM_006847; |
| | NM_001278428; XM_017026216; XM_047438100; NM_001394935; |
| | NM_001081438; XM_047438102; XM_047438103; NM_001394938; |
| | XM_047438101; NM_001278426; NM_001394933; NM_001394937; |
| | NM_001278427; NM_001278430; NM_001394936 |
| LIM2 | NM_001161748; NM_030657 |
| LIMD1 | NM_014240; XM_011534207 |
| LIN28B | NM_001004317; XM_011535818; XM_006715477 |
| LIPA | NR_110233; XM_024448023; NM_001127605; NM_000235; NM_001288979 |
| LIPC | XM_017022176; XM_005254374; XM_006720502; XM_024449916; |
| | XM_005254372; XM_047432491; NM_000236; XM_024449917 |
| LIPE | XM_047438835; XM_006723218; XM_005258938; XM_005258939; |
| | XM_017026810; XM_047438836; XM_005258940; XM_024451514; |
| | XM_005258937; NM_005357 |
| LOX | NM_001317073; NM_001178102; NM_002317 |
| LOXL1 | XM_047432498; XM_017022179; XM_011521555; NM_005576 |
| LRIG1 | NM_001377348; NM_001377347; NM_001377344; XM_017006135; |
| | XM_047447939; NM_001377345; NM_001377346; NM_001377349; |
| | XM_011533578; NM_015541 |
| LRP1 | NM_002332 |
| LRP5 | XM_011545030; XM_017017736; NM_002335; XM_047426948; |
| | XM_047426949; XM_005273994; XM_011545031; NM_001291902; |
| | XM_047426950; XM_011545029; XM_017017735; XR_001747874 |
| LRP6 | XM_047428844; XR_429035; NM_002336; XM_006719078; |
| | XM_011520671; XR_002957325 |
| LRRC25 | XM_005259739; NM_145256 |
| LRRN3 | NM_018334; NM_001099660; NM_001099658 |
| LSP1 | NM_001242932; NM_001013255; NM_001289005; NM_001013254; |
| | NM_002339; NM_001013253 |
| LST1 | NM_205838; NM_001166538; NR_029461; NM_205839; XM_006715209; |
| | XM_006715210; NM_205837; XM_006715206; XM_047419357; |
| | NM_007161; XM_011514914; NR_029462; NM_205840 |
| LTA | NM_000595; NM_001159740; XM_047418773 |
| LTB | NM_002341; NM_009588 |
| LTBR | NM_002342; NM_001270987; XM_006718983; XM_005253688 |
| LTF | NM_001199149; NM_001321122; NM_001321121; NM_002343 |
| LTK | XM_024449919; XR_007064447; XM_047432503; NM_002344; |
| | XM_011521557; NM_206961; XM_047432499; XM_047432500; |
| | XM_047432506; NM_001135685; XR_007064446; XM_047432501; |
| | XM_047432502; XM_047432505 |
| LUM | NM_002345 |
| LY75 | NM_002349.4 |
| LY9 | XM_011509556; XM_047420762; NM_001261457; XM_047420755; |
| | XM_017001303; XM_047420771; NM_001033667; NM_001261456; |
| | XM_047420753; XM_047420764; XM_017001304; XM_017001299; |
| | NM_002348; XM_011509549; XM_011509560; XM_017001301; |
| | XM_047420765 |
| LYN | XM_011517529; NM_001111097; NM_002350 |
| LYVE1 | NM_006691 |
| LYZ | NM_000239 |
| MACC1 | NM_182762 |
| MAD2L1 | NM_002358 |
| MAD2L2 | XM_047430782; NM_001127325; NM_006341 |
| MAF | XM_024450279; NM_001031804; XM_017023233; XR_002957802; |
| | XR_002957804; XR_001751902; XM_017023235; XM_017023234; |
| | NM_005360; XR_002957803 |
| MAFB | NM_005461 |
| MAGEA1 | NM_004988 |
| MAGEA10 | NM_001251828; NM_021048; NM_001011543 |
| MAGEA12 | NM_001166386; NM_001166387; NM_005367 |
| MAGEA2 | NM_001386130.2; NM_005361.3; NM_175742.2; NM_175743.2; |
| | NM_001282501.2; NM_001282502.1; NM_001282504.1; NM_001282505.1 |
| MAGEA2B | NM_001282501; NM_001282505; NM_005361; NM_001282502; |
| | NM_001282504; NM_001386130; NM_175742; NM_175743 |
| MAGEA3 | XM_011531161; XM_005274676; XM_006724818; XM_011531160; |
| | NM_005362 |
| MAGEA4 | NM_001386196; NM_001386197; NM_001386200; NM_002362; |
| | NM_001011550; NM_001386202; NM_001011548; NM_001011549; |
| | NM_001386198; NM_001386203; NM_001386199 |
| MAGEA6 | NM_175868; NM_005363 |
| MALAT1 | NR_002819.4; NR_144567.1; NR_144568.1 |
| MALT1 | NM_173844; NM_006785; XM_011525794; XR_007066087 |
| MAML1 | NM_014757 |
| MAML2 | XM_011543023; XM_047427710; NM_032427 |
| MAML3 | XM_047415930; NM_018717; XM_047415929 |
| MAP1LC3A | XM_047440559; XM_011529085; XM_047440558; XM_011529084; |
| | NM_181509; NM_032514 |
| MAP1LC3B | NM_022818 |
| MAP2K1 | XM_011521783; XM_017022412; XM_017022411; NM_002755 |
| MAP2K2 | NM_030662; XM_047439100; XM_006722799 |
| MAP3K1 | XM_047417220; XM_047417218; XM_047417219; NM_005921 |
| MAP3K14 | XM_047436998; XM_011525441; XM_047436997; NM_003954 |
| MAP3K7 | XM_006715553; NM_145332; NM_145333; NM_003188; NM_145331 |
| MAP4K1 | XM_011526404; NM_001042600; NM_007181 |
| MAPK1 | NM_138957; NM_002745 |
| MAPK3 | XR_243293; NM_001109891; NM_001040056; NM_002746 |
| MAPKAP1 | NM_001006621; NM_024117; NM_001006617; NM_001006619; |
| | NM_001006620; NM_001006618 |
| MARCO | NM_006770; XM_011512082; XM_011512083; XM_017005171 |
| MASP2 | XR_001736931; NM_006610; XM_017000097; NM_139208; XM_047439758 |
| MATIA | NM_000429 |
| MAX | NM_001271068; NM_001271069; NM_001320415; NM_145114; |
| | XR_007064012; NM_145112; NM_145113; NM_145116; NM_197957; |
| | XR_943452; NR_073137; XM_011536773; NM_002382; XR_943450; |
| | NR_073138 |
| MBD1 | XM_011526002; XM_047437514; XM_047437518; NM_001204137; |
| | NM_001204139; NM_001323949; NM_001399892; NM_001399903; |
| | NM_001399912; NM_001399921; NM_001399932; NM_001399936; |
| | NM_00 1399940; NM_001399944; NM_001399952; NM_001399953; |
| | NM_001399960; XM_017025770; XM_047437510; XM_047437513; |
| | NM_001323951; NM_001323953; NM_001388139; NM_001388141; |
| | NM_001388144; NM_001388143; NM_001388145; NM_001388146; |
| | NM_001388156; NM_001388162; NM_001388163; NM_001388165; |
| | NM_001399879; NM_001399880; NM_001399881; NM_001399882; |
| | NM_001399887; NM_001399893; NM_001399916; NM_001399922; |
| | NM_001399926; NM_001399929; NM_001399942; NM_001399954; |
| | NM_001399970; NM_001399974; XM_017025776; XM_047437520; |
| | NM_001204141; NM_001204142; NM_001323952; NM_001323954; |
| | NM_001388140; NM_001388147; NM_001388159; NM_001399891; |
| | NM_001399906; NM_001399909; NM_001399913; NM_001399934; |
| | NM_001399935; NM_001399941; NM_001399947; NM_001399949; |
| | NM_001399957; XM_005258271; XM_024451180; NM_001204136; |
| | NM_001388138; NM_001388149; NM_001388166; NM_001399888; |
| | NM_001399890; NM_001399894; NM_001399898; NM_001399899; |
| | NM_001399901; NM_001399900; NM_001399902; NM_001399914; |
| | NM_001399917; NM_001399924; NM_001399937; NM_001399939; |
| | NM_001399967; NM_015846; XM_047437511; XM_047437519; |
| | NM_001204143; NM_001388148; NM_001388151; NM_001388158; |
| | NM_001399884; NM_001399886; NM_001399889; NM_001399904; |
| | NM_001399907; NM_001399910; NM_001399918; NM_001399923; |
| | NM_001399927; NM_001399933; NM_001399948; NM_001399950; |
| | NM_001399958; NM_001399959; NM_001399963; NM_001399975; |
| | NM_015844; XM_047437512; NM_001204138; NM_001388142; |
| | NM_001388152; NM_001388155; NM_001388160; NM_001388161; |
| | NM_001388167; NM_001399883; NM_001399896; NM_001399908; |
| | NM_001399911; NM_001399920; NM_001399925; NM_001399930; |
| | NM_001399966; NM_001399973; NM_001399971; NM_001399976; |
| | NM_015845; XM_017025757; NM_001204140; NM_001388154; |
| | NM_001388157; NM_001388164; NM_001399885; NM_001399897; |
| | NM_001399915; NM_001399919; NM_001399938; NM_001399943; |
| | NM_001399945; NM_002384; NM_015847; XM_011526007; |
| | XM_047437515; XM_047437516; XM_047437517; NM_001204151; |
| | NM_001323942; NM_001323947; NM_001323950; NM_001388150; |
| | NM_001388153; NM_001399895; NM_001399905; NM_001399928; |
| | NM_001399931; NM_001399946; NM_001399955; NM_001399956; |
| | NM_001399961; NM_001399962; NM_001399964; NM_001399965; |
| | NM_001399968 |
| MBD4 | NM_001276271; XM_047449153; NM_003925; NM_001276273; |
| | NM_001276270; NM_001276272; XM_024453810 |
| MCAM | XM_017017762; XM_017017759; XM_017017760; NM_006500; |
| | XM_017017761 |
| MCL1 | NM_001197320; NM_182763; NM_021960 |
| MCM2 | NM_004526; XM_024453531; NR_073375 |
| MCM6 | NM_005915 |
| MCRS1 | XM_005268572; NM_001278341; XM_047428083; NM_001012300; |
| | XM_017018690; XM_011537760; NM_006337 |
| MDC1 | XM_005249497; XM_047419580; XM_047419586; XM_047419589; |
| | XM_005249494; XM_047419587; XM_017011522; XM_047419588; |
| | XM_047419585; XM_047419583; XM_047419584; NM_014641; |
| | XM_011515003; XM_047419582; XM_005249498; XM_005249492; |
| | XM_011515004; XM_047419581 |
| MDM2 | NM_006880; NM_006882; XM_047428853; NM_006878; NM_001145340; |
| | NM_001278462; NM_001367990; NM_006879; NM_001145337; |
| | NM_002392; NM_006881; NM_032739; NM_001145339; NM_001145336 |
| MDM4 | XM_024447114; XM_024447115; NM_002393; XM_017001311; |
| | XM_017001312; XM_047420955; NM_001204172; NM_001278518; |
| | XM_047420961; NM_001204171; NM_001278516; NM_001278519; |
| | XM_017001313; XM_047420951; NM_001278517; XM_047420952 |
| MECOM | XM_005247214; XM_047447680; XM_047447684; XM_047447688; |
| | XM_047447692; XM_047447693; XM_047447690; XM_047447694; |
| | NM_001366469; NM_001366470; NM_001366474; XM_047447682; |
| | XM_047447686; NM_001366466; NM_001366468; NM_001366473; |
| | XM_005247213; XM_005247221; XM_011512546; NM_001105077; |
| | NM_004991; XM_047447678; NM_001366472; XM_047447677; |
| | NM_001164000; XM_047447679; XM_047447691; NM_001366467; |
| | NM_005241; XM_047447681; XM_047447683; XM_047447685; |
| | XM_047447687; XM_047447689; NM_001105078; NM_001163999; |
| | NM_001205194; NM_001366471 |
| MEF2B | NM_001145785.2; NM_001367282.1 |
| MEFV | NM_001198536; NM_000243 |
| MEN1 | NM_130800; NM_130803; XM_017017768; NM_001370260; |
| | XM_017017765; NM_001370251; NM_001370259; NM_130804; |
| | XM_011545041; NM_130802; XM_017017766; NM_001370262; |
| | NM_001370263; XM_011545040; XM_017017767; NM_001370261; |
| | NM_000244; NM_130799; NM_130801 |
| MERTK | NM_006343 |
| MET | NM_001324402; NM_001324401; NM_001127500; NM_000245; |
| | XM_011516223; XM_047420400 |
| MFAP5 | NM_001297709; NR_123733; NR_123734; NM_001297711; NM_003480; |
| | NM_001297710; NM_001297712 |
| MFGE8 | XM_047432533; NM_001310321; XM_047432535; XM_017022206; |
| | NM_005928; XR_931838; NM_001310319; NM_001310320; |
| | XM_047432534; NM_001114614 |
| MGA | XM_005254243; XM_011521397; XM_011521398; XM_047432295; |
| | XM_047432296; XM_047432308; NM_001400246; XM_047432276; |
| | XM_047432300; XM_047432309; XM_047432312; NM_001080541; |
| | XM_005254254; XM_047432281; XM_047432288; XM_047432291; |
| | XM_047432293; XM_047432307; XM_047432310; XM_047432311; |
| | XM_005254249; XM_005254252; XM_011521399; XM_017022029; |
| | XM_047432283; XM_047432285; XM_047432302; XM_047432306; |
| | NM_001164273; XM_047432280; XM_047432287; XM_047432292; |
| | XM_047432298; XM_047432305; NM_001400243; NM_001400244; |
| | NM_001400247; XM_006720443; XM_047432289; XM_047432290; |
| | XM_047432297; XM_047432303; XM_047432313; XM_047432278; |
| | XM_047432282; XM_047432304; XM_005254246; XM_005254253; |
| | XM_006720445; XM_047432279; XM_047432286; XM_047432299; |
| | XM_047432301; NM_001400225; NM_001400242; NM_001400245 |
| MGAT5 | XM_011511201; XM_047444398; XM_047444406; NM_002410; |
| | XM_047444394; XM_047444397; XM_005263669; XM_047444402; |
| | XM_047444405; XM_006712534; XM_047444401; XM_047444403; |
| | XM_011511202; XM_047444399; XM_047444395; XM_047444404; |
| | XM_011511199; XM_017004148; XM_047444396; XM_047444400; |
| | XM_017004147; XM_017004149; NM_001371457 |
| MGMT | NM_002412 |
| MIF | NM_002415 |
| MITF | NM_001184968; NM_001354605; NM_001354604; NM_001354607; |
| | NM_001354608; NM_006722; NM_198159; NM_198178; NM_198158; |
| | NM_198177; NM_000248; NM_001184967; NM_001354606 |
| MKI67 | NM_002417; NM_001145966; XM_006717864; XM_011539818 |
| MKL1 | NM_001282662; NM_020831; NM_001282660; NM_001318139; |
| | NM_001282661 |
| MLH1 | NM_001258274; XM_047448154; NM_001167619; NM_001258273; |
| | NM_001354618; NM_001354619; NM_001354629; NM_001354630; |
| | NM_000249; NM_001258271; NM_001354624; NM_001354628; |
| | XM_047448152; NM_001354623; NM_001354627; XM_047448153; |
| | NM_001167617; NM_001167618; NM_001354616; NM_001354620; |
| | NM_001354626; XM_005265161; NM_001354617; XM_047448155; |
| | NM_001354615; NM_001354621; NM_001354622; NM_001354625 |
| MLH3 | XR_245681; XM_005267532; XM_047431269; XM_017021219; |
| | XM_005267533; XM_047431268; XR_001750225; XR_007064004; |
| | XM_006720116; XM_024449538; NM_014381; XR_007064005; |
| | XM_005267534; XM_047431265; XM_047431266; NM_001040108; |
| | XM_047431267 |
| MLKL | XM_005255834; XM_047433710; XM_011522936; XM_047433705; |
| | XM_047433708; XM_047433709; XM_047433707; NM_152649; |
| | XM_047433706; NM_001142497; XM_047433704 |
| MLLT3 | NM_001286691; NM_004529 |
| MLST8 | XM_047434466; NM_001199173; NM_001199175; NM_001352057; |
| | XM_047434467; XM_005255479; NM_001199174; NR_147904; |
| | NM_022372; XM_005255475; NR_147905; NR_147906; NM_001352059; |
| | NM_001352060; NR_147907 |
| MLX | NM_198204; NM_198205; NM_170608; NM_170607; NM_013383 |
| MLXIP | NM_014938; XM_047428538; XM_006719290; XM_047428537; |
| | XM_006719293; XM_006719291; XM_006719292 |
| MLXIPL | XM_011516279; XM_047420436; XM_047420437; NM_032953; |
| | NM_032994; XM_011516277; XM_047420433; NM_032951; NR_134541; |
| | XM_017012263; XM_047420435; XM_047420432; NM_032952; |
| | XR_007060040; XM_011516278; NM_032954; XM_047420434 |
| MME | XM_047448157; NM_000902; XM_011512857; NM_001354644; |
| | NM_007288; XM_011512856; NM_001354642; NM_001354643; |
| | XM_006713647; NM_007287; NM_007289 |
| MMP1 | NM_001145938; NM_002421 |
| MMP11 | NM_005940; NR_133013 |
| MMP12 | NM_002426 |
| MMP14 | NM_004995 |
| MMP19 | NM_022790; NR_073606; NM_001272101; XM_011538359; |
| | XM_047428863; XM_047428864; NM_022792; NM_002429; |
| | NM_001032360 |
| MMP2 | NM_001302509; NM_001127891; NM_001302508; NM_001302510; |
| | NM_004530 |
| MMP25 | NM_024302; XM_011525227; NM_001032278; NM_032950; |
| | XM_011525225; XM_011525230; XM_024450943; XM_011525226; |
| | NR_111988; XM_011525229; XM_011525231; XM_011525232; |
| | XM_017025063; XM_017025064; XM_047436731 |
| MMP3 | NM_002422 |
| MMP7 | NM_002423 |
| MMP9 | NM_004994 |
| MMRN1 | NM_001371403; XM_047449832; XM_047449831; NM_007351 |
| MMRN2 | NM_024756 |
| MMS22L | XM_011535678; XM_011535680; NM_001350599; XR_942376; |
| | XM_011535670; XM_047418576; XM_011535672; XM_011535676; |
| | NM_001350600; NM_198468; XM_011535671; XM_011535679; |
| | XM_047418574; XM_047418575; XM_047418582; XM_047418579; |
| | XM_011535675; XM_047418580; XM_006715432; XM_011535674; |
| | XM_047418577; XM_047418581; XM_047418583; XM_047418578 |
| MNAT1 | XM_005267688; NM_002431; XM_017021332; XM_017021334; |
| | XM_047431413; NM_001177963 |
| MNDA | NM_002432 |
| MNT | NM_020310; XM_011523869; XM_047436092; XM_024450758; |
| | XM_011523868; XM_017024654 |
| MOB1A | NM_001317111; NM_018221; NM_001317110; XM_047444894; |
| | NM_001317112 |
| MOB1B | NM_173468; XM_011532412; XM_017008837; NM_001244766; |
| | NM_001244767; XM_005265709 |
| MPG | NM_001015052; NM_002434; NM_001015054 |
| MPL | NM_005373 |
| MPO | NM_000250 |
| MPP1 | XM_047442123; XM_047442124; NM_001166462; XM_011531167; |
| | XM_011531169; NM_001166461; XM_024452385; NM_001166460; |
| | NM_002436 |
| MRC1 | NM_002438; NM_001009567 |
| MRPL36 | NM_032479; XM_011514080; XM_017009752; XM_017009751; |
| | XM_017009750; XM_011514079 |
| MS4A1 | NM_021950; NM_152866; NM_152867 |
| MS4A2 | XM_011544850; NM_001142303; NM_000139; XM_005273846; |
| | XM_017017362; NM_001256916 |
| MS4A4A | NM_001243266; NM_148975; NM_024021 |
| MS4A4E | NM_001393391.1; NM_001351235.2 |
| MS4A6A | XM_011545209; NM_001330275; NM_022349; NM_152851; |
| | XM_005274177; XM_017018125; XM_047427403; NM_001247999; |
| | XM_047427402; NM_152852; XM_024448652; XM_006718660; |
| | XM_006718661 |
| MS4A7 | NM_021201; NM_206940; NM_206939; NM_206938 |
| MSH2 | XM_011532867; NM_000251; XR_001738747; XM_047444416; |
| | NM_001258281; XR_939685; XM_005264332 |
| MSH3 | NM_002439 |
| MSH5 | NM_172165; NM_172166; NM_002441; NM_025259 |
| MSH6 | NM_000179; NM_001281493; NM_001281492; NM_001281494 |
| MSLN | NM_001177355; NM_005823; NM_013404 |
| MSR1 | NM_138716; NM_002445; XM_024447161; NM_138715; NM_001363744 |
| MST1 | XM_017006460; NM_001393584; XM_047448160; NM_001393581; |
| | NM_001393583; NM_001393582; XM_047448159; XM_047448161; |
| | NM_001393585; NR_146060; XM_011533738; XM_047448158; NM_020998 |
| MST1R | XM_005265170; XM_011533742; XM_047448172; XM_047448175; |
| | NR_134919; XM_047448170; XM_047448178; XM_011533744; |
| | XM_047448167; XM_047448180; XM_011533743; NM_002447; |
| | XR_001740155; XM_011533739; XM_011533741; XM_047448162; |
| | XM_047448177; NM_001244937; XM_011533740; XM_047448169; |
| | XM_047448176; XM_047448164; XM_047448165; XM_047448171; |
| | XM_047448174; XM_047448179; XM_047448163; XM_047448166; |
| | XM_047448168; XM_047448173; NM_001318913 |
| MTA1 | XM_047431900; XM_047431902; XM_047431903; XM_011537310; |
| | XM_047431899; XM_047431910; XM_047431904; XM_047431907; |
| | XM_011537305; XM_047431905; NM_001203258; XM_011537307; |
| | XM_047431906; NM_004689; XM_011537306; XM_047431908; |
| | XM_011537308; XM_047431898; XM_011537309; XM_047431901; |
| | XM_047431909 |
| MTAP | NM_001396042; NM_001396044; NM_001396043; NM_001396041; |
| | NR_173242; NM_001396040; NM_001396045; NM_002451 |
| MTOR | NM_001386501; XM_017000900; XM_011541166; NM_001386500; |
| | XR_007058581; XM_047416721; XM_047416724; NM_004958 |
| MTSS1 | XM_017014091; NM_001282974; NM_001363295; NM_001363297; |
| | NM_001363298; XM_006716703; XM_017014088; XM_047422491; |
| | NM_014751; XM_006716702; XM_006716706; XM_047422490; |
| | XM_017014087; XM_017014089; XM_017014090; XM_006716705; |
| | XM_011517403; NM_001363294; NM_001363296; NM_001363301; |
| | NM_001363302; XM_006716701; XM_005251111; XM_006716700; |
| | XM_017014092; XM_006716704; XM_047422488; XM_047422492; |
| | XM_005251113; XM_017014086; XM_047422493; NM_001282971; |
| | NM_001363299; NM_001363300 |
| MUC1 | NM_001018017; NM_001044391; NM_001044393; NM_001204291; |
| | NM_001044390; NM_001204285; NM_182741; NM_001371720; |
| | NM_001204289; NM_001204290; NM_001204293; NM_001018016; |
| | NM_001044392; NM_001204286; NM_001204287; NM_001204288; |
| | NM_001204295; NM_001018021; NM_001204292; NM_001204294; |
| | NM_001204297; NM_001204296; NM_002456 |
| MUC16 | NM_001401501; NM_024690 |
| MUM1 | NM_001195286; NR_046000; NR_036585; XM_006715090; |
| | XM_047418730; NM_002460 |
| MUS81 | NM_025128; XM_047427637; NM_001350283; XM_011545270; |
| | XM_047427635; XM_047427638; XM_047427636; NR_146598; |
| | XM_011545269 |
| MUTYH | XM_047421198; NM_001048173; XM_011541497; XM_011541499; |
| | XM_047421202; NM_001293190; NM_001293192; NR_146882; |
| | XM_047421200; XM_047421201; XM_011541503; XM_047421191; |
| | XM_047421194; XM_047421196; NM_001128425; XM_017001334; |
| | XM_047421192; NM_001048172; NR_146883; XM_011541502; |
| | XM_017001332; XM_017001333; XM_047421203; NM_001293196; |
| | NM_001350650; XM_047421195; XM_047421199; NM_001048171; |
| | NM_001048174; NM_001293191; NM_012222; XM_047421193; |
| | XM_047421197; XM_047421204; NM_001293195; NM_001350651 |
| MVP | NM_017458; NM_001293204; NM_005115; NM_001293205 |
| MXD1 | NM_001202513; NM_001202514; NM_002357 |
| MXD3 | NM_001142935; NM_001394987; NM_031300; NM_001394986 |
| MXD4 | NM_006454 |
| MXI1 | NM_005962; NM_001008541; NM_130439 |
| MYB | NM_001161660; NR_134958; NM_001130173; NM_001130172; |
| | NM_001161656; NR_134959; NM_001161657; XM_047418834; |
| | NR_134963; NR_134965; NR_134962; XR_942444; NM_001161659; |
| | NR_134961; NM_001161658; NM_005375; NR_134960; NR_134964 |
| MYBL2 | NM_002466; NM_001278610 |
| MYC | NM_002467; NM_001354870 |
| MYCL | NM_001033082; NM_001033081; NM_005376 |
| MYCN | XM_047444434; NM_005378; NM_001293231; NM_001293228; |
| | NM_001293233 |
| MYH11 | XM_017023250; NM_002474; NM_022844; NM_001040113; |
| | NM_001040114 |
| MYO1F | XM_011528028; XR_936181; NM_001348355; XM_047438852; |
| | XM_011528027; XR_936182; XR_001753692; NM_012335; XM_011528024 |
| MYO1G | XR_007060129; NM_033054 |
| MZB1 | XM_047417264; NM_016459 |
| NAE1 | NM_001018160; XM_047434835; NM_003905; NM_001286500; |
| | NM_001018159 |
| NBN | NM_002485; XM_011517046; XM_024447163; XM_011517045; |
| | XM_047421796; NM_001024688; XM_047421795 |
| NCAM1 | NM_001386289; NM_001400605; NM_001400607; NM_001386290; |
| | NM_001386291; NM_001400617; NM_001386292; NM_001400603; |
| | NM_001400609; NM_001400611; NM_001076682; NM_001400612; |
| | NM_001400615; NM_001400619; NM_001400621; NM_000615; |
| | NM_001400610; NM_001400618; NM_001400620; NM_001400622; |
| | NM_001400624; NM_001400608; NM_001400614; NM_001242608; |
| | NM_001400613; NM_001400616; NM_001400623; NM_181351; |
| | NM_001242607; NM_001400604; NM_001400606 |
| NCF2 | XM_047421222; XM_047421229; XM_047421238; NM_001190789; |
| | XM_005245207; XM_047421231; NM_001127651; NM_001190794; |
| | NM_000433; XM_011509580; XM_011509581 |
| NCKAP1L | NM_001184976; NM_005337 |
| NCOA4 | NM_001145262; NM_001145261; NM_005437; NM_001145260; |
| | NM_001145263 |
| NCOR1 | XM_005256872; XM_006721602; XM_006721604; XM_017025396; |
| | XM_017025419; XM_047437121; XM_047437128; XM_047437135; |
| | NM_006311; XM_005256868; XM_006721601; XM_017025415; |
| | XM_047437127; XM_047437131; XM_047437132; XM_047437133; |
| | XM_047437136; XM_047437139; XM_047437144; XM_005256866; |
| | XM_011524084; XM_011524085; XM_047437125; XM_047437129; |
| | XM_005256871; XM_017025403; XM_017025409; XM_017025418; |
| | XM_017025420; XM_047437124; XM_047437126; XM_047437141; |
| | XM_006721603; XM_017025417; XM_047437138; XM_047437143; |
| | NM_001190440; XM_011524086; XM_047437122; XM_047437130; |
| | XM_047437142; NM_001190438; XM_005256874; XM_017025397; |
| | XM_017025400; XM_047437123; XM_047437137; XM_047437140; |
| | XM_005256873; XM_005256875; XM_017025401; XM_047437134 |
| NCOR2 | NM_001077261; NM_001206654; NM_006312 |
| NCR1 | NM_004829; XM_011527530; XM_047439727; NM_001145457; |
| | NM_001242357; XM_011527529; NM_001242356; NM_001145458 |
| NCR2 | XM_017011500; NM_001199510; NM_001199509; NM_004828 |
| NCR3 | NM_001145467; XM_011514459; XM_006715049; NM_001145466; |
| | NM_147130 |
| NCSTN | NM_001290184; NM_015331; NM_001290186; NM_001349729; |
| | XM_005245053 |
| NDRG1 | NM_001258433; NM_001374844; NM_001135242; NM_001258432; |
| | NM_001374846; NM_006096; NM_001374845; NM_001374847 |
| NDRG2 | NM_016250; NM_001354567; NM_201538; NM_001282215; |
| | NM_001354560; NM_001354561; NM_001354569; NM_201535; |
| | NM_001282216; NM_001354564; NM_001354565; NM_001354566; |
| | NM_201536; NM_201539; NM_201541; NM_001354558; NM_001354562; |
| | NM_001282213; NM_001354570; NM_201540; NM_001282211; |
| | NM_001320329; NM_001282212; NM_001282214; NM_001354559; |
| | NM_001354568; NM_201537 |
| NDRG4 | NM_001242833; NM_001378335; NM_001242836; NM_001378334; |
| | NM_001378338; NM_001378341; NR_040072; XM_006721253; |
| | XM_011523290; NM_001378343; NM_020465; NM_001378347; |
| | XM_006721256; XM_017023582; NM_001130487; NM_001242834; |
| | NM_001242835; NM_001378332; NM_001378333; NM_001378339; |
| | NM_001378345; NM_001378346; NM_022910; XM_047434509; |
| | XM_011523293; NM_001378342; NM_001378344; XM_017023583; |
| | XM_011523291; NM_001363869; NM_001378336; NM_001378337; |
| | NM_001378340 |
| NDUFA13 | NM_015965 |
| NEDD9 | NM_001271033; NM_001142393; NM_182966; NR_073131; NM_006403 |
| NEIL1 | NM_024608; NM_001352519; NM_001256552; NM_001352520; NR_046311 |
| NEIL2 | NM_001349441; NM_001135747; NR_146180; NM_001135746; |
| | NR_146181; NM_001349440; NM_001349442; NM_001349439; |
| | NM_145043; NR_146182; NM_001135748 |
| NEIL3 | NM_018248; XM_047415894 |
| NELL2 | NM_001145107; XM_011538396; NM_001145109; NM_001145110; |
| | NM_006159; NM_001145108 |
| NF1 | NM_001042492; NM_000267; NM_001128147 |
| NF2 | XM_047441386; NM_181828; NM_181830; NM_181826; NM_000268; |
| | NR_156186; NM_181827; NM_181834; NM_016418; NM_181829; |
| | NM_181825; NM_181831; NM_181835; XM_017028809; NM_181832; |
| | NM_181833 |
| NFATC1 | XM_017025783; NM_001278669; NM_001278675; NM_172389; |
| | XM_047437538; NM_001278670; NM_001278672; NM_001278673; |
| | NM_172387; NM_006162; NM_172388; NM_172390 |
| NFATC2 | NM_001258292; XM_017027851; NM_173091; XM_011528826; |
| | NM_001258294; NM_001258296; XM_011528825; NM_001258297; |
| | XM_011528824; NM_001136021; NM_001258295; NM_012340 |
| NFATC3 | NM_173164; NM_173165; NM_004555; NM_173163 |
| NFE2L2 | NM_001313902; NM_001313901; NM_001145413; NM_001313904; |
| | NM_001313903; NM_001145412; NM_001313900; NM_006164 |
| NFKB1 | XM_024454069; NM_001319226; NM_003998; XM_024454068; |
| | XM_047415744; XM_047415742; NM_001382625; NM_001382628; |
| | NM_001165412; NM_001382626; XM_047415743; NM_001382627 |
| NFKB2 | NM_001261403; NM_001077494; NM_001322935; NM_002502; |
| | NM_001288724; NM_001322934 |
| NFKBIA | NM_020529 |
| NFKBIE | NM_004556 |
| NHEJ1 | NM_001377498; NM_001377499; NM_024782; NR_165304 |
| NINJ1 | NM_004148; XM_011518716 |
| NKG7 | XM_006723228; XM_005258955; NM_001363693; NM_005601 |
| NLRC3 | XM_047433771; NM_178844; XM_047433769; NR_075083; XM_047433770 |
| NLRC5 | NM_001330552; NM_001384961; NM_001384969; NM_001384972; |
| | NM_001384973; NR_169518; NM_001384951; NM_001384959; |
| | NM_032206; NR_169513; XR_001752000; XM_006721300; |
| | XM_017023770; XM_047434760; XM_047434765; NM_001384950; |
| | NM_001384958; NM_001384964; NR_169520; NM_001384954; |
| | NM_001384966; NR_169514; NR_169517; XM_047434761; |
| | XM_047434763; NM_001384967; NM_001384971; XM_047434766; |
| | NM_001384965; NM_001384970; NR_169512; NR_169519; |
| | XM_047434762; XM_047434764; NM_001384953; NM_001384957; |
| | NM_001384956; NM_001384962; NM_001384963; NR_169515; |
| | NR_169516; NM_001384952; NM_001384955; NM_001384960; |
| | NM_0013 84968 |
| NME1 | NM_000269; NM_198175 |
| NME2 | NM_002512.4; NM_001018137.3; NM_001018138.1; NM_001018139.2; |
| | NM_001198682.2 |
| NMI | XM_047446270; XM_005246941; NM_004688 |
| NMUR1 | XM_011510488; XM_006712196; XM_011510487; NM_006056; |
| | XM_006712195; XM_011510489 |
| NOS2 | NM_153292; NM_000625 |
| NOS3 | NM_001160110; NM_000603; NM_001160109; NM_001160111 |
| NOTCH1 | NM_017617.5 |
| NOTCH2 | NM_017617; XM_011518717 |
| NOTCH3 | NM_024408; NM_001200001 |
| NOTCH4 | XM_005259924; NM_000435 |
| NOX4 | NM_001291926; NM_016931; NM_001143837; NM_001143836; |
| | NM_001291927; XM_017017843; NM_001291929; NM_001300995; |
| | NR_120406 |
| NPAS2 | XM_005263959; XM_005263960; XM_011511242; NM_002518; |
| | XM_017004217; XM_047444503; XM_047444510; XM_047444506; |
| | XM_047444512; XM_047444513; XM_005263961; XM_017004214; |
| | XM_017004216; XM_047444511; XR_007075420; XM_047444504; |
| | XM_047444505; XM_047444515; XM_005263953; XM_011511243; |
| | XM_047444502 |
| NPL | NM_001200051; NM_001200052; NM_030769; NM_001200050; |
| | NM_001200056 |
| NPM1 | NM_001037738; NR_149149; NM_001355007; NM_001355006; |
| | NM_001355009; NM_002520; NM_199185; NM_001355010 |
| NPRL2 | XM_011533288; XM_047447310; XM_047447314; XM_017005556; |
| | XM_047447312; XM_047447311; NM_006545 |
| NPRL3 | NM_001243248; NM_001039476; NM_001243247; NM_001243249; |
| | NM_001077350; NM_012075 |
| NR1I3 | NM_001077474; NM_001077472; NM_001077476; NM_001077481; |
| | NM_005122; XM_005245697; NM_001077470; NM_001077478; |
| | NM_001077471; NM_001077479; NM_001077473; NM_001077477; |
| | NM_001077469; XM_005245693; NM_001077475; NM_001077480; |
| | NM_001077482 |
| NR3C2 | NR_148974; XM_047415706; XM_047415709; NM_000901; |
| | NM_001166104; XM_011531975; XM_047415708; XM_047415707; |
| | NM_001354819; XM_011531978 |
| NRARP | NM_001004354 |
| NRAS | NM_002524 |
| NRG1 | XM_011544512; XM_017013367; NM_001160007; NM_001322197; |
| | NM_001322201; NM_013958; NM_001160001; NM_001160004; |
| | NM_001322202; NM_001322205; NM_001322207; NM_004495; |
| | NM_013956; NM_001322206; NM_013959; XM_017013371; |
| | XM_017013372; NM_001159996; NM_013960; NM_001160008; |
| | NM_013962; XM_017013368; NM_001159995; NM_001159999; |
| | NM_001160002; NM_001160005; NM_013957; NM_013964 |
| NRP1 | NM_001024628; NR_045259; XM_006717526; XM_011519756; |
| | NM_001244972; NM_001330068; XM_011519755; XM_017016865; |
| | NM_001244973; XM_006717521; XM_047425978; XM_006717525; |
| | XM_047425976; XM_047425977; XM_006717522; XM_006717524; |
| | NM_001024629; NM_003873 |
| NT5C2 | XM_024447901; XM_017015947; XM_047424847; XM_047424851; |
| | XM_047424852; NM_001351172; NM_001351176; NM_001351195; |
| | XM_047424844; XM_047424845; XM_047424846; XM_047424849; |
| | XM_047424854; NM_001351178; NM_001351182; NM_001351184; |
| | NM_001351185; NM_001351188; NM_001351193; NM_001351196; |
| | XM_047424858; NM_001351171; NM_001351183; NM_001351194; |
| | NM_012229; XM_024447903; XM_047424855; NM_001351169; |
| | NM_001351173; NM_001351186; NM_001351187; NM_001351191; |
| | XM_047424856; XM_047424857; NM_001134373; NM_001351174; |
| | NM_001351181; NM_001351197; NM_001351177; NM_001351189; |
| | NM_001351190; NM_001351192; XM_047424853; NM_001351175; |
| | NM_001351179; NM_001351180; XM_005269637; XM_011539537; |
| | XM_024447902; XM_047424848; XM_047424850; XM_047424859; |
| | NM_001351170 |
| NT5E | NM_001204813; NM_002526 |
| NTHL1 | NM_001318193; XM_047434171; NM_002528; NM_001318194 |
| NTRK1 | NM_001007792; NM_002529; NM_001012331 |
| NTRK2 | XM_005252001; XM_047423432; NM_001369536; NM_001369539; |
| | NM_001369549; XM_005252003; NM_001018066; NM_001369532; |
| | NM_001369533; NM_001369534; NM_001369544; XM_005252004; |
| | XM_017014751; NM_001369535; NM_001369546; NM_001369550; |
| | XM_011518718; XM_017014752; XM_017014760; NM_001291937; |
| | NM_001369551; NM_001369545; NM_001007097; NM_001018064; |
| | NM_001369541; NM_001369552; XM_017014753; XM_017014755; |
| | NM_001018065; NM_001369542; NM_006180; XM_047423433; |
| | NM_001369537; NM_001369538; NM_001369540; NM_001369543; |
| | NM_001369547; NM_001369548 |
| NTRK3 | XM_006720550; XR_001751292; XM_024449935; XM_047432602; |
| | NM_001375813; XR_002957645; XM_017022245; XM_017022252; |
| | XM_024449934; NM_001375812; XM_006720549; XM_017022241; |
| | XM_017022250; NM_001320135; XM_017022240; XM_047432603; |
| | NM_001012338; XM_006720545; XM_011521638; XM_017022244; |
| | XM_017022251; XM_047432604; NM_001007156; NM_001243101; |
| | XM_017022242; NM_001320134; NM_001375810; NM_001375814; |
| | NM_002530; XM_006720548; XM_017022243; XM_017022254; |
| | NM_001375811; XR_001751293 |
| NUMB | NM_001005745; NM_001320114; NM_001005743; NM_001005744; |
| | NM_003744 |
| NUP214 | NM_005085; NM_001318325; NM_001318324 |
| OCLN | NM_002538; XM_017008914; NM_001205255; XM_047416594; |
| | NM_001205254; XM_017008913; XM_047416593 |
| OGG1 | XM_017006497; XM_017006494; XM_047448201; NM_001354648; |
| | NM_001354652; NM_016820; NM_016829; XM_017006493; NM_016819; |
| | NM_002542; NM_016828; NR_148931; XM_047448200; XM_047448202; |
| | NM_001354650; NM_016821; NM_016827; XM_017006495; NM_016826; |
| | NM_001354649; NR_148930; NR_148932; XM_047448203; NM_001354651 |
| OLR1 | XM_047428908; XM_047428909; NM_002543; NM_001172632; |
| | NM_001172633; XM_047428907 |
| ORM1 | NM_000607 |
| ORM2 | NM_000608 |
| OSCAR | NM_130771; NM_206817; NM_133169; NM_206818; NM_001282349; |
| | NM_133168; NM_001282350 |
| OSMR | XM_005248387; NM_001323504; NM_001323506; NM_001323507; |
| | XM_011514161; XR_007058659; XR_007058660; XM_005248384; |
| | XM_047417871; NM_001168355; NM_003999; XM_017010019; |
| | XM_047417870; XM_047417872; NM_001323505; XR_925661; |
| | XM_005248386; XM_047417873 |
| P2RX1 | XM_006721529; XM_011523898; XM_047436159; XM_047436160; |
| | XM_047436161; NM_002558; XM_011523897; XM_047436158; |
| | XM_011523899; XM_011523900 |
| P2RY10 | NM_001324221; NM_001324225; NM_014499; NM_001324218; |
| | NM_198333; XM_047441998 |
| P2RY13 | XM_006713664; NM_023914; NM_176894 |
| P2RY8 | NM_178129; XM_011545632; XM_047442031; XM_047442729; |
| | XM_005274429; XM_006724864; XM_006724443; XM_011546179; |
| | XM_005274778 |
| PADI2 | NM_007365; XM_017000148; XM_047442975 |
| PADI4 | XM_047416610; XM_011541153; XM_011541151; XM_011541154; |
| | XM_011541150; XM_011541152; XM_011541155; XM_011541156; |
| | NM_012387 |
| PAH | NM_001354304; NM_000277; XM_017019370 |
| PAK1 | XM_024448560; NM_001376272; NM_001376278; NM_001376283; |
| | NM_001376291; NM_001376294; NM_001376304; NM_001376305; |
| | NR_164797; XM_024448558; NM_001376274; NM_001376286; |
| | NM_001376290; NM_001376292; NM_001376295; NM_001376302; |
| | NM_002576; XM_047427053; NM_001376271; NM_001376288; |
| | NM_001376301; XM_024448557; XM_047427047; XM_047427051; |
| | NM_001376275; NM_001376293; NR_164798; XM_024448559; |
| | XM_047427046; NM_001376281; NM_001376284; NM_001376285; |
| | XM_047427052; NM_001128620; NM_001376269; NM_001376273; |
| | XM_047427049; XM_047427054; NM_001376268; NM_001376277; |
| | NM_001376303; XM_047427048; XM_047427050; NM_001376270; |
| | NM_001376276; NM_001376279; NM_001376280; NM_001376282; |
| | NM_001376287; NM_001376289 |
| PAK4 | XM_024451314; NM_001014832; NM_001014835; XM_047438036; |
| | XM_047438040; NM_001014833; XM_011526316; NM_001014831; |
| | XM_047438034; XM_047438035; NM_001014834; NM_001394501; |
| | XM_047438038; XM_047438037; XM_011526317; XM_047438041; |
| | NM_005884; XM_011526320 |
| PALB2 | NM_024675; XM_011545946; XM_011545947; XM_017023673; |
| | XM_011545948 |
| PALLD | XM_011531768; XM_011531769; NM_001166110; NM_001367567; |
| | NM_016081; XM_011531771; XM_011531773; XM_047449862; |
| | NM_001166109; XM_024453940; XM_047449868; NM_001166108; |
| | NM_001367568; XM_011531774; XM_011531775; XM_047449867; |
| | NM_001367569; XM_047449861; XM_047449864; XM_047449865; |
| | NM_001367570; XM_011531772; XM_024453939; XM_047449863; |
| | XM_047449869; XM_047449870; XM_047449866 |
| PAPPA | XM_017014784; NM_002581; XM_006717129 |
| PARD3 | NM_001184793; NM_001184785; NM_001184789; NM_001184790; |
| | NM_001184792; NM_019619; NM_001184786; NM_001184787; |
| | NM_001184788; NM_001184791; NM_001184794 |
| PARP1 | NM_001618 |
| PARP15 | XM_005247160; XM_011512476; XM_005247159; XM_017005791; |
| | XM_017005792; XM_047447580; XM_047447584; XM_011512475; |
| | NM_001113523; XM_047447582; NM_001308320; XM_011512480; |
| | XM_011512479; XM_011512477; XM_047447583; NM_001308321; |
| | NM_152615 |
| PARP2 | XM_047430869; XM_005267247; NM_005484; NM_001042618; |
| | XM_017020912 |
| PARPBP | NM_001382722; NM_001400863; NM_001400879; NM_001400883; |
| | NM_001400893; NM_001400897; NM_001400907; XM_017019541; |
| | NM_001382721; NM_001382723; NM_001382724; NM_001382726; |
| | NM_001400861; NM_001400868; NM_001400874; NM_001400875; |
| | NM_001400889; NM_001400926; XM_047429056; NM_001319996; |
| | NM_001382732; NM_001382735; NM_001400857; NM_001400859; |
| | NM_001400884; XM_047429051; XM_047429052; NM_001382725; |
| | NM_001400856; NM_001400858; NM_001400862; NM_001400867; |
| | NM_001400870; NM_001400871; NM_001400873; NM_001400888; |
| | NM_001400890; NM_001400892; NM_001400902; NM_001400928; |
| | XR_007063095; XR_007063096; XR_007063097; XM_047429057; |
| | NM_001319988; NM_001400854; NM_001400855; NM_001400866; |
| | NM_001400872; NM_001400878; NM_001400895; NM_001400927; |
| | NM_017915; XM_047429048; XM_011538515; XM_047429050; |
| | NM_001319993; NM_001400853; NM_001400860; NM_001400881; |
| | NM_001400886; NM_001400887; NM_001400904; XM_011538514; |
| | XM_047429053; XM_047429055; NM_001319994; NM_001319995; |
| | NM_001400864; NM_001400865; NM_001400876; NM_001400877; |
| | NM_001400885; NM_001400891; NM_001400894; XM_047429049; |
| | XM_047429054; NM_001382728; NM_001382729; NM_001382731; |
| | NM_001400869; NM_001400880; NM_001400896; NM_001400903; |
| | NM_001400905; NM_001400906; NM_001400925; NM_001400929 |
| PARVG | NM_001254742; NM_022141; NM_001137605; NM_001254743; |
| | XM_047441455; NM_001254741; NM_001137606 |
| PAX5 | NM_001280547; NM_001280553; NM_016734; NM_001280548; |
| | NR_103999; NM_001280551; NM_001280555; NM_001280554; |
| | NM_001280552; NM_001280556; NM_001280549; NM_001280550; |
| | NR_104000 |
| PBK | NM_018492; NM_001278945; NM_001363040 |
| PBRM1 | XM_017006734; XM_017006742; XM_047448451; XM_047448465; |
| | NM_001394872; NM_001394874; NM_001400496; NM_001405560; |
| | NM_001405561; NM_001405568; NM_001405571; NM_001405579; |
| | NM_001405581; NM_001405601; NM_001405607; NM_001405614; |
| | NM_001405618; NM_001405622; NM_001405628; NM_001405633; |
| | NM_001405635; NM_181042; XM_011533900; XM_017006725; |
| | XM_017006744; XM_047448447; XM_047448449; XM_047448456; |
| | XM_047448459; NM_001350076; NM_001366070; NM_001366074; |
| | NM_001394868; NM_001394876; NM_001394877; NM_001394878; |
| | NM_001394880; NM_001400474; NM_001400504; NM_001405553; |
| | NM_001405557; NM_001405563; NM_001405564; NM_001405569; |
| | NM_001405573; NM_001405588; NM_001405592; NM_001405597; |
| | NM_001405602; NM_001405610; NM_001405613; NM_001405621; |
| | NM_001405623; NM_001405627; NM_001405630; NM_001405631; |
| | NM_001405634; NM_001405640; NM_018165; XM_017006726; |
| | XM_017006728; XM_047448443; XM_047448448; XM_047448457; |
| | XM_047448460; NM_001350074; NM_001350077; NM_001366072; |
| | NM_001366073; NM_001394870; NM_001400470; NM_001400487; |
| | NM_001405572; NM_001405576; NM_001405582; NM_001405585; |
| | NM_001405589; NM_001405600; NM_001405616; NM_001405620; |
| | NM_001405626; NR_174502; XM_017006741; XM_047448442; |
| | XM_047448445; XM_047448458; XM_047448464; NM_001394873; |
| | NM_001394881; NM_001400472; NM_001400484; NM_001405555; |
| | NM_001405559; NM_001405565; NM_001405570; NM_001405596; |
| | NM_001405608; NM_001405612; NM_001405619; NM_001405641; |
| | XM_047448452; XM_047448455; NM_001350075; NM_001366076; |
| | NM_001394867; NM_001394879; NM_001400479; NM_001400501; |
| | NM_001405574; NM_001405578; NM_001405580; NM_001405583; |
| | NM_001405587; NM_001405590; NM_001405593; NM_001405611; |
| | NM_001405625; NM_001405632; NM_001405638; NM_001405643; |
| | NM_018313; NR_175959; NM_181041; XM_017006730; XM_017006731; |
| | XM_047448446; XM_047448453; XM_047448463; NM_001350078; |
| | NM_001366071; NM_001394869; NM_001394875; NM_001400481; |
| | NM_001405556; NM_001405603; NM_001405605; NM_001405609; |
| | NM_001405637; NM_001405642; XM_011533902; XM_047448444; |
| | XM_047448461; XM_047448462; NM_001350079; NM_001366075; |
| | NM_001400471; NM_001400475; NM_001400490; NM_001400500; |
| | NM_001405554; NM_001405558; NM_001405566; NM_001405567; |
| | NM_001405584; NM_001405591; NM_001405595; NM_001405598; |
| | NM_001405629; NM_001405639; NM_001405636; XM_011533903; |
| | XM_005265280; XM_017006727; XM_024453619; XM_047448450; |
| | XM_047448454; NM_001394871; NM_001400473; NM_001405552; |
| | NM_001405575; NM_001405577; NM_001405586; NM_001405594; |
| | NM_001405599; NM_001405604; NM_001405606; NM_001405615; |
| | NM_001405617; NM_001405624 |
| PBX3 | XM_047423442; XM_047423444; NM_006195; XM_011518755; |
| | XM_047423445; NM_001134778; NM_001330782; NR_024122; |
| | XM_006717130; NR_024123; XM_047423443; XM_047423441 |
| PCNA | NM_182649; NM_002592 |
| PCOLCE | NM_002593 |
| PDCD1 | NM_005018; XM_006712573 |
| PDCD1LG2 | XM_005251600; NM_025239 |
| PDCD4 | NM_014456; NM_145341; NM_001199492 |
| PDE4C | NM_001330172; XM_047438917; NM_001369701; NM_001098819; |
| | XM_047438918; NR_040546; XM_047438919; XM_047438916; |
| | NM_000923; NM_001098818; NM_001395274 |
| PDGFC | XM_047415970; XM_017008455; NM_016205; XM_047415971; |
| | XM_047415969; XM_047415972; NR_036641 |
| PDGFRA | XM_047415767; NM_001347828; NM_001347829; XM_005265743; |
| | XM_017008281; NM_001347827; XM_047415766; NM_001347830; |
| | NM_006206; XM_006714041 |
| PDGFRB | NM_001355016; NM_002609; NM_001355017; NR_149150 |
| PDPK1 | XM_047434199; XM_011522523; XM_047434198; NM_002613; |
| | NM_031268; XM_047434201; XM_047434202; NM_001261816; |
| | XM_011522521; XM_024450296; XM_047434200 |
| PDPN | XM_047434471; NM_001006625; NM_198389; NM_001385053; |
| | NM_001006624; XM_006710295; NM_006474; NM_013317; |
| | XM_024451404 |
| PEBP1 | NM_002567 |
| PECAM1 | XM_047436251; NM_000442; XM_005276883; XM_017024741; |
| | XM_017024739; XM_005276880; XM_005276881; XM_005276882 |
| PER1 | NM_002616 |
| PFKFB3 | XM_047425346; NM_001282630; XM_005252464; XM_017016329; |
| | NM_004566; NM_001363545; XM_047425341; XM_011519493; |
| | XM_047425345; XM_047425349; NR_136554; NM_001145443; |
| | XM_047425343; XM_047425344; NM_001323016; NM_001323017; |
| | XM_047425342; XM_047425347; NM_001314063 |
| PGF | NM_001293643; NM_002632; NM_001207012; XM_047431476 |
| PGLYRP1 | NM_005091 |
| PGLYRP2 | NM_052890; NM_001363546 |
| PGR | XM_011542869; NM_001271161; NR_073142; XM_006718858; |
| | NM_000926; NM_001202474; NM_001271162; NR_073141; NR_073143 |
| PHLPP1 | NM_194449 |
| PHOSPHO1 | XM_047435505; NM_001143804; XM_047435504; NM_178500; |
| | XM_047435506 |
| PIAS3 | NM_006099 |
| PIK3AP1 | XM_005269499; XM_047424566; NM_152309; XM_011539248 |
| PIK3CA | NM_006218; XM_006713658 |
| PIK3CB | XM_011512895; XM_047448307; NM_001256045; XM_017006619; |
| | NM_006219; XM_047448311; XM_047448312; XM_047448314; |
| | XM_047448315; XM_047448309; XM_006713659; XM_047448308; |
| | XM_047448310; XM_047448313 |
| PIK3CD | XM_024447663; XM_047422552; XM_047422561; XM_047422568; |
| | XM_047422573; XM_047422574; XM_047422575; XM_047422577; |
| | XM_024447664; XM_047422553; XM_047422564; XM_047422566; |
| | NM_005026; XM_047422567; XM_047422569; NM_001350234; |
| | XM_047422554; XM_047422555; XM_047422589; XM_006710689; |
| | XM_047422550; XM_047422557; XM_006710687; XM_047422558; |
| | XM_047422559; XM_047422563; XM_047422565; XM_047422580; |
| | XM_047422551; XM_047422556; XM_047422562; XM_047422570; |
| | XM_047422571; NM_001350235; XM_047422560; XM_047422572; |
| | XM_047422576; XM_047422578 |
| PIK3CG | XM_017012328; XM_005250443; XM_047420479; NM_001282426; |
| | XM_011516317; XM_047420481; XM_047420480; NM_001282427; |
| | XM_011516316; NM_002649 |
| PIK3IP1 | NM_001135911; NM_052880 |
| PIK3R1 | XM_017009585; XM_047417315; NM_181504; NM_181524; |
| | XM_047417316; NM_001242466; NM_181523; XM_005248542; |
| | XM_047417317 |
| PIK3R2 | NR_073517; NM_005027; NR_162071 |
| PIK3R3 | NM_001114172; NM_001328651; NM_001303429; NM_001328653; |
| | NM_001328649; NR_137329; NM_001328648; NM_001328652; |
| | NM_001328650; NM_001328654; NM_001303428; NM_003629 |
| PIK3R5 | NM_001388398; NM_001388400; NM_001388396; NM_014308; |
| | XM_047435711; NM_001142633; NM_001251853; XM_047435710; |
| | NM_001251852; NM_001388397; XM_047435709; NM_001251851; |
| | NM_001251855; NM_001388399 |
| PILRA | XM_047420291; NM_178273; NM_178272; NM_013439; XM_047420292 |
| PIM1 | NM_002648; NM_001243186 |
| PIM3 | NM_001001852 |
| PINX1 | NM_001284356; NM_017884 |
| PIPOX | NM_016518 |
| PKP1 | NM_000299; NM_001005337 |
| PKP2 | NM_001005242; NM_004572 |
| PLA2G7 | XM_047419360; NM_001168357; XM_005249408; NM_005084; |
| | XM_047419359 |
| PLCB2 | XM_047432672; XM_047432683; XM_017022317; XM_047432676; |
| | XM_047432679; NM_004573; XR_007064458; XM_017022314; |
| | XM_047432670; NM_001284297; XM_047432678; XM_047432681; |
| | NM_001284298; NM_001284299; XM_047432669; XM_047432671; |
| | XM_047432673; XM_047432674; XM_047432677; XM_047432682; |
| | XM_047432689; XM_017022319; XM_047432675; XM_047432684; |
| | XM_047432686; XM_047432667; XM_047432668; XM_047432680; |
| | XM_047432687; XM_047432685; XM_047432688 |
| PLCG2 | NM_002661 |
| PLEK | NM_002664; XM_047444772 |
| PLG | NM_000301; NM_001168338 |
| PLGLB1 | NM_001032392.4 |
| PLGLB2 | NM_002665.4 |
| PLIN1 | NM_002666; XM_005254934; NM_001145311 |
| PLK1 | NM_005030 |
| PLOD2 | XM_017006625; NM_000935; XM_047448320; NM_182943; |
| | XM_047448319 |
| PLXNC1 | XM_047428050; XM_011537730; NR_037687; NM_005761; |
| | XM_006719186; XM_011537731 |
| PMAIP1 | NM_021127; NM_001382616; NM_001382615; NM_001382617; |
| | NM_001382618; NM_001382623 |
| PMEL | NM_001200054; NM_001200053; NM_001320121; NM_001384361; |
| | NM_001320122; NM_006928 |
| PML | NM_033238; NM_033240; NM_033249; NM_033244; NM_033247; |
| | NM_033250; NM_002675; NM_033239; NM_033246 |
| PMS1 | XM_017004344; NM_001128143; NM_001321045; NM_001321044; |
| | XM_006712596; XM_017004347; NM_000534; NM_001289408; |
| | NM_001321049; NM_001321051; NR_110332; XM_017004348; |
| | XM_017004350; XM_024452967; XM_047444777; NM_001321046; |
| | XM_047444775; XM_047444776; NM_001321047; XM_024452966; |
| | XM_047444778; NM_001128144; NM_001289409; NM_001321048 |
| PMS2 | XM_024446800; NM_001322004; NM_001322006; NM_001322013; |
| | NM_001322007; NM_001322010; NM_001322014; XM_047420483; |
| | NM_001322003; NM_001322008; XM_047420485; XM_047420486; |
| | NM_001322005; NM_001322009; NM_001322015; XM_047420484; |
| | XM_047420482; NM_001322011; NM_001322012; NM_000535; NR_136154 |
| PNKD | XM_017003771; XM_017003772; NM_015488; NM_001077399; |
| | NM_022572 |
| PNPLA2 | NM_020376 |
| POLB | XM_005273538; XM_017013583; XM_005273537; XM_005273540; |
| | XM_017013584; XM_047421901; XM_047421900; XM_005273536; |
| | XM_005273539; NM_002690; XM_005273535; XR_428311 |
| POLD1 | XM_005259008; XM_047438946; NM_001256849; XM_011527038; |
| | XM_047438948; XM_047438949; XM_047438950; NM_002691; |
| | XR_935835; NM_001308632; XM_017026882; XM_047438947; NR_046402 |
| POLD2 | XM_047420498; XM_047420501; NM_006230; NM_001256879; |
| | XM_024446802; XM_047420497; XM_047420500; NM_001127218; |
| | XM_047420499 |
| POLD3 | XM_011544734; NM_001363597; NM_006591; NR_046409; NR_046410; |
| | XM_005273716; XM_047426295 |
| POLD4 | NM_001256870; NR_046412; NM_021173; NR_046411; NR_046413 |
| POLE | XM_011534797; XM_011534799; NM_006231; XM_047429018; |
| | XR_941395; XM_011534795; XM_011534802 |
| POLE2 | XM_047431483; XM_047431484; NM_001197330; NM_001348384; |
| | XR_007064015; NM_001348385; NM_002692; NM_001197331; |
| | XR_007064016 |
| POLE3 | NM_001278255; NM_017443; NR_027261 |
| POLE4 | NM_019896 |
| POLG | NM_001126131; NM_002693 |
| POLH | NM_001291970; NM_006502; XM_047418900; NM_001291969 |
| POLI | NM_001351612; NM_001351615; NM_001351620; NM_001351621; |
| | NM_007195; NM_001351617; NM_001351611; NM_001351618; |
| | XM_011525797; NM_001351613; NM_001351632; NR_147257; |
| | NM_001351616; NM_001351610; NM_001351614; XM_024451081; |
| | XM_005258192; NM_001351619 |
| POLK | NM_001387111; NM_001395900; NM_016218; NM_001387112; |
| | NM_001395894; NR_170560; NM_001387110; NR_170559; |
| | NM_001395893; NM_001395899; NM_001395901; NM_001345922; |
| | NM_001395897; NR_144315; NM_001345921; NM_001387113; |
| | NM_001395902 |
| POLL | XM_011539664; XM_017016088; XM_047425089; XM_011539650; |
| | XM_011539656; XM_024447943; XM_047425087; XM_047425092; |
| | XM_047425095; XM_011539657; XM_024447942; XM_047425094; |
| | NR_033406; XM_011539662; XM_017016090; XM_024447945; |
| | XM_047425090; NM_001174085; XM_006717775; XM_017016085; |
| | XM_017016091; XM_047425093; NM_001174084; XM_047425086; |
| | XM_047425097; XM_047425098; NM_013274; XM_006717777; |
| | XM_047425088; XM_047425091; XM_047425096; NM_001308382; |
| | XM_011539651; XM_011539654; XM_011539655; XM_017016084 |
| POLM | NR_156113; NR_156112; NM_001284330; NM_001284331; |
| | NM_001362683; NM_013284; NR_104299 |
| POLN | NM_181808 |
| POLQ | NM_199420; NM_006596 |
| PON1 | NM_000446 |
| PORCN | XM_047442359; XM_047442364; XM_047442368; XM_047442361; |
| | XM_047442372; NM_203475; XM_047442360; XM_047442362; |
| | NM_203474; NM_203476; NM_001282167; XM_047442369; |
| | XM_047442371; XM_047442358; XM_047442363; XM_047442367; |
| | NM_203473; XM_047442365; XM_047442370; XM_047442373; |
| | NM_022825; XM_047442357; XM_047442366 |
| POU2AF1 | XM_006718860; XM_017017932; XM_006718859; XM_005271593; |
| | XM_047427137; NM_006235 |
| POU5F1 | NM_001173531; NM_203289; NM_002701; NM_001285986; |
| | NM_001285987 |
| PPARG | NM_001354669; NM_001354670; NM_001374263; NM_001330615; |
| | NM_001374262; NM_005037; NM_001374261; NM_138711; NM_138712; |
| | NM_001374264; NM_001374266; NM_001354668; NM_015869; |
| | NM_001354666; NM_001354667; NM_001374265 |
| PPM1D | NM_003620; XR_934577; XR_007065507 |
| PPP1R13B | XM_017021117; XM_047431170; XR_001750206; XM_047431171; |
| | NM_015316; XM_011536593; XR_007063997; XR_001750205; |
| | XM_017021116; XM_017021119; XM_005267487; XM_047431172; |
| | XR_001750204 |
| PPP1R16B | NM_001172735; NM_015568; XM_011528768; XM_047440086 |
| PPP1R9B | NM_032595 |
| PPP2R1A | NM_001363656; NM_014225; NR_033500 |
| PPP3CC | XM_047421941; XM_047421942; NM_001243975; NM_005605; |
| | XR_007060744; NM_001243974 |
| PRC1 | XM_011522187; XM_047433307; NM_003981; XM_017022712; |
| | XM_011522189; XM_011522191; XM_047433299; XM_047433301; |
| | XM_047433306; XM_047433308; NM_199413; XM_011522190; |
| | XM_017022713; XM_017022714; XM_047433297; XM_047433304; |
| | XM_047433314; XM_017022716; XM_047433313; NM_199414; |
| | XM_005254987; XM_006720759; XM_006720760; XM_011522192; |
| | XM_047433295; XM_047433303; XM_047433305; XM_047433310; |
| | XM_011522188; XM_047433298; XM_047433300; XM_047433309; |
| | XM_047433311; NM_001267580 |
| PRDM1 | XM_047419248; XM_047419247; XM_011536064; XM_017011187; |
| | XM_011536062; XM_047419246; XM_006715550; NM_182907; |
| | NM_001198 |
| PRF1 | NM_005041; NM_001083116 |
| PRG2 | NM_001302926; NM_002728; NM_001243245; NM_001302927 |
| PRG3 | NM_006093 |
| PRKACA | NM_207518; NM_002730; XM_047439070; NM_001304349; |
| | XM_017026948 |
| PRKCA | XM_024450830; XR_007065317; XM_017024836; XR_007065316; |
| | XR_001752558; XM_047436389; XM_017024837; XM_047436388; |
| | NM_002737; XM_024450829; XR_007065315 |
| PRKCB | NM_212535; NM_002738; XM_047434365 |
| PRKCD | XM_047448564; NM_212539; NM_001316327; NM_001354676; |
| | NM_001354680; NM_001354678; NM_006254; NM_001354679; |
| | XR_007095706 |
| PRKCI | XM_047448574; NM_002740; XM_047448575 |
| PRKCQ | XM_005252497; NM_001282645; NM_001282644; NM_001323267; |
| | XM_005252496; NM_001323266; NM_006257; NM_001242413; |
| | NM_001323265 |
| PRKDC | NM_001081640; NM_006904 |
| PRLR | XM_011514068; NM_001204315; NM_001204318; XM_047417390; |
| | NM_001204317; NR_037910; XM_047417388; NM_000949; |
| | NM_001204316; XM_006714484; XM_047417391; NM_001204314; |
| | XM_024446131 |
| PRMT7 | XM_017023304; XM_047434217; XM_047434218; XM_047434222; |
| | XM_047434223; XM_047434235; XM_047434239; XM_011523112; |
| | XM_011523121; XM_047434219; XM_047434225; XM_047434227; |
| | NM_001378022; XR_007064886; XR_007064887; XR_007064888; |
| | XM_011523113; XM_011523115; XM_017023297; XM_047434237; |
| | NM_001378020; NM_001378023; NM_019023; NR_165365; |
| | XR_001751915; XM_047434224; XM_047434226; NM_001184824; |
| | XM_011523116; XM_017023298; XM_017023301; XM_047434228; |
| | XM_047434232; NM_001351143; NM_001378021; NR_165366; |
| | XM_047434229; XM_047434231; NM_001290018; NR_147058; |
| | NR_165368; NR_165369; NR_165373; XR_007064885; XM_017023300; |
| | XM_047434220; XM_047434221; XM_047434233; XM_047434234; |
| | XM_047434238; NM_001351144; NR_147056; XM_017023292; |
| | XM_017023296; XM_017023299; XM_047434230; XM_047434236; |
| | NM_001378018; NR_147057; NR_165367; NR_165370; NR_165371; |
| | NR_165372; XR_002957814 |
| PROC | NM_000312; NM_001375603; NM_001375608; NM_001375604; |
| | NM_001375605; XM_024453002; XM_047445118; NM_001375607; |
| | XM_017004505; XM_024453003; NM_001375606; XM_047445117; |
| | NM_001375613; NM_001375602; NM_001375609; NM_001375610; |
| | NM_001375611 |
| PRODH2 | NM_001195226; NM_001368249; NM_016335; NM_001368250 |
| PROX1 | XM_005273194; XM_047425575; XM_047425585; NM_002763; |
| | XM_017001833; NM_001270616; XM_005273195; XM_011509773; |
| | XM_047425558; XM_047425572; XM_047425574; XM_011509771; |
| | XM_011509772; XM_017001832 |
| PROZ | XM_047430720; NM_003891; XM_047430721; NM_001256134; |
| | XM_017020813 |
| PRSS33 | NM_001385462; NM_001385463; NM_001385464; NM_152891; NR_169625 |
| PRTN3 | XM_011528136; NM_002777 |
| PSAP | NM_001042466; NM_001042465; NM_002778 |
| PSCA | NM_005672; NR_033343 |
| PSEN2 | XM_017001836; XM_047425596; XM_047425597; XM_047425601; |
| | NM_000447; NM_012486; XR_001737316; XR_007061979; XR_949150; |
| | XM_005273199; XR_007061980; XM_017001835 |
| PSENEN | NM_001281532; NM_172341; NM_018468 |
| PSMB1 | NM_002793 |
| PSMB10 | NM_002801 |
| PSMB2 | NM_001199780; NM_001199779; NM_002794 |
| PSMB5 | NM_001144932; NM_002797; NM_001130725 |
| PSMB8 | NM_004159; NM_148919 |
| PSMB9 | NM_148954; NM_002800 |
| PTAFR | NM_001164722; NM_000952; NM_001164723; NM_001164721 |
| PTCH1 | NR_149061; NM_001083604; NM_001083605; NM_001083607; |
| | NM_001083606; NM_001354918; NM_001354919; NM_000264; |
| | NM_001083602; NM_001083603 |
| PTEN | NM_000314; NM_001304718; NM_001304717 |
| PTGDR | XM_005267891; NM_000953; NM_001281469 |
| PTGDS | NM_000954 |
| PTGS2 | NM_000963 |
| PTK2 | XM_024447202; XM_024447209; XM_047422025; XM_047422035; |
| | NM_001352699; NM_001352700; NM_001352706; NM_001352712; |
| | NM_001352717; NM_001352721; NM_001352733; NM_001352738; |
| | NM_001352739; NM_001352742; NM_001352744; NM_001352746; |
| | NM_001387607; NM_001387632; NM_001387639; NM_001387644; |
| | NM_001387648; NM_001387650; NM_001387656; NM_001387660; |
| | NM_005607; NM_153831; NR_148036; XM_047422020; XM_047422021; |
| | XM_047422032; XM_047422039; XM_047422046; XM_047422048; |
| | XM_047422053; NM_001352708; NM_001352710; NM_001352727; |
| | NM_001352729; NM_001352731; NM_001352736; NM_001352737; |
| | NM_001352745; NM_001352749; NM_001387588; NM_001387591; |
| | NM_001387592; NM_001387613; NM_001387612; NM_001387615; |
| | NM_001387616; NM_001387622; NM_001387627; NM_001387635; |
| | NM_001387652; NM_001387653; NR_170670; XM_024447208; |
| | XM_047422030; XM_047422040; XM_047422044; NM_001352696; |
| | NM_001352707; NM_001352709; NM_001352711; NM_001352724; |
| | NM_001352728; NM_001387584; NM_001387587; NM_001387630; |
| | NM_001387657; NM_001387659; NR_148038; NR_170672; |
| | XM_047422016; XM_047422018; XM_047422038; XM_047422050; |
| | NM_001352702; NM_001352713; NM_001352722; NM_001352723; |
| | NM_001352743; NM_001352747; NM_001352751; NM_001387586; |
| | NM_001387603; NM_001387604; NM_001387611; NM_001387620; |
| | NM_001387625; NM_001387628; NM_001387631; NM_001387640; |
| | NM_001387641; NM_001387647; NM_001387654; NM_001387661; |
| | XM_024447203; XM_047422017; XM_047422027; XM_047422034; |
| | XM_047422037; XM_047422041; XM_047422054; NM_001352698; |
| | NM_001352719; NM_001352726; NM_001352735; NM_001352741; |
| | NM_001387585; NM_001387610; NM_001387617; NM_001387618; |
| | NM_001387629; NM_001387636; NM_001387638; NM_001387642; |
| | NM_001387645; NM_001387646; NM_001387655; NM_001387658; |
| | NR_148037; NR_148039; XM_024447207; XM_047422019; |
| | XM_047422026; XM_047422033; XM_047422047; XM_047422049; |
| | NM_001352716; NM_001352730; NM_001352732; NM_001352740; |
| | NM_001387589; NM_001387590; NM_001387608; NM_001387619; |
| | NM_001387633; NM_001387634; NM_001387643; XM_047422023; |
| | XM_047422031; XM_047422045; NM_001199649; NM_001352695; |
| | NM_001352701; NM_001352703; NM_001352704; NM_001352705; |
| | NM_001352715; NM_001352720; NM_001352750; NM_001352752; |
| | NM_001387605; NM_001387609; NM_001387614; NM_001387624; |
| | NM_001387662; NR_170671; NR_170673; XM_047422022; |
| | XM_047422024; XM_047422028; XM_047422029; XM_047422036; |
| | XM_047422043; XM_047422052; NM_001316342; NM_001352694; |
| | NM_001352697; NM_001352714; NM_001352718; NM_001352725; |
| | NM_001352734; NM_001352748; NM_001387606; NM_001387621; |
| | NM_001387623; NM_001387637; NM_001387649; NM_001387651 |
| PTK7 | NM_152880; NM_152882; NM_152881; XM_047419157; NM_002821; |
| | NR_072997; NR_072998; NM_152883; NM_001270398; XM_011514766; |
| | XM_011514765 |
| PTPN11 | NM_001374625; NM_001330437; NM_080601; NM_002834; NM_018508; |
| | XM_011538613 |
| PTPN14 | NM_005401; XM_047426374; XM_047426370; XM_024448759; |
| | XM_017001941; XM_047426367 |
| PTPN22 | XM_011541225; NM_001193431; XM_047417632; XM_011541223; |
| | XM_017001006; NM_015967; XM_011541221; XM_011541222; |
| | NM_012411; XM_017001005; XM_047417630; XM_047417631; |
| | NM_00 1308297 |
| PTPN6 | XM_011520988; NM_002831; XM_047429231; XM_024449106; |
| | NM_080548; XM_047429232; NM_080549 |
| PTPRB | XM_017019724; NM_001206971; NM_001109754; NM_001330204; |
| | XM_006719529; NM_002837; XR_944651; XM_006719528; |
| | XM_011538614; NM_001206972 |
| PTPRC | XM_047426420; NM_001267798; NM_002838; NR_052021; NM_080922; |
| | XM_006711473; XM_006711474; XM_047426417; XM_047426409; |
| | XM_047426381; XM_006711472; XM_047426398; XM_047426415; |
| | NM_080921 |
| PTPRCAP | NM_005608 |
| PTPRD | XM_017014958; XM_017014963; XM_017014968; XM_017014976; |
| | XM_047423645; XM_047423646; XM_047423649; NM_001040712; |
| | NM_001377947; NM_130391; XM_006716827; XM_017014970; |
| | XM_017014971; XM_017014983; XM_047423643; XM_047423644; |
| | XM_047423660; NM_001378058; XM_017014965; XM_017014967; |
| | XM_017014979; XM_047423650; NM_001377958; XM_017014964; |
| | XM_017014974; XM_017014977; XM_017014978; XM_047423648; |
| | XM_047423652; XM_047423655; XM_047423658; NM_001377946; |
| | NM_002839; NM_130392; XM_017014966; XM_047423654; NM_130393; |
| | XM_017014972; XM_017014980; XM_024447625; XM_047423642; |
| | XM_047423647; XM_047423656; XM_017014961; XM_017014969; |
| | XM_017014982; XM_047423641; XM_047423651; XM_047423653; |
| | XM_047423657; XM_047423659; NM_001171025; XM_006716817; |
| | XM_006716823; XM_006716825 |
| PTTG1 | NM_001282382; NM_001282383; NM_004219 |
| PTTG1IP | NM_001286822; NR_104597; NM_004339 |
| PVRIG | NM_024070; NM_001397246; NM_001387134 |
| PYHIN1 | XM_005244930; NM_198930; NM_152501; NM_198928; XM_011509243; |
| | NM_198929; XM_011509242 |
| RAB22A | NM_020673 |
| RAB42 | XM_047443959; NM_152304; NM_001385188; NM_001193532 |
| RAB7B | NM_001304839; XM_006711288; NM_001164522; NM_177403 |
| RAC1 | NM_198235; NM_002933; NM_198232; NM_198234 |
| RAC2 | XM_006724286; NM_002872 |
| RACGAP1 | XM_017019221; XM_017019226; XM_017019227; NM_001126103; |
| | NM_001319999; XM_047428748; NM_001320002; XM_017019222; |
| | XM_017019223; NM_001320005; XM_024448958; NM_001126104; |
| | NM_001320003; XM_047428746; XM_047428750; NM_001320000; |
| | NM_001320006; XM_006719359; XM_011538238; XM_047428747; |
| | XM_047428751; NM_013277; XM_017019224; XM_017019225; |
| | NM_001320004; XM_047428745; XM_047428749; NM_001320001; |
| | NM_00 1320007 |
| RAD1 | NM_133282; NM_002853; NM_133377; NM_001033673; NR_026591 |
| RAD17 | XM_017009681; XM_047417459; XM_047417462; NM_133344; |
| | XM_047417461; NM_133338; XM_047417456; XM_047417457; |
| | NM_002873; NM_133341; XM_047417458; NM_001278622; NM_133339; |
| | NM_133342; XM_047417460; NM_133340; NM_133343 |
| RAD23A | NR_072976; NM_001270362; NM_001270363; NM_005053 |
| RAD23B | NM_001244713; NM_001244724; NM_002874 |
| RAD50 | NM_005732; NM_133482 |
| RAD51 | XM_011521859; XM_011521861; NM_002875; XM_011521857; |
| | XM_011521858; NM_001164269; XM_047432925; XM_011521860; |
| | NM_001164270; NM_133487 |
| RAD51B | NM_001321809; NM_001321817; NM_001321821; NM_001321814; |
| | NM_001321815; NM_001321819; NM_002877; NM_133510; |
| | NM_001321812; NM_001321810; NM_001321818; NM_133509 |
| RAD51C | XM_006722001; NR_103873; XM_006722002; NR_103872; |
| | XM_006722004; XM_011525094; XM_047436505; NM_058216; |
| | NM_002876 |
| RAD51D | NR_037711; NM_001142571; NM_133629; NR_037712; NM_133628; |
| | NM_133630; NM_002878; NM_133627 |
| RAD9A | XM_047427381; XM_047427382; NM_001243224; NM_004584; |
| | XM_006718652; XM_047427383; XM_017018098 |
| RAF1 | XM_017006966; XM_047448649; NR_148942; XM_011533974; |
| | NM_001354691; NR_148940; NM_002880; NM_001354690; |
| | NM_001354692; NM_001354695; NM_001354689; NM_001354693; |
| | NM_001354694; NR_148941; XM_047448650; XM_047448651 |
| RALBP1 | NM_006788; XM_047437280; XM_047437284; XM_047437278; |
| | XM_047437281; XM_047437282; XM_047437283; XM_047437279 |
| RALGPS2 | XM_047423755; XM_006711410; XM_047423766; NM_018037; |
| | NR_174383; NM_001400042; NM_001286247; NR_174384; NM_152663; |
| | XM_047423777 |
| RARA | XM_047436508; NM_001024809; NM_001145302; XM_005257552; |
| | NM_001033603; XM_005257553; XM_005257554; XM_047436507; |
| | NM_000964; NM_001145301; XM_011525095; XM_011525096; |
| | XM_047436506 |
| RASA1 | NM_002890; NM_022650 |
| RASAL2 | XM_047434860; XM_005245622; XM_011510166; XM_017002850; |
| | XM_017002854; XM_047434849; XM_047434857; XM_047434859; |
| | NM_170692; XM_017002849; XM_017002853; XM_017002852; |
| | XM_017002855; XM_047434838; XM_011510167; NM_004841; |
| | XM_017002851; XM_047434837; XM_047434839 |
| RASAL3 | XM_047439231; NR_174477; NR_174478; XM_011528187; |
| | NM_001400377; XM_011528186; NM_001400378; NM_001400381; |
| | NM_022904; NM_001348027; NM_001348028; NM_001400379; |
| | NM_0014003 80 |
| RASGRP1 | XM_047432077; NM_001128602; NM_005739; XM_047432073; |
| | XM_047432076; XM_047432078; XM_047432074; NM_001306086; |
| | XM_047432075 |
| RASGRP2 | XM_017017082; XM_047426231; NM_001098671; XM_011544723; |
| | XM_011544725; NM_153819; XM_011544718; NM_001318398; |
| | XM_047426233; XM_011544721; NM_001098670; XM_011544720; |
| | NM_005825; XM_047426232 |
| RASGRP3 | XM_047443878; NM_001139488; NM_001349978; XM_047443879; |
| | NM_001349977; NM_001349981; NM_001349976; NM_001349979; |
| | NM_001349980; XM_011532746; XM_047443877; NM_001349975; |
| | NM_170672; XM_011532748; XM_017003761; NM_015376 |
| RASGRP4 | NM_001146205; NM_001146204; XR_935732; NM_001146207; |
| | NM_170604; NM_052949; NM_001146203; NM_170603; NM_001146202; |
| | NM_001146206; NM_170602 |
| RASIP1 | NM_017805 |
| RASSF1 | NM_007182; XM_011533316; NM_170716; NM_001206957; NM_170712; |
| | NM_170713; NM_170714; XM_047447372; NM_170717; NM_170715 |
| RASSF10 | NM_001080521 |
| RASSF5 | NM_182663; NM_031437; NM_182664; NM_182665 |
| RB1 | NM_000321 |
| RB1CC1 | XM_011517649; XM_017014107; NM_001083617; XM_017014109; |
| | XM_047422495; XM_011517643; XM_011517644; XR_928826; |
| | XM_011517645; XM_017014108; XM_047422497; XM_017014105; |
| | XM_011517647; XM_047422494; NM_014781; XM_017014103; |
| | XM_017014104; XM_017014106; XM_047422496 |
| RBBP8 | XM_006722520; XM_047437728; NM_002894; XM_047437730; |
| | XM_005258325; XM_011526132; XM_047437729; NM_203291; |
| | XM_047437731; XM_047437732; NM_203292; XM_006722519; |
| | XM_047437727; XM_006722521 |
| RBM10 | NM_152856; XM_047442556; NM_001204466; NM_005676; |
| | XM_047442555; XM_047442551; NM_001204467; XM_047442554; |
| | XM_047442552; XM_047442553; XM_005272677; XM_005272678; |
| | XM_005272679; NM_001204468 |
| RBM15 | NM_014092; NM_022768; NM_001201545 |
| RBP5 | NM_001329454; NM_031491; XR_007063133; XM_047429633; |
| | XR_007063132; XM_017020002; XR_001748882 |
| RBPJ | XM_047415658; NM_203283; NM_001374400; NM_001379408; |
| | XM_017008171; XM_047415656; NM_001379406; NM_001379409; |
| | NM_001363577; NM_001374401; NM_001374402; NM_005349; |
| | NM_001374403; XM_011513840; XM_047415657; NM_015874; |
| | NM_203284; NM_001379407 |
| RBX1 | NM_014248 |
| RCC2 | NM_001136204; NM_018715 |
| RCE1 | NM_001032279; NM_005133 |
| RCSD1 | NR_136519; NM_052862; NM_001322923; NM_001322924 |
| RDH16 | NM_001320108; NM_003708 |
| RECQL4 | XM_047422440; XM_047422445; XM_047422446; XM_047422439; |
| | XM_047422448; XM_047422437; XM_047422438; XM_047422443; |
| | XM_047422447; XM_047422442; NM_004260; XM_047422444; |
| | XM_011517384; XM_047422441 |
| RECQL5 | XM_005257818; XM_006722186; XM_047437087; NM_001003715; |
| | NM_004259; XM_011525486; XM_047437089; XM_011525485; |
| | XM_047437086; XM_047437090; XM_005257822; XM_047437091; |
| | XM_047437088; XM_047437085; NM_001003716; XM_011525484; |
| | XM_047437092 |
| REL | XM_011533010; XM_017004627; NM_002908; NM_001291746 |
| RELA | XM_011545206; NM_001404659; NM_001404660; NM_021975; |
| | XM_011545207; NM_001145138; NM_001404662; NM_001404663; |
| | NM_001404657; NM_001243984; XM_047427392; NM_001404658; |
| | NM_001404661; NM_001243985 |
| RELB | XM_005259128; XM_005259127; XM_047439189; NM_006509; |
| | XM_047439190 |
| REPS2 | XM_011545605; XM_047442627; XM_024452479; XM_005274625; |
| | XM_011545603; XM_011545604; XM_047442626; XM_005274626; |
| | XM_011545607; XM_017029955; XM_017029956; NM_001080975; |
| | NM_004726; XM_017029958; XM_011545606; XM_017029957; |
| | XM_047442628 |
| RET | NM_020975; NM_001355216; NM_020630; NM_020629; NM_000323 |
| REV3L | XM_011536028; XM_011536036; XM_047419216; NM_001372078; |
| | XM_047419217; XM_047419218; NM_001286431; XM_011536029; |
| | NM_001286432; XM_047419215; XM_017011155; XM_047419220; |
| | XM_011536030; XM_011536032; XM_047419219; NM_002912 |
| RGN | XM_006724568; XM_017029954; NM_004683; NM_001282848; |
| | NM_152869; NM_001282849; XM_006724567 |
| RHEB | XM_047420685; XM_024446854; XM_011516457; NM_005614 |
| RHOBTB2 | XM_047421607; NM_001160036; XM_047421609; XM_047421608; |
| | NM_001160037; NM_001374791; XM_047421610; XM_047421611; |
| | NM_015178 |
| RHOH | XM_047415675; NM_001278361; NM_001278364; XM_017008189; |
| | NM_001278360; NM_001278363; NM_001278359; NM_001278365; |
| | NM_001278362; XM_047415674; NM_001278369; NM_001278367; |
| | NM_001278368; XM_011513692; NM_001278366; NM_004310 |
| RHOT1 | XM_047436363; NM_001288758; XM_047436353; XM_047436357; |
| | XM_047436359; XM_047436364; XM_047436354; XM_047436356; |
| | NM_001033566; NM_018307; XM_047436362; NM_001033568; |
| | NM_001288754; XM_011524973; XM_047436361; XM_047436355; |
| | XM_047436358; NM_001033567; NM_001288755; NR_110083; |
| | XM_011524969; XM_047436360 |
| RICTOR | XM_006714463; XM_017009311; NM_001285440; NM_152756; |
| | NM_001285439; XM_017009312; XM_011514005; XM_047417068; |
| | XM_047417069; XM_011514006; XM_047417070 |
| RIF1 | XM_047444869; XR_007077531; XR_007077537; XR_007077541; |
| | XR_007077545; XM_047444871; XM_047444873; XM_047444880; |
| | XR_007077542; XR_007077543; XR_007077546; XR_007077547; |
| | XM_047444883; XM_047444886; XR_007077529; XR_007077540; |
| | XM_005246665; XM_017004422; XM_047444872; NM_018151; |
| | XR_007077532; XM_017004424; XM_047444874; XM_047444877; |
| | XM_047444884; NM_001177664; NM_001177665; XR_007077534; |
| | XR_007077536; XR_007077544; XM_047444876; XM_047444879; |
| | XM_047444881; XM_047444887; NM_001177663; XR_007077538; |
| | XR_007077539; XM_047444868; XM_047444870; XM_047444875; |
| | XM_047444878; XM_047444882; XR_007077530; XR_007077533; |
| | XM_017004423; XM_047444867; XM_047444885; XR_007077535 |
| RIT1 | NM_001256820; NM_006912; NM_001256821 |
| RLTPR | XM_011522874; XM_047433644; XM_011522875; XM_017022953; |
| | NM_001013838; XM_047433645; NM_001317026 |
| RMI1 | NM_001358293; XM_017015140; NM_001358292; NM_024945; |
| | NM_001358291; NM_001358294 |
| RNASE2 | NM_002934 |
| RNASE3 | NM_002935 |
| RNASE6 | XM_017021566; NM_005615 |
| RNF43 | XM_011524956; XM_047436330; NM_017763; XM_047436331; |
| | NM_001305544; XM_017024800; XM_047436332; NM_001305545 |
| ROBO4 | NM_019055; XM_006718861; NM_001301088; XM_011542875 |
| ROCK1 | NM_005406 |
| ROR1 | NM_005012; XM_011541526; XM_017001376; XM_017001377; |
| | NM_001083592 |
| ROR2 | XM_005252008; XM_017014762; XM_047423434; XM_047423436; |
| | XM_006717121; XM_047423435; NM_004560; XM_005252009; |
| | XM_047423437; NM_001318204 |
| RORC | XM_006711484; NM_001001523; NM_005060; XM_047427201 |
| ROS1 | XM_011536053; XM_011536055; XM_011536054; XM_011536057; |
| | XM_011536049; XM_011536058; XM_047419232; NM_001378891; |
| | XM_006715548; NM_002944; XM_011536050; XM_017011173; |
| | XM_047419231; XM_011536051; XM_011536056; XM_017011172; |
| | NM_001378902 |
| RP2 | NM_006915 |
| RPN2 | XM_006723852; NM_001135771; NM_001324299; XM_006723851; |
| | NM_001324301; NM_001324302; NM_001324306; NM_002951; |
| | NM_001324303; NM_001324304; NM_001324305 |
| RPS6 | NM_001010 |
| RPS6KA3 | XM_017029719; XM_047442335; XM_011545561; XM_011545556; |
| | XM_011545562; XM_047442333; NM_004586; XM_005274577; |
| | XM_011545557; XM_011545560; XM_017029718; XM_047442332; |
| | XM_047442334; XM_047442336; XM_017029717; XM_005274573; |
| | XM_011545555 |
| RPS6KB1 | NM_001369679; NR_161459; NM_001369673; NR_161455; |
| | NM_001272043; NR_161462; XM_011525102; NM_001272060; |
| | NM_001369674; NM_001369678; NR_161461; NM_001272044; |
| | NM_001369669; NM_001369670; NM_001369671; NM_001369677; |
| | NM_001272042; NM_001369672; NR_161456; NR_161458; NR_161460; |
| | NM_001369675; NM_001369676; NR_161457; NM_003161 |
| RPTOR | NM_001163034; NM_020761 |
| RRAGC | NM_022157; NM_001271851 |
| RRM1 | NM_001033; NM_001330193; NM_001318065; NM_001318064 |
| RRM2 | NM_001034; NR_164157; NR_161344; NM_001165931 |
| RRP1B | NM_015056 |
| RSF1 | NM_016578; XM_005274051; XM_017017923 |
| RSPO1 | XM_006710583; NM_001242909; NM_001242908; NM_001242910; |
| | NM_173640; NM_001038633 |
| RUNX1 | XM_047441010; XM_047441012; XM_047441013; XM_005261069; |
| | XM_047441007; XM_047441016; XM_005261068; XM_011529767; |
| | XM_011529770; XM_047441015; XM_011529766; XM_047441009; |
| | NM_001001890; NM_001122607; XM_047441014; XM_011529768; |
| | XM_047441011; NM_001754 |
| RUNX2 | NM_001278478; NM_001369405; NM_004348; NR_103533; |
| | NM_001024630; NR_103532; NM_001015051 |
| RUNX3 | NM_001031680; NM_004350; XM_011542351; XM_005246024; |
| | XM_047433131; NM_001320672 |
| RYBP | NM_012234 |
| RYK | NM_002958; NM_001005861; XR_007095716 |
| S1PR2 | NM_004230 |
| S1PR5 | NM_001166215; NM_030760 |
| SAA4 | NM_006512 |
| SAMD3 | XM_024446333; XM_024446335; NM_001277185; XM_017010309; |
| | XR_001743174; NM_152552; NM_001258275; XM_017010308; |
| | XM_024446337; XM_047418239; XM_017010305; XM_017010310; |
| | NM_001017373 |
| SAMSN1 | XM_011529684; NM_001256370; NM_001395858; XM_047440942; |
| | NM_001286523; NM_022136; XM_011529685; XM_011529686; |
| | XM_047440941; NM_001256579; NM_001395856; NM_001395857 |
| SASH1 | XM_017010598; XM_017010600; XM_017010605; XM_024446384; |
| | XM_047418497; XM_047418498; NM_015278; XM_017010599; |
| | XM_024446385; NM_001346505; NM_001346506; NM_001346507; |
| | NM_001346508; XM_047418499; XM_047418496; XM_047418500; |
| | NM_001346509 |
| SASH3 | NM_018990; XM_006724763 |
| SATB1 | XM_011533990; NM_001322872; NM_001322874; XM_011533988; |
| | NM_002971; NM_001322873; NM_001131010; NM_001195470; |
| | NM_001322876; NM_001322871; XM_047448683; XM_011533989; |
| | NM_001322875 |
| SATB2 | NM_015265; NM_001172517; NM_001172509; NR_134967; |
| | XM_005246396; XM_047443775 |
| SAV1 | XM_011537057; NM_021818; XM_047431659 |
| SDC1 | NM_001006946; XM_005262620; XM_005262621; NM_002997; |
| | XM_005262622 |
| SEC11C | NM_001307941; NM_033280; XM_011526260 |
| SELE | NM_000450 |
| SELL | NR_029467; NM_000655 |
| SEMA3A | XM_005250110; XM_047419751; NM_006080 |
| SEMA3F | XM_047448701; NM_004186; XM_047448700; NM_001318800; |
| | NM_001318798; XM_005265381; XM_006713289; XM_006713290; |
| | XM_005265382; XM_011533998; XM_011534000; XM_047448699 |
| SERPINA10 | XM_005267733; XM_017021353; NM_016186; NM_001100607 |
| SERPINA7 | XM_005262180; NM_000354; XM_006724683 |
| SERPINB2 | XM_024451192; NM_002575; NM_001143818 |
| SERPINB5 | NM_002639; XM_006722483 |
| SERPINC1 | NM_000488; NM_001386306; NM_001386304; NM_001386305; |
| | NM_001386303; NM_001365052; NM_001386302 |
| SERPIND1 | NM_000185 |
| SERPINE1 | NM_000602; NM_001386463; NM_001386465; NM_001386457; |
| | NM_001386466; NM_001165413; NM_001386456; NM_001386460; |
| | NM_001386461; NM_001386462; NM_001386459; NM_001386464; |
| | NM_001386458 |
| SERPINF2 | XM_005256701; NM_001165920; XM_047436303; NM_000934; |
| | XM_017024765; NM_001165921 |
| SERPINH1 | XM_047427800; NM_001207014; XM_024448756; XM_011545327; |
| | NM_001235 |
| SETD2 | XR_007095670; NR_146158; XR_007095672; XM_024453488; |
| | NM_001349370; XM_024453487; XR_002959514; XR_007095673; |
| | XM_024453489; XM_047448045; NM_014159; XR_007095671; NM_012271 |
| SF3B1 | XM_047443838; NM_012433; XM_047443841; XM_047443839; |
| | XM_047443840; NM_001005526; NM_001308824 |
| SFRP1 | NM_003012 |
| SFRP2 | NM_003013 |
| SFRP4 | NM_003014 |
| SFRP5 | NM_003015 |
| SH2D1A | NM_001114937; NM_002351 |
| SH2D1B | NM_053282 |
| SH3BP2 | NM_001145856; NM_001122681; NM_001145855; NM_003023 |
| SHH | NR_132319; NM_000193; NR_132318; XM_011516480; XM_011516479; |
| | XM_047420718; NM_001310462 |
| SIGLEC1 | NM_001367089; NM_023068 |
| SIGLEC14 | XM_047437991; NM_001098612 |
| SIGLEC5 | NM_001384708; NM_001384709; NM_003830; XM_047446914; |
| | XM_047446915 |
| SIGLEC7 | NM_001277201; XM_011526721; XM_047438605; NM_014385; |
| | XM_047438604; NR_102350; NM_016543 |
| SIGLEC8 | XM_011526734; NM_014442; NM_001363548 |
| SIGLEC9 | XM_011526732; NM_014441; NM_001198558; XM_047438615; |
| | XM_047438616 |
| SIRPA | XM_011529173; XM_047439919; XM_047439917; XM_024451836; |
| | XM_005260670; XM_047439915; XM_047439916; NM_001330728; |
| | XM_047439918; XM_047439920; NM_001040023; NM_080792; |
| | NM_001040022 |
| SIRPB2 | XM_047440129; XM_047440130; NR_021484; XR_007067453; |
| | XM_005260709; XM_047440127; NM_001122962; XM_047440128; |
| | XR_007067452; XM_005260708; NM_001134836; XR_007067451 |
| SIRPG | XM_011529286; NM_018556; XM_011529287; XM_005260749; |
| | NM_001039508; NM_080816 |
| SIRT1 | NM_001314049; NM_012238; NM_001142498 |
| SIRT3 | NM_001370314; NM_001370317; NR_163396; NR_163397; XR_007062467; |
| | XM_017017431; NM_001370310; NM_001370318; NM_001370320; |
| | NR_163387; NR_163392; NM_001017524; NM_001370322; NR_163400; |
| | NM_001370319; NR_163388; NR_163398; NR_163401; XM_011519957; |
| | NR_163386; NR_163390; NR_163391; NR_163393; NR_163394; |
| | NM_001370312; NM_001370323; NR_163395; XM_011519956; |
| | XM_047426677; NM_001370316; NM_001370321; NM_001370324; |
| | NM_001370325; NM_012239; NR_163389; NR_163399; NR_163402; |
| | NM_001370315 |
| SIRT4 | NM_001385735; NM_001385734; NM_001385733; NM_012240; |
| | XM_006719309 |
| SIT1 | NM_014450 |
| SIX2 | NM_016932; XM_005264100 |
| SKAP1 | XM_047436975; XM_047436978; XM_047436979; XM_047436980; |
| | XM_005257755; XM_047436976; XM_047436977; NM_001075099; |
| | XM_017025258; XM_047436972; XM_047436974; XM_047436973; |
| | NM_003726 |
| SKP2 | NM_032637; NM_001243120; NM_005983; XM_047417536; XR_001742203 |
| SLA | NM_006748; XM_047422110; NM_001045556; XM_047422108; |
| | NM_001045557; XM_047422109; NM_001282964; XM_047422107; |
| | NM_001282965 |
| SLA2 | NM_175077; NM_032214 |
| SLAMF6 | XM_047443866; NM_001184714; NM_001184715; NM_052931; |
| | NM_001184716; XM_017000216 |
| SLAMF7 | XM_011509828; XM_011509829; NM_001282589; NM_001282590; |
| | NM_001282596; NM_001282591; NM_001282593; NM_001282588; |
| | NM_001282595; XM_047426359; NM_001282592; NM_001282594; |
| | NM_021181 |
| SLC10A1 | NM_003049 |
| SLC13A5 | NM_177550; XM_011523795; NM_001284510; NM_001143838; |
| | NM_001284509 |
| SLC16A4 | NM_001319220; NM_004696; XM_006711033; XM_047433990; |
| | XM_005271317; XM_047433986; NM_001201547; NM_001201549; |
| | NM_001201548; NM_001201546; XM_047433967; XM_047433980 |
| SLC22A1 | NM_153187; XM_006715552; XM_005267103; NM_003057 |
| SLC22A7 | XM_011514257; NM_153320; XM_024446313; XM_047418105; |
| | XM_017010199; XM_047418102; NM_006672; XM_047418103; |
| | XM_047418104; XM_011514262; XM_017010198; XM_011514256 |
| SLC25A18 | XM_011546154; XM_011546149; XM_017028968; NM_001303484; |
| | XM_011546150; XM_011546151; XM_047441535; XM_011546152; |
| | XM_011546153; NM_031481 |
| SLC27A5 | NM_012254; XM_011526364; XM_017026214; NM_001321196 |
| SLC2A1 | NM_006516 |
| SLC2A2 | XM_011513087; NM_000340; NM_001278659; XM_047448761; |
| | NM_001278658 |
| SLC38A3 | XM_006712954; NM_006841; XR_001739987 |
| SLC38A6 | XM_017021021; XM_047431005; XM_047431006; XM_047431009; |
| | XM_011536469; XM_017021022; XM_047431007; NM_153811; |
| | XR_001750164; XR_007063987; XM_047431001; XR_007063988; |
| | XM_047431003; XM_017021020; XM_017021025; XM_047431004; |
| | NM_001172702; XM_024449496; XM_017021023; XM_047431002; |
| | XM_047431008; XM_047431010; NR_033344 |
| SLC39A6 | NM_001099406; NM_012319 |
| SLC6A2 | XM_011523295; XM_047434510; XM_047434511; XM_011523299; |
| | XM_011523300; XM_047434512; XM_047434513; NM_001172502; |
| | NM_001043; NM_001172501; XM_006721263; NM_001172504 |
| SLX1A | NM_001014999.3; NM_001015000.2 |
| SLX1B | NM_024044.4; NM_178044.3 |
| SLX4 | XM_024450471; XM_011522715; XR_007064923; XM_047434801; |
| | NM_032444 |
| SMAD1 | XM_047415691; NM_001354814; NM_001354811; NM_001354812; |
| | NM_005900; XM_011531962; XM_011531964; XM_047415690; |
| | NM_001003688; XM_047415688; XM_047415689; NM_001354813; |
| | NM_001354817; NM_001354816 |
| SMAD2 | XM_047437508; NM_001003652; NM_005901; XM_017025749; |
| | NM_001135937; XM_047437507 |
| SMAD3 | NM_001145103; NM_001145104; XM_011521559; NM_005902; |
| | NM_001145102 |
| SMAD4 | NM_005359 |
| SMAD5 | NM_001001420; XM_017009470; XM_024446046; XM_024446047; |
| | NM_005903; NM_001001419 |
| SMAD6 | NM_005585; XR_931827; NM_001142861; XM_011521561; NR_027654 |
| SMAD7 | NM_005904; NM_001190821; NM_001190822; NM_001190823; |
| | XM_047437509 |
| SMARCA4 | XM_024451667; NM_001128845; NM_001387283; NR_164683; |
| | XM_047439249; NM_001128848; XM_047439243; XM_047439246; |
| | XM_047439247; XM_047439251; XM_006722846; XM_024451661; |
| | XM_047439245; NM_001374457; XM_047439250; NM_001128846; |
| | XM_011528198; XM_024451663; NM_001128847; XM_047439244; |
| | NM_001128844; NM_001128849; NM_003072; XM_024451658; |
| | XM_047439248 |
| SMO | NM_005631; XM_047420759 |
| SMUG1 | XM_011538119; XM_047428641; XM_047428652; XM_047428654; |
| | NM_001243787; NM_001351239; NM_001351240; NM_001351242; |
| | NM_001351245; NM_001351250; NM_001351254; NM_001351261; |
| | XR_007063064; XM_011538116; XM_047428643; XM_047428650; |
| | XM_047428651; NM_001351256; NM_001351262; NM_014311; |
| | XM_047428639; XM_047428645; NM_001351241; NM_001351243; |
| | NM_001351252; XM_011538121; XM_047428635; XM_047428637; |
| | XM_047428640; XM_047428646; XM_047428655; NM_001243789; |
| | NM_001351237; NM_001351258; NM_001351259; XM_047428636; |
| | XM_047428642; XM_047428644; NM_001243790; NM_001351244; |
| | NM_001351248; NM_001351253; XM_006719319; XM_047428632; |
| | XM_047428649; XM_047428653; NM_001243788; NM_001351249; |
| | NM_001351251; NM_001351255; NM_001351260; XM_011538112; |
| | XM_047428633; XM_047428634; XM_047428638; XM_047428648; |
| | NM_001243791; NM_001351257; XM_011538118; NM_001351238; |
| | NM_001351246; NM_001351247 |
| SMURF1 | XM_047420635; XM_047420637; NM_020429; XM_017012457; |
| | NM_181349; NM_001199847; XM_047420636 |
| SMURF2 | XM_005257585; XR_007065425; NM_022739; XM_047436546 |
| SNAI1 | NM_005985 |
| SNAI2 | NM_003068 |
| SNCA | XM_011532204; NM_001146054; NM_000345; NM_001375287; |
| | XM_011532206; NM_007308; XM_047416097; NR_164675; |
| | XM_011532207; NM_001375288; NM_001375290; NR_164676; |
| | XM_011532203; XM_011532205; NR_164674; NM_001146055; |
| | NM_001375286; NM_001375285 |
| SNCG | NM_003087; XM_047425681; NM_001330120 |
| SNX20 | NM_001144972; NM_153337; NM_182854 |
| SOCS1 | NM_003745 |
| SOCS3 | NM_001378933; NM_003955; NM_001378932 |
| SOD2 | NM_001322817; NM_001322820; NM_001322815; NM_001322814; |
| | NM_000636; NM_001322816; NM_001024465; NM_001024466; |
| | NM_001322819 |
| SOS1 | XM_047445586; XM_011533064; XM_047445584; XM_047445585; |
| | XM_047445582; NM_001382395; NM_005633; XM_047445581; |
| | XM_047445583; NM_001382394 |
| SOST | NM_025237 |
| SOX11 | NM_003108 |
| SOX18 | NM_018419 |
| SOX30 | NM_001308165; NM_178424; NM_007017 |
| SOX9 | NM_000346 |
| SP140 | XM_017003249; XM_047443078; XM_011510515; XM_011510516; |
| | XM_011510517; XM_017003250; XM_017003253; XM_047443073; |
| | XM_047443076; XM_047443077; NM_001278452; NM_001278453; |
| | XM_011510520; XM_017003245; XM_017003246; XM_017003252; |
| | XM_047443074; XM_005246253; XM_005246255; XM_011510518; |
| | XM_017003247; XM_005246252; XM_005246256; XM_017003248; |
| | XM_047443079; XM_047443080; NM_001278451; XM_017003242; |
| | XM_005246254; XM_006712223; XM_017003240; XM_017003243; |
| | XM_047443072; XM_047443081; NM_007237; XM_011510519; |
| | XM_017003239; NM_001005176 |
| SPARC | NM_001309443; NM_001309444; NM_003118 |
| SPEN | NM_015001 |
| SPHK1 | NM_021972; NM_001355139; NM_001142602; NM_182965; |
| | NM_001142601 |
| SPI1 | XM_017018173; NM_003120; XM_047427487; NM_001080547 |
| SPIB | NM_001243999; NM_001243998; NM_001244000; NM_003121 |
| SPN | NM_001030288; NM_003123 |
| SPOCK1 | NM_004598 |
| SPP1 | NM_001251829; NM_001040060; NM_001251830; NM_000582; |
| | NM_001040058 |
| SPP2 | NM_006944; XM_011511699; XM_005246102; XM_011511698; |
| | XM_011511700 |
| SPRY2 | NM_001318537; NM_005842; NM_001318536; NM_001318538 |
| SQLE | NM_003129; XM_011517246 |
| SSBP2 | XM_047417058; NM_001400348; NM_001400358; NR_174545; |
| | NR_174546; NR_174563; XM_047417059; XM_047417066; |
| | NM_001345886; NM_001400343; NM_001400345; NM_001400346; |
| | NM_001400366; NR_174529; NR_174531; NR_174536; NR_174548; |
| | NR_174558; XM_017009308; XM_047417065; NM_001400356; |
| | NM_001400368; NM_001400369; NM_001400375; NM_012446; |
| | NR_174525; NR_174528; NR_174535; NR_174537; NR_174544; |
| | NR_174547; NR_174561; NM_001400347; NM_001400349; |
| | NM_001400351; NM_001400352; NM_001400364; NM_001400371; |
| | NR_174538; NR_174540; NR_174552; XM_047417061; NM_001256733; |
| | NM_001256736; NM_001394351; NM_001400344; NM_001400359; |
| | NM_001400360; NM_001400363; NM_001400367; NM_001400374; |
| | NR_174530; NR_174539; NR_174543; NR_174553; NR_174555; |
| | NR_174560; XM_047417062; NM_001256732; NM_001400340; |
| | NM_001400350; NM_001400355; XM_047417060; NM_001256734; |
| | NM_001256735; NM_001394350; NM_001394352; NM_001400341; |
| | NM_001400342; NM_001400353; NM_001400357; NM_001400361; |
| | NM_001400362; NR_174526; NR_174541; NR_174542; NR_174550; |
| | NR_174554; NR_174562; XM_017009309; XM_047417064; |
| | NM_001400354; NR_174527; NR_174532; NR_174533; NR_174534; |
| | NR_174549; NR_174551; NR_174556; NR_174557; NR_174559 |
| SSR4 | XM_047442390; XM_047442389; NM_001204527; NM_006280; |
| | NR_037927; NM_001204526 |
| SSTR2 | NM_001050 |
| STAB1 | χM_047447774; XM_006713065; XM_005264974; XM_047447777; |
| | NM_015136; XM_005264973; XM_047447775; XM_047447776 |
| STAP1 | NM_001317769; NM_012108 |
| STAT1 | NM_001384890; NM_139266; NM_001384881; NM_007315; |
| | NM_001384891; XM_006712718; NM_001384882; NM_001384889; |
| | NM_001384883; NM_001384885; NM_001384880; NM_001384884; |
| | NM_001384888; NM_001384886; NM_001384887 |
| STAT2 | XM_047429470; XM_047429471; XR_007063122; XM_047429472; |
| | NM_001385111; NM_001385115; NM_005419; NM_001385110; |
| | NM_001385113; XM_011538699; XM_011538698; XM_047429469; |
| | NM_001385114; XM_011538697; XR_245953; XM_047429468; NM_198332 |
| STAT3 | XM_047436586; NM_001384993; XM_017024973; NM_001384989; |
| | NM_001384990; NM_001384992; NM_003150; NM_001369512; |
| | NM_001384986; NM_001384991; NM_001369517; XM_047436585; |
| | NM_001369513; NM_001369518; NM_001369519; NM_001384984; |
| | NM_001384987; NM_001384988; NM_001369514; NM_001369516; |
| | NM_001369520; NM_001384985; NM_139276; NM_213662 |
| STAT4 | XM_047445601; XM_047445609; XM_047445602; XM_047445604; |
| | NM_003151; XM_006712719; XM_047445606; XM_047445605; |
| | XM_047445607; NM_001243835; XM_047445603; XM_047445608; |
| | XM_047445600 |
| STAT5A | NM_001288719; XM_047436591; XM_047436590; NM_001288720; |
| | NM_003152; XM_047436589; NM_001288718; XM_047436588; |
| | XM_005257624 |
| STAT5B | XM_005257626; XM_047436593; XM_024450898; XM_024450897; |
| | NM_012448; XM_017024977 |
| STAT6 | NM_001178078; NM_001178080; NM_001178081; XM_047429475; |
| | NM_001178079; XM_047429476; XM_047429473; XM_047429477; |
| | NM_003153; XM_047429474; NR_033659 |
| STC2 | NM_003714 |
| STK11 | NM_000455 |
| STK3 | XM_017013756; XM_047422133; XR_007060752; NM_001256312; |
| | XM_011517248; XM_047422137; NM_006281; XR_007060753; |
| | XM_011517257; XM_017013757; XM_017013761; XM_047422132; |
| | XM_047422135; NM_001256313; XR_007060755; XM_011517252; |
| | XM_047422134; XM_047422136; XR_007060754; XM_011517251; |
| | XM_017013758 |
| STK4 | NM_001352385; XM_017028033; XM_011529018; XM_017028031; |
| | NM_006282; NR_147974; NR_147975; XM_005260532; XM_047440425; |
| | XM_047440426 |
| STMN1 | NM_203399; NM_203401; NM_152497; NM_005563; NM_001145454 |
| STUB1 | NM_001293197; NM_005861 |
| STX11 | XM_047419437; XM_011536213; XM_011536217; XM_047419436; |
| | XM_011536214; XM_047419438; NM_003764; XM_047419440; |
| | XM_011536218; XM_047419439; XM_047419441 |
| STYK1 | XM_005253417; NM_018423; XM_011520738; XM_011520736; |
| | XM_011520737; XM_047429099 |
| SUFU | XM_011539864; XM_047425335; XM_047425339; NM_016169; |
| | XM_011539863; XM_047425337; NM_001178133; XM_011539858; |
| | XM_047425336; XM_011539860; XM_011539861; XM_047425338 |
| SULT2A1 | NM_003167 |
| SUSD6 | NM_014734 |
| SWSAP1 | NM_175871 |
| SYCP3 | NM_001177949; XM_005268922; XM_005268927; NM_001177948; |
| | NM_153694; XM_011538421; XM_047428913; XM_005268924; |
| | XM_005268926 |
| SYK | XM_047423811; XM_005252147; XM_047423809; NM_001174168; |
| | XM_011518946; XM_047423810; NM_001174167; NM_001135052; |
| | NM_003177 |
| SYNE1 | XM_006715416; XM_006715417; XM_006715420; XM_017010611; |
| | XM_017010614; XM_017010617; XM_047418508; XM_006715414; |
| | XM_017010608; XM_017010615; NM_033071; XM_006715410; |
| | XM_047418503; XM_047418505; XM_047418513; NM_001347702; |
| | XM_006715411; XM_006715425; XM_047418504; NM_001134379; |
| | NM_015293; XM_006715412; XM_006715422; XM_006715424; |
| | XM_011535642; XM_017010613; XM_017010616; XM_017010618; |
| | XM_017010619; XM_047418502; XM_047418507; XM_047418509; |
| | NM_182961; NM_133650; XM_006715407; XM_006715421; |
| | XM_011535644; XM_047418501; XM_047418512; NM_001099267; |
| | XM_006715408; XM_017010609; XM_017010612; XM_047418506; |
| | XM_047418510; XM_047418511; XM_006715409; XM_006715413; |
| | XM_006715415; XM_006715423; XM_011535641; XM_011535643; |
| | XM_011535645; XM_017010610; NM_001347701 |
| SYP | NM_003179 |
| TACSTD2 | NM_002353 |
| TAF1 | XM_047442392; NM_001286074; XM_047442400; XM_047442396; |
| | XM_047442403; NR_104389; NR_104392; XM_047442391; |
| | XM_047442399; XM_047442404; XM_047442405; NR_104391; |
| | NR_104393; NR_104388; NR_104394; XM_024452430; XM_047442393; |
| | XM_047442406; NM_004606; NR_104387; NR_104390; NR_104395; |
| | NR_104396; XM_005262300; XM_047442394; XM_047442397; |
| | XM_047442398; XM_047442401; NM_138923; XM_047442395; |
| | XM_047442402 |
| TAGAP | NM_001278733; NM_138810; NM_054114; NM_152133 |
| TAGLN | NM_001001522; NM_003186 |
| TAOK1 | NM_025142; NM_020791 |
| TAOK2 | XM_011545983; XM_011545984; XM_011545986; NM_004783; |
| | XM_047434922; NM_016151; NM_001252043; XM_011545985; |
| | XM_047434918; XM_047434920; XM_047434921; XM_011545982; |
| | XM_047434917; XM_047434919 |
| TAOK3 | XM_017019410; NM_001346487; NM_001346494; NM_016281; |
| | XM_047428945; XM_047428947; NM_001346489; NM_001346490; |
| | XM_006719445; XM_017019409; XM_047428952; NM_001346488; |
| | NM_001346492; NM_001346495; XM_047428941; XM_047428948; |
| | XM_047428951; XM_047428953; NM_001346491; NM_001346493; |
| | XM_017019408; XM_047428942; XM_047428944; NM_001346497; |
| | XM_005253898; XM_011538437; XM_024449010; XM_047428943; |
| | XM_047428946; XM_047428950; XM_047428954; NM_001346496 |
| TAP1 | NM_000593; NM_001292022 |
| TAP2 | NM_001290043; NM_000544; NM_018833 |
| TAPBP | XM_017011227; XM_047419272; XM_047419271; XM_011514828; |
| | NM_003190; NM_172208; NM_172209 |
| TARDBP | NM_007375; XR_007058559; XR_007058563; XR_007058564; |
| | XR_007058558; XR_007058560; XR_007058562; XR_007058561 |
| TAT | NM_000353 |
| TBC1D10C | XM_011545002; NM_001369495; XM_047426913; NM_001369492; |
| | NM_001256508; NM_001369494; NM_198517; XM_006718539; |
| | XM_047426910; NM_001369498; XM_006718538; XM_047426911; |
| | XM_047426914; NM_001369496; NR_046266; XM_047426909; |
| | NM_001369497 |
| TBX21 | NM_013351 |
| TBX3 | NM_005996; NM_016569 |
| TCF21 | NM_003206; NM_198392 |
| TCF7 | XM_006714679; XM_006714686; XM_006714685; XM_047417637; |
| | XM_047417638; XM_047417639; XM_011543606; XM_006714684; |
| | XM_047417636; XM_047417640; XM_047417643; NM_001134852; |
| | XM_006714682; NM_201634; XM_006714678; XM_047417634; |
| | XM_047417642; NM_001346425; NM_213648; XM_011543604; |
| | XM_047417641; NM_001366502; NM_003202; XM_047417633; |
| | XM_047417635; NM_001134851; NM_001346450; NM_201632; NR_033449 |
| TCF7L1 | XM_006712109; NM_031283 |
| TCF7L2 | NM_001146286; NM_001198528; NM_001363501; NM_001349871; |
| | NM_001198530; NM_001198527; NM_001198529; NM_001349870; |
| | NM_030756; NM_001198526; NM_001367943; NM_001198531; |
| | NM_001146274; NM_001146284; NM_001198525; NM_001146283; |
| | NM_001146285 |
| TDG | XM_047429489; XM_047429488; XM_047429487; NM_001363612; |
| | NM_003211; XM_047429486; NM_001008411 |
| TDO2 | NM_005651 |
| TEAD1 | NM_021961 |
| TEAD2 | XM_011527404; XM_047439523; XM_011527403; XM_047439531; |
| | XM_047439535; XM_047439537; XM_047439540; NM_001256658; |
| | NM_003598; XM_047439538; XM_047439541; XM_005259334; |
| | XM_011527402; XM_047439525; XM_047439530; NM_001256662; |
| | XM_047439526; XM_047439529; XM_047439533; XM_047439534; |
| | XM_047439536; XM_047439542; XM_047439543; NM_001256660; |
| | XM_006723428; XM_011527400; XM_047439520; XM_047439522; |
| | XM_047439524; XM_047439527; XM_011527401; XM_011527405; |
| | XM_011527406; XM_047439532; XM_047439539; XM_047439544; |
| | NM_001256659; NM_001256661; XM_006723429; XM_011527399; |
| | XM_047439521; XM_047439528 |
| TEAD3 | NM_001395214; NM_003214 |
| TEAD4 | NM_201443; NM_003213; NM_201441 |
| TEK | NM_001375475; NM_000459; NM_001290077; NM_001290078; |
| | NM_00 1375476 |
| TERT | NR_149162; NM_198255; NM_198253; NR_149163; NM_001193376; |
| | NM_198254 |
| TES | NM_015641; NM_152829 |
| TESPA1 | XM_006719715; XM_047429930; NM_001136030; NR_147068; |
| | XR_007063147; XM_011539035; NR_147064; NR_147065; NR_147072; |
| | NR_147073; XM_017020262; XM_047429929; NM_001261844; |
| | NM_001351152; NR_147066; NR_147071; XM_017020263; |
| | NM_001351149; NR_147069; XM_011539037; NM_001098815; |
| | NM_001351151; NM_014796; NR_147067; NM_001351150; |
| | NM_001351154; NM_001351155; NR_147062; NR_147063; NR_147070; |
| | XM_047429931; NM_001351148; NM_001351153; XR_007063146 |
| TF | NM_001063; NM_001354703; NM_001354704 |
| TFEB | XM_006715212; NM_001271943; NM_001271945; NM_001167827; |
| | XM_047419361; NM_007162; NM_001271944; XM_005249411 |
| TFF3 | NM_003226 |
| TFR2 | NM_003227; NM_001206855 |
| TGFB1 | NM_000660; XM_011527242 |
| TGFB2 | NM_003238; NR_138149; NR_138148; NM_001135599 |
| TGFB3 | NM_001329938; NM_003239; NM_001329939 |
| TGFBR1 | NM_004612; XM_011518948; NM_001130916; XM_011518949; |
| | NM_001306210 |
| TGFBR2 | NM_003242; XM_011534045; XM_011534043; NM_001024847; |
| | XM_047448787 |
| TGM3 | NM_003245 |
| THBD | NM_000361 |
| THBS2 | NM_003247; NM_001381940; NM_001381941; NM_001381939; |
| | NR_167745; NM_001381942; NR_167744 |
| THEMIS | XM_047418763; XM_047418766; XM_047418767; NM_001164687; |
| | XM_047418764; NM_001318531; NM_001394521; XM_047418765; |
| | NM_001164685; NM_001394520; NM_001394522; NM_001010923 |
| THRSP | NM_003251 |
| THY1 | NM_006288; NM_001311162; NR_164077; NM_001311160; NM_001372050 |
| TIAM1 | NM_001353685; NM_001353686; NM_001353688; NM_001353692; |
| | NM_001353689; NM_001353687; NM_001353690; NM_001353691; |
| | NM_003253; NM_001353694; NM_001353693; XM_005261040; |
| | XM_047440969; NM_001353684 |
| TIE1 | XM_047429354; XM_005271163; NM_001253357; XM_017002207; |
| | XM_047429343; NM_005424 |
| TIGIT | XM_047447672; XM_047447671; NM_173799 |
| TIMP1 | NM_003254 |
| TIMP2 | NM_003255 |
| TJP1 | XM_011521972; XM_047432982; XM_047432984; NM_001330239; |
| | XM_005254619; XM_017022523; XM_017022524; XM_047432988; |
| | XM_047432990; NM_001301025; NM_001355014; XM_017022525; |
| | XM_047432991; NM_003257; XM_017022522; XM_017022526; |
| | XM_047432983; XM_047432986; XM_047432989; XM_047432981; |
| | XM_047432985; NM_001301026; XM_017022521; XM_017022527; |
| | NM_001355013; XM_005254620; XM_047432987; NM_001355012; |
| | NM_001355015; NM_175610 |
| TJP2 | XM_011519206; NM_001369871; NM_001369872; XM_011519208; |
| | XM_011519209; NM_001369870; NM_004817; XM_047424090; |
| | XM_011519207; XM_047424094; NM_001369874; NM_001170630; |
| | XM_047424092; NM_001369875; XM_047424091; NM_001170415; |
| | NM_001170416; XM_047424095; NM_001170414; NM_001369873; |
| | NM_201629 |
| TJP3 | XM_047438611; NM_001267560; NM_001267561; NM_014428 |
| TLE1 | XM_005252151; XM_005252153; XM_011518951; NM_001303103; |
| | NM_001303104; XM_005252154; XM_005252163; XM_006717260; |
| | XM_047423814; XM_047423816; XM_006717263; XM_047423813; |
| | XM_047423818; XM_005252152; XM_047423815; XM_047423817; |
| | XM_006717258; XM_005252156; XM_005252162; XM_006717259; |
| | XM_006717261; XM_006717262; NM_005077 |
| TLE2 | NM_001144761; XM_006722864; NM_001144762; NM_001300846; |
| | XM_011528230; NM_003260 |
| TLE3 | XM_005254625; XM_011521977; XM_011521979; NM_001105192; |
| | NM_001282982; XM_047432993; XM_011521978; NM_001282980; |
| | NM_001282981; NM_005078; XM_005254633; XM_011521980; |
| | XM_011521982; XM_005254623; XM_017022532; NM_001282979; |
| | XM_005254622; XM_011521983; XM_005254628; XM_011521976; |
| | XM_006720665; XM_011521981; XM_047432992; NM_020908 |
| TLE4 | XM_011518952; NM_001282753; XM_011518956; XM_011518969; |
| | XM_011518970; XM_017015068; XM_047423820; XM_047423823; |
| | XM_047423824; NM_001351546; XM_011518955; XM_011518957; |
| | XM_011518962; XM_011518965; XM_011518966; XM_047423821; |
| | XM_011518972; NM_001351542; NM_001351543; NM_001351547; |
| | NM_001351564; NM_007005; XM_011518953; XM_011518967; |
| | NM_001282748; NM_001282749; NM_001282760; NM_001351541; |
| | XM_006717264; XM_006717268; XM_011518961; XM_017015067; |
| | XM_017015074; XM_047423819; XM_047423822; NM_001351550; |
| | NR_104239; XM_011518958; XM_011518959; XM_011518963; |
| | XM_011518964; NM_001351552; NM_001351556; NM_001351563; |
| | XM_011518954; XM_011518960; XM_011518968; XM_017015083; |
| | NM_001351558; NM_001351560; NM_001351562 |
| TLN1 | NM_006289 |
| TLR2 | XM_017008575; NM_001318791; NM_001318796; NM_001318795; |
| | XM_017008573; XM_047416111; XM_047416113; NM_001318793; |
| | XM_047416114; NM_001318787; NM_003264; XM_011532216; |
| | XM_047416112; NM_001318790; XM_011532215; NM_001318789 |
| TMC6 | XM_047435250; XM_047435270; XM_047435271; XM_047435274; |
| | NM_001321185; NM_007267; NR_168289; XM_011524257; |
| | XM_047435252; XM_047435265; NM_001374594; NR_168288; |
| | XM_024450556; XM_047435256; XM_047435263; XM_047435278; |
| | NM_001374593; NR_168290; XM_011524256; XM_047435261; |
| | XM_047435268; XM_047435269; XM_047435277; NM_001127198; |
| | NM_001375354; NR_168291; XM_047435251; XM_047435253; |
| | XM_047435258; XM_047435272; XM_047435273; XM_047435255; |
| | XM_047435257; XM_047435266; XM_047435275; NM_001375353; |
| | XM_047435254; XM_047435259; XM_047435276; NM_001374596; |
| | XM_047435260; XM_047435264; XM_047435267 |
| TMC8 | XM_024450618; XM_024450620; XM_047435479; XM_047435494; |
| | XM_047435488; XR_007065273; XM_024450623; XM_047435492; |
| | XM_017024244; XM_024450619; XM_024450624; XM_047435482; |
| | XM_047435489; XR_002957973; XR_007065271; XM_024450622; |
| | XM_047435484; XM_047435485; XM_047435487; XM_047435491; |
| | XM_047435493; XR_007065274; XM_024450621; XR_007065276; |
| | XM_024450617; XM_024450626; XM_024450627; XM_047435478; |
| | XM_047435480; XM_047435481; XM_047435486; XM_047435490; |
| | XR_007065272; XM_024450625; NM_152468; XR_007065275 |
| TMED7- | NM_001164468; NM_001164469 |
| TICAM2 | |
| TMIGD2 | XM_047438167; NR_172632; NM_001395549; NM_001308232; |
| | NR_172630; NM_001169126; NM_144615; NR_172631 |
| TNC | XM_005251975; XM_006717096; XM_011518628; XM_017014681; |
| | XM_047423311; XM_047423321; XM_047423323; XM_047423328; |
| | XM_011518626; XM_047423312; XM_047423313; XM_047423317; |
| | XM_047423318; XM_005251973; XM_006717098; XM_047423322; |
| | XM_047423324; XM_047423327; XM_005251972; XM_006717097; |
| | XM_011518629; XM_047423309; XM_047423314; XM_047423325; |
| | XM_017014680; XM_047423315; XM_047423329; XM_011518625; |
| | XM_017014679; XM_047423310; XM_047423320; XM_047423330; |
| | XM_047423331; XM_005251974; XM_006717101; XM_047423316; |
| | XM_047423319; XM_047423326; XM_047423332; XM_017014678; |
| | XM_024447530; NM_002160 |
| TNF | NM_000594 |
| TNFAIP3 | XM_024446533; XM_047419285; XM_011536095; XM_024446532; |
| | XM_047419282; XM_047419283; NM_006290; XM_011536096; |
| | NM_001270507; XM_005267119; XM_047419284; NM_001270508 |
| TNFAIP6 | XM_047445635; NM_007115 |
| TNFAIP8 | XM_047417081; NM_001077654; NM_001286815; NM_014350; |
| | NM_001286814; NM_001286817; NM_001286813; XM_017009327; |
| | XM_047417082; XM_017009328 |
| TNFAIP8F2 | NM_024575 |
| TNFRSF10A | NM_003844 |
| TNFRSF10B | NM_003842; NR_027140; NM_147187 |
| TNFRSF10C | NM_003841 |
| TNFRSF10D | NM_003840 |
| TNFRSF11A | NM_001270950; XM_011526244; XM_017026066; NM_001270949; |
| | NM_001278268; XM_011526245; XM_017026064; NM_001270951; |
| | NM_003839; XM_017026065 |
| TNFRSF13B | NM_012452 |
| TNFRSF13C | NM_052945 |
| TNFRSF14 | XM_047433423; XM_047433412; XM_047433413; XM_047433421; |
| | NM_003820; XM_047433418; XM_047433419; XM_047433422; |
| | NM_001297605; XM_006711019; XM_011542383; XM_047433416; |
| | XM_047433414 |
| TNFRSF17 | NM_001192 |
| TNFRSF18 | NM_148901; NM_004195; XM_017002722; NM_148902 |
| TNFRSF1A | NM_001346091; NM_001065; NM_001346092; NR_144351 |
| TNFRSF4 | XM_011542074; NM_003327; XR_007063145; XM_011542077; |
| | XM_011542075; XM_011542076 |
| TNFRSF8 | NM_001243; NM_152942; XM_011542441; XM_047434799; |
| | XM_011542443; XM_047434793; NM_001281430 |
| TNFRSF9 | XM_047419672; NM_001561; XM_006710618 |
| TNFSF10 | NR_033994; NM_001190943; NM_003810; NM_001190942 |
| TNFSF13 | NM_001198622; NM_172088; NR_073490; NM_001198624; NM_003808; |
| | NM_172087; NM_001198623 |
| TNFSF13B | NM_006573; XM_047430055; NM_001145645 |
| TNFSF18 | NM_005092 |
| TNFSF4 | XM_047429908; NM_003326; XM_047429896; NM_001297562; |
| | XM_017002228; XM_047429902; XM_011509964 |
| TNFSF8 | NM_001252290; NM_001244 |
| TNFSF9 | NM_003811 |
| TNIP1 | XM_047416622; XM_047416623; XM_047416624; XM_047416627; |
| | NM_001252391; NM_001252393; XM_005268355; XM_006714752; |
| | XM_047416615; XM_047416620; XM_047416626; NM_001258455; |
| | NM_001258456; XM_047416617; XM_047416625; NM_001364487; |
| | XM_047416616; XM_047416618; NM_001252392; NM_001258454; |
| | NM_001364486; NM_001252385; XM_047416621; NM_001252386; |
| | XM_047416619; NM_001252390; NM_006058 |
| TNIP3 | NM_001244764; XM_017008625; NM_001128843; XM_047416181; |
| | XM_047416182; NM_024873; XM_011532256; XM_011532257 |
| TNS4 | XM_017025237; XM_047436949; XM_005257744; NM_032865; |
| | XM_017025236; XM_047436950 |
| TONSL | XM_011517050; XM_011517048; XM_011517049; NM_013432 |
| TOP1 | NM_003286 |
| TOP2A | XM_005257632; XM_011525165; NM_001067 |
| TOPBP1 | XM_047447356; NM_001363889; XM_047447355; XM_047447357; |
| | XM_017005636; XM_047447358; NM_007027; XM_011512357 |
| TOX3 | NM_001080430; XM_047433909; NM_001146188; XM_005255892; |
| | XM_011523002 |
| TP53 | NM_000546; NM_001126112; NM_001276695; NM_001126115; |
| | NM_001126116; NM_001126118; NM_001276697; NM_001276698; |
| | NM_001276760; NM_001276761; NM_001126114; NM_001276696; |
| | NM_001126113; NM_001126117; NM_001276699 |
| TP53BP1 | XM_047432994; XM_047432999; XM_047432997; NM_001141979; |
| | XR_007064488; XM_047432995; XM_047432998; XM_047432996; |
| | NM_001355001; NM_005657; NM_001141980 |
| TP63 | NM_001114978; NM_001329144; NM_001329146; NM_001329964; |
| | NM_001329145; NM_003722; NM_001114979; NM_001114982; |
| | NM_001329149; NM_001114980; NM_001114981; NM_001329150; |
| | NM_001329148 |
| TPBG | NM_001166392; NM_001376922; NM_006670 |
| TPSAB1 | NM_003294 |
| TRAC | NG_001332.3 |
| TRAF2 | XM_011518977; NM_021138; XM_011518976; XM_047423828; |
| | XM_047423829 |
| TRAF3 | XM_011537117; XM_017021618; NM_145726; XM_047431739; |
| | XM_047431741; NM_003300; NM_145725; XM_047431742; |
| | NM_001385143; NM_001199427; XM_011537116; XM_017021617; |
| | XM_017021619; XM_017021620; XM_011537118; XM_047431738; |
| | XM_047431740; NM_001385142 |
| TRAF3IP3 | NM_025228; NR_109871; XM_047430963; NM_001287754; |
| | NM_001320143; XM_005273279; XM_047430964; XM_011510018; |
| | XM_017002400; XM_011510019; NM_001320144; XM_047430976 |
| TRAF6 | NM_145803; NM_004620 |
| TRAT1 | NM_016388; NM_001317747 |
| TRBC2 | NG_001333.2 |
| TRDC | NG_001332.3 |
| TRDV1 | NG_001332.3 |
| TRDV2 | NG_001332.3 |
| TREM2 | NM_001271821; NM_018965 |
| TRGC1 | NG_001336.2 |
| TRGC2 | NG_001336.2 |
| TRIM37 | XM_005257385; XM_017024669; XM_047436106; XM_047436113; |
| | NM_001320988; NM_001320990; NM_001353082; XM_017024662; |
| | XM_047436109; XM_047436112; XM_047436120; XM_047436124; |
| | NM_001320987; NM_001353084; NM_001353086; NR_148346; |
| | XM_017024670; XM_047436110; XM_047436119; NM_001005207; |
| | NM_015294; XM_011524831; XM_047436123; NM_001353083; |
| | NM_001353085; XM_011524833; XM_047436114; XM_047436117; |
| | XM_047436121; XM_047436122; XM_047436125; XM_047436126; |
| | NM_001320989; XM_017024667; XM_047436107; XM_047436108; |
| | XM_047436115; XM_011524834; XM_011524836; XM_017024663; |
| | XM_011524832; XM_017024665; XM_017024673; XM_047436111; |
| | XM_047436116; XM_047436118; NR_148347 |
| TRIP10 | NM_001288962; XM_005259683; NM_004240; NR_110231; |
| | XM_006722940; NM_001288963 |
| TRPM1 | NM_001252024; NM_001252030; NM_001252020; NM_002420 |
| TSC1 | XM_017015096; NM_000368; XM_006717271; XM_017015098; |
| | XM_011518979; XM_047423836; NM_001162426; XM_017015097; |
| | XM_047423830; XM_047423835; XM_005272211; NM_001162427; |
| | NM_001362177; XM_047423834; XM_047423831; XM_047423832; |
| | XM_047423837; NM_001008567; XM_047423833 |
| TSC2 | XM_047434556; NM_021056; NM_001318831; XM_047434555; |
| | XM_011522637; NM_001077183; NM_001318832; NM_001363528; |
| | XM_011522639; XM_017023615; XM_047434557; NM_001318827; |
| | NM_001370405; XM_011522636; XM_011522640; NM_000548; |
| | NM_001370404; NM_021055; XM_011522638; NM_001114382; |
| | NM_001318829 |
| TUBB3 | NM_006086; NM_001197181 |
| TWIST1 | NR_149001; NM_000474 |
| TWIST2 | NM_057179; XR_007069137; NM_001271893 |
| TXNDC11 | NM_015914; XM_047434192; NM_001324024; NR_136671; NR_136673; |
| | NR_136674; XM_047434191; NM_001324022; NM_001324025; |
| | NM_001303447; NR_136672; XM_011522515 |
| TXNDC5 | NM_030810; NM_022085; NM_001145549 |
| TXNRD1 | NM_001261446; NM_182742; NM_182743; NM_003330; NM_182729; |
| | NM_001093771; NM_001261445 |
| TYK2 | XM_011528246; XM_011528247; XM_047439304; NM_001385201; |
| | NM_001385204; XM_011528245; NM_003331; NM_001385197; |
| | NM_001385206; XM_011528249; NM_001385207; XM_047439305; |
| | NM_001385198; NM_001385200; NM_001385202; XM_047439306; |
| | XM_047439307; NM_001385199; NM_001385203; NM_001385205 |
| TYMP | NM_001257988; NM_001113755; NM_001257989; NM_001953; |
| | NM_001113756 |
| TYMS | NM_001354867; NM_001354868; XM_024451242; NM_001071 |
| TYRO3 | NM_001330264; XM_017022543; NM_006293 |
| TYROBP | NM_001173515; NM_003332; NR_033390; NM_001173514; NM_198125 |
| U2AF1 | XM_017028468; XM_024452129; XM_024452130; NM_001025204; |
| | NM_001025203; XM_024452131; XM_011529743; NM_006758 |
| UBASH3A | XM_047440833; XM_047440834; XM_047440835; XM_047440838; |
| | XM_047440836; XM_047440840; NM_001001895; NM_001243467; |
| | NM_018961; XM_047440837; XM_047440839; XM_011529609; |
| | XM_047440831; XM_047440832; XR_007067789 |
| UBE2C | NM_001281742; NM_001281741; NM_181802; NM_181803; NR_104036; |
| | NR_104037; NM_007019; NM_181800; NM_181801; NM_181799 |
| UBE2V1 | NM_001257397; NM_001257395; NM_001282579; NM_022442; |
| | NR_047554; NM_001257394; NM_001282575; NM_001257399; |
| | NM_199144; NM_001032288; NM_001257396; NM_001282576; |
| | NM_021988; NM_001257398; NM_001282577; NM_001282580; |
| | NR_104218; NM_001257393; NM_001282578 |
| UBE2V2 | NM_003350; XM_017013808; XM_011517583 |
| UBR5 | XM_047421847; NM_001282873; XM_047421849; XM_005250962; |
| | XM_047421848; XM_024447178; XM_024447179; NM_015902 |
| UCP1 | XM_005263206; NM_021833; XM_011532228 |
| UGCG | NM_003358; XM_047423844; XM_017015107 |
| UGP2 | XM_024453120; NM_001377526; NM_001377527; NM_001001521; |
| | NM_001377525; NM_001377528; NM_001377524; NM_001377529; |
| | NM_006759 |
| UGT1A4 | NM_007120 |
| UGT2B10 | NM_001075; NM_001290091; NM_001144767; XM_017008585 |
| UGT2B4 | NM_001297616; NM_021139; NM_001297615 |
| UHRF1 | XM_047438707; XM_011527942; XM_047438709; NM_001048201; |
| | NM_001290051; NM_001290052; NM_001290050; XM_047438708; |
| | NM_013282 |
| ULK1 | XM_011538799; XM_011538798; NM_003565; XR_007063134 |
| UMPS | NR_033437; XR_001740253; NR_033434; NM_000373 |
| UNC5B | NM_170744; NM_001244889 |
| UNG | NM_003362; NM_080911 |
| UPB1 | NM_016327; XM_047441404; XR_001755249; XM_011530223; |
| | XM_047441405 |
| USP22 | XM_005256575; XM_047435703; NM_015276 |
| UVSSA | XM_017008490; XM_017008494; XM_047416025; NM_020894; |
| | XM_017008492; XM_017008493; XM_017008497; XR_001741302; |
| | XM_017008499; XR_001741304; XR_007057948; XM_017008498; |
| | XM_017008500; NM_001317934; XR_001741303; XM_017008495; |
| | XM_017008496; XM_024454162; XM_047416026; XR_007057949; |
| | XM_047416027; XM_047416028; NM_001317935 |
| VANGL1 | NM_138959; NM_001172412; NM_001172411 |
| VAV1 | XM_005259642; NM_005428; NM_001258206; NM_001258207 |
| VAV3 | XM_017000053; NM_001079874; NM_006113; XM_017000054; |
| | XM_047430439; XM_005270360; XM_017000055; XM_047430543; |
| | XM_005270361; XM_017000056; XM_024450319; XM_047430476; |
| | XM_047430555; XR_007063680 |
| VCAM1 | NM_080682; NM_001078; NM_001199834 |
| VCAN | NM_001126336; NM_001164098; NM_004385; NM_001164097 |
| VCL | NM_003373; NM_014000 |
| VEGFA | NM_001171625; NM_003376; NM_001033756; NM_001171624; |
| | NM_001171626; NM_001171630; NM_001025366; NM_001317010; |
| | NM_001025368; NM_001025370; NM_001171623; NM_001171622; |
| | NM_001171628; NM_001171629; NM_001204385; NM_001025367; |
| | NM_001025369; NM_001171627; NM_001204384; NM_001287044 |
| VEGFB | NM_003377; NM_001243733 |
| VEGFC | NM_005429 |
| VHL | NM_198156; NM_000551; NM_001354723 |
| VIM | NM_003380 |
| VMP1 | NM_001329396; NM_001329398; NM_001329394; NM_001329395; |
| | NM_001329397; NM_001329401; NM_030938; NM_001329399; |
| | NM_001329400; NM_001329402 |
| VNN2 | XM_006715593; NR_110143; NR_110146; XM_011536231; XR_007059352; |
| | NM_001242350; XM_047419477; XM_047419480; NM_004665; |
| | NM_078488; NR_034173; NR_110144; NR_110145; XM_047419479; |
| | XM_047419481; NR_034174; XM_047419478 |
| VNN3 | NM_001291703; NM_001368152; NM_001368154; NR_173393; |
| | NR_173395; NM_018399; NR_173392; NM_001368156; NM_001291702; |
| | NR_173396; NM_001368151; NM_001368155; NM_001368149; |
| | NM_001368150; NR_173391; NM_078625; NR_173394 |
| VPREB3 | NM_013378 |
| VSIG4 | NM_007268; NM_001184830; NM_001184831; NM_001100431; |
| | NM_001257403 |
| VTN | NM_000638 |
| VWF | NM_000552; XM_047429501 |
| WAS | XM_011543977; XM_047442434; XM_047442432; XM_017029786; |
| | XM_047442433; NM_000377 |
| WHSC1 | XM_047416137; XM_047416144; NM_001042424; NM_133335; |
| | NM_014919; XM_005248002; XM_005248005; XM_047416141; |
| | NM_133330; XM_047416139; XM_011513560; XM_047416138; |
| | NM_133333; XM_005248001; XM_047416142; NM_133332; NM_133336; |
| | XM_047416143; NM_007331; NM_133331; NM_133334 |
| WIFI | NM_007191 |
| WIPI1 | NR_135471; NM_017983; XM_017024808; NM_001320772; NR_135470 |
| WNT1 | NM_005430 |
| WNT10A | XM_011511930; XM_011511929; NM_025216 |
| WNT10B | NM_003394 |
| WNT11 | XM_047427546; NM_004626; XM_005274231; XM_011545239; |
| | XM_047427547; XM_011545240; XM_011545238; XM_047427548 |
| WNT16 | NM_016087; NM_057168 |
| WNT2 | NM_003391; NR_024047 |
| WNT2B | NM_004185; NM_001291880; NM_024494 |
| WNT3 | NM_030753 |
| WNT3A | NM_033131 |
| WNT4 | XM_011541599; NM_030761; XM_011541597 |
| WNT5A | XM_011534086; XM_017007128; XM_047448855; XM_047448858; |
| | NM_001256105; NM_001377272; XM_047448856; XM_047448857; |
| | XM_047448859; XM_047448862; NM_003392; XM_011534089; |
| | XM_047448860; XM_047448852; XM_011534088; XM_047448861; |
| | XM_017007127; XM_047448853; XM_047448854; NM_001377271 |
| WNT5B | NM_030775; NM_032642 |
| WNT6 | NM_006522 |
| WNT7A | XM_011534091; NM_004625; XM_047448863 |
| WNT7B | XM_011530366; NM_058238 |
| WNT8A | XM_047417692; NM_058244; NM_001300939; XM_017009826; |
| | NM_031933; XM_047417693; NR_125351; NM_001300938 |
| WNT8B | NM_003393 |
| WNT9A | NM_003395; XM_011544271 |
| WNT9B | XM_011525178; NM_003396; NM_001320458 |
| WRN | XM_011544639; XR_949472; NM_000553; XR_949471; XR_949470; |
| | XM_011544640 |
| WT1 | NM_000378; NR_160306; NM_001367854; NM_001198551; |
| | NM_001198552; NM_024424; NM_024426; NM_024425 |
| WTIP | XM_011526453; XM_006723014; NM_001080436; XM_011526452 |
| WWC1 | XM_011534487; XM_011534489; XM_047417020; XM_047417021; |
| | NM_015238; XM_005265850; XM_011534485; XM_011534486; |
| | XM_047417017; XM_047417018; XM_005265853; XM_011534488; |
| | XM_011534490; XM_011534491; XM_017009276; XM_047417019; |
| | NM_001161662; NM_001161661 |
| WWTR1 | XM_047447930; NM_001168278; XM_047447933; XM_047447932; |
| | NM_001168280; NM_001348362; XM_017006122; XM_047447931; |
| | NM_015472 |
| XAB2 | NM_020196 |
| XAF1 | XM_011523948; XM_011523949; NM_001353136; NM_001353140; |
| | XR_007065309; NM_001353137; NM_001353138; NM_199139; |
| | XM_047436318; NM_017523; XR_007065310; XR_934053; |
| | XM_047436316; NM_001353134; NM_001353135; NM_001353139; |
| | NR_046398; XR_007065308; XM_047436317 |
| XBP1 | NM_001394000; NM_005080; NM_001079539; NM_001393999 |
| XCL1 | NM_002995 |
| XCL2 | NM_003175 |
| XCR1 | NM_001024644; NM_005283; NR_170111; NM_001381860 |
| XIAP | NR_037916; NM_001204401; NR_165803; NM_001167; NM_001378592; |
| | NM_001378591; NM_001378590 |
| XPA | NR_149093; XM_006717278; NM_001354975; NR_149092; NM_000380; |
| | NR_027302; NR_149094; NR_149091 |
| XPC | NM_001354729; NM_001145769; NM_001354730; NM_004628; |
| | XM_047448864; XM_047448865; NM_001354726; NM_001354727; |
| | NR_148950; NR_148951 |
| XRCC1 | NM_006297 |
| XRCC2 | NM_005431 |
| XRCC3 | XM_005268046; NM_001371231; XM_047431767; XM_047431768; |
| | NM_001100119; NM_001371229; XM_047431766; NM_001371232; |
| | NM_001100118; NM_005432 |
| XRCC4 | XM_047417695; XM_047417696; XM_047417698; XM_017009828; |
| | NM_003401; NM_022550; XM_011543626; XM_017009827; |
| | NM_001318012; NM_022406; XM_047417694; NM_001318013; |
| | XM_047417697 |
| XRCC5 | NM_021141 |
| XRCC6 | NM_001469; NM_001288976; NM_001288977; NM_001288978; |
| | XM_047441304 |
| YAP1 | XM_005271380; XM_005271381; NM_001130145; NM_001282099; |
| | NM_006106; NM_001282097; XM_005271378; XM_005271383; |
| | XM_011542555; NM_001282098; NM_001195045; NM_001282100; |
| | NM_001195044; NM_001282101 |
| YWHAZ | NM_145690; XM_005251061; NM_001135700; XM_005251063; |
| | NM_001135702; XM_017013810; NM_001135699; NM_003406; |
| | XM_017013811; NM_001135701 |
| ZAP70 | NM_001378594; NM_207519; XR_007081582; NM_001079; |
| | XM_047445775; XM_047445774; XM_047445776; XR_007081583 |
| ZBED2 | NM_024508 |
| ZBTB16 | XM_047427563; NM_001354751; NM_001354752; NM_006006; |
| | XM_005271658; NM_001018011; NM_001354750 |
| ZBTB7A | NM_015898; NM_001317990; XM_005259570; NM_020224; |
| | XM_005259571 |
| ZEB1 | XM_047425691; NM_001174096; NM_001323650; NM_001323655; |
| | NM_001323660; NM_001323677; XM_047425683; XM_047425688; |
| | NM_001174095; NM_001323638; NM_001323645; NM_001323647; |
| | NM_001323663; NM_001323666; NM_001323672; NM_030751; |
| | XM_047425684; XM_047425686; NM_001323662; XM_047425682; |
| | XM_047425685; XM_047425689; XM_047425693; XM_047425695; |
| | NM_001128128; NM_001174094; NM_001323641; NM_001323644; |
| | NM_001323659; NM_001323674; NM_001323675; NM_001323676; |
| | XM_047425687; NM_001323642; NM_001323648; NM_001323649; |
| | NM_001323652; NM_001323654; NM_001323671; XM_047425690; |
| | NM_001323643; NM_001323658; NM_001323665; NM_001323651; |
| | NM_001323656; NM_001323661; NM_001323664; NM_001323673; |
| | NM_001323678; XM_047425692; XM_047425694; XM_047425696; |
| | NM_001174093; NM_001323646; NM_001323653; NM_001323657 |
| ZEB2 | NM_001171653; NM_014795; NR_033258 |
| ZFYVE9 | XM_011542437; XM_047434682; NM_007324; XM_047434674; |
| | NM_007323; NM_004799 |
| ZMYND11 | NM_001202465; NM_001370103; NM_001370110; NM_001370115; |
| | NM_001370101; NM_001370104; NM_001370108; NM_001370122; |
| | NM_001202468; NM_001330057; NM_001370098; NM_001370099; |
| | NM_001370106; XM_017015594; NM_001370102; NM_001370119; |
| | NM_001370107; NM_001370109; NM_001370114; NM_001370117; |
| | NM_001370118; NM_006624; NM_001202466; NM_001202467; |
| | NM_001370100; NM_001370113; NM_001370121; NM_001370123; |
| | NM_212479; NM_001370097; NM_001370111; NM_001370120; |
| | NM_001370124; NM_001202464; NM_001370105; NM_001370112; |
| | NM_001370116; NR_163254 |
| ZNF101 | XM_047439722; XM_047439723; NM_033204; NM_001300949; |
| | XM_024451785 |
| ZNRF3 | NM_001206998; NM_032173 |
| ZSWIM7 | XM_047435366; XR_007065263; XM_047435364; XM_047435365; |
| | NM_001042698; XR_007065264; NM_001042697 |

TABLE 3
Examples of genes in the Biologically Meaningful Group
Excluding ‘Problematic’ Genes (BMGEP) Group.
BMGEP

| | |
|---|---|
| A1BG | NM_130786 |
| ABCA2 | XM_047422921; NM_001606; XM_006716996; NM_212533 |
| ABCA3 | NM_001089 |
| ABCB1 | NM_001348946; NM_001348944; NM_000927; NM_001348945 |
| ABCB4 | XM_011516308; XR_007060049; XM_011516309; NM_018849; |
| | XR_007060045; XR_007060046; XR_007060053; XM_047420477; |
| | XR_007060051; XR_007060055; XR_007060047; XM_047420476; |
| | XR_007060048; XM_047420475; NM_000443; NM_018850; |
| | XR_001744810; XR_007060050; XR_007060054; XR_007060052 |
| ABCB5 | NM_001163941; NM_001163993; NM_178559; NM_001163942 |
| ABCC1 | XM_047434140; XM_047434149; XM_047434132; XM_047434131; |
| | XM_047434143; XM_047434145; XM_047434154; NM_019898; |
| | NM_019902; XM_047434133; XM_047434141; XM_047434144; |
| | NM_019899; NM_019901; XM_011522497; XM_047434142; NM_004996; |
| | XM_047434148; NM_019862; NM_019900; XM_047434134; |
| | XM_047434136; XM_047434137; XM_047434147; XM_047434152; |
| | XM_017023237; XM_047434135; XM_047434138; XM_047434146; |
| | XM_047434151; XM_047434153 |
| ABCC10 | XM_011514974; XM_047419500; XM_047419494; XM_047419496; |
| | NM_001350518; NR_146762; XM_011514986; XM_047419501; |
| | XM_047419502; NM_033450; XM_011514985; XM_047419495; |
| | XR_007059383; NM_001198934; XM_047419497; XM_047419499; |
| | XR_007059384; XM_047419498 |
| ABCC11 | XM_017023802; NM_001370496; NM_032583; XM_017023798; |
| | XM_017023797; XM_017023800; XM_017023799; XR_007064925; |
| | XM_017023801; NM_001370497; XM_011523398; NM_145186; |
| | XM_024450475; XM_047434818; NM_033151 |
| ABCC2 | XM_047424598; XM_011539291; XM_017015675; XM_006717631; |
| | XR_945604; NM_000392; XM_006717630 |
| ABCC3 | NM_001144070; NM_003786; NM_020037; NM_020038 |
| ABCC4 | NM_001301829; XM_047430035; NM_005845; NM_001301830; |
| | NM_001105515; XM_047430034; XM_017020320 |
| ABCC5 | XM_017005493; XM_011512314; XM_047447099; NM_001320032; |
| | NM_005688; XM_005247059; XM_011512315; XM_047447098; |
| | XM_047447100; NM_001023587; NR_135125 |
| ABCG2 | NM_001348989; NM_001348985; NM_001348988; XM_017008852; |
| | NM_001348987; NM_001257386; XM_011532420; NM_001348986; |
| | NM_004827 |
| ABL1 | NM_005157; NM_007313 |
| ACACA | XM_006721853; XM_047435889; NM_198834; XM_005257267; |
| | XM_047435878; XM_047435885; XM_047435890; XM_047435894; |
| | NM_198837; NM_000664; XM_047435881; NM_198839; XM_011524701; |
| | XM_047435879; XM_047435882; XM_047435886; XM_047435891; |
| | XM_047435892; XM_047435893; NM_198838; XM_011524703; |
| | XM_011524704; XM_047435888; NM_198836; NM_198835; |
| | XM_047435884; XM_047435880; XM_047435883 |
| ACACB | XM_011538265; XM_047428764; NM_001093; XM_011538264; |
| | XM_047428768; XR_007063072; XM_006719367; XM_011538263; |
| | XM_017019252; XM_047428762; XM_047428765; XM_047428766; |
| | XM_047428767; XM_047428763 |
| ACAP1 | NM_014716; XM_047437152; XM_047437151; XM_047437150 |
| ACP5 | XM_047438944; NM_001111035; NM_001322023; NM_001611; |
| | NM_001111034; NM_001111036; XM_047438945; XM_005259938; |
| | XM_011528069 |
| ACSM2A | XM_017022925; XM_017022923; NM_001010845; NM_001308169; |
| | NM_001308954; XM_047433592; XM_017022926; XM_017022924; |
| | XM_047433591; NM_001308172; XR_001751833 |
| ACSM5 | NM_001324371; NM_001324372; NM_001324373; NM_017888 |
| ACTA2 | NM_001141945; NM_001320855; NM_001613 |
| ACVR1B | XM_047429857; NM_004302; XM_047429856; XM_011538967; |
| | XM_017020201; NM_020327; NM_020328; XM_011538966; |
| | XM_047429858 |
| ACVR1C | NM_001111032; NM_001111033; NM_001111031; NM_145259 |
| ACVR2A | NM_001278580; NM_001616; XM_047446292; NM_001278579 |
| ADAM15 | NM_003815; NM_207191; NR_048577; NR_048578; NM_207197; |
| | NM_001261464; NM_207196; NM_207195; NR_048579; NM_001261465; |
| | NM_001261466; NM_207194 |
| ADAM17 | XM_047445610; XM_047445612; NM_001382778; NM_003183; |
| | NM_001382777; NM_021832; XM_047445611 |
| ADAM28 | XM_047421270; XM_005273382; XM_047421272; NM_021777; |
| | XM_006716273; XM_017012975; XM_047421273; XM_011544367; |
| | NM_001304351; XM_005273380; XM_011544368; XM_011544369; |
| | XM_011544371; NM_014265; XM_006716274; XM_017012974; |
| | NR_130709; XM_047421271; XM_047421274; NR_130710 |
| ADAMTS2 | XM_047417895; NM_021599; NM_014244; XM_047417896 |
| ADAMTS5 | XM_047440680; NM_007038 |
| ADAP2 | XM_024450834; NM_001346714; NR_144488; XM_024450832; |
| | XM_024450833; NM_001346712; XM_024450835; NM_001346716; |
| | NM_018404 |
| ADGRE1 | NM_001974; NM_001256254; XM_011527794; NM_001256252; |
| | NM_001256253; NM_001256255 |
| ADGRE2 | XM_047438731; XM_011527955; XM_047438726; NM_001271052; |
| | NM_152916; XM_011527953; XM_011527954; XM_047438720; |
| | XM_047438727; NM_152918; XM_017026727; XM_047438721; |
| | XM_047438733; XM_047438736; XM_011527952; XM_011527948; |
| | XM_011527951; XM_017026726; XM_047438722; XM_047438724; |
| | NM_152919; XM_011527949; XM_047438723; XM_047438725; |
| | XM_047438729; XM_047438730; XM_047438735; XM_047438732; |
| | NM_013447; NM_152917; NM_152920; XM_047438728; XM_047438734; |
| | NM_152921 |
| ADGRE3 | NM_032571; NM_152939; XR_001753772; XM_011528374; |
| | XM_047439546; NM_001289158; NM_001289159 |
| ADGRG3 | XM_047433782; XM_011522954; XM_047433781; XM_047433783; |
| | XM_005255842; XM_011522953; XM_047433780; XM_006721170; |
| | NM_001308360; NM_170776 |
| ADIPOQ | NM_004797; NM_001177800 |
| ADORA1 | NM_001048230; XM_047446499; NM_000674; NM_001365065; |
| | NM_001365066 |
| ADORA2B | XM_017024197; XM_011523661; XM_047435375; NM_000676; |
| | XM_047435374; XM_011523659; XM_047435373 |
| AES | NM_198970; XM_006722664; NM_001130; NM_198969 |
| AFF2 | NM_001170628; NM_001169125; NM_001169123; NM_002025; |
| | NM_001169122; NM_001169124 |
| AFM | NM_001133; XM_017007843; XM_017007844; XM_017007842 |
| AGR2 | XM_005249581; NM_006408 |
| AGXT | NM_000030 |
| AHSG | NM_001354571; NM_001354572; NM_001622; NM_001354573 |
| AIFM1 | NM_001130846; NM_145812; NM_145813; NM_001130847; NM_004208; |
| | NR_132647 |
| AJUBA | NM_032876; NM_198086; NM_001289097 |
| AKT1 | NM_001014432; XM_047431069; XM_047431075; NM_005163; |
| | XM_047431071; NM_001382430; XM_047431072; NM_001382433; |
| | NM_001014431; NM_001382431; XM_047431073; XM_047431074; |
| | XM_047431070; NM_001382432 |
| AKT1S1 | NM_001278159; NM_001278160; NM_001098633; NM_001098632; |
| | NM_032375 |
| AKT2 | XM_011526616; XM_047438397; NM_001626; XM_047438398; |
| | XM_047438403; XM_011526619; XM_047438399; XM_047438401; |
| | NM_001243027; XM_011526618; NM_001243028; NM_001330511; |
| | XM_011526614; XM_047438400; XM_047438402; XM_011526615 |
| AKT3 | XM_047420641; XM_011544014; XM_047415643; XM_047422391; |
| | XM_047424332; NM_001370074; NM_005465; XM_047417359; |
| | NM_001206729; XM_024446892; NM_181690; XM_024447938; |
| | XM_047419186 |
| ALB | NM_000477 |
| ALDH1A1 | NM_000689 |
| ALDOB | NM_000035 |
| ALK | NM_004304; NM_001353765; XR_001738688 |
| ALKBH3 | NM_139178 |
| AMER1 | NM_152424 |
| AMHR2 | XM_011538179; XM_011538184; XM_017019179; NM_020547; |
| | XM_011538178; XM_011538176; XM_047428700; XM_011538181; |
| | XM_011538185; NM_001164691; XM_011538174; XM_011538183; |
| | XM_011538186; NM_001164690; XM_011538173; XM_011538180; |
| | XM_024448938 |
| ANGPT1 | NM_001314051; NM_001199859; NM_001146; XM_047421699; |
| | NM_139290 |
| ANGPT2 | NM_001118888; NM_001386335; NM_001386337; NM_001118887; |
| | NM_001147; NM_001386336 |
| ANGPTL3 | NM_014495 |
| ANXA1 | XM_017014657; NM_000700 |
| AOAH | NM_001177507; XM_011515335; XM_011515341; XM_011515336; |
| | XM_011515340; XM_011515342; XM_017012105; XM_011515333; |
| | XM_011515334; XM_047420297; NM_001177506; NM_001637; |
| | XM_011515338; XM_011515339; XM_017012104; XM_017012102 |
| APAF1 | XM_047428759; NM_013229; XM_047428758; NM_181868; NM_181869; |
| | XM_047428760; XM_017019250; NM_001160; NM_181861 |
| APC | NM_001354895; NM_001354896; NM_001354906; NM_001354899; |
| | NM_001354901; NM_000038; NM_001354902; NM_001354903; |
| | NM_001127510; NM_001354898; NM_001354900; NM_001354905; |
| | NM_001127511; NM_001354897; NM_001354904 |
| APCS | NM_001639 |
| APEX1 | NM_080648; NM_001244249; NM_001641; NM_080649 |
| APEX2 | NM_001271748; NM_014481 |
| APH1A | NR_045035; NM_016022; NR_045033; NM_001077628; NM_001243771; |
| | NM_001243772; XM_047422066; NR_045034 |
| APLN | NM_017413 |
| APOA5 | NM_001371904; NM_001166598; NM_052968 |
| APOB | NM_000384 |
| APOBEC3D | NM_145699; NM_001270406 |
| APOBEC3G | NM_001349436; NM_001349437; NR_146179; NM_021822; NM_001349438 |
| APOC3 | NM_000040 |
| APOC4- | NR_037932.1 |
| APOC2 | |
| AQP1 | NM_001185061; NM_198098; NM_000385; NM_001329872; |
| | NM_001185062; NM_001185060 |
| AR | NM_001348064; NM_001011645; NM_001348063; NM_001348061; |
| | NM_000044 |
| ARAF | NM_001256196; NM_001256197; NM_001654 |
| ARAP1 | NM_001369489; NM_015242; NM_001135190; NR_161388; NM_001040118 |
| AREG | NM_001657 |
| ARHGAP15 | NM_018460; XM_011511482; XM_024453000; XM_017004500; |
| | XM_011511483; XM_047445110; XM_047445112; XR_007078554; |
| | XM_011511484; XM_047445109; XM_047445111; XM_047445114; |
| | XM_047445113 |
| ARHGAP24 | NM_001025616; XM_047416235; NM_001042669; NM_001346093; |
| | XM_011532300; XM_024454238; NM_001287805; NM_031305 |
| ARHGAP30 | NM_001025598; NM_001287602; XM_005245070; NM_001287600; |
| | NM_181720; XM_011509391; XM_047417140; XM_005245073 |
| ARHGAP9 | XM_011538656; XM_011538659; XM_047429340; XM_047429337; |
| | XM_047429339; NM_001367422; NM_001367424; XM_047429334; |
| | NM_001367423; NM_001367425; NM_001367426; NM_001319851; |
| | XM_047429329; XM_047429332; XM_047429333; NM_001319852; |
| | XM_005269083; NM_001080157; NM_001319850; XM_047429330; |
| | XM_047429336; XM_047429335; NM_001080156; XM_047429331; |
| | XM_047429338; NM_032496 |
| ARHGDIB | NM_001175; NR_135637; NM_001321423; NM_001321421; |
| | NM_001321420; NM_001321422 |
| ARID1A | NM_018450; NM_006015; NM_139135 |
| ARNTL | NM_001297719; NM_001351819; NR_147787; NR_147790; |
| | XM_024448522; NM_001351808; NM_001351810; NM_001351813; |
| | NM_001351824; NR_147785; XM_047426958; NM_001351804; |
| | NM_001351805; NM_001351807; NM_001351811; NM_001351814; |
| | NM_001351817; NM_001351818; XM_011520107; NM_001351821; |
| | XM_017017739; XM_047426952; XM_047426953; XM_047426954; |
| | NM_001030272; NM_001297722; NM_001297724; NM_001351823; |
| | NM_001030273; NM_001351806; NM_001351809; NM_001351812; |
| | NM_001351815; NM_001351816; NM_001351822; NR_147786; |
| | XM_011520105; XM_011520109; XM_017017738; XM_047426955; |
| | XM_047426956; XM_047426957; NM_001351820; NR_147788; |
| | XM_017017741; NM_001178; NR_147789; NR_147791 |
| ARRDC1 | XM_006717320; XM_047424069; NM_001317968; NM_152285; |
| | XM_006717322; XM_006717321; XM_005266119; XM_017015288 |
| ATF3 | XM_047421211; NM_001206488; NM_001674; NM_001206484; |
| | NM_004024; XM_005273146; NM_001040619; NM_001206486; |
| | NM_001030287; XM_011509579; NM_001206485 |
| ATG12 | NM_004707; NM_001277783; NR_033362; NR_073603; NR_033363; |
| | NR_073604 |
| ATG13 | NM_001346319; NM_001346328; NM_001346352; NM_001346312; |
| | NM_001346317; NM_001346323; NM_001346325; NM_001346329; |
| | NM_001346335; NM_001346336; NM_001346338; NM_001346357; |
| | NM_001346360; NM_001346359; NR_144424; NM_001346314; |
| | NM_001346318; NM_001346333; NM_001346340; NM_001346351; |
| | NM_001346353; NM_001205119; NM_001346315; NM_001346322; |
| | NM_001346326; NM_001346346; NR_144422; NM_001205122; |
| | NM_001346311; NM_001346320; NM_001346321; NM_001346330; |
| | NM_001346350; NM_001346358; NM_001142673; NM_001205121; |
| | NM_001346324; NM_001346334; NM_001346349; NM_001346356; |
| | NM_014741; NR_144423; NM_001205120; NM_001346313; |
| | NM_001346327; NM_001346342; NM_001346344; NM_001346354; |
| | NM_001346316; NM_001346332; NM_001346331; NM_001346337; |
| | NM_001346348; NM_001346355 |
| ATG14 | NM_014924; XM_011536563 |
| ATG4B | XM_047443739; XM_047443738; XM_047443742; XM_047443741; |
| | XM_005246992; NM_178326; XM_047443740; NM_013325 |
| ATG5 | NM_001286108; NM_004849; NM_001286107; XM_047419574; |
| | NR_104403; NM_001286106; XM_024446590; XM_047419573; |
| | NM_001286111; NR_104402 |
| ATG7 | XM_024453312; XM_047447296; XM_047447309; NM_001349238; |
| | XM_006712931; XM_047447294; XM_047447298; XM_047447302; |
| | NM_001349234; XM_017005543; XM_017005550; XM_047447293; |
| | XM_047447307; NM_001349233; XR_007095622; XR_007095623; |
| | XM_047447304; XM_047447306; NM_001349232; XM_047447299; |
| | XM_047447308; NM_001349237; NM_006395; XM_017005542; |
| | XM_011533277; XM_047447297; XM_047447301; XM_047447305; |
| | NM_001349236; XM_047447300; NM_001349235; XM_006712932; |
| | XM_017005548; XM_017005551; XM_047447295; XM_047447303; |
| | NM_001136031; NM_001144912 |
| ATG9A | NM_001077198; NR_104255; NM_024085 |
| ATM | XM_011542844; XM_047426976; XM_047426978; NM_001351834; |
| | XM_011542840; XM_011542842; XM_047426975; NM_138293; |
| | XM_005271562; XM_006718843; XM_047426979; NM_000051; |
| | NM_001351835; XM_006718845; XM_047426981; NM_001351836; |
| | XM_011542843; XM_017017790; XM_047426977; NM_138292 |
| ATP7A | NM_000052; NR_104109; NM_001282224 |
| ATP7B | XM_047430388; XM_005266424; XM_047430393; NM_000053; |
| | XM_006719837; XM_047430386; XM_047430385; XM_047430392; |
| | NM_001330578; XM_005266423; XM_011535117; XM_005266431; |
| | XM_047430389; XM_047430391; NM_001005918; NM_001330579; |
| | XM_005266430; XM_017020627; XM_047430387; XM_047430390; |
| | NM_001243182 |
| ATR | XM_047448362; XM_011512925; NM_001354579; XM_011512924; |
| | XM_047448361; XM_047448363; NM_001184; XM_047448364; |
| | XM_047448360 |
| ATRIP | NM_001271023; NM_032166; NM_130384; NM_001271022 |
| AURKA | XM_047440428; XM_047440427; NM_001323304; NM_001323303; |
| | NM_198435; NM_198437; NM_198433; NM_198434; NM_198436; |
| | XM_017028034; XM_017028035; NM_001323305; NM_003600 |
| AURKB | NM_001313950; NM_001313953; XM_017025311; XM_047437050; |
| | NM_001313952; NM_004217; NM_001313954; NR_132730; NR_132731; |
| | NM_001284526; XM_011524072; XM_047437051; NM_001256834; |
| | NM_001313951; NM_001313955 |
| AXIN1 | XM_011522682; XM_011522686; XM_017023747; XM_047434731; |
| | NM_003502; NM_181050; XM_017023745; NR_134879; XR_001751996; |
| | XM_011522683; XM_017023746; XM_047434732; XM_017023748 |
| AXIN2 | XM_011525319; XM_047436873; XM_011525320; NM_001363813; |
| | XM_017025192; XM_047436871; XM_047436872; XM_017025193; |
| | XM_011525321; XM_047436870; XM_047436874; NM_004655 |
| AXL | NM_001699; NM_021913; NM_001278599 |
| B2M | XM_005254549; NM_004048 |
| B3GAT1 | XM_017017551; XM_024448439; XM_047426772; XM_047426773; |
| | NM_001367973; NM_018644; XM_011542753; XM_047426771; |
| | NM_054025 |
| BAAT | NM_001127610; NM_001701; NM_001374715 |
| BACH1 | NM_206866; NM_001011545; NR_027655; NM_001186 |
| BACH2 | NM_001170794; NM_021813 |
| BANK1 | NM_001127507; NM_001083907; NM_017935 |
| BAP1 | XM_011534149; XM_011534150; XM_017007303; XM_011534151; |
| | XM_047449044; XM_011534152; NM_004656 |
| BAX | NM_001291429; NM_138763; NM_001291428; NM_001291430; |
| | NR_027882; XM_047439168; NM_004324; NM_001291431; NM_138761; |
| | NM_138764; NM_138762 |
| BBC3 | XM_006723141; XM_011526722; NM_001127241; NM_001127242; |
| | NM_001127240; NM_014417; XM_047438606 |
| BCHE | NR_137636; NM_000055; NR_137635 |
| BCL11A | XM_047444756; NM_001405721; NM_001405728; XM_047444761; |
| | NM_001365609; NM_001405718; NM_001405724; NM_022893; |
| | NM_138553; XM_017004335; XM_024452962; NM_001405709; |
| | NM_001405714; NM_001405715; NM_001405722; NM_001405726; |
| | NM_001405735; NM_001405736; NR_175964; NM_001405723; |
| | XM_024452963; XM_047444760; NM_001405708; NM_001405711; |
| | XM_017004333; XM_047444757; XM_047444758; XM_047444759; |
| | NM_001405712; NM_001405719; NM_001405732; NM_138559; |
| | XM_017004336; XM_011532910; NM_001405713; NM_001405731; |
| | NM_001405733; NM_018014; NM_001363864; NM_001405717; |
| | NM_001405720; NM_001405730; NM_001405734; NM_001405710; |
| | NM_001405716; NM_001405725; NM_001405727; NM_001405729 |
| BCL2 | XM_047437734; NM_000657; XM_011526135; XM_017025917; |
| | NM_000633; XM_047437733 |
| BCL2L1 | XM_047440353; NM_001317919; NM_001322240; NM_001322242; |
| | XM_011528964; XM_047440351; NM_001191; NM_001317920; |
| | NR_134257; XM_017027993; NM_001317921; NM_138578; |
| | XM_047440352; NM_001322239 |
| BCL2L11 | NM_001204107; NM_138621; NM_138622; NM_207003; XR_007068189; |
| | XM_005263553; XM_047442104; NM_138624; NM_138625; NM_138626; |
| | XM_005263559; XM_011510461; NM_138627; XR_007068187; |
| | XM_005263552; XM_005263557; XM_017003101; XM_047442101; |
| | NM_006538; NM_207002; XM_047442102; NM_001204108; |
| | NM_001204113; NM_138623; XM_011510464; XM_047442099; |
| | NM_001204109; NM_001204112; XM_005263555; NM_001204106; |
| | NM_001204110; NM_001204111; XM_005263556 |
| BCL2L2 | NM_004050; NM_001199839 |
| BCL6 | NM_001130845; XM_011513062; NM_001706; XM_047448655; |
| | NM_001134738; NM_138931; XM_005247694 |
| BCR | NM_004327; NM_021574 |
| BECN1 | XM_017025263; XM_005257760; NM_001314000; XM_017025264; |
| | NM_001313998; NM_001313999; NM_003766; XM_005257759 |
| BHMT | NM_001713 |
| BHMT2 | NM_017614; NM_001178005 |
| BID | NM_001244567; NM_001196; NM_001244569; NM_197967; |
| | NM_001244572; NM_197966; NM_001244570 |
| BIRC2 | NM_001256166; NM_001256163; NM_001166 |
| BIRC3 | NM_001165; NM_182962 |
| BIRC5 | NM_001168; NM_001012270; NM_001012271 |
| BLK | XM_047422081; NM_001330465; XM_011543829; XM_011543824; |
| | XM_011543827; XM_047422083; XM_047422084; XM_011543828; |
| | XM_047422082; NM_001715; XM_011543825 |
| BLM | NM_001287247; NM_001287248; XM_006720632; XM_047432934; |
| | XM_011521882; NM_000057; NM_001287246 |
| BLNK | NR_047682; NM_001114094; NM_001258441; NM_013314; NR_047681; |
| | NM_001258440; NM_001258442; NR_047680; NR_047683 |
| BMI1 | NM_005180.9 |
| BMP2 | NM_001200 |
| BMP3 | NM_001201; XM_006714291 |
| BMP4 | NM_001347915; NM_001347916; NM_130851; NM_001347917; |
| | NM_130850; NM_001202; NM_001347914; NM_001347912; |
| | NM_001347913 |
| BMP5 | XM_011514817; NM_001329756; XM_024446524; NM_001329754; |
| | NM_021073 |
| BMP6 | NM_001718 |
| BMP7 | NM_001719 |
| BMP8B | XM_024449299; XR_946748; XR_946749; XM_017002155; XR_946750; |
| | XM_011542025; XM_005271149; XM_017002156; NM_001720; |
| | XM_011542024; XM_011542022 |
| BMPR1B | XM_017008558; XM_047416095; NM_001203; NM_001256793; |
| | XM_011532201; XM_047416093; NM_001256794; NM_001256792; |
| | XM_017008559; XM_047416091; XM_017008560; XM_047416094 |
| BMPR2 | NM_001204; NM_033346; XM_011511687 |
| BRAF | XM_047420766; XM_047420768; NM_001374244; NM_001374258; |
| | NM_001378471; NM_001378473; NR_148928; XM_047420767; |
| | XM_047420769; XM_047420770; NM_001378467; NM_001378468; |
| | XM_017012559; NM_001378470; NM_001378472; NM_001378475; |
| | NM_001354609; NM_001378469; NM_001378474; NM_004333 |
| BRCA1 | NM_007299; NM_007303; NM_007294; NM_007306; NM_007298; |
| | NM_007295; NM_007301; NM_007300; NR_027676; NM_007305; |
| | NM_007296; NM_007297; NM_007302 |
| BRCA2 | NM_000059 |
| BRD4 | XM_047438540; XM_047438541; XM_047438542; XM_047438543; |
| | NM_001379292; XM_047438544; NM_001379291; NM_014299; |
| | NM_058243; NM_001330384; XR_007066712 |
| BRD7 | XM_017023180; NM_001173984; XM_011523047; NM_013263; |
| | XM_011523049; XM_011523046; XM_017023179; XM_047434006; |
| | XM_047434007; XM_047434008; XM_011523048; XM_011523050 |
| BRMS1 | XM_024448425; XM_024448426; NM_015399; NM_001024958; |
| | NM_001024957 |
| BTG2 | NM_006763 |
| BTG3 | XM_011529441; NM_006806; XM_047440672; XM_047440670; |
| | XM_047440671; NM_001130914; XM_047440673 |
| BTK | NM_001287344; NM_001287345; NM_000061 |
| BTLA | NM_001085357; NM_181780; XM_011512447; XM_017005748; |
| | XM_047447496 |
| BUB1 | NM_004336; NM_001278617; XM_047445616; NM_001278616 |
| C10orf54 | NM_022153 |
| C1QA | NM_001347465; NM_001347466; NM_015991 |
| C1QC | NM_001114101; NM_001347619; NM_001347620; NM_172369 |
| C3AR1 | NM_004054; NM_001326475; NM_001326477 |
| C5AR1 | XM_047439300; NM_001736 |
| C8A | NM_000562; XM_011542079; XM_017002234 |
| C8B | NM_000066; NM_001278544; XM_047429957; NM_001278543 |
| C9 | NM_001737 |
| CA9 | XM_047423849; NM_001216; XM_047423850 |
| CADM1 | XM_047426693; NM_001098517; XM_017017457; XM_047426690; |
| | XM_047426694; NM_001301043; NM_001301045; XM_047426692; |
| | XM_047426695; NM_001301044; XM_005271494; NM_014333; |
| | XM_047426691 |
| CADM3 | NM_001346510; XM_024448760; NM_021189; NM_001127173 |
| CALR | NM_004343 |
| CAMK4 | NM_001744; NM_001323376; XR_948303; NM_001323375; |
| | XM_047417783; NM_001323374; NM_001323377 |
| CANX | XM_011534665; XM_047417792; NM_001746; NM_001363998; |
| | NR_157048; NM_001024649; NM_001363997; NM_001364000; |
| | NM_001363996; NM_001363993; NM_001363994; NM_001364001; |
| | NM_001363995; NM_001363999 |
| CARD11 | NM_032415; NM_001324281 |
| CASP3 | XM_047416237; NM_032991; NM_001354783; XM_047416239; |
| | NM_001354779; NM_001354780; NM_004346; XM_047416236; |
| | NM_001354782; NM_001354784; XM_047416238; NM_001354777; |
| | NM_001354781 |
| CASP8 | NM_001080124; NM_001080125; NM_001400651; NM_001400653; |
| | NM_001400669; NM_033355; NR_174599; NM_033358; NM_001400642; |
| | NM_001400665; NM_001400751; NR_174581; NR_174588; NR_174593; |
| | NR_174602; NM_001400663; NM_001400666; NM_001400667; |
| | NM_001400676; NM_001400680; NR_174584; NR_174589; NR_174600; |
| | NM_033357; XM_011511969; NM_001372051; NM_001400645; |
| | NM_001400660; NM_001400662; NM_001400671; NR_174592; |
| | NR_174594; XM_005246893; XM_047445959; XM_047445961; |
| | NM_001400654; NM_001400658; NM_001400659; NM_001400661; |
| | NM_001400668; NM_001400670; NM_001400672; NR_174565; |
| | NR_174583; NR_174585; NR_174586; NR_174598; NM_001228; |
| | NM_001400648; NM_001400655; NM_001400664; NM_001400674; |
| | NM_001400679; NM_033356; NR_174564; NR_174590; NR_174601; |
| | NM_001400673; NM_001400675; NM_001400677; NM_001400678; |
| | NM_001400750; NR_111983; NR_174591; NR_174595; XM_047445960; |
| | NM_001400656; NM_001400657; NR_174582; NR_174596; XR_007082538; |
| | XR_007082539 |
| CASP9 | XM_005246014; XM_011542273; NM_001229; NM_001278054; |
| | NR_102732; NR_102733; XM_047432034; NM_032996; XR_007064158 |
| CAV1 | NM_001753; NM_001172895; NM_001172897; NM_001172896 |
| CBFB | NM_001755; NM_001368707; NM_001368709; NM_022845; |
| | NM_001368708; NM_001368710 |
| CBL | NM_005188 |
| CBLB | XM_017007396; XM_047449114; XM_047449117; NM_001321798; |
| | NM_001321806; NM_001321813; NR_135811; XM_017007397; |
| | NM_001321791; NM_001321795; NR_135806; XM_011513257; |
| | NM_001321789; NM_001321808; XM_017007395; XM_047449112; |
| | XM_047449113; NM_001321788; NM_001321799; NM_001321807; |
| | XR_007095761; XR_007095762; XM_011513259; NM_001321790; |
| | NM_001321797; NM_001321820; NM_170662; NR_135807; NR_135812; |
| | NM_004351; XM_047449115; NM_001321811; NR_135809; NR_135810; |
| | XM_017007400; XM_047449116; XM_047449118; NM_001321794; |
| | NM_001321796; NM_001321816; NR_135808; XM_017007398; |
| | NM_001321786; NM_001321793; NM_001321822 |
| CBX4 | NM_003655; XM_011525399 |
| CCDC69 | NM_015621 |
| CCL18 | NM_002988 |
| CCL2 | NM_002982 |
| CCL22 | XM_047434450; XM_047434449; NM_002990 |
| CCL28 | NM_001301874; NM_001301873; NM_019846; XR_007058611; |
| | XR_007058613; XR_925633; NM_148672; NM_001301875; XR_007058610; |
| | XR_007058612; XR_427660; XR_241706 |
| CCNB1 | NM_001354844; NM_031966; NM_001354845 |
| CCND1 | NM_053056 |
| CCND2 | NM_001759 |
| CCND3 | XM_047419491; NM_001287434; NM_001136017; NM_001760; |
| | NM_001136125; NM_001136126; XM_011514971; NM_001287427 |
| CCNE1 | XM_011527440; NM_001238; NM_001322259; NM_001322261; |
| | XM_047439606; NM_001322262; NM_057182 |
| CCNE2 | XM_047422411; XM_017013958; NM_057749; XM_011517366; |
| | XM_017013959; NM_004702; NM_057735 |
| CCNH | NR_157070; NM_001363539; NR_157071; NM_001239; NM_001364076; |
| | XM_047417863; NM_001199189; NR_157068; NR_157069; NM_001364075 |
| CCR1 | NM_001295 |
| CCR2 | NM_001123041; NM_001123396 |
| CCR3 | NM_001164680; NM_001837; NM_178328; NM_178329; XM_017005685; |
| | XM_006712960 |
| CCR4 | XM_017005687; NM_005508 |
| CCR5 | NM_001100168; NM_001394783; NM_000579 |
| CCR6 | NM_001394582; NM_031409; NM_004367 |
| CCR7 | NM_001301716; NM_001301717; NM_001838; NM_001301718; |
| | NM_001301714 |
| CD101 | XM_047434715; NM_004258; NM_001256109; NM_001256106; |
| | NM_001256111; XM_047434718 |
| CD14 | NM_001040021; NM_000591; NM_001174105; NM_001174104 |
| CD160 | NM_007053; XM_005272929; XM_011509104; NR_103845 |
| CD163 | XM_047429895; XM_024449278; NM_203416; NM_001370145; |
| | NM_001370146; NM_004244; NR_163255 |
| CD177 | XM_017027021; XM_017027022; NM_020406 |
| CD19 | NM_001178098; NM_001385732; NM_001770; XR_950871; NR_169755; |
| | XM_011545981 |
| CD1A | NM_001320652; NM_001763; XM_024450738 |
| CD1D | NM_001371762; XM_047433945; NM_001371761; NM_001319145; |
| | NM_001371763; NM_001766 |
| CD2 | NM_001328609; NM_001767 |
| CD200 | NM_001318830; NR_158642; NM_001004197; NM_001365853; |
| | NM_001365855; NM_001318826; NM_001365852; NM_001004196; |
| | NM_001318828; NM_001365851; NM_005944; NM_001365854 |
| CD207 | XM_011532876; XM_011532875; NM_015717 |
| CD209 | NR_026692; NM_001144895; NM_001144894; NM_001144893; |
| | NM_021155; NM_001144896; NM_001144897; NM_001144899 |
| CD22 | NM_001185100; NM_001185099; NM_024916; NM_001185101; |
| | NM_001771; NM_001278417 |
| CD226 | NM_006566; XM_047437274; NM_001303619; XM_047437275; |
| | XM_047437276; XM_006722374; XM_005266642; XM_047437277; |
| | NM_001303618 |
| CD244 | NM_001166663; XM_047422535; XM_011509622; NM_016382; |
| | NM_001166664; XM_011509623; XM_011509621 |
| CD247 | NM_001378516; NM_198053; XM_011510144; XM_011510145; |
| | NM_000734; NM_001378515 |
| CD248 | NM_020404 |
| CD274 | XM_047423262; NM_001314029; NM_001267706; NR_052005; NM_014143 |
| CD276 | XM_005254700; XM_047433148; XM_047433147; NM_025240; |
| | XM_017022638; NM_001329628; XM_011522095; NM_001024736; |
| | NM_001329629 |
| CD300A | XM_005256991; NM_001330457; NM_001330456; XM_005256990; |
| | NM_007261; NM_001256841 |
| CD300E | NM_181449 |
| CD300LB | NM_174892; XM_005257027 |
| CD302 | NM_014880; NM_001198764; NM_001198763 |
| CD36 | XM_024447002; NM_000072; NM_001289909; NM_001371081; |
| | NR_110501; NM_001001548; NM_001127443; XM_005250715; |
| | NM_001371074; XM_047421045; NM_001001547; XM_047421042; |
| | XM_047421043; XM_047421044; XM_047421048; XM_047421049; |
| | NM_001371075; XM_047421046; XM_047421047; NM_001127444; |
| | NM_001371077; NM_001371078; NM_001371079; NM_001371080; |
| | XM_024447003; XM_047421041; NM_001289908; NM_001289911 |
| CD37 | XM_005259435; XM_011527542; NM_001774; XM_011527543; |
| | NM_001040031 |
| CD3E | NM_000733 |
| CD3G | XM_005271724; XM_006718941; NM_000073 |
| CD4 | NM_001195014; NM_001382707; NM_001382714; NM_001195016; |
| | NM_000616; NR_036545; NM_001195015; NM_001382705; |
| | NM_001195017; NM_001382706 |
| CD40 | NM_001302753; NM_001322422; NM_152854; NM_001322421; |
| | NM_001362758; NM_001250; XM_047440601; NR_136327; |
| | XM_011529109; XM_005260619; XM_017028135; XM_017028136; |
| | NR_126502 |
| CD40LG | NM_000074 |
| CD46 | XM_047420901; XM_011509563; NM_172351; NM_172359; NM_172352; |
| | NM_172361; NM_002389; NM_172355; NM_172358; XM_047420909; |
| | NM_153826; NM_172353; XM_047420888; XM_047420894; NM_172356; |
| | NM_172354; NM_172360; NM_172350; NM_172357 |
| CD48 | XM_017002867; XM_047435011; NM_001778; XM_005245625; |
| | NM_001256030 |
| CD5 | NM_014207; NM_001346456 |
| CD53 | NM_000560; NM_001040033; XM_047435014; XM_047435015; |
| | NM_001320638; XM_047435013 |
| CD55 | NM_001300904; NM_001114543; NM_001114544; NM_001114752; |
| | NM_001300902; NM_001300903; XR_007095644; NM_000574; NR_125349 |
| CD58 | NM_001779; NM_001144822; NR_026665 |
| CD59 | NM_000611; NM_203330; NM_001127223; NM_203331; NM_001127227; |
| | NM_001127225; NM_001127226; NM_203329 |
| CD6 | XM_047427875; XM_047427876; XM_047427879; XM_011545360; |
| | XM_047427878; XM_047427881; NM_001254750; NM_001254751; |
| | NM_006725; NR_045638; XM_006718738; XM_006718739; |
| | XM_047427877; XM_006718740; XM_011545362; XM_047427874; |
| | XM_047427880 |
| CD63 | NM_001267698; NM_001257389; NM_001257400; XM_024449283; |
| | NM_001780; NM_001257401; NM_001257391; NM_001257390; |
| | NM_001257392; NM_001040034 |
| CD68 | NM_001251; NM_001040059 |
| CD69 | NR_026672; NR_026671; NM_001781 |
| CD74 | NM_001364083; NM_001364084; NR_157074; NM_001025159; |
| | NM_001025158; NM_004355 |
| CD82 | XM_011520067; XM_047426901; XM_047426904; XM_047426900; |
| | NM_002231; XM_047426903; NM_001024844 |
| CD83 | NM_001040280; NM_001251901; NM_004233 |
| CD84 | NM_003874; XM_011510095; NM_001184882; NM_001330742; |
| | NM_001184881; NM_001184879; XR_921991 |
| CD86 | NM_001206924; NM_006889; NM_176892; NM_001206925; NM_175862 |
| CD8A | NM_001145873; NM_001382698; NM_001768; NR_168478; NR_168479; |
| | NM_171827; NR_168480; NR_168481; NR_027353 |
| CD9 | NM_001769; NM_001330312; XM_005253814 |
| CD96 | XR_007093316; NR_134917; XR_007093335; XR_241462; XM_006713470; |
| | NM_005816; XR_924090; XM_006713469; NM_198196; XR_007093273; |
| | XR_007093326; XR_007093307; XM_047447184; NM_001318889; |
| | XR_001739977; XR_007093366 |
| CDC25A | XM_047449366; NM_001789; XM_047449364; XM_047449367; |
| | XM_047449363; XM_047449365; NM_201567 |
| CDC6 | XM_047437207; XM_011525542; XM_011525541; NM_001254 |
| CDCA7 | NM_031942; NM_145810; XM_047445957 |
| CDH1 | NM_001317186; NM_004360; NM_001317185; NM_001317184 |
| CDH10 | XR_001753699; XM_047438909; XM_017026863; NM_001136197; |
| | XM_005259573; XM_047438908; NM_016263; NM_001136198 |
| CDH12 | NM_001308392; NM_001797; XM_047433486; NM_001330576; |
| | NM_033664 |
| CDH13 | NM_001317227; NM_001364105; NM_001364106; NM_001364104; |
| | NM_004061; NM_001317228; NM_001364109; NM_001364107; |
| | NM_001364108; XM_047416602 |
| CDH15 | XM_017022849; NM_001220488; XM_017022848; NM_001220492; |
| | NM_001220491; NM_001257; NM_001220489; XM_011522804; |
| | NM_001220490 |
| CDH16 | XM_047433490; NM_001204746; XM_011522807; NM_004062; |
| | XM_005255770; NM_001204744; NM_001204745 |
| CDH18 | XM_011513930; XM_017008924; XM_017008926; XM_017008927; |
| | NM_001349556; NM_001349559; XM_017008930; NM_001349560; |
| | XM_017008929; NM_001349563; XM_017008928; NM_001167667; |
| | NM_004934; XM_006714435; XM_005248228; NM_001291956; |
| | NM_001349562; NM_001291957; NM_001349558; NM_001349561 |
| CDH19 | XM_011525932; XM_011525931; NM_021153; NR_073130; |
| | XM_047437485; NM_001271028; XM_047437484 |
| CDH2 | XM_011525788; NM_001308176; XM_017025514; NM_001792 |
| CDH20 | XR_001753187; NM_031891; XR_001753186; XM_024451165 |
| CDH22 | XM_024451966; XM_047440374; XM_047440373; XM_011528994; |
| | XM_024451967; NM_021248 |
| CDH23 | NM_001171932; NM_001171936; NM_001171930; NM_001171933; |
| | NM_001171935; NM_001171934; NM_022124; NM_052836; |
| | NM_001171931 |
| CDH3 | NM_001793; XM_011522800; XM_047433450; NM_001317195; |
| | NM_001317196 |
| CDH4 | NM_001252339; XM_047439813; NM_001794; XM_047439812; |
| | NM_001252338 |
| CDH5 | XM_047433469; XM_047433470; NM_001114117; NM_001795; |
| | XM_047433471; XM_011522801 |
| CDH6 | NM_004932; NM_001362435; XM_011513921; XM_047416591 |
| CDH7 | NM_001317214; NM_004361; NM_001362438; NM_033646 |
| CDH8 | XM_005255760; XM_047433482; XM_047433484; XM_047433483; |
| | NM_001796 |
| CDK1 | NM_001320918; NM_033379; NM_001170406; NM_001786; |
| | NM_001130829; XM_005270303; NM_001170407 |
| CDK4 | NM_000075; NM_052984 |
| CDK6 | XM_047419716; NM_001145306; NM_001259 |
| CDK7 | NM_001324074; NM_001324072; NM_001324078; XM_011543094; |
| | NM_001324069; NM_001324070; NM_001324075; NM_001324077; |
| | NM_001324071; XM_047416609; NM_001799; NR_136690 |
| CDK8 | NM_001346501; XM_011534865; NM_001260; XM_047430033; |
| | NM_001318368 |
| CDKN1A | NM_001220778; NM_001374510; NM_078467; NR_164655; |
| | NM_001291549; NM_001374511; NM_001374509; NR_164656; |
| | NM_000389; NM_001220777; NM_001374512; NM_001374513 |
| CDKN1B | NM_004064 |
| CDKN2A | XM_011517676; XM_011517675; NM_001363763; NM_001195132; |
| | XM_047422597; NM_058195; XM_047422596; XM_047422598; |
| | NM_000077; NM_058196; NM_058197 |
| CDKN2B | NM_078487; NM_004936 |
| CDKN2C | NM_001262; NM_078626 |
| CDKN3 | NM_001330173; NM_005192; NM_001130851 |
| CDO1 | NM_001323565; NR_136619; NM_001323567; NM_001801; NR_136618; |
| | XM_047416629; NR_136620; XM_047416628; NM_001323566; NR_136621 |
| CDT1 | NM_030928 |
| CDX2 | XM_011534876; NM_001354700; XM_011534879; XM_011534875; |
| | XM_011534878; NM_001265 |
| CEACAM1 | NM_001024912; NM_001184813; NM_001205344; XM_011527206; |
| | NM_001712; NM_001184815; NM_001184816 |
| CEACAM5 | XM_011526322; XM_017026146; NM_001291484; NM_004363; |
| | XM_017026145; NM_001308398 |
| CECR1 | XM_047441407; NM_001282228; NM_017424; XM_047441406; |
| | NM_001282225; NM_001282227; NM_177405; XM_011546133; |
| | NM_001282226; NM_001282229 |
| CELF2 | XM_047424490; XM_047424491; XM_047424492; XM_047424494; |
| | XM_047424505; NM_001326330; NM_001326334; NM_001326336; |
| | NM_001326338; NM_001326342; NM_001326346; XM_047424482; |
| | XM_047424486; XM_047424508; NM_001326317; NM_001326318; |
| | NM_001326323; NM_001326327; NM_001326349; NM_001394513; |
| | XM_047424489; NM_001025077; NM_001326320; NM_001326321; |
| | NM_001326326; NM_006561; XM_047424488; XM_047424496; |
| | XM_047424503; XM_047424504; XM_047424510; NM_001025076; |
| | NM_001326319; NM_001326328; NM_001326339; NM_001326343; |
| | NM_001326344; XM_047424484; XM_047424487; XM_047424500; |
| | XM_047424501; XM_047424502; NM_001083591; NM_001326324; |
| | NM_001326325; NM_001326335; NM_001326341; NM_001394518; |
| | XM_024447776; XM_047424495; XM_047424499; XM_047424506; |
| | XM_047424507; NM_001326329; NM_001326347; NM_001326348; |
| | NM_001394502; NM_001394517; XM_047424483; XM_047424485; |
| | XM_047424498; NM_001326331; NM_001326333; NM_001394519; |
| | XM_047424493; XM_047424509; NM_001326332; NM_001326337; |
| | NM_001326340; NM_001326345 |
| CEP55 | XM_017016373; XM_011539920; NM_001127182; NM_018131; |
| | XM_017016372; XM_011539919; XM_047425416; XM_011539918 |
| CETN2 | NM_004344 |
| CETN3 | NM_004365; NM_001297765; NM_001297768 |
| CFC1 | NM_001270421; NM_001270420; XM_011511486; NM_032545 |
| CFH | NM_001014975; XM_017001108; XR_007059267; NM_000186; |
| | XM_047418835 |
| CFHR2 | XM_011509460; XM_011509458; XM_011509459; NM_005666; |
| | XM_017001109; XM_005245113; NM_001312672 |
| CFHR5 | NM_030787; XM_011510020 |
| CFI | NM_001375281; NM_000204; NM_001375282; NR_164673; |
| | XM_006714210; NM_001375280; NM_001318057; NM_001375279; |
| | XM_011531920; NM_001375284; NR_164671; XM_047415654; |
| | NM_001331035; NM_001375278; NM_001375283; NR_164672; |
| | XM_047415653 |
| CFL1 | NM_005507 |
| CFLAR | NM_003879; NR_147251; XR_007083740; XR_007083755; XM_047446185; |
| | NM_001351593; NR_147244; NR_147253; XR_007083702; NM_001202517; |
| | NM_001351590; NM_001351592; NR_147243; NR_147248; NR_147252; |
| | NR_147255; NM_001202515; NM_001202516; NR_147245; NR_147246; |
| | NR_147250; XM_047446197; NM_001127184; NM_001308042; |
| | NR_147241; XR_001739013; XR_007083715; NM_001308043; |
| | NM_001351594; NR_147242; NR_147249; XR_007083735; XR_007083742; |
| | NM_001202518; NM_001351591; XR_007083723; XM_047446191; |
| | NM_001127183; NM_001202519; NR_147247; XR_007083684; |
| | XR_007083713; XR_007083745 |
| CHD1L | NM_001256336; NM_001348459; NM_001348463; NM_001348465; |
| | NR_145681; NR_145684; NR_145685; NR_145695; XM_047435001; |
| | NM_001348453; NM_001348457; NM_001348462; NM_004284; |
| | NM_001348451; NR_046070; NR_145687; NM_001256338; |
| | NM_001348464; NR_145682; NR_145688; NR_145694; NR_145683; |
| | NR_145686; NR_145689; XM_024451051; NM_001348455; |
| | NM_001348458; NM_001348460; NM_024568; NR_145692; NR_145693; |
| | NM_001348454; NM_001348456; NM_001348466; NR_145690; |
| | NR_145691; NM_001256337; NM_001348452; NM_001348461 |
| CHEK1 | NM_001114121; NM_001114122; XR_007062447; XM_047426313; |
| | NM_001244846; NR_045205; XM_047426311; NM_001330427; |
| | XM_024448337; XM_047426312; NM_001274; XM_011542560; |
| | NM_001330428; NR_045204 |
| CHEK2 | XM_006724114; XM_011529845; XM_024452148; XM_047441105; |
| | XM_047441106; NM_001349956; XM_006724116; XR_007067954; |
| | XM_017028560; XM_047441104; NM_001257387; NM_007194; |
| | XM_011529842; XM_047441108; NM_145862; XM_011529839; |
| | XM_011529844; XM_024452149; XM_047441107; XR_937806; |
| | XR_937807; XM_011529840; NM_001005735; XR_007067955 |
| CHST15 | XM_047425327; XM_047425325; XM_047425332; XM_005269892; |
| | XM_047425328; XM_047425329; XM_047425334; NM_014863; |
| | XM_006717891; XM_017016321; NM_001270764; XM_017016319; |
| | XM_047425330; XM_047425331; XM_017016320; XM_047425324; |
| | NM_001270765; XM_011539857; XM_005269893; XM_047425326; |
| | NM_015892; XM_047425333 |
| CHUK | XM_047424543; XM_047424542; XM_047424540; NM_001278; |
| | NM_001320928; XM_017015612; XM_047424541 |
| CIDEA | NM_001279; NR_134607; NM_001318383 |
| CIITA | XM_047434115; NM_001379332; XR_007064880; XM_006720880; |
| | XM_011522491; XM_047434119; NM_001379334; XM_047434118; |
| | XM_047434120; XM_047434123; NM_001379333; XM_011522486; |
| | NM_000246; NM_001286402; XM_047434122; XM_047434126; |
| | XR_001751904; XR_007064879; XM_047434117; XM_047434114; |
| | XM_047434125; NM_001286403; NM_001379331; XM_011522485; |
| | XM_047434127; XM_047434128; NR_104444; XM_011522484; |
| | XM_011522490; XM_047434116; XM_047434124; NM_001379330 |
| CITED2 | NM_001168389; NM_006079; NM_001168388 |
| CLCA2 | NM_006536; XM_011542448 |
| CLDN1 | NM_021101 |
| CLDN12 | NM_001185072; NM_012129; NM_001185073 |
| CLDN15 | NM_014343; NM_138429; NM_001185080 |
| CLDN16 | NM_006580; XM_047447333; NM_001378492; NM_001378493 |
| CLDN17 | NM_012131 |
| CLDN18 | NM_001002026; NM_016369 |
| CLDN19 | NM_001123395; NM_001185117; NM_148960 |
| CLDN2 | NM_001171095; NM_020384; NM_001171092 |
| CLDN20 | NM_001001346 |
| CLDN22 | NM_001111319 |
| CLDN23 | NM_194284 |
| CLDN3 | NM_001306 |
| CLDN4 | NM_001305 |
| CLDN7 | NM_001307; NM_001185022; NM_001185023 |
| CLDN8 | NM_199328; NM_012132 |
| CLEC10A | NM_001330070; NM_182906; NM_006344; XM_011523613; |
| | XM_011523615 |
| CLEC14A | NM_175060 |
| CLEC17A | XM_017026788; XM_017026785; NM_207390; NR_109785; |
| | XM_017026787; NR_109784; XM_017026793; XM_047438796; |
| | XM_017026786; XM_017026791; XM_017026792; XM_017026794; |
| | NM_001204118 |
| CLEC5A | XR_007059995; XM_011515995; NM_013252; NM_001301167 |
| CLEC9A | NM_207345 |
| CLK2 | NM_001294339; XM_047444414; NM_001294338; XM_047444422; |
| | XM_047444417; NM_003993; NM_001363704; XM_011509143; |
| | NM_001291; XM_047444409 |
| CMKLR1 | NM_001142343; XM_047428313; NM_001142345; NM_001142344; |
| | NM_004072 |
| CNR1 | NM_001160259; NM_001370546; XM_011535425; XM_047418173; |
| | NM_001365870; NM_001370547; NM_001365872; NM_016083; |
| | XM_047418172; NM_001160226; NM_001160258; NM_001370545; |
| | NM_001365874; NM_033181; NM_001160260; XM_047418171; |
| | NM_001365869 |
| CNTN6 | NM_001289081; NM_001349352; NM_001349356; XM_017006174; |
| | NM_001349361; XM_011533591; XM_047447972; NM_001349358; |
| | NM_014461; NM_001289080; NM_001349353; NM_001349359; |
| | XM_011533590; XM_047447974; NM_001349350; NM_001349357; |
| | NM_001349354; NM_001349351; NM_001349355; NM_001349360; |
| | XM_017006172; XM_017006177; NM_001349362 |
| COL11A1 | XM_017000337; XM_017000335; XM_017000336; NR_134980; |
| | NM_080629; XR_007085257; XM_017000334; NM_001190709; |
| | NM_001854; NM_080630 |
| COL16A1 | XM_005270481; XM_017000338; XM_047446431; XM_011540729; |
| | XM_011540728; XM_011540722; XM_011540723; XM_011540727; |
| | XM_017000339; XM_011540730; XM_047446432; XM_047446439; |
| | XM_047446440; XR_001736983; XM_047446435; XM_011540724; |
| | XM_017000340; XM_017000341; NM_001856 |
| COL1A2 | NM_000089 |
| COL3A1 | NM_000090; NM_001376916 |
| COL4A1 | NM_001845; NM_001303110 |
| COL5A1 | NM_000093; XM_017014266; NM_001278074 |
| COL6A1 | NM_001848 |
| COL6A2 | NM_001849; NM_058175; NM_058174 |
| COL6A3 | NM_057164; NM_057167; NM_057166; NM_004369; NM_057165 |
| CORO1A | NM_007074; NM_001193333 |
| CPA3 | NM_001870 |
| CPNE5 | NM_001314019; NM_020939; XM_047419193; NM_001376893; |
| | XM_005249247; XM_011514769; NR_164866; XR_007059285; |
| | XM_047419191; NM_001376888; NM_001376890; NM_001376894; |
| | NM_001314017; NM_001376892; XM_011514773; XM_047419192; |
| | NM_001314020; XM_017011139; NM_001376891; XM_011514770; |
| | XM_011514768; XM_011514771; XM_011514772; XM_047419190; |
| | NM_001314018; NM_001376889; NM_001376895 |
| CR1 | NM_001381851; NM_000651; NM_000573 |
| CR2 | NM_001877; NM_001006658 |
| CRB1 | XM_017000852; XM_047416574; XM_047416575; NR_047563; |
| | NM_012076; XM_047416572; NR_047564; XM_011509367; |
| | XM_011509369; XM_047416573; NM_001257966; NM_201253; |
| | NM_001257965; XM_011509365; NM_001193640 |
| CRB2 | XM_047423244; XM_011518556; NR_104603; XM_011518558; |
| | XM_005251934; XM_011518557; NM_173689; XM_047423245 |
| CRBN | XM_005265202; XM_011533791; NM_001173482; NM_016302; |
| | XM_047448254; XM_047448253 |
| CREBBP | XM_017022944; XM_005255125; XM_047433625; XM_005255124; |
| | XM_011522381; XM_047433624; XM_006720848; NM_001079846; |
| | XM_011522382; NM_004380 |
| CRTAM | XM_011542900; NM_019604; NM_001304782 |
| CSF1 | NM_000757; NM_172210; XM_017000369; XM_047446752; NM_172211; |
| | NM_172212 |
| CSF1R | NM_001375320; NM_005211; NR_164679; NM_001349736; |
| | NM_001288705; NM_001375321; NR_109969 |
| CSF2RA | XM_047441847; XM_047441851; XM_047442715; NM_001379164; |
| | NM_001379166; XM_047441849; XM_047442710; XM_047442713; |
| | NM_001161530; NM_001379165; NM_006140; NM_172247; NM_172248; |
| | XM_011545627; XM_047441850; NM_001161531; NM_001161532; |
| | NM_001379168; NM_172245; XM_047441846; NM_001161529; |
| | NM_001379158; NM_001379161; NM_001379162; NM_172246; |
| | XM_011545620; XM_011546167; XM_047442711; XM_047442712; |
| | XM_047442716; NM_001379155; NM_001379163; NM_172249; |
| | XM_047441845; XM_047441848; NM_001379160; NM_001379167; |
| | NR_027760; XM_011545628; XM_011546174; XM_047441852; |
| | XM_047442718; NM_001379153; XM_011546175; XM_047441853; |
| | XM_047442714; XM_047442717; NM_001379154; NM_001379156; |
| | NM_001379159; NM_001379169 |
| CSF2RB | XM_011529904; XM_005261340; XM_047441149; XM_011529903; |
| | XM_047441150; XM_047441148; NM_000395 |
| CSF3 | NR_168489; NR_168491; NM_000759; NM_001178147; NM_172219; |
| | NM_172220; NR_168490; NR_033662 |
| CSF3R | NM_000760; XM_005270493; NM_156039; XM_011540749; NM_156038; |
| | NM_172313; XM_047446753 |
| CSMD3 | XM_017013010; XM_017013009; NM_001363185; XM_047421314; |
| | XM_011516815; NM_052900; XM_017013008; NM_198123; |
| | XM_011516816; NM_198124 |
| CSNK1D | XM_047435379; NM_139062; NM_001893; NR_110578; XR_007065265; |
| | XM_047435381; XM_047435380; NM_001363749; XM_005256336 |
| CSNK1E | NM_152221.3; NM_001894.5 |
| CSPG4 | XM_047432196; NM_001897 |
| CTBP2 | XM_005269567; XM_047424673; XM_011539355; XM_011539358; |
| | XM_047424666; XM_047424669; XM_047424676; NM_001321013; |
| | NM_022802; XM_047424667; XM_047424670; XM_047424671; |
| | NM_001363508; XM_024447830; XM_047424672; XM_047424675; |
| | XM_047424677; XM_047424678; NM_001290215; XM_047424680; |
| | NM_001083914; NM_001290214; NM_001329; XM_047424664; |
| | XM_047424679; NM_001321012; NM_001321014; XM_047424665; |
| | XM_047424668; XM_047424674 |
| CTNNA1 | NM_001323982; NM_001323989; NM_001290312; NM_001323988; |
| | NM_001323990; NM_001323992; NM_001323998; NM_001324002; |
| | NM_001324008; NM_001324012; NM_001290310; NM_001323985; |
| | NM_001323987; NM_001323991; NM_001323995; NM_001323997; |
| | NM_001323999; NM_001324003; NM_001323984; NM_001323996; |
| | NM_001324005; NM_001324013; NM_001903; NM_001290309; |
| | NM_001323983; NM_001323986; NM_001324006; NM_001323993; |
| | NM_001290307; NM_001323994; NM_001324001; NM_001324004; |
| | NM_001324007; NM_001324010; NM_001324011; NM_001324000; |
| | NM_001324009 |
| CTNNA2 | XM_011532557; XM_011532556; XM_024452715; NM_001282598; |
| | NM_001282600; NM_001282599; NM_004389; XM_047443447; |
| | NM_001282597; NM_001399737; XM_017003403; XM_017003405; |
| | NM_001164883; XM_024452714; NM_001320810 |
| CTNNB1 | NM_001330729; NM_001904; XM_047447478; XM_047447482; |
| | XM_047447483; XM_024453356; XM_017005738; XM_047447480; |
| | XM_047447481; XM_047447477; XM_006712985; NM_001098209; |
| | XM_047447479; NM_001098210 |
| CTNND1 | NM_001206890; NM_001085458; NM_001085461; NM_001085463; |
| | NM_001085465; NM_001085467; NM_001085469; NM_001206891; |
| | NM_001331; NM_001206883; NM_001206885; NM_001206886; |
| | NM_001206887; NM_001206889; NM_001085460; NM_001085464; |
| | NM_001085468; NM_001206884; NM_001206888; NM_001085459; |
| | NM_001085462; NM_001085466 |
| CTNND2 | NM_001332; XM_005248251; XM_017009072; XM_047416777; |
| | NM_001288715; NM_001288716; NM_001288717; NM_001364128; |
| | XM_011513967; XM_017009074; XM_005248253; NR_109988 |
| CTSS | NM_004079; NM_001199739 |
| CUL1 | NM_001370663; NM_001370664; NM_003592; NM_001370661; |
| | NM_001370662; NM_001370660 |
| CUL3 | NM_001257198; NM_003590; XM_011511996; XM_006712800; |
| | XM_011511995; NM_001257197; XM_047446024 |
| CUL4A | NM_001354941; NM_003589; NM_001354942; NM_001278514; |
| | NM_001278513; NM_001354938; NM_001354939; NM_001354940; |
| | NM_001008895; NM_001354943; NM_001354944 |
| CUL4B | NM_001330624; NM_001079872; NM_001369145; NM_003588 |
| CX3CL1 | NM_001304392; NM_002996 |
| CX3CR1 | NM_001171174; NM_001337; XM_047447538; NM_001171171; |
| | NM_001171172 |
| CXADR | NM_001207066; NM_001338; XR_001754814; XM_011529478; |
| | XM_011529479; XM_011529477; NM_001207065; XM_011529476; |
| | NM_001207063; NM_001207064 |
| CXCL1 | NM_001511; NR_046035 |
| CXCL10 | NM_001565; NR_168520 |
| CXCL11 | NM_001302123; NM_005409 |
| CXCL12 | NM_000609; NM_001277990; NM_199168; NM_001178134; |
| | NM_001033886 |
| CXCL13 | NM_001371558; NM_006419 |
| CXCL16 | NM_001100812; NM_001386809; NM_022059 |
| CXCL2 | NM_002089 |
| CXCL5 | NM_002994 |
| CXCL6 | NM_002993 |
| CXCL8 | NM_000584; NM_001354840 |
| CXCL9 | NM_002416 |
| CXCR1 | NM_000634 |
| CXCR2 | XM_047444190; XM_047444188; NM_001557; NM_001168298; |
| | XM_005246530; XM_047444189; XM_017003991; XM_047444191; |
| | XM_047444187 |
| CXCR4 | NM_001348059; NM_001348060; XM_047445802; NM_001348056; |
| | NM_003467; NM_001008540 |
| CXCR5 | NM_032966; NM_001716 |
| CXCR6 | NM_001386435; NM_001386436; NM_006564; NM_001386437 |
| CYBB | XM_047441855; NM_000397 |
| CYFIP2 | XM_047417100; XM_047417101; XM_047417102; XM_011534516; |
| | NM_001291721; NM_001037332; NM_001037333; NM_001291722; |
| | NM_014376 |
| CYP24A1 | XM_047439936; NM_001128915; XM_017027692; NM_000782; |
| | XM_047439937; XM_017027691; XM_017027693; XM_047439938; |
| | XM_005260304 |
| CYP2C8 | NM_001198854; NM_001198855; NM_030878; NM_000770; |
| | NM_001198853 |
| CYP2C9 | NM_000771 |
| CYP3A5 | NM_001291830; NM_001190484; NR_033807; NR_033812; |
| | NM_001291829; NM_000777; NR_033810; NR_033811 |
| CYP4A11 | NR_134994; NR_134991; XM_017000465; XM_005270539; NM_000778; |
| | NM_001319155; NR_134992; NR_134993; NR_134989; NR_134990; |
| | NM_001363587; NR_134988 |
| CYP8B1 | NM_004391 |
| CYTH4 | NM_013385; NM_001318024 |
| CYTIP | NM_004288; XM_017005386 |
| DCK | XM_047449689; NM_000788 |
| DCLRE1C | NM_001033858; NM_022487; NR_110297; XM_011519621; |
| | XM_047425651; NR_146962; XM_047425649; NM_001033855; |
| | NM_001289076; NM_001350965; NR_146961; XM_047425652; |
| | XM_047425648; NM_001289078; NM_001350967; NM_001033857; |
| | NM_001350966; XM_011519620; XM_047425650; NM_001289077; |
| | NM_001289079; NR_146960; XR_930515 |
| DCN | NM_133503; NM_133504; NM_133505; NM_001920; NM_133506; |
| | NM_133507 |
| DDB1 | NM_001923 |
| DDB2 | NR_174611; NM_001300734; NM_001399874; XM_047426487; |
| | NM_000107; NM_001399875; NM_001399876; NM_001399878; NR_174610 |
| DDR2 | XM_047421565; NM_001014796; XM_011509587; XM_011509588; |
| | NM_001354982; NM_006182; XM_047421554; NM_001354983 |
| DDX6 | NM_004397; XM_005271417; XM_011542645; XM_047426488; |
| | XM_047426490; XM_017017251; XM_024448377; XM_047426489; |
| | NM_001257191; XM_011542644 |
| DEF6 | NM_022047; XM_047418838 |
| DENND1C | XM_047439458; XM_047439459; XM_047439460; XM_024451727; |
| | NM_001290331; XM_006722906; XM_011528318; XM_006722905; |
| | NM_024898 |
| DEPTOR | NM_001283012; NM_022783 |
| DES | NM_001927; NM_001382708; NM_001382710; NM_001382713; |
| | NM_001382709; NM_001382711; NM_001382712 |
| DGKZ | NM_003646; NM_201533; NM_001199267; NM_001199266; |
| | NM_001199268; NM_201532; NM_001105540 |
| DIABLO | NM_019887; NM_138929; NM_001278304; NM_001278303; |
| | NM_001278302; NM_138930; NM_001278342; NM_001371333 |
| DKK1 | NM_012242 |
| DKK2 | NM_014421 |
| DKK3 | XM_047426775; NM_001018057; NM_013253; XM_017017555; |
| | XM_047426774; XM_006718178; NM_001330220; NM_015881 |
| DLC1 | NM_001316668; NM_182643; XM_005273374; NM_001348081; |
| | NM_001348083; NM_001348084; NM_001164271; NM_006094; |
| | NM_024767; NM_001348082 |
| DLL1 | NM_005618; XM_005266934 |
| DLL4 | NM_019074 |
| DNA2 | XM_006717680; NM_001080449; XM_017015799; XM_011539417; |
| | NR_102264 |
| DNER | XM_005246950; NM_139072 |
| DOCK2 | XM_011534450; XM_011534451; NM_004946; XM_017009190; |
| | XM_011534448; NR_156756; XM_005265830; XM_011534449 |
| DPYD | XM_006710397; XM_017000507; XM_047448077; NM_000110; |
| | NM_001160301; XM_047448076; XR_001737014; XM_005270562 |
| DSC1 | NM_004948; NM_024421 |
| DSC2 | NM_004949; XM_005258206; NM_024422 |
| DSG1 | NM_001942 |
| DSG2 | XM_047437315; NM_001943 |
| DSG3 | XM_011525850; NM_001944 |
| DSP | NM_001008844; NM_004415; NM_001319034 |
| DUSP22 | XM_017011062; NM_020185; XM_011514757; NR_104473; |
| | NM_001286555; NR_104474; NR_104475 |
| DUSP6 | NM_022652; NM_001946 |
| DVL1 | XM_005244733; XM_047448090; NM_004421; NM_182779; |
| | XM_005244732; NM_001330311; NM_181870 |
| DVL2 | NM_004422; XM_047435519; XM_005256502; XM_047435518; |
| | XM_047435520; XM_047435522 |
| DVL3 | NM_004423 |
| DYRK2 | XM_017020032; NM_003583; NM_006482 |
| E2F1 | XM_047439961; NM_005225 |
| EBF1 | XM_024454390; XM_024454391; XM_047416890; NM_001324101; |
| | XM_017009194; XM_017009199; XM_047416892; NM_001364156; |
| | NM_001364158; NM_001324103; NM_001324109; NM_001324111; |
| | NM_001364157; NM_001364159; XM_017009200; XM_047416889; |
| | XM_047416891; NM_001324106; NM_001324107; XM_017009197; |
| | XM_047416888; NM_001364155; NM_024007; NM_182708; |
| | XM_017009202; NM_001290360; XM_017009203; NM_001324108 |
| ECT2 | XM_047447608; XM_047447628; NM_001349101; NM_018098; |
| | XM_006713524; XM_047447610; XM_047447613; XM_047447607; |
| | XM_047447620; XM_047447623; XM_047447630; XM_047447636; |
| | XM_047447637; NM_001349094; NM_001349100; XM_047447616; |
| | XM_047447618; XM_047447625; XM_047447629; XM_047447635; |
| | XM_047447638; NM_001258316; NM_001349097; XM_047447611; |
| | XM_047447634; XM_047447639; NM_001258315; NM_001349096; |
| | NM_001349099; NM_001349104; XM_047447614; XM_047447617; |
| | XM_047447631; XM_047447633; NM_001349102; NM_001349103; |
| | XM_047447609; XM_047447615; XM_047447619; XM_047447621; |
| | XM_047447622; XM_047447624; XM_047447626; NM_001349098; |
| | XM_047447612; XM_047447627; XM_047447632; NM_001349095 |
| EDNRB | NM_001122659; NM_003991; NM_001201397; NM_000115; NR_047024 |
| EEF1A2 | NM_001958 |
| EFNB1 | NM_004429 |
| EGF | XM_005262796; XM_011531707; XM_017007848; XM_017007850; |
| | XM_047449723; NM_001178131; XM_047449725; XM_017007847; |
| | XM_017007855; XM_047449726; XM_047449727; XM_047449729; |
| | XM_017007854; NM_001963; XR_001741156; XM_017007845; |
| | XM_017007849; XM_047449728; NM_001178130; XM_017007846; |
| | XM_017007853; NM_001357021; XM_017007851; XM_047449724; |
| | XM_047449730 |
| EGFR | XM_047419953; NM_001346899; NM_201282; XM_047419952; |
| | NM_201284; NM_001346898; NM_001346900; NM_001346897; |
| | NM_201283; NM_001346941; NM_005228 |
| EGR1 | NM_001964 |
| EHF | XM_047426755; XM_047426760; NM_001378041; NM_001378045; |
| | NM_001378046; NM_001378053; XM_047426757; XM_047426762; |
| | NM_001378048; NM_001378050; NR_165391; XM_024448434; |
| | NM_001378047; NM_001378056; NR_165390; NM_001378052; |
| | XM_047426759; XM_047426761; NM_001378044; NM_012153; |
| | XM_005252861; XM_047426756; NM_001206615; NM_001378051; |
| | XM_047426754; XM_047426753; XM_047426758; NM_001378042; |
| | NM_001378043; NM_001378049; NM_001378055; NM_001206616; |
| | NM_001378054 |
| EIF1AY | NM_001278612; NM_004681 |
| EIF4E | NM_001130678; NM_001968; NM_001130679; NM_001331017 |
| EIF4EBP1 | NM_004095 |
| ELF1 | XM_047430122; NM_001370330; NM_001370331; XM_047430123; |
| | XM_047430124; NM_001145353; XM_047430120; NM_001370332; |
| | XM_047430121; XM_047430118; NM_001370329; NM_172373; |
| | XM_047430119 |
| ELMO1 | XM_011515654; XM_047421091; XM_005249919; XM_047421086; |
| | XM_047421090; NM_001206480; NM_130442; XM_006715805; |
| | NM_001039459; XM_047421087; NR_038120; XM_017012839; |
| | XM_024447008; XM_047421088; NM_001206482; NM_014800; |
| | XM_047421089 |
| ELN | XM_011515869; XM_011515873; XM_017011814; XM_047419961; |
| | XM_047419973; XM_047419978; XM_005250187; XM_011515871; |
| | XM_011515872; XM_047419958; XM_047419962; XM_047419963; |
| | XM_047419965; NM_001278914; XM_005250188; XM_011515874; |
| | XM_047419957; XM_047419964; XM_047419966; XM_047419974; |
| | XM_047419979; NM_000501; NM_001278912; NM_001278939; |
| | XM_011515877; XM_047419960; XM_047419967; XM_047419971; |
| | XM_047419977; NM_001081753; XM_011515876; XM_047419955; |
| | XM_047419970; XM_047419975; NM_001081754; NM_001278917; |
| | XM_017011813; XM_047419956; XM_047419954; XM_047419968; |
| | XM_047419969; XM_047419972; XM_047419980; NM_001278915; |
| | NM_001278918; XM_011515868; XM_011515870; XM_047419981; |
| | NM_001081755; NM_001278916; XM_011515875; XM_047419959; |
| | XM_047419976; NM_001081752; NM_001278913 |
| EMCN | NM_001159694; XM_017008290; NM_016242; XM_011532024 |
| EME1 | XM_005257081; XM_017024236; XM_047435472; NM_001166131; |
| | XM_047435471; NM_152463; XR_007065270; XM_047435470 |
| EMILIN2 | NM_032048; XM_047437887; XM_047437886; XM_047437884; |
| | XM_047437885 |
| ENG | NM_000118; NM_001114753; NM_001278138 |
| ENO1 | NM_001201483; NM_001353346; NM_001428 |
| ENPP3 | NR_133007; NM_005021; XM_017010932; XM_011535897 |
| EOMES | NM_001278182; XM_005265510; NM_005442; NM_001278183 |
| EPAS1 | NM_001430; XM_011532698 |
| EPHA1 | NM_005232 |
| EPHA10 | XM_047418403; XM_017001080; XR_001737123; XR_946610; |
| | XM_017001081; XM_047418391; NM_001099439; NM_001004338; |
| | XR_001737124; XR_001737125; NM_173641; XR_001737127; XR_946613; |
| | XR_001737126 |
| EPHA2 | XM_017000537; XM_047448267; XM_047448259; NM_001329090; |
| | XM_047448272; NM_004431 |
| EPHA3 | NM_005233; XM_005264716; NM_182644; XM_005264715; |
| | XM_047447673 |
| EPHA4 | NM_001304537; NM_001363748; NM_004438; NM_001304536 |
| EPHA5 | XM_005265653; XM_047449763; XM_017007880; XM_017007881; |
| | NM_001318761; NM_001281765; XM_011531735; NM_001281767; |
| | NM_004439; XM_047449762; XM_017007878; NM_001281766; |
| | NM_182472 |
| EPHA6 | XM_017006210; XM_017006212; XM_017006213; XM_017006219; |
| | XM_017006218; XM_047448008; NM_001278300; XM_017006211; |
| | XM_017006214; XM_017006217; NM_001080448; XR_001740110; |
| | XM_006713592; XM_011512706; XM_047448009; NM_173655; |
| | XR_924126; XM_011512707; XM_011512705; NM_001278301; |
| | XM_017006215; XM_017006216 |
| EPHA7 | NM_001288630; XM_047418274; NM_001376467; XM_017010366; |
| | NM_001288629; NM_001376466; NM_001376471; NM_004440; |
| | NM_001376465; NM_001376470; NR_164810; NM_001376468; |
| | NM_001376469 |
| EPHA8 | XR_946576; NM_020526; XM_011540970; XM_011540975; |
| | XM_011540973; XM_011540969; XM_011540972; NM_001006943 |
| EPHB1 | NM_004441 |
| EPHB2 | XM_006710441; XM_047449104; NM_001309192; NM_004442; |
| | NM_001309193; NM_017449; XM_006710442 |
| EPHB3 | NM_004443 |
| EPHB6 | XM_024446675; XM_047419983; NM_001280795; XM_047419984; |
| | XM_011515879; NM_001280794; NR_104001; XM_011515881; |
| | XM_011515880; XM_011515882; NM_004445 |
| ERBB2 | NM_001005862; NM_001382784; NM_001382785; NM_001382788; |
| | NM_001382792; NM_001382793; NM_001382803; XM_047435590; |
| | NM_001289937; NM_001382786; NM_001382800; NM_001382802; |
| | NM_001382806; NM_001382782; NM_001382789; NM_001382795; |
| | NM_001289936; NM_001382797; NM_001382805; NM_004448; |
| | NR_110535; NM_001289938; NM_001382791; NM_001382801; |
| | NM_001382783; NM_001382790; NM_001382794; NM_001382798; |
| | NM_001382799; NM_001382787; NM_001382796; NM_001382804 |
| ERBB3 | XM_047428500; NM_001005915; XM_047428501; NM_001982 |
| ERBB4 | XM_005246376; XM_017003577; XM_017003578; XM_005246377; |
| | NM_001042599; XM_017003581; XM_006712364; XM_017003582; |
| | XM_017003579; XM_017003580; NM_005235 |
| ERCC1 | NM_001369419; NM_001369409; NM_001166049; NM_001369412; |
| | NM_001369417; NM_202001; NM_001369415; NM_001369418; |
| | NM_001369408; NM_001369410; NM_001369411; NM_001369413; |
| | NM_001369414; NM_001369416; NM_001983 |
| ERCC2 | XM_047438393; XR_007066680; NM_000400; NM_001130867; |
| | XR_001753633; XM_011526611 |
| ERCC3 | NM_001303416; NM_000122; XM_011510794; XM_011510795; |
| | NM_001303418 |
| ERCC5 | NM_000123 |
| ERCC6 | NM_000124; NM_001277058; NM_001277059; NM_001346440 |
| EREG | NM_001432 |
| ERG | NM_001243429; NM_001136155; NR_111949; NM_182918; |
| | NM_001243428; NM_001243433; NM_004449; NM_001291391; |
| | NM_001331025; NM_001136154; NM_001243432 |
| ERRFI1 | XM_047422701; XM_005263477; XM_047422698; NM_018948 |
| ESAM | NM_138961 |
| ESCO2 | NM_001017420; XR_949378; XR_007060703; XM_011544422; |
| | XM_011544421 |
| ESM1 | NM_001135604; NM_007036 |
| ESR1 | XM_011535545; XM_017010378; XM_047418293; NM_001385568; |
| | XM_017010381; XM_047418294; XM_047418297; NM_001122741; |
| | NM_001328100; NM_001385570; XM_047418291; XM_017010383; |
| | XM_047418289; XM_047418299; NM_001385572; XM_011535547; |
| | XM_011535549; XM_017010377; XM_047418290; NM_001385571; |
| | XM_017010380; XM_047418298; NM_000125; NM_001122740; |
| | NM_001122742; NM_001291230; NM_001291241; XM_011535543; |
| | XM_017010379; XM_047418296; XM_047418292; XM_047418295; |
| | NM_001385569 |
| ESRP1 | XM_005250991; NM_001122827; NM_017697; NM_001122826; |
| | NM_001034915; XM_047421916; NM_001122825 |
| ETS1 | XM_047426526; NM_001143820; NM_005238; XM_011542650; |
| | XM_017017314; XM_047426527; XM_047426525; NM_001162422; |
| | NM_001330451; XM_017017315 |
| EVI2B | NM_006495 |
| EVL | XM_011536828; XR_007064013; XR_007064014; XM_047431463; |
| | NM_016337; XM_047431460; NM_001330221; XM_047431462; |
| | XM_047431464; XM_005267749; XM_047431465 |
| EZH2 | XM_011515890; XM_047419992; XM_047420004; XM_005249964; |
| | XM_011515889; XM_011515895; XM_047419994; NM_001203248; |
| | XM_011515883; XM_005249963; XM_017011817; XM_017011820; |
| | XM_047419993; XM_047419995; XM_047420000; NM_152998; |
| | XM_011515899; XM_047419990; XM_047419991; XM_047419997; |
| | XM_047419998; NM_001203249; XM_011515896; XM_047419996; |
| | XM_011515893; XM_005249962; XM_011515885; XM_017011819; |
| | XM_047419999; XM_047420001; XM_047420002; XM_047420005; |
| | XM_047420008; XM_047420009; NM_004456; XM_011515887; |
| | XM_011515892; XM_011515894; XM_011515901; XM_047419989; |
| | XM_047420006; XM_047420007; NM_001203247 |
| F11 | XM_047449812; XM_047449817; XM_047449816; XM_005262823; |
| | XM_005262821; XM_005262822; XM_047449814; NM_019559; |
| | XM_047449811; XM_047449815; NM_000128; NM_001354804; |
| | XM_017007886; XM_047449813; XM_017007885; XM_006714137; |
| | XM_017007884 |
| F2R | NM_001311313; NM_001992 |
| F3 | NM_001178096; NM_001993 |
| F7 | XM_011537476; XM_047430125; XM_011537475; NM_001267554; |
| | XM_011537474; NR_051961; XM_006719963; NM_019616; NM_000131 |
| F9 | NM_001313913; XM_005262397; NM_000133 |
| FABP4 | NM_001442 |
| FADD | NM_003824 |
| FAM129C | XM_011527789; XM_011527781; XM_017026454; NM_001321828; |
| | XM_017026453; XM_011527786; XM_047438389; NM_001098524; |
| | XM_047438388; NM_173544; XM_017026457; XM_047438390; |
| | NM_001321826; XM_011527787; NM_001321827; NM_001363609 |
| FAM78A | NM_001400581; NM_001400583; NM_001400588; XM_011518568; |
| | NM_001400584; NM_001400585; NM_001400593; NM_001400591; |
| | XM_047423250; NM_001400589; NM_001400590; NM_001400592; |
| | NM_001400595; NM_033387; NM_001400582; NM_001400586; |
| | NM_001400594; NM_001399459; NM_001400587 |
| FANCA | NM_000135; NM_001018112; NM_001286167; NM_001351830 |
| FAP | XM_011510797; NM_004460; XM_011510796; XM_017003585; |
| | XR_001738668; XR_922891; NM_001291807 |
| FAS | XM_047425180; NR_028033; XM_011539766; XM_011539765; |
| | NR_028034; NR_135314; NR_135315; NM_152877; XM_011539764; |
| | XM_047425179; NM_152873; NM_152876; XM_006717819; |
| | NM_001320619; NR_028035; XM_047425178; NM_152871; NM_152874; |
| | NM_152872; NR_028036; NM_152875; XM_011539767; NM_000043; |
| | NR_135313 |
| FASLG | NM_001302746; NM_000639 |
| FBLN1 | NM_006485; NM_006486; NM_001996; NM_006487 |
| FBLN2 | NM_001004019; NM_001998; NM_001165035 |
| FBN1 | NM_000138 |
| FBXW7 | XM_011532087; XM_047415900; NM_001257069; XM_011532084; |
| | XM_011532085; XM_047415899; XM_047415901; NM_001013415; |
| | XM_024454126; XM_047415898; XM_047415897; XM_047415902; |
| | XM_011532086; XM_024454123; NM_001349798; NM_018315; |
| | NM_033632 |
| FCER2 | NM_002002; NM_001220500; XM_005272462; NM_001207019 |
| FCGR1A | NM_001378804; NM_001378805; NM_001378807; NM_001378810; |
| | NR_166122; NR_166123; NM_001378809; NM_001378811; |
| | NM_001378808; NR_166121; NM_000566; NM_001378806 |
| FCGR2B | NM_001002273; NM_001386004; NR_169827; XM_024454047; |
| | NM_001002275; NM_001386006; NM_001190828; NM_004001; |
| | NM_001386002; NM_001002274; NM_001386001; NM_001386005; |
| | NM_001386000; NM_001386003; NM_001394477 |
| FCGR3A | XM_047449443; NM_001127595; NM_001329122; XM_047449444; |
| | NM_001127596; NM_001127592; NM_000569; NM_001386450; |
| | NM_001127593; NM_001329120 |
| FCRL1 | XM_005244869; XM_047444001; XM_011509125; XM_011509136; |
| | NM_001159397; XM_011509128; XM_011509133; XM_011509134; |
| | NM_001159398; XM_005244866; XM_005244867; XM_011509127; |
| | NM_052938; XR_921738; XM_011509135; XM_011509126; |
| | XM_011509129; XM_011509130 |
| FCRL2 | XR_001737404; XM_011509974; XM_017002318; XM_017002319; |
| | XM_047430273; XM_006711535; XM_011509976; NM_001159488; |
| | NM_138738; XM_017002316; XM_017002317; NR_125358; NM_138739; |
| | XM_011509975; XM_047430270; XM_047430275; NM_030764; |
| | XR_001737403 |
| FCRL3 | NR_135216; NR_135217; XM_006711145; NM_001320333; NM_052939; |
| | NR_135214; NR_135215; NM_001024667 |
| FCRL5 | XM_011510032; XM_047431422; XM_011510030; XM_011510033; |
| | XM_011510031; NM_031281; NM_001195388 |
| FCRLA | NM_001184867; NM_001184870; NM_001184866; NM_032738; |
| | XM_006711581; XM_011510065; NM_001184873; NM_001184871; |
| | NM_001184872; NM_001366195; NM_001366196 |
| FEN1 | NM_004111 |
| FERMT3 | NM_001382362; NM_001382363; NM_001382364; NM_001382448; |
| | NM_031471; XM_047427676; NM_001382361; NM_178443 |
| FFAR2 | XM_047438699; NM_005306; NM_001370087; XM_017026711; |
| | XM_047438700 |
| FGA | NM_000508; NM_021871 |
| FGF2 | NM_001361665; NM_002006 |
| FGFR1 | XM_024447097; XM_047421569; XM_047421570; NM_001174065; |
| | NM_001354370; NM_0231U; XM_006716310; XM_006716303; |
| | XM_006716304; XM_011544445; XM_011544449; XM_017013221; |
| | XM_017013225; NM_001354368; NM_001354369; NM_015850; |
| | NM_023106; XM_006716307; XM_011544444; XM_047421571; |
| | XM_047421572; NM_001354367; NM_023105; XM_006716311; |
| | XM_011544446; XM_011544452; XM_017013219; XM_017013226; |
| | XM_047421573; XM_047421574; NM_023107; NM_023109; |
| | XM_011544447; XM_011544451; NM_023110; XM_006716312; |
| | XM_011544450; XM_017013220; XM_017013227; XM_017013231; |
| | NM_001174067; NM_032191; XM_006716314; XM_011544448; |
| | XM_047421575; NM_001174063; NM_001174064; NM_001174066; |
| | XM_047421576; NM_023108 |
| FGFR2 | XM_017015924; NM_001144919; XM_006717708; XM_017015925; |
| | NM_001144915; NM_001144917; NM_022975; NM_023028; |
| | XM_024447890; NM_000141; NM_001144913; NM_001320654; |
| | NM_022970; NR_073009; NM_022971; NM_022973; NM_023030; |
| | XM_006717710; XM_024447887; XM_024447888; NM_001320658; |
| | NM_022976; XM_017015920; NM_001144918; NM_022974; NM_023031; |
| | XM_024447889; XM_024447891; NM_023029; XM_017015921; |
| | NM_001144914; NM_001144916; NM_022972 |
| FGFR3 | XM_047449823; XM_047449824; XM_006713869; XM_006713873; |
| | NM_022965; XM_006713868; NM_001354810; XM_011513422; |
| | XM_047449821; XM_047449822; NM_000142; XM_011513420; |
| | XM_047449820; XM_006713870; XM_006713871; NM_001163213; |
| | NM_001354809; NR_148971 |
| FGG | NM_000509; NM_021870 |
| FGL1 | NM_201552; XM_047421577; NM_201553; NM_004467; NM_147203 |
| FGL2 | NM_006682 |
| FGR | NM_005248; NM_001042729; NM_001042747 |
| FKBP11 | XM_047428939; NM_001143782; NM_016594; XM_047428940; |
| | NM_001143781 |
| FKBP15 | XM_006717018; NM_015258; XM_006717019 |
| FLT1 | NM_001160030; NM_001159920; XM_011535014; XM_017020485; |
| | NM_001160031; NM_002019 |
| FLT3 | XM_011535015; NM_004119; XM_011535018; XM_017020488; |
| | NR_130706; XM_017020486; XM_047430216 |
| FLT4 | XM_011534478; XM_047417002; NM_001354989; XM_047417003; |
| | XM_017009266; XM_017009268; NM_002020; NM_182925; |
| | XM_017009263; XM_011534484 |
| FMNL1 | XM_006722064; XM_047436644; NM_005892; XM_006722062; |
| | XM_006722069; XM_047436641; XM_006722070; XM_047436637; |
| | XM_047436642; XM_047436643; XM_011525179; XM_047436640; |
| | XM_006722065; XM_047436639; XM_011525180; XM_047436638; |
| | XM_006722063; XM_047436646; XM_006722066; XM_047436645 |
| FMO3 | XM_047416207; NM_001002294; NM_006894; NM_001319173; |
| | NM_001319174 |
| FN1 | NM_001306129; NM_001365519; NM_212474; NM_001306132; |
| | NM_001365517; NM_001365522; NM_001306131; NM_001365521; |
| | NM_212476; NM_212478; NM_212475; NM_001365523; NM_001365524; |
| | NM_002026; NM_001365520; NM_212482; NM_001365518; NM_054034; |
| | NM_001306130 |
| FNBP1 | XM_005251830; XM_005251834; XM_011518400; XM_005251820; |
| | XM_005251825; XM_011518401; XM_011518402; XM_047423011; |
| | XM_047423013; XM_047423017; XM_005251815; XM_005251823; |
| | XM_005251824; XM_005251827; XM_011518399; XM_047423015; |
| | XM_047423022; NM_001363755; XM_005251821; XM_005251832; |
| | XM_006717016; XM_047423012; XM_047423016; NM_015033; |
| | XM_005251826; XM_017014488; XM_047423020; XM_005251831; |
| | XM_005251833; XM_005251828; XM_047423018; XM_047423021; |
| | XM_005251822; XM_017014492; XM_047423014; XM_047423019 |
| FOS | NM_005252 |
| FOXC2 | NM_005251 |
| FOXL1 | NM_005250 |
| FOXM1 | XM_011520932; XM_011520934; XM_047428547; XM_047428548; |
| | NM_001243088; XM_011520930; XM_011520933; XM_011520935; |
| | XR_931507; XM_047428549; XM_047428551; NM_202003; |
| | XM_047428546; NM_202002; XM_005253676; XM_011520931; |
| | XM_047428550; NM_001243089; NM_021953 |
| FOXO1 | XM_047430204; XM_011535008; XM_011535010; NM_002015 |
| FOXP3 | NM_001114377; NM_014009 |
| FOXQ1 | NM_033260 |
| FPR1 | NM_002029; NM_001193306 |
| FPR3 | NM_002030; XM_011526687 |
| FRAT1 | NM_181355; NM_005479 |
| FRAT2 | NM_012083 |
| FSCN1 | NM_003088 |
| FZD1 | NM_003505 |
| FZD10 | NM_007197 |
| FZD2 | NM_001466 |
| FZD3 | XM_047422239; NM_145866; XM_047422238; XM_047422240; |
| | XM_017013842; NM_017412; XM_017013841; XM_017013844; |
| | XM_017013843 |
| FZD4 | NM_012193 |
| FZD5 | NM_030804; NM_003468 |
| FZD6 | NM_003506; NM_001164615; NM_001164616; NM_001317796; NR_133921 |
| FZD7 | NM_003507 |
| FZD8 | NM_031866 |
| FZD9 | NM_003508 |
| G6PC | NM_000151; NM_001270397 |
| GABARAP | NM_007278 |
| GABARAPL1 | NM_031412; NM_001363598 |
| GABARAPL2 | NM_007285 |
| GAS6 | NM_001143945; NM_001143946; NM_000820 |
| GATA3 | XM_005252443; XM_047425044; NM_002051; XM_005252442; |
| | XM_047425045; NM_001002295 |
| GEN1 | XM_011532821; XM_047444156; XM_006712005; XM_047444147; |
| | NM_182625; XM_011532822; XM_047444155; XM_047444158; |
| | XM_047444159; XM_005262613; NM_001130009; XM_011532820; |
| | XM_047444157 |
| GHR | NM_001242401; NM_001242460; NM_001242462; NM_001242402; |
| | NM_001242406; NM_001242399; NM_001242403; NM_001242461; |
| | NM_001242405; NM_000163; NM_001242400; NM_001242404 |
| GINS2 | NM_016095 |
| GJA10 | NM_032602 |
| GJA3 | NM_021954; XM_011535048 |
| GJA5 | NM_005266; NM_181703 |
| GJB1 | NM_000166; XM_011530907; NM_001097642 |
| GJB2 | XM_011535049; NM_004004 |
| GJB3 | NM_024009; NM_001005752 |
| GJB4 | XM_011540679; NM_153212 |
| GJB6 | NM_001110219; NM_001110221; NM_001370090; XM_047430057; |
| | NM_001370091; NM_001110220; NM_001370092; XM_047430056; |
| | NM_006783 |
| GJC1 | XM_047435078; XM_047435079; NM_005497; XM_024450526; |
| | XR_007065255; XM_047435080; XM_024450527; XR_934346; |
| | XM_047435077; NM_001080383 |
| GJD2 | NM_153368 |
| GJD4 | NM_153368.3 |
| GLCCI1 | NM_138426 |
| GLI1 | NM_001160045; NM_005269; XM_011538190; NM_001167609; |
| | XM_011538189 |
| GLI2 | XM_011510969; XM_017003818; XM_011510971; XM_047443947; |
| | NM_030380; NM_030379; NM_001374354; NM_005270; NM_030381; |
| | NM_001371271; NM_001374353 |
| GLI3 | XM_047420208; XM_047420207; NM_000168; XM_011515274; |
| | XM_017011997; XM_047420209; XM_047420206; XM_047420205 |
| GLT1D1 | NR_159493; NM_144669; XM_047428371; XM_047428372; |
| | XM_047428373; XR_001748588; XM_011537957; NM_001366886; |
| | NM_001366887; NM_001366888; NM_001366889; NR_133646 |
| GNA13 | NM_006572; NM_001282425 |
| GNRHR | NM_001012763; NM_000406 |
| GPC3 | NM_004484; XM_017029413; NM_001164618; NM_001164617; |
| | NM_001164619 |
| GPC5 | XM_017020437; XM_047430153; XM_017020435; XM_017020436; |
| | XM_011521055; XM_011521054; XM_011521057; XM_011521058; |
| | XM_011521060; XM_011521056; XM_011521059; NM_004466 |
| GPNMB | XM_047419776; XM_005249578; NM_001005340; XM_017011678; |
| | NM_002510 |
| GPR34 | NM_005300; NM_001033513; XM_005272597; NM_001033514; |
| | NM_001097579 |
| GPR35 | NM_001195382; NM_001195381; NM_001394730; NM_005301 |
| GRAMD1A | XM_011527153; XM_047439133; XM_047439134; XM_017027035; |
| | XM_011527155; XM_024451622; NM_001320035; NM_020895; |
| | XM_011527149; NM_001136199; NM_001320036; XM_017027034; |
| | NM_001320034 |
| GRAP2 | NM_001291825; NM_001291826; XM_047441608; NM_001291824; |
| | XM_047441607; NM_004810; XR_007067996; NM_001291828; |
| | XR_007067995 |
| GRB2 | NM_203506; NM_002086 |
| GSK3B | NM_001354596; NM_002093; XM_006713610; NM_001146156 |
| GSTP1 | NM_000852 |
| GTF2H1 | XM_006718208; NM_001142307; XM_024448457; NM_005316; |
| | XM_024448458 |
| GTF2H3 | NM_001271866; NM_001516; XM_017019228; NM_001271867; |
| | NM_001271868 |
| GZMA | NM_006144 |
| GZMB | NM_001346011; NM_004131; NR_144343 |
| GZMK | NM_002104 |
| HAO1 | NM_017545 |
| HAO2 | XM_024447486; XM_024447484; XM_011541561; XM_024447495; |
| | NM_001377472; XM_047422154; XM_024447492; NM_001005783; |
| | NM_016527; XM_024447485 |
| HAVCR2 | NM_032782 |
| HCK | NM_001172132; NM_001172133; NM_001172130; NM_002110; |
| | NM_001172131; NM_001172129 |
| HCLS1 | NM_005335; NM_001292041 |
| HDAC1 | XM_011541309; NM_004964 |
| HDAC2 | NR_033441; XM_047418692; NR_073443; NM_001527 |
| HES1 | NM_005524 |
| HES2 | NM_019089 |
| HES5 | XM_005244751; NM_001010926 |
| HEY1 | NM_001040708; NM_001282851; NM_012258 |
| HEY2 | NM_012259; XM_017010627; XM_017010628 |
| HEYL | NM_014571; XM_005270745 |
| HGF | NM_000601; NM_001010933; XM_047420293; NM_001010934; |
| | NM_001010931; NM_001010932 |
| HGFAC | NM_001297439; XM_047450155; NM_001528 |
| HHIP | XM_005263178; NM_022475; XM_006714288 |
| HIC1 | NM_006497; NM_001098202 |
| HIPK2 | XM_011516081; NM_001113239; XM_011516078; XM_047420260; |
| | XM_047420261; XM_047420262; XM_047420265; NM_022740; |
| | XM_011516080; XM_011516077; XM_011516079; XM_047420263; |
| | XM_047420264 |
| HK3 | XM_047417134; XM_011534540; NM_002115; XR_941102 |
| HMHA1 | XM_011527858; XM_047438547; NM_001282334; NM_001321232; |
| | XM_047438548; NM_001282335; NM_012292; XM_047438549; |
| | NM_001258328; XM_047438545; XM_047438546 |
| HOXA1 | NM_005522; NM_153620 |
| HOXB9 | NM_024017 |
| HOXC10 | NM_017409 |
| HPSE | NM_001199830; NM_001098540; NM_006665; NM_001166498 |
| HRAS | NM_005343; NM_001318054; NM_001130442; NM_176795 |
| HRG | NM_000412; XM_005247415 |
| HRK | NM_003806; NR_073189 |
| HRSP12 | NM_005836 |
| HSPA5 | NM_005347 |
| HSPB1 | NM_001540 |
| HSPH1 | XM_005266236; XM_017020364; XM_047430058; XM_011534888; |
| | NM_006644; XM_017020363; XM_017020362; XM_011534887; |
| | NM_001286504; NM_001286503; NM_001286505; NM_001349704 |
| ICAM1 | NM_000201 |
| ICAM3 | NM_001395374; NM_001395376; NM_001320605; NM_001320606; |
| | NM_002162; NM_001395375; NM_001320608 |
| ICOS | XR_007073112; XM_047444022; NM_012092 |
| ICOSLG | NM_001395918; XM_011529514; NM_001283051; NM_001283050; |
| | NM_001283052; XM_047440732; NM_015259; XM_047440731; |
| | NM_001365759; XM_011529516; XM_047440729; XM_047440730 |
| IDO1 | NM_002164 |
| IFI30 | NM_006332 |
| IFITM2 | NM_006435 |
| IFNAR1 | NM_000629; NM_001384499; NM_001384502; NM_001384498; |
| | NM_001384503; NM_001384501; NM_001384504; NM_001384500 |
| IFNAR2 | NM_001289126; NM_001385054; NM_001289128; NM_207584; |
| | NM_001385055; NM_000874; NM_001289125; NM_207585 |
| IFNG | NM_000619 |
| IFNGR1 | NM_001363526; XM_011535794; XM_047418726; XM_011535793; |
| | NM_000416; XM_006715470; NM_001363527 |
| IFNGR2 | NM_001329128; NM_005534 |
| IFNLR1 | NM_173064; XM_006710394; XM_017000479; NM_173065; NM_170743 |
| IGBP1 | XM_047442084; NM_001370193; NM_001551; XM_047442083; |
| | NM_001370194; NM_001370192 |
| IGF1R | XM_047432444; XM_011521517; NM_000875; XM_011521516; |
| | XM_017022137; XM_047432442; NM_152452; XM_047432443; |
| | XM_047432445; NM_001291858 |
| IGF2 | NM_001291862; NM_001291861; NM_000612; NM_001007139; |
| | NM_001127598 |
| IGFBP2 | NM_001313990; NM_000597; NM_001313992; NM_001313993 |
| IGKC | NG_000834.1 |
| IGLL5 | NM_001178126; NM_001256296 |
| IKBKB | NM_001190720; NR_033818; XM_047421757; NM_001242778; |
| | NR_040009; NM_001190722; XM_047421764; XM_047421760; |
| | XM_047421758; XM_011544520; XM_047421759; XM_047421761; |
| | XM_047421763; NM_001556; NR_033819; XM_011544517; |
| | XM_047421762; NM_001190721 |
| IKBKG | NM_001099856; NM_003639; NM_001099857; NM_001377315; |
| | NM_001377312; NM_001145255; NM_001321397; NM_001321396; |
| | NM_001377313; NR_165197; NM_001377314 |
| IKZF1 | XM_011515064; XM_011515071; XM_011515073; XM_017011668; |
| | XM_047419729; XM_047419732; XM_047419733; XM_047419741; |
| | NM_001220767; NM_001291841; NM_001291842; NM_001220775; |
| | XM_011515061; XM_011515063; XM_011515065; XM_011515072; |
| | XM_011515078; XM_047419723; XM_047419730; XM_047419736; |
| | XM_047419742; XM_047419749; NM_001291837; NM_001291846; |
| | NM_001220774; XM_011515062; XM_011515066; XM_047419726; |
| | XM_047419739; XM_047419740; XM_047419743; XM_047419746; |
| | XM_047419747; NM_006060; NM_001220772; XM_047419748; |
| | NM_001220768; NM_001220771; NM_001291843; NM_001291845; |
| | XM_011515060; XM_011515067; XM_047419731; XM_047419738; |
| | NM_001291838; NM_001291840; NM_001220769; XM_011515058; |
| | XM_011515059; XM_011515070; XM_047419728; XM_047419734; |
| | XM_047419735; XM_047419745; NM_001220765; NM_001220770; |
| | NM_001291839; NM_001291844; XM_011515077; XM_047419724; |
| | XM_047419744; XM_047419750; NM_001220773; XM_011515074; |
| | XM_047419725; XM_047419727; NM_001291847; NM_001220766; |
| | NM_001220776 |
| IKZF2 | XM_011510804; XM_011510815; NM_001371277; XM_011510803; |
| | XM_011510808; XM_011510816; NM_001371275; XM_005246386; |
| | XM_011510807; XM_011510810; XM_047443723; XM_011510811; |
| | NM_001371276; XM_011510809; XM_047443722; XM_047443724; |
| | NM_001079526; NM_001371274; NM_001387220; NM_016260; |
| | XM_011510817; XM_011510818; XM_011510805; XM_011510812; |
| | XM_011510819; XM_047443721; XM_047443725; XM_047443727; |
| | XM_011510802; XM_047443726 |
| IKZF3 | NM_001257411; NM_001284516; NM_001257414; NM_001284515; |
| | NM_183230; NM_183231; NM_183232; NM_001257412; NM_001257413; |
| | NM_001257408; NM_001257409; NM_001284514; NM_183228; |
| | XM_047435625; NM_001257410; NM_012481; NM_183229 |
| IKZF4 | XM_017019810; XM_047429342; XM_047429345; XM_047429347; |
| | NM_001351089; NM_022465; XM_047429341; XM_047429346; |
| | XM_047429349; XM_047429351; NM_001351090; NM_001351091; |
| | XM_011538664; XM_011538669; XM_047429350; XM_047429352; |
| | XM_047429353; XM_017019806; XM_047429348; XM_005269089; |
| | XM_047429344; NM_001351092; XM_017019812 |
| IL10RA | XM_047426883; NM_001558; XM_047426884; XM_047426882; NR_026691 |
| IL10RB | NR_175973; NM_001405849; NM_001405850; NM_000628 |
| IL11RA | NR_052010; NM_004512; NM_147162; NM_001142784 |
| IL12B | NM_002187 |
| IL12RB2 | NR_047584; XM_011541384; XM_047419669; XM_047419670; |
| | XM_005270827; XM_006710617; NM_001374259; XM_011541383; |
| | XM_047419667; NM_001258215; NM_001258216; XM_047419665; |
| | XM_047419666; XM_047419668; NM_001258214; NM_001319233; |
| | XM_005270828; XM_017001203; NM_001559; NR_047583 |
| IL13RA1 | XM_047442096; NM_001560 |
| IL13RA2 | NM_000640 |
| IL15RA | XM_011519471; XM_047425181; NM_002189; NR_046362; |
| | XM_011519463; XM_011519465; XM_011519468; XM_011519469; |
| | XM_017016197; XM_047425182; XM_047425184; XM_011519467; |
| | XM_017016198; XM_017016199; NM_001351095; XM_011519462; |
| | XM_011519464; XM_011519466; XM_047425185; XM_047425190; |
| | XM_011519475; XM_017016195; XM_047425187; XM_047425189; |
| | XM_047425191; NM_001243539; NM_001351096; NM_001351097; |
| | NM_172200; XM_011519470; XM_047425186; NM_001256765; |
| | XM_011519461; XM_011519474; XM_011519477; XM_047425183; |
| | XM_011519472; XM_011519476; XM_047425188 |
| IL16 | XM_047432448; NM_004513; NM_172217; XM_047432451; |
| | XM_047432458; NM_001172128; NM_001352684; NR_148035; |
| | XM_047432450; XM_047432457; NM_001352686; XM_047432452; |
| | NM_001352685; XM_047432447; XM_047432454; XM_047432449; |
| | XM_047432453; XM_047432455; XM_047432456 |
| IL18RAP | NM_001393489; XM_047446162; XM_011512088; XM_024453197; |
| | NM_001393487; NM_001393486; XR_007083519; XM_024453199; |
| | XM_024453201; NM_003853; XM_024453198; XM_047446163; |
| | NM_001393488 |
| IL1A | NM_001371554; NM_000575 |
| IL1RN | NM_001318914; XM_047444186; XM_047444184; XM_011511121; |
| | XM_047444185; NM_001379360; NM_173841; NM_173843; NM_173842; |
| | NM_000577 |
| IL21R | XM_017023257; NM_181078; XM_047434180; NM_021798; NM_181079; |
| | XM_011545857 |
| IL23R | XM_011540791; XM_047447227; NM_144701; XM_011540790 |
| IL2RA | NM_001308243; NM_001308242; NM_000417 |
| IL2RB | NM_000878; NM_001346223; NM_001346222 |
| IL2RG | XM_047442089; NM_000206 |
| IL33 | XM_047424061; XM_047424063; NM_001314046; NM_001314048; |
| | XM_047424062; NM_001314047; NM_001353802; NM_033439; |
| | XM_047424064; NM_001199641; NM_001314045; XM_047424060; |
| | NM_001199640; XM_017015285; NM_001314044 |
| IL3RA | XM_047442090; XM_047442730; XM_005274431; XM_017030043; |
| | NM_002183; XM_005274781; NM_001267713; XM_005274432; |
| | XM_017029491; XM_005274780; XM_047442731; XM_047442091 |
| IL4I1 | NM_001385639; NM_172374; NM_152899; NR_047577; NM_001258018; |
| | NM_001258017 |
| IL4R | XM_047434066; NM_001257406; NM_001257407; NM_001008699; |
| | XM_011545827; XM_011545834; XM_011545825; XM_011545833; |
| | XM_017023211; XM_005255308; XM_011545828; XM_047434068; |
| | NM_000418; XM_011545826; NM_001257997; XM_047434067 |
| IL5RA | NM_175726; NM_175724; NM_175727; XM_011533677; NM_001243099; |
| | XM_011533678; NM_175725; NM_175728; NM_000564 |
| IL6R | XM_047419649; XM_047419654; NM_001382771; XM_005245139; |
| | NM_001206866; NM_001382770; NM_181359; XM_047419650; |
| | XM_047419656; NM_001382773; XM_047419657; NM_000565; |
| | NM_001382769; NM_001382774; NM_001382772; XM_017001199; |
| | XM_047419648; XM_047419655 |
| IL6ST | NM_175767; NM_001364279; NR_157112; NM_001364276; |
| | NM_001364275; NM_001364277; NM_002184; NM_001190981; |
| | NM_001364278; NR_120480 |
| IL7R | XM_047417149; XM_005248299; XM_047417150; NR_120485; NM_002185 |
| ING4 | XM_047428932; NM_001127585; NM_016162; NM_198287; |
| | XM_011520965; NM_001127584; NM_001127586; NM_001127582; |
| | NM_001127583; XM_011520964; XM_047428931 |
| INHBA | NM_002192; XM_047420335; XM_017012174 |
| INHBB | NM_002193 |
| INPP4B | XM_047416356; XM_047416357; XM_047416365; NM_001385334; |
| | NM_001385339; NM_001385341; NM_001385348; NM_001385460; |
| | NM_001385461; NM_003866; NR_169615; XM_017008798; |
| | XM_024454274; NM_001385357; NM_001385362; NM_001385381; |
| | NM_001385452; NR_169614; XR_007057981; XM_047416352; |
| | XM_047416367; NM_001101669; NM_001385344; NM_001385379; |
| | NM_001385457; XM_047416354; XM_047416358; XM_047416361; |
| | NM_001385337; NM_001385342; NM_001385351; NM_001385458; |
| | NR_169619; NR_169623; NR_169624; XM_024454273; XM_011532391; |
| | XM_047416363; XM_047416366; NM_001385336; XM_017008797; |
| | XM_047416353; XM_047416359; XM_047416368; NM_001331040; |
| | NM_001385382; NM_001385383; NM_001385450; NM_001385454; |
| | NM_001385455; NR_169599; NR_169617; NR_169618; XM_047416362; |
| | NM_001385335; NM_001385340; NM_001385347; NM_001385459; |
| | XM_047416360; NM_001385338; NM_001385343; NM_001385350; |
| | NM_001385380; NR_169616 |
| INPP5D | XM_047444219; NM_005541; NM_001017915; XM_047444220 |
| INPPL1 | XM_047426890; XM_005273979; XM_047426889; XM_047426892; |
| | XM_047426888; XM_047426893; XM_047426887; NM_001567; |
| | XM_011544999; XM_047426891 |
| INSIG2 | NM_016133; NM_001321333; NM_001321330; XM_047444640; |
| | NM_001321331; NM_001321332; NM_001321329 |
| INSR | NM_001079817; NM_000208; XM_011527989; XM_011527988 |
| INSRR | NM_014215 |
| IRAK1 | XM_005274668; XM_047442097; NM_001025242; XM_047442098; |
| | NM_001025243; NM_001569 |
| IRAK2 | NM_001570 |
| IRF1 | XM_047417153; NR_149068; NM_001354924; NR_149069; |
| | XM_011543379; XM_047417154; NM_002198; NM_001354925 |
| IRF4 | NM_002460.4; NM_001195286.2; NR_046000.3 |
| IRF5 | XM_047420336; NM_001242452; XM_006715974; NM_032643; |
| | NM_001364314; XM_011516160; XM_011516158; XM_047420340; |
| | NM_001347928; XM_047420337; NM_001098629; XM_011516159; |
| | XM_047420338; NM_001098627; NM_001098630; XM_047420339 |
| IRF9 | NM_006084; NM_001385402; NM_001385400; NM_001385401 |
| ITGA1 | NM_181501 |
| ITGA10 | XM_017002623; XM_047432904; XM_047432906; XM_017002626; |
| | XM_047432895; XM_047432909; XM_047432910; NM_001303041; |
| | XR_007064482; XM_047432915; NM_001303040; XM_017002622; |
| | XM_017002625; NM_003637; XM_047432924; XR_001737504; |
| | XM_017002624; XM_011510083; XM_011510084; XM_017002627 |
| ITGA11 | XM_011521363; NM_012211; XM_005254228; NM_001004439 |
| ITGA2 | NR_073103; NR_073104; NR_073105; NR_073106; NR_073107; |
| | NM_002203 |
| ITGA3 | XM_005257308; NM_002204; NM_005501; XM_047435922 |
| ITGA4 | NM_001316312; NM_000885 |
| ITGA5 | NM_002205; XM_024448970 |
| ITGA6 | XM_047444221; XM_017004006; NM_000210; NM_001079818; |
| | NM_001394928; NM_001365530; NM_001316306; XM_017004005; |
| | NM_001365529; XM_047444222 |
| ITGA7 | XM_005268844; XM_005268848; XM_047428792; XM_047428799; |
| | XM_005268839; XM_017019265; XM_047428794; NM_002206; |
| | XM_047428793; NM_001374465; XM_047428798; XM_047428791; |
| | XM_005268840; XM_005268841; XM_047428795; XM_047428797; |
| | NM_001367993; NM_001367994; XM_047428796; NM_001144996; |
| | NM_001144997 |
| ITGA8 | NM_003638; XM_011519752; NM_001291494 |
| ITGA9 | NM_002207 |
| ITGAD | XM_047434071; NM_005353; XM_011545836; XM_011545838; |
| | XM_011545839; XM_011545844; XM_011545835; XM_011545837; |
| | XM_011545842; XR_950791; XM_011545845; XM_011545846; |
| | XM_011545847; XM_017023215; NM_001318185; XM_011545841; |
| | XM_011545843; XM_011545848 |
| ITGAE | XM_047435924; XM_011523825; XM_047435923; XM_011523828; |
| | XM_024450740; XM_017024586; NM_002208; XM_011523827 |
| ITGAL | XM_005255313; XM_006721044; NM_001114380; XR_950794; |
| | XM_047434073; XM_047434072; NM_002209 |
| ITGAM | XR_950796; NM_000632; XM_011545850; XM_011545851; |
| | XM_017023216; NM_001145808; XM_006721045; XR_007064878 |
| ITGAV | XM_047444225; NM_001144999; NM_002210; NM_001145000 |
| ITGAX | NM_001286375; XM_024450263; XM_011545852; XM_011545854; |
| | XM_047434075; NM_000887; XM_047434074 |
| ITGB1 | NM_033666; NM_033669; NM_002211; NM_033667; NM_033668; |
| | NM_133376 |
| ITGB2 | XM_047440763; NM_000211; NM_001303238; XM_006724001; |
| | NM_001127491 |
| ITGB3 | NM_000212 |
| ITGB4 | XM_047435927; XM_005257311; XM_006721866; XM_006721870; |
| | NM_000213; NM_001005619; NM_001005731; XM_005257309; |
| | XM_011524752; XM_006721867; XM_011524751; XM_047435929; |
| | NM_001321123; XM_047435926; XM_047435928; XM_006721868 |
| ITGB5 | NM_001354766; XM_047448087; XM_005247436; XM_006713630; |
| | XM_047448089; NM_002213; XM_017006353; NM_001354765; |
| | XM_047448088; NM_001354764 |
| ITGB6 | NM_001282354; NM_001282353; NM_000888; NM_001282389; |
| | NM_001282390; NM_001282355; NM_001282388 |
| ITGB7 | XM_005268851; XM_005268852; NM_000889; NR_104181; XM_047428800 |
| ITGB8 | XM_011515393; XM_047420343; NM_002214; XM_047420341; |
| | XM_047420344; XM_011515394; XM_017012182; XM_017012178; |
| | XM_017012183; XM_047420345; XM_017012179; XM_017012180; |
| | XM_047420342 |
| ITIH1 | NM_001166436; NM_002215; NM_001166434; NM_001166435 |
| ITIH2 | NM_002216 |
| ITIH3 | NM_001392021; NM_001392022; NM_002217; NM_001392024; |
| | NM_001392023; NM_001392025; NM_001392026; NM_001392019; |
| | NM_001392020; NM_001392027 |
| ITIH4 | NM_001166449; NM_002218 |
| ITIH5 | NM_001001851; NM_032817; XM_011519713; NM_030569; |
| | XM_011519714 |
| ITK | NM_005546 |
| ITM2A | NM_004867; NM_001171581 |
| ITPKB | NM_002221; NM_001388404; XM_017001211 |
| ITPR1 | NM_002222; NM_001099952; NM_001378452; NM_001168272 |
| ITPR2 | XM_047428801; XM_017019266; XR_001748687; XM_017019269; |
| | NM_002223; XR_001748686 |
| ITPR3 | XM_047418731; XM_017010832; XM_047418734; XM_011514577; |
| | XM_047418733; NM_002224; XM_047418732 |
| JAG1 | NM_000214 |
| JAG2 | NM_145159; XM_047431352; XM_047431354; NM_002226; |
| | XM_047431353 |
| JAK2 | NM_001322195; NM_001322196; NM_001322194; NM_001322198; |
| | NM_004972; NM_001322199; NM_001322204; NR_169763; NR_169764 |
| JAK3 | NM_000215; XR_007066796; XM_011527991; XM_047438786 |
| JAM2 | NM_001270408; NM_021219; NR_072999; NM_001270407 |
| JAM3 | NM_032801; NM_001205329 |
| JUP | XM_047435934; XM_047435935; XM_047435940; XM_017024590; |
| | XM_006721875; XM_047435942; NM_001352773; NM_001352776; |
| | XM_047435938; NM_001352777; NM_021991; XM_047435939; |
| | NM_001352775; NM_002230; XM_006721874; XM_011524758; |
| | XM_047435937; XM_047435941; NM_001352774 |
| KAT2B | NM_003884; XM_005265528; XM_047449147 |
| KAT5 | XM_047426253; NM_182710; XM_047426252; NM_001206833; |
| | NM_006388; NM_182709; XM_006718421 |
| KCNA3 | NM_002232; NR_109846; NR_109845 |
| KCNAB2 | XM_047432867; XM_047432870; XM_047432872; NM_003636; |
| | XM_047432878; XM_011542322; NM_001199860; NM_001199863; |
| | XM_017002620; XM_005263514; XM_011542321; NM_001199861; |
| | NM_001199862; XM_047432866; NM_172130 |
| KCNH2 | XM_017012195; XM_017012196; NM_000238; XM_011516185; |
| | NM_172056; XM_047420349; XM_047420348; NM_001204798; |
| | NM_172057 |
| KCNJ15 | NM_170736; NM_170737; NM_001276438; NM_001276439; NM_002243; |
| | NM_001276435; NM_001276436; NM_001276437 |
| KDM3A | XM_047445104; XM_047445105; NM_001146688; XM_006712051; |
| | XM_047445101; XM_047445102; NM_018433; XM_047445103 |
| KDM4A | NM_014663 |
| KDM5A | NM_005056; NM_001042603 |
| KDR | NM_002253 |
| KEAP1 | NM_203500; XM_005260174; XM_011528452; XM_005260173; |
| | NM_012289; XM_047439788 |
| KIF20A | NM_005733 |
| KIT | NM_001385284; NM_000222; NM_001385286; NM_001093772; |
| | NM_001385288; NM_001385292; NM_001385290; NM_001385285 |
| KLF4 | NM_004235; NM_001314052 |
| KLF6 | NM_001160125; NM_001008490; NM_001160124; NM_001300; NR_027653 |
| KLK10 | XM_047439102; XM_006723289; XM_005259061; NM_002776; |
| | NM_145888; NM_001077500; XM_017026993; XM_006723287; |
| | XM_005259062 |
| KLK3 | NM_001030050; NM_001030047; NM_145864; NM_001030049; |
| | NM_001030048; NM_001648 |
| KMT2A | XM_011542831; XM_006718839; XM_047426963; XM_011542829; |
| | XM_011542830; XM_047426964; NM_005933; NM_024891; |
| | NM_001197104; XM_011542833 |
| KMT2C | NM_021230; NM_170606 |
| KNG1 | NM_000893; NM_001102416; NM_001166451 |
| KRAS | XM_047428826; NM_001369786; NM_033360; NM_004985; |
| | NM_001369787 |
| KRT19 | NM_002276 |
| LACC1 | XM_011534935; XM_047430105; NM_001350643; NM_001350641; |
| | XM_024449319; XM_047430101; XM_047430109; NM_001350647; |
| | XM_047430103; XM_047430102; XM_047430104; XM_047430106; |
| | NM_001350639; NM_001128303; NM_001350646; NM_001350645; |
| | NM_153218; XM_047430100; XM_047430099; XM_047430108; |
| | NM_001350642; NM_001350644; NM_001350648; XM_047430107; |
| | NM_001350638; NM_001350640 |
| LAMA3 | XM_011525981; XM_017025743; XM_047437504; NM_001127717; |
| | NM_000227; XM_011525978; XM_011525979; XM_047437503; |
| | NM_198129; XM_011525980; XM_017025744; XM_047437506; |
| | XM_011525982; XM_047437505; NM_001302996; NR_130106; |
| | NM_001127718 |
| LAMA4 | XM_047418771; NM_001105206; XM_005266984; NM_002290; |
| | XM_047418769; NM_001105207; NM_001105208; NM_001105209; |
| | XM_047418770; XM_017010854; XM_005266983 |
| LAMB3 | XM_005273124; XM_047420351; NM_001127641; XM_017001272; |
| | NM_000228; NM_001017402 |
| LAMC2 | NM_005562; NM_018891; XM_047420361; XM_047420358; |
| | XM_017001273 |
| LAMP3 | XM_011512688; XM_047447967; XM_005247360; NM_014398 |
| LAPTM5 | XM_011542098; NM_006762 |
| LATS1 | XM_017011477; XM_017011479; NM_001270519; XM_011536252; |
| | NM_001350340; NR_073033; NM_001350339; XM_017011474; |
| | XM_047419521; NM_004690; XR_007059386; XM_047419518; |
| | XM_047419519; XM_047419522; XM_006715603; XM_047419517; |
| | NM_001350392; XM_024446583 |
| LATS2 | XM_017020541; XM_017020542; XM_011535042; XM_047430267; |
| | NM_014572; XM_005266342; XM_047430266 |
| LAX1 | NM_001282878; NM_001136190; NM_017773; XM_006711397 |
| LCK | XM_047420403; XM_011541453; XM_024447046; XM_047420399; |
| | NM_001330468; XM_024447047; NM_005356; NM_001042771 |
| LCP1 | XM_047430303; XM_047430305; NM_002298; XM_047430304; |
| | XM_005266374 |
| LDHA | NR_028500; NM_001165414; NM_001165415; NM_001165416; |
| | NM_005566; NM_001135239 |
| LEF1 | XM_005263047; XR_007057926; XR_007057925; XM_005263046; |
| | XM_006714233; XM_005263048; NM_001130713; NM_001130714; |
| | XR_007057927; NM_001166119; NM_016269 |
| LEP | XM_005250340; NM_000230 |
| LGALS1 | NM_002305 |
| LGALS3 | XM_047431367; XM_047431368; NM_001357678; NR_003225; |
| | NM_002306; XM_047431369; NM_001177388 |
| LGR4 | NM_001346432; NM_018490 |
| LGR5 | XM_047429801; NR_110596; XM_047429800; NM_001277227; |
| | NM_001277226; NM_003667 |
| LGR6 | XM_011509842; XM_047426930; XM_005245404; XM_011509843; |
| | XM_047426929; XM_047426931; NM_021636; XM_047426932; |
| | XM_011509844; XM_011509840; XM_011509838; XM_017001997; |
| | NM_001017403; XM_011509841; XM_017001996; XM_047426928; |
| | NM_001017404; XM_011509839; XM_011509846 |
| LHX6 | XM_047423223; NM_001348190; NM_199160; XM_005251916; |
| | XM_047423224; NM_014368; NM_001242334; NM_001242333; |
| | NM_001242335 |
| LIFR | XM_017009463; XM_047417172; NM_001127671; NM_001364298; |
| | NM_002310; XM_011514042; NM_001364297 |
| LIG4 | NM_001352598; NM_001379095; NM_002312; NM_001352601; |
| | NM_206937; NM_001352602; NM_001352603; NM_001352600; |
| | NM_001330595; NM_001352604; NM_001098268; NM_001352599 |
| LIM2 | NM_001161748; NM_030657 |
| LIMD1 | NM_014240; XM_011534207 |
| LIN28B | NM_001004317; XM_011535818; XM_006715477 |
| LIPA | NR_110233; XM_024448023; NM_001127605; NM_000235; NM_001288979 |
| LIPC | XM_017022176; XM_005254374; XM_006720502; XM_024449916; |
| | XM_005254372; XM_047432491; NM_000236; XM_024449917 |
| LIPE | XM_047438835; XM_006723218; XM_005258938; XM_005258939; |
| | XM_017026810; XM_047438836; XM_005258940; XM_024451514; |
| | XM_005258937; NM_005357 |
| LOX | NM_001317073; NM_001178102; NM_002317 |
| LOXL1 | XM_047432498; XM_017022179; XM_011521555; NM_005576 |
| LRIG1 | NM_001377348; NM_001377347; NM_001377344; XM_017006135; |
| | XM_047447939; NM_001377345; NM_001377346; NM_001377349; |
| | XM_011533578; NM_015541 |
| LRP1 | NM_002332 |
| LRP5 | XM_011545030; XM_017017736; NM_002335; XM_047426948; |
| | XM_047426949; XM_005273994; XM_011545031; NM_001291902; |
| | XM_047426950; XM_011545029; XM_017017735; XR_001747874 |
| LRRC25 | XM_005259739; NM_145256 |
| LRRN3 | NM_018334; NM_001099660; NM_001099658 |
| LSP1 | NM_001242932; NM_001013255; NM_001289005; NM_001013254; |
| | NM_002339; NM_001013253 |
| LTBR | NM_002342; NM_001270987; XM_006718983; XM_005253688 |
| LTF | NM_001199149; NM_001321122; NM_001321121; NM_002343 |
| LTK | XM_024449919; XR_007064447; XM_047432503; NM_002344; |
| | XM_011521557; NM_206961; XM_047432499; XM_047432500; |
| | XM_047432506; NM_001135685; XR_007064446; XM_047432501; |
| | XM_047432502; XM_047432505 |
| LUM | NM_002345 |
| LY75 | NM_002349.4 |
| LY9 | XM_011509556; XM_047420762; NM_001261457; XM_047420755; |
| | XM_017001303; XM_047420771; NM_001033667; NM_001261456; |
| | XM_047420753; XM_047420764; XM_017001304; XM_017001299; |
| | NM_002348; XM_011509549; XM_011509560; XM_017001301; |
| | XM_047420765 |
| LYN | XM_011517529; NM_001111097; NM_002350 |
| LYVE1 | NM_006691 |
| LYZ | NM_000239 |
| MACC1 | NM_182762 |
| MAD2L2 | XM_047430782; NM_001127325; NM_006341 |
| MAF | XM_024450279; NM_001031804; XM_017023233; XR_002957802; |
| | XR_002957804; XR_001751902; XM_017023235; XM_017023234; |
| | NM_005360; XR_002957803 |
| MAFB | NM_005461 |
| MAGEA1 | NM_004988 |
| MAGEA10 | NM_001251828; NM_021048; NM_001011543 |
| MAGEA12 | NM_001166386; NM_001166387; NM_005367 |
| MAGEA2 | NM_001386130.2; NM_005361.3; NM_175742.2; NM_175743.2; |
| | NM_001282501.2; NM_001282502.1; NM_001282504.1; NM_001282505.1 |
| MAGEA2B | NM_001282501; NM_001282505; NM_005361; NM_001282502; |
| | NM_001282504; NM_001386130; NM_175742; NM_175743 |
| MAGEA3 | XM_011531161; XM_005274676; XM_006724818; XM_011531160; |
| | NM_005362 |
| MAGEA4 | NM_001386196; NM_001386197; NM_001386200; NM_002362; |
| | NM_001011550; NM_001386202; NM_001011548; NM_001011549; |
| | NM_001386198; NM_001386203; NM_001386199 |
| MAGEA6 | NM_175868; NM_005363 |
| MALT1 | NM_173844; NM_006785; XM_011525794; XR_007066087 |
| MAML1 | NM_014757 |
| MAML2 | XM_011543023; XM_047427710; NM_032427 |
| MAML3 | XM_047415930; NM_018717; XM_047415929 |
| MAP1LC3B | NM_022818 |
| MAP2K1 | XM_011521783; XM_017022412; XM_017022411; NM_002755 |
| MAP2K2 | NM_030662; XM_047439100; XM_006722799 |
| MAP3K14 | XM_047436998; XM_011525441; XM_047436997; NM_003954 |
| MAP3K7 | XM_006715553; NM_145332; NM_145333; NM_003188; NM_145331 |
| MAP4K1 | XM_011526404; NM_001042600; NM_007181 |
| MAPK1 | NM_138957; NM_002745 |
| MAPK3 | XR_243293; NM_001109891; NM_001040056; NM_002746 |
| MAPKAP1 | NM_001006621; NM_024117; NM_001006617; NM_001006619; |
| | NM_001006620; NM_001006618 |
| MARCO | NM_006770; XM_011512082; XM_011512083; XM_017005171 |
| MASP2 | XR_001736931; NM_006610; XM_017000097; NM_139208; XM_047439758 |
| MATIA | NM_000429 |
| MBD1 | XM_011526002; XM_047437514; XM_047437518; NM_001204137; |
| | NM_001204139; NM_001323949; NM_001399892; NM_001399903; |
| | NM_001399912; NM_001399921; NM_001399932; NM_001399936; |
| | NM_001399940; NM_001399944; NM_001399952; NM_001399953; |
| | NM_001399960; XM_017025770; XM_047437510; XM_047437513; |
| | NM_001323951; NM_001323953; NM_001388139; NM_001388141; |
| | NM_001388144; NM_001388143; NM_001388145; NM_001388146; |
| | NM_001388156; NM_001388162; NM_001388163; NM_001388165; |
| | NM_001399879; NM_001399880; NM_001399881; NM_001399882; |
| | NM_001399887; NM_001399893; NM_001399916; NM_001399922; |
| | NM_001399926; NM_001399929; NM_001399942; NM_001399954; |
| | NM_001399970; NM_001399974; XM_017025776; XM_047437520; |
| | NM_001204141; NM_001204142; NM_001323952; NM_001323954; |
| | NM_001388140; NM_001388147; NM_001388159; NM_001399891; |
| | NM_001399906; NM_001399909; NM_001399913; NM_001399934; |
| | NM_001399935; NM_001399941; NM_001399947; NM_001399949; |
| | NM_001399957; XM_005258271; XM_024451180; NM_001204136; |
| | NM_001388138; NM_001388149; NM_001388166; NM_001399888; |
| | NM_001399890; NM_001399894; NM_001399898; NM_001399899; |
| | NM_001399901; NM_001399900; NM_001399902; NM_001399914; |
| | NM_001399917; NM_001399924; NM_001399937; NM_001399939; |
| | NM_001399967; NM_015846; XM_047437511; XM_047437519; |
| | NM_001204143; NM_001388148; NM_001388151; NM_001388158; |
| | NM_001399884; NM_001399886; NM_001399889; NM_001399904; |
| | NM_001399907; NM_001399910; NM_001399918; NM_001399923; |
| | NM_001399927; NM_001399933; NM_001399948; NM_001399950; |
| | NM_001399958; NM_001399959; NM_001399963; NM_001399975; |
| | NM_015844; XM_047437512; NM_001204138; NM_001388142; |
| | NM_001388152; NM_001388155; NM_001388160; NM_001388161; |
| | NM_001388167; NM_001399883; NM_001399896; NM_001399908; |
| | NM_001399911; NM_001399920; NM_001399925; NM_001399930; |
| | NM_001399966; NM_001399973; NM_001399971; NM_001399976; |
| | NM_015845; XM_017025757; NM_001204140; NM_001388154; |
| | NM_001388157; NM_001388164; NM_001399885; NM_001399897; |
| | NM_001399915; NM_001399919; NM_001399938; NM_001399943; |
| | NM_001399945; NM_002384; NM_015847; XM_011526007; |
| | XM_047437515; XM_047437516; XM_047437517; NM_001204151; |
| | NM_001323942; NM_001323947; NM_001323950; NM_001388150; |
| | NM_001388153; NM_001399895; NM_001399905; NM_001399928; |
| | NM_001399931; NM_001399946; NM_001399955; NM_001399956; |
| | NM_001399961; NM_001399962; NM_001399964; NM_001399965; |
| | NM_001399968 |
| MBD4 | NM_001276271; XM_047449153; NM_003925; NM_001276273; |
| | NM_001276270; NM_001276272; XM_024453810 |
| MCAM | XM_017017762; XM_017017759; XM_017017760; NM_006500; |
| | XM_017017761 |
| MCL1 | NM_001197320; NM_182763; NM_021960 |
| MCM2 | NM_004526; XM_024453531; NR_073375 |
| MCM6 | NM_005915 |
| MDM2 | NM_006880; NM_006882; XM_047428853; NM_006878; NM_001145340; |
| | NM_001278462; NM_001367990; NM_006879; NM_001145337; |
| | NM_002392; NM_006881; NM_032739; NM_001145339; NM_001145336 |
| MECOM | XM_005247214; XM_047447680; XM_047447684; XM_047447688; |
| | XM_047447692; XM_047447693; XM_047447690; XM_047447694; |
| | NM_001366469; NM_001366470; NM_001366474; XM_047447682; |
| | XM_047447686; NM_001366466; NM_001366468; NM_001366473; |
| | XM_005247213; XM_005247221; XM_011512546; NM_001105077; |
| | NM_004991; XM_047447678; NM_001366472; XM_047447677; |
| | NM_001164000; XM_047447679; XM_047447691; NM_001366467; |
| | NM_005241; XM_047447681; XM_047447683; XM_047447685; |
| | XM_047447687; XM_047447689; NM_001105078; NM_001163999; |
| | NM_001205194; NM_001366471 |
| MEFV | NM_001198536; NM_000243 |
| MEN1 | NM_130800; NM_130803; XM_017017768; NM_001370260; |
| | XM_017017765; NM_001370251; NM_001370259; NM_130804; |
| | XM_011545041; NM_130802; XM_017017766; NM_001370262; |
| | NM_001370263; XM_011545040; XM_017017767; NM_001370261; |
| | NM_000244; NM_130799; NM_130801 |
| MERTK | NM_006343 |
| MET | NM_001324402; NM_001324401; NM_001127500; NM_000245; |
| | XM_011516223; XM_047420400 |
| MFGE8 | XM_047432533; NM_001310321; XM_047432535; XM_017022206; |
| | NM_005928; XR_931838; NM_001310319; NM_001310320; |
| | XM_047432534; NM_001114614 |
| MGA | XM_005254243; XM_011521397; XM_011521398; XM_047432295; |
| | XM_047432296; XM_047432308; NM_001400246; XM_047432276; |
| | XM_047432300; XM_047432309; XM_047432312; NM_001080541; |
| | XM_005254254; XM_047432281; XM_047432288; XM_047432291; |
| | XM_047432293; XM_047432307; XM_047432310; XM_047432311; |
| | XM_005254249; XM_005254252; XM_011521399; XM_017022029; |
| | XM_047432283; XM_047432285; XM_047432302; XM_047432306; |
| | NM_001164273; XM_047432280; XM_047432287; XM_047432292; |
| | XM_047432298; XM_047432305; NM_001400243; NM_001400244; |
| | NM_001400247; XM_006720443; XM_047432289; XM_047432290; |
| | XM_047432297; XM_047432303; XM_047432313; XM_047432278; |
| | XM_047432282; XM_047432304; XM_005254246; XM_005254253; |
| | XM_006720445; XM_047432279; XM_047432286; XM_047432299; |
| | XM_047432301; NM_001400225; NM_001400242; NM_001400245 |
| MGAT5 | XM_011511201; XM_047444398; XM_047444406; NM_002410; |
| | XM_047444394; XM_047444397; XM_005263669; XM_047444402; |
| | XM_047444405; XM_006712534; XM_047444401; XM_047444403; |
| | XM_011511202; XM_047444399; XM_047444395; XM_047444404; |
| | XM_011511199; XM_017004148; XM_047444396; XM_047444400; |
| | XM_017004147; XM_017004149; NM_001371457 |
| MGMT | NM_002412 |
| MIF | NM_002415 |
| MITF | NM_001184968; NM_001354605; NM_001354604; NM_001354607; |
| | NM_001354608; NM_006722; NM_198159; NM_198178; NM_198158; |
| | NM_198177; NM_000248; NM_001184967; NM_001354606 |
| MKI67 | NM_002417; NM_001145966; XM_006717864; XM_011539818 |
| MKL1 | NM_001282662; NM_020831; NM_001282660; NM_001318139; |
| | NM_001282661 |
| MLH1 | NM_001258274; XM_047448154; NM_001167619; NM_001258273; |
| | NM_001354618; NM_001354619; NM_001354629; NM_001354630; |
| | NM_000249; NM_001258271; NM_001354624; NM_001354628; |
| | XM_047448152; NM_001354623; NM_001354627; XM_047448153; |
| | NM_001167617; NM_001167618; NM_001354616; NM_001354620; |
| | NM_001354626; XM_005265161; NM_001354617; XM_047448155; |
| | NM_001354615; NM_001354621; NM_001354622; NM_001354625 |
| MLH3 | XR_245681; XM_005267532; XM_047431269; XM_017021219; |
| | XM_005267533; XM_047431268; XR_001750225; XR_007064004; |
| | XM_006720116; XM_024449538; NM_014381; XR_007064005; |
| | XM_005267534; XM_047431265; XM_047431266; NM_001040108; |
| | XM_047431267 |
| MLKL | XM_005255834; XM_047433710; XM_011522936; XM_047433705; |
| | XM_047433708; XM_047433709; XM_047433707; NM_152649; |
| | XM_047433706; NM_001142497; XM_047433704 |
| MLLT3 | NM_001286691; NM_004529 |
| MLST8 | XM_047434466; NM_001199173; NM_001199175; NM_001352057; |
| | XM_047434467; XM_005255479; NM_001199174; NR_147904; |
| | NM_022372; XM_005255475; NR_147905; NR_147906; NM_001352059; |
| | NM_001352060; NR_147907 |
| MLX | NM_198204; NM_198205; NM_170608; NM_170607; NM_013383 |
| MLXIP | NM_014938; XM_047428538; XM_006719290; XM_047428537; |
| | XM_006719293; XM_006719291; XM_006719292 |
| MLXIPL | XM_011516279; XM_047420436; XM_047420437; NM_032953; |
| | NM_032994; XM_011516277; XM_047420433; NM_032951; NR_134541; |
| | XM_017012263; XM_047420435; XM_047420432; NM_032952; |
| | XR_007060040; XM_011516278; NM_032954; XM_047420434 |
| MME | XM_047448157; NM_000902; XM_011512857; NM_001354644; |
| | NM_007288; XM_011512856; NM_001354642; NM_001354643; |
| | XM_006713647; NM_007287; NM_007289 |
| MMP1 | NM_001145938; NM_002421 |
| MMP11 | NM_005940; NR_133013 |
| MMP12 | NM_002426 |
| MMP14 | NM_004995 |
| MMP19 | NM_022790; NR_073606; NM_001272101; XM_011538359; |
| | XM_047428863; XM_047428864; NM_022792; NM_002429; |
| | NM_001032360 |
| MMP2 | NM_001302509; NM_001127891; NM_001302508; NM_001302510; |
| | NM_004530 |
| MMP25 | NM_024302; XM_011525227; NM_001032278; NM_032950; |
| | XM_011525225; XM_011525230; XM_024450943; XM_011525226; |
| | NR_111988; XM_011525229; XM_011525231; XM_011525232; |
| | XM_017025063; XM_017025064; XM_047436731 |
| MMP3 | NM_002422 |
| MMP7 | NM_002423 |
| MMP9 | NM_004994 |
| MMRN1 | NM_001371403; XM_047449832; XM_047449831; NM_007351 |
| MMRN2 | NM_024756 |
| MMS22L | XM_011535678; XM_011535680; NM_001350599; XR_942376; |
| | XM_011535670; XM_047418576; XM_011535672; XM_011535676; |
| | NM_001350600; NM_198468; XM_011535671; XM_011535679; |
| | XM_047418574; XM_047418575; XM_047418582; XM_047418579; |
| | XM_011535675; XM_047418580; XM_006715432; XM_011535674; |
| | XM_047418577; XM_047418581; XM_047418583; XM_047418578 |
| MNDA | NM_002432 |
| MNT | NM_020310; XM_011523869; XM_047436092; XM_024450758; |
| | XM_011523868; XM_017024654 |
| MOB1B | NM_173468; XM_011532412; XM_017008837; NM_001244766; |
| | NM_001244767; XM_005265709 |
| MPG | NM_001015052; NM_002434; NM_001015054 |
| MPO | NM_000250 |
| MPP1 | XM_047442123; XM_047442124; NM_001166462; XM_011531167; |
| | XM_011531169; NM_001166461; XM_024452385; NM_001166460; |
| | NM_002436 |
| MRC1 | NM_002438; NM_001009567 |
| MRPL36 | NM_032479; XM_011514080; XM_017009752; XM_017009751; |
| | XM_017009750; XM_011514079 |
| MS4A1 | NM_021950; NM_152866; NM_152867 |
| MS4A4A | NM_001243266; NM_148975; NM_024021 |
| MS4A6A | XM_011545209; NM_001330275; NM_022349; NM_152851; |
| | XM_005274177; XM_017018125; XM_047427403; NM_001247999; |
| | XM_047427402; NM_152852; XM_024448652; XM_006718660; |
| | XM_006718661 |
| MS4A7 | NM_021201; NM_206940; NM_206939; NM_206938 |
| MSH2 | XM_011532867; NM_000251; XR_001738747; XM_047444416; |
| | NM_001258281; XR_939685; XM_005264332 |
| MSH3 | NM_002439 |
| MSH6 | NM_000179; NM_001281493; NM_001281492; NM_001281494 |
| MSLN | NM_001177355; NM_005823; NM_013404 |
| MSR1 | NM_138716; NM_002445; XM_024447161; NM_138715; NM_001363744 |
| MST1R | XM_005265170; XM_011533742; XM_047448172; XM_047448175; |
| | NR_134919; XM_047448170; XM_047448178; XM_011533744; |
| | XM_047448167; XM_047448180; XM_011533743; NM_002447; |
| | XR_001740155; XM_011533739; XM_011533741; XM_047448162; |
| | XM_047448177; NM_001244937; XM_011533740; XM_047448169; |
| | XM_047448176; XM_047448164; XM_047448165; XM_047448171; |
| | XM_047448174; XM_047448179; XM_047448163; XM_047448166; |
| | XM_047448168; XM_047448173; NM_001318913 |
| MTA1 | XM_047431900; XM_047431902; XM_047431903; XM_011537310; |
| | XM_047431899; XM_047431910; XM_047431904; XM_047431907; |
| | XM_011537305; XM_047431905; NM_001203258; XM_011537307; |
| | XM_047431906; NM_004689; XM_011537306; XM_047431908; |
| | XM_011537308; XM_047431898; XM_011537309; XM_047431901; |
| | XM_047431909 |
| MTAP | NM_001396042; NM_001396044; NM_001396043; NM_001396041; |
| | NR_173242; NM_001396040; NM_001396045; NM_002451 |
| MTOR | NM_001386501; XM_017000900; XM_011541166; NM_001386500; |
| | XR_007058581; XM_047416721; XM_047416724; NM_004958 |
| MTSS1 | XM_017014091; NM_001282974; NM_001363295; NM_001363297; |
| | NM_001363298; XM_006716703; XM_017014088; XM_047422491; |
| | NM_014751; XM_006716702; XM_006716706; XM_047422490; |
| | XM_017014087; XM_017014089; XM_017014090; XM_006716705; |
| | XM_011517403; NM_001363294; NM_001363296; NM_001363301; |
| | NM_001363302; XM_006716701; XM_005251111; XM_006716700; |
| | XM_017014092; XM_006716704; XM_047422488; XM_047422492; |
| | XM_005251113; XM_017014086; XM_047422493; NM_001282971; |
| | NM_001363299; NM_001363300 |
| MUC1 | NM_001018017; NM_001044391; NM_001044393; NM_001204291; |
| | NM_001044390; NM_001204285; NM_182741; NM_001371720; |
| | NM_001204289; NM_001204290; NM_001204293; NM_001018016; |
| | NM_001044392; NM_001204286; NM_001204287; NM_001204288; |
| | NM_001204295; NM_001018021; NM_001204292; NM_001204294; |
| | NM_001204297; NM_001204296; NM_002456 |
| MUM1 | NM_001195286; NR_046000; NR_036585; XM_006715090; |
| | XM_047418730; NM_002460 |
| MUS81 | NM_025128; XM_047427637; NM_001350283; XM_011545270; |
| | XM_047427635; XM_047427638; XM_047427636; NR_146598; |
| | XM_011545269 |
| MUTYH | XM_047421198; NM_001048173; XM_011541497; XM_011541499; |
| | XM_047421202; NM_001293190; NM_001293192; NR_146882; |
| | XM_047421200; XM_047421201; XM_011541503; XM_047421191; |
| | XM_047421194; XM_047421196; NM_001128425; XM_017001334; |
| | XM_047421192; NM_001048172; NR_146883; XM_011541502; |
| | XM_017001332; XM_017001333; XM_047421203; NM_001293196; |
| | NM_001350650; XM_047421195; XM_047421199; NM_001048171; |
| | NM_001048174; NM_001293191; NM_012222; XM_047421193; |
| | XM_047421197; XM_047421204; NM_001293195; NM_001350651 |
| MVP | NM_017458; NM_001293204; NM_005115; NM_001293205 |
| MXD1 | NM_001202513; NM_001202514; NM_002357 |
| MXD3 | NM_001142935; NM_001394987; NM_031300; NM_001394986 |
| MXD4 | NM_006454 |
| MXI1 | NM_005962; NM_001008541; NM_130439 |
| MYB | NM_001161660; NR_134958; NM_001130173; NM_001130172; |
| | NM_001161656; NR_134959; NM_001161657; XM_047418834; |
| | NR_134963; NR_134965; NR_134962; XR_942444; NM_001161659; |
| | NR_134961; NM_001161658; NM_005375; NR_134960; NR_134964 |
| MYCL | NM_001033082; NM_001033081; NM_005376 |
| MYH11 | XM_017023250; NM_002474; NM_022844; NM_001040113; |
| | NM_001040114 |
| MYO1F | XM_011528028; XR_936181; NM_001348355; XM_047438852; |
| | XM_011528027; XR_936182; XR_001753692; NM_012335; XM_011528024 |
| MYO1G | XR_007060129; NM_033054 |
| MZB1 | XM_047417264; NM_016459 |
| NAE1 | NM_001018160; XM_047434835; NM_003905; NM_001286500; |
| | NM_001018159 |
| NBN | NM_002485; XM_011517046; XM_024447163; XM_011517045; |
| | XM_047421796; NM_001024688; XM_047421795 |
| NCAM1 | NM_001386289; NM_001400605; NM_001400607; NM_001386290; |
| | NM_001386291; NM_001400617; NM_001386292; NM_001400603; |
| | NM_001400609; NM_001400611; NM_001076682; NM_001400612; |
| | NM_001400615; NM_001400619; NM_001400621; NM_000615; |
| | NM_001400610; NM_001400618; NM_001400620; NM_001400622; |
| | NM_001400624; NM_001400608; NM_001400614; NM_001242608; |
| | NM_001400613; NM_001400616; NM_001400623; NM_181351; |
| | NM_001242607; NM_001400604; NM_001400606 |
| NCF2 | XM_047421222; XM_047421229; XM_047421238; NM_001190789; |
| | XM_005245207; XM_047421231; NM_001127651; NM_001190794; |
| | NM_000433; XM_011509580; XM_011509581 |
| NCKAP1L | NM_001184976; NM_005337 |
| NCOA4 | NM_001145262; NM_001145261; NM_005437; NM_001145260; |
| | NM_001145263 |
| NCOR1 | XM_005256872; XM_006721602; XM_006721604; XM_017025396; |
| | XM_017025419; XM_047437121; XM_047437128; XM_047437135; |
| | NM_006311; XM_005256868; XM_006721601; XM_017025415; |
| | XM_047437127; XM_047437131; XM_047437132; XM_047437133; |
| | XM_047437136; XM_047437139; XM_047437144; XM_005256866; |
| | XM_011524084; XM_011524085; XM_047437125; XM_047437129; |
| | XM_005256871; XM_017025403; XM_017025409; XM_017025418; |
| | XM_017025420; XM_047437124; XM_047437126; XM_047437141; |
| | XM_006721603; XM_017025417; XM_047437138; XM_047437143; |
| | NM_001190440; XM_011524086; XM_047437122; XM_047437130; |
| | XM_047437142; NM_001190438; XM_005256874; XM_017025397; |
| | XM_017025400; XM_047437123; XM_047437137; XM_047437140; |
| | XM_005256873; XM_005256875; XM_017025401; XM_047437134 |
| NCOR2 | NM_001077261; NM_001206654; NM_006312 |
| NCSTN | NM_001290184; NM_015331; NM_001290186; NM_001349729; |
| | XM_005245053 |
| NDRG1 | NM_001258433; NM_001374844; NM_001135242; NM_001258432; |
| | NM_001374846; NM_006096; NM_001374845; NM_001374847 |
| NDRG2 | NM_016250; NM_001354567; NM_201538; NM_001282215; |
| | NM_001354560; NM_001354561; NM_001354569; NM_201535; |
| | NM_001282216; NM_001354564; NM_001354565; NM_001354566; |
| | NM_201536; NM_201539; NM_201541; NM_001354558; NM_001354562; |
| | NM_001282213; NM_001354570; NM_201540; NM_0012822U; |
| | NM_001320329; NM_001282212; NM_001282214; NM_001354559; |
| | NM_001354568; NM_201537 |
| NDUFA13 | NM_015965 |
| NEIL2 | NM_001349441; NM_001135747; NR_146180; NM_001135746; |
| | NR_146181; NM_001349440; NM_001349442; NM_001349439; |
| | NM_145043; NR_146182; NM_001135748 |
| NEIL3 | NM_018248; XM_047415894 |
| NF1 | NM_001042492; NM_000267; NM_001128147 |
| NF2 | XM_047441386; NM_181828; NM_181830; NM_181826; NM_000268; |
| | NR_156186; NM_181827; NM_181834; NM_016418; NM_181829; |
| | NM_181825; NM_181831; NM_181835; XM_017028809; NM_181832; |
| | NM_181833 |
| NFATC1 | XM_017025783; NM_001278669; NM_001278675; NM_172389; |
| | XM_047437538; NM_001278670; NM_001278672; NM_001278673; |
| | NM_172387; NM_006162; NM_172388; NM_172390 |
| NFATC2 | NM_001258292; XM_017027851; NM_173091; XM_011528826; |
| | NM_001258294; NM_001258296; XM_011528825; NM_001258297; |
| | XM_011528824; NM_001136021; NM_001258295; NM_012340 |
| NFATC3 | NM_173164; NM_173165; NM_004555; NM_173163 |
| NFE2L2 | NM_001313902; NM_001313901; NM_001145413; NM_001313904; |
| | NM_001313903; NM_001145412; NM_001313900; NM_006164 |
| NFKB1 | XM_024454069; NM_001319226; NM_003998; XM_024454068; |
| | XM_047415744; XM_047415742; NM_001382625; NM_001382628; |
| | NM_001165412; NM_001382626; XM_047415743; NM_001382627 |
| NFKB2 | NM_001261403; NM_001077494; NM_001322935; NM_002502; |
| | NM_001288724; NM_001322934 |
| NFKB1E | NM_004556 |
| NHEJ1 | NM_001377498; NM_001377499; NM_024782; NR_165304 |
| NINJ1 | NM_004148; XM_011518716 |
| NLRC3 | XM_047433771; NM_178844; XM_047433769; NR_075083; XM_047433770 |
| NME1 | NM_000269; NM_198175 |
| NMI | XM_047446270; XM_005246941; NM_004688 |
| NMUR1 | XM_011510488; XM_006712196; XM_011510487; NM_006056; |
| | XM_006712195; XM_011510489 |
| NOS2 | NM_153292; NM_000625 |
| NOS3 | NM_001160110; NM_000603; NM_001160109; NM_001160111 |
| NOTCH1 | NM_017617.5 |
| NOTCH2 | NM_017617; XM_011518717 |
| NOTCH3 | NM_024408; NM_001200001 |
| NOX4 | NM_001291926; NM_016931; NM_001143837; NM_001143836; |
| | NM_001291927; XM_017017843; NM_001291929; NM_001300995; |
| | NR_120406 |
| NPAS2 | XM_005263959; XM_005263960; XM_011511242; NM_002518; |
| | XM_017004217; XM_047444503; XM_047444510; XM_047444506; |
| | XM_047444512; XM_047444513; XM_005263961; XM_017004214; |
| | XM_017004216; XM_047444511; XR_007075420; XM_047444504; |
| | XM_047444505; XM_047444515; XM_005263953; XM_011511243; |
| | XM_047444502 |
| NPL | NM_001200051; NM_001200052; NM_030769; NM_001200050; |
| | NM_001200056 |
| NPRL3 | NM_001243248; NM_001039476; NM_001243247; NM_001243249; |
| | NM_001077350; NM_012075 |
| NR3C2 | NR_148974; XM_047415706; XM_047415709; NM_000901; |
| | NM_001166104; XM_011531975; XM_047415708; XM_047415707; |
| | NM_001354819; XM_011531978 |
| NRARP | NM_001004354 |
| NRAS | NM_002524 |
| NRG1 | XM_011544512; XM_017013367; NM_001160007; NM_001322197; |
| | NM_001322201; NM_013958; NM_001160001; NM_001160004; |
| | NM_001322202; NM_001322205; NM_001322207; NM_004495; |
| | NM_013956; NM_001322206; NM_013959; XM_017013371; |
| | XM_017013372; NM_001159996; NM_013960; NM_001160008; |
| | NM_013962; XM_017013368; NM_001159995; NM_001159999; |
| | NM_001160002; NM_001160005; NM_013957; NM_013964 |
| NRP1 | NM_001024628; NR_045259; XM_006717526; XM_011519756; |
| | NM_001244972; NM_001330068; XM_011519755; XM_017016865; |
| | NM_001244973; XM_006717521; XM_047425978; XM_006717525; |
| | XM_047425976; XM_047425977; XM_006717522; XM_006717524; |
| | NM_001024629; NM_003873 |
| NT5C2 | XM_024447901; XM_017015947; XM_047424847; XM_047424851; |
| | XM_047424852; NM_001351172; NM_001351176; NM_001351195; |
| | XM_047424844; XM_047424845; XM_047424846; XM_047424849; |
| | XM_047424854; NM_001351178; NM_001351182; NM_001351184; |
| | NM_00135U85; NM_001351188; NM_001351193; NM_001351196; |
| | XM_047424858; NM_001351171; NM_001351183; NM_001351194; |
| | NM_012229; XM_024447903; XM_047424855; NM_001351169; |
| | NM_00135U73; NM_001351186; NM_001351187; NM_001351191; |
| | XM_047424856; XM_047424857; NM_001134373; NM_001351174; |
| | NM_00135U81; NM_001351197; NM_001351177; NM_001351189; |
| | NM_001351190; NM_001351192; XM_047424853; NM_001351175; |
| | NM_001351179; NM_001351180; XM_005269637; XM_011539537; |
| | XM_024447902; XM_047424848; XM_047424850; XM_047424859; |
| | NM_001351170 |
| NT5E | NM_001204813; NM_002526 |
| NTRK1 | NM_001007792; NM_002529; NM_001012331 |
| NTRK2 | XM_005252001; XM_047423432; NM_001369536; NM_001369539; |
| | NM_001369549; XM_005252003; NM_001018066; NM_001369532; |
| | NM_001369533; NM_001369534; NM_001369544; XM_005252004; |
| | XM_017014751; NM_001369535; NM_001369546; NM_001369550; |
| | XM_011518718; XM_017014752; XM_017014760; NM_001291937; |
| | NM_001369551; NM_001369545; NM_001007097; NM_001018064; |
| | NM_001369541; NM_001369552; XM_017014753; XM_017014755; |
| | NM_001018065; NM_001369542; NM_006180; XM_047423433; |
| | NM_001369537; NM_001369538; NM_001369540; NM_001369543; |
| | NM_001369547; NM_001369548 |
| NTRK3 | XM_006720550; XR_001751292; XM_024449935; XM_047432602; |
| | NM_001375813; XR_002957645; XM_017022245; XM_017022252; |
| | XM_024449934; NM_001375812; XM_006720549; XM_017022241; |
| | XM_017022250; NM_001320135; XM_017022240; XM_047432603; |
| | NM_001012338; XM_006720545; XM_011521638; XM_017022244; |
| | XM_017022251; XM_047432604; NM_001007156; NM_001243101; |
| | XM_017022242; NM_001320134; NM_001375810; NM_001375814; |
| | NM_002530; XM_006720548; XM_017022243; XM_017022254; |
| | NM_001375811; XR_001751293 |
| NUMB | NM_001005745; NM_001320114; NM_001005743; NM_001005744; |
| | NM_003744 |
| NUP214 | NM_005085; NM_001318325; NM_001318324 |
| OGGI | XM_017006497; XM_017006494; XM_047448201; NM_001354648; |
| | NM_001354652; NM_016820; NM_016829; XM_017006493; NM_016819; |
| | NM_002542; NM_016828; NR_148931; XM_047448200; XM_047448202; |
| | NM_001354650; NM_016821; NM_016827; XM_017006495; NM_016826; |
| | NM_001354649; NR_148930; NR_148932; XM_047448203; NM_001354651 |
| OLR1 | XM_047428908; XM_047428909; NM_002543; NM_001172632; |
| | NM_001172633; XM_047428907 |
| OSMR | XM_005248387; NM_001323504; NM_001323506; NM_001323507; |
| | XM_011514161; XR_007058659; XR_007058660; XM_005248384; |
| | XM_047417871; NM_001168355; NM_003999; XM_017010019; |
| | XM_047417870; XM_047417872; NM_001323505; XR_925661; |
| | XM_005248386; XM_047417873 |
| P2RX1 | XM_006721529; XM_011523898; XM_047436159; XM_047436160; |
| | XM_047436161; NM_002558; XM_011523897; XM_047436158; |
| | XM_011523899; XM_011523900 |
| P2RY10 | NM_001324221; NM_001324225; NM_014499; NM_001324218; |
| | NM_198333; XM_047441998 |
| P2RY13 | XM_006713664; NM_023914; NM_176894 |
| PADI2 | NM_007365; XM_017000148; XM_047442975 |
| PAH | NM_001354304; NM_000277; XM_017019370 |
| PAK1 | XM_024448560; NM_001376272; NM_001376278; NM_001376283; |
| | NM_001376291; NM_001376294; NM_001376304; NM_001376305; |
| | NR_164797; XM_024448558; NM_001376274; NM_001376286; |
| | NM_001376290; NM_001376292; NM_001376295; NM_001376302; |
| | NM_002576; XM_047427053; NM_001376271; NM_001376288; |
| | NM_001376301; XM_024448557; XM_047427047; XM_047427051; |
| | NM_001376275; NM_001376293; NR_164798; XM_024448559; |
| | XM_047427046; NM_001376281; NM_001376284; NM_001376285; |
| | XM_047427052; NM_001128620; NM_001376269; NM_001376273; |
| | XM_047427049; XM_047427054; NM_001376268; NM_001376277; |
| | NM_001376303; XM_047427048; XM_047427050; NM_001376270; |
| | NM_001376276; NM_001376279; NM_001376280; NM_001376282; |
| | NM_001376287; NM_001376289 |
| PAK4 | XM_024451314; NM_001014832; NM_001014835; XM_047438036; |
| | XM_047438040; NM_001014833; XM_011526316; NM_001014831; |
| | XM_047438034; XM_047438035; NM_001014834; NM_001394501; |
| | XM_047438038; XM_047438037; XM_011526317; XM_047438041; |
| | NM_005884; XM_011526320 |
| PALB2 | NM_024675; XM_011545946; XM_011545947; XM_017023673; |
| | XM_011545948 |
| PALLD | XM_011531768; XM_011531769; NM_001166110; NM_001367567; |
| | NM_016081; XM_011531771; XM_011531773; XM_047449862; |
| | NM_001166109; XM_024453940; XM_047449868; NM_001166108; |
| | NM_001367568; XM_011531774; XM_011531775; XM_047449867; |
| | NM_001367569; XM_047449861; XM_047449864; XM_047449865; |
| | NM_001367570; XM_011531772; XM_024453939; XM_047449863; |
| | XM_047449869; XM_047449870; XM_047449866 |
| PAPPA | XM_017014784; NM_002581; XM_006717129 |
| PARD3 | NM_001184793; NM_001184785; NM_001184789; NM_001184790; |
| | NM_001184792; NM_019619; NM_001184786; NM_001184787; |
| | NM_001184788; NM_001184791; NM_001184794 |
| PARP1 | NM_001618 |
| PARP15 | XM_005247160; XM_011512476; XM_005247159; XM_017005791; |
| | XM_017005792; XM_047447580; XM_047447584; XM_011512475; |
| | NM_001113523; XM_047447582; NM_001308320; XM_011512480; |
| | XM_011512479; XM_011512477; XM_047447583; NM_001308321; |
| | NM_152615 |
| PARP2 | XM_047430869; XM_005267247; NM_005484; NM_001042618; |
| | XM_017020912 |
| PARPBP | NM_001382722; NM_001400863; NM_001400879; NM_001400883; |
| | NM_001400893; NM_001400897; NM_001400907; XM_017019541; |
| | NM_001382721; NM_001382723; NM_001382724; NM_001382726; |
| | NM_001400861; NM_001400868; NM_001400874; NM_001400875; |
| | NM_001400889; NM_001400926; XM_047429056; NM_001319996; |
| | NM_001382732; NM_001382735; NM_001400857; NM_001400859; |
| | NM_001400884; XM_047429051; XM_047429052; NM_001382725; |
| | NM_001400856; NM_001400858; NM_001400862; NM_001400867; |
| | NM_001400870; NM_001400871; NM_001400873; NM_001400888; |
| | NM_001400890; NM_001400892; NM_001400902; NM_001400928; |
| | XR_007063095; XR_007063096; XR_007063097; XM_047429057; |
| | NM_001319988; NM_001400854; NM_001400855; NM_001400866; |
| | NM_001400872; NM_001400878; NM_001400895; NM_001400927; |
| | NM_017915; XM_047429048; XM_011538515; XM_047429050; |
| | NM_001319993; NM_001400853; NM_001400860; NM_001400881; |
| | NM_001400886; NM_001400887; NM_001400904; XM_011538514; |
| | XM_047429053; XM_047429055; NM_001319994; NM_001319995; |
| | NM_001400864; NM_001400865; NM_001400876; NM_001400877; |
| | NM_001400885; NM_001400891; NM_001400894; XM_047429049; |
| | XM_047429054; NM_001382728; NM_001382729; NM_001382731; |
| | NM_001400869; NM_001400880; NM_001400896; NM_001400903; |
| | NM_001400905; NM_001400906; NM_001400925; NM_001400929 |
| PARVG | NM_001254742; NM_022141; NM_001137605; NM_001254743; |
| | XM_047441455; NM_001254741; NM_001137606 |
| PAX5 | NM_001280547; NM_001280553; NM_016734; NM_001280548; |
| | NR_103999; NM_001280551; NM_001280555; NM_001280554; |
| | NM_001280552; NM_001280556; NM_001280549; NM_001280550; |
| | NR_104000 |
| PBK | NM_018492; NM_001278945; NM_001363040 |
| PBRM1 | XM_017006734; XM_017006742; XM_047448451; XM_047448465; |
| | NM_001394872; NM_001394874; NM_001400496; NM_001405560; |
| | NM_001405561; NM_001405568; NM_001405571; NM_001405579; |
| | NM_001405581; NM_001405601; NM_001405607; NM_001405614; |
| | NM_001405618; NM_001405622; NM_001405628; NM_001405633; |
| | NM_001405635; NM_181042; XM_011533900; XM_017006725; |
| | XM_017006744; XM_047448447; XM_047448449; XM_047448456; |
| | XM_047448459; NM_001350076; NM_001366070; NM_001366074; |
| | NM_001394868; NM_001394876; NM_001394877; NM_001394878; |
| | NM_001394880; NM_001400474; NM_001400504; NM_001405553; |
| | NM_001405557; NM_001405563; NM_001405564; NM_001405569; |
| | NM_001405573; NM_001405588; NM_001405592; NM_001405597; |
| | NM_001405602; NM_001405610; NM_001405613; NM_001405621; |
| | NM_001405623; NM_001405627; NM_001405630; NM_001405631; |
| | NM_001405634; NM_001405640; NM_018165; XM_017006726; |
| | XM_017006728; XM_047448443; XM_047448448; XM_047448457; |
| | XM_047448460; NM_001350074; NM_001350077; NM_001366072; |
| | NM_001366073; NM_001394870; NM_001400470; NM_001400487; |
| | NM_001405572; NM_001405576; NM_001405582; NM_001405585; |
| | NM_001405589; NM_001405600; NM_001405616; NM_001405620; |
| | NM_001405626; NR_174502; XM_017006741; XM_047448442; |
| | XM_047448445; XM_047448458; XM_047448464; NM_001394873; |
| | NM_001394881; NM_001400472; NM_001400484; NM_001405555; |
| | NM_001405559; NM_001405565; NM_001405570; NM_001405596; |
| | NM_001405608; NM_001405612; NM_001405619; NM_001405641; |
| | XM_047448452; XM_047448455; NM_001350075; NM_001366076; |
| | NM_001394867; NM_001394879; NM_001400479; NM_001400501; |
| | NM_001405574; NM_001405578; NM_001405580; NM_001405583; |
| | NM_001405587; NM_001405590; NM_001405593; NM_001405611; |
| | NM_001405625; NM_001405632; NM_001405638; NM_001405643; |
| | NM_018313; NR_175959; NM_181041; XM_017006730; XM_017006731; |
| | XM_047448446; XM_047448453; XM_047448463; NM_001350078; |
| | NM_001366071; NM_001394869; NM_001394875; NM_001400481; |
| | NM_001405556; NM_001405603; NM_001405605; NM_001405609; |
| | NM_001405637; NM_001405642; XM_011533902; XM_047448444; |
| | XM_047448461; XM_047448462; NM_001350079; NM_001366075; |
| | NM_001400471; NM_001400475; NM_001400490; NM_001400500; |
| | NM_001405554; NM_001405558; NM_001405566; NM_001405567; |
| | NM_001405584; NM_001405591; NM_001405595; NM_001405598; |
| | NM_001405629; NM_001405639; NM_001405636; XM_011533903; |
| | XM_005265280; XM_017006727; XM_024453619; XM_047448450; |
| | XM_047448454; NM_001394871; NM_001400473; NM_001405552; |
| | NM_001405575; NM_001405577; NM_001405586; NM_001405594; |
| | NM_001405599; NM_001405604; NM_001405606; NM_001405615; |
| | NM_001405617; NM_001405624 |
| PBX3 | XM_047423442; XM_047423444; NM_006195; XM_011518755; |
| | XM_047423445; NM_001134778; NM_001330782; NR_024122; |
| | XM_006717130; NR_024123; XM_047423443; XM_047423441 |
| PCNA | NM_182649; NM_002592 |
| PCOLCE | NM_002593 |
| PDCD1 | NM_005018; XM_006712573 |
| PDCD1LG2 | XM_005251600; NM_025239 |
| PDE4C | NM_001330172; XM_047438917; NM_001369701; NM_001098819; |
| | XM_047438918; NR_040546; XM_047438919; XM_047438916; |
| | NM_000923; NM_001098818; NM_001395274 |
| PDGFC | XM_047415970; XM_017008455; NM_016205; XM_047415971; |
| | XM_047415969; XM_047415972; NR_036641 |
| PDGFRA | XM_047415767; NM_001347828; NM_001347829; XM_005265743; |
| | XM_017008281; NM_001347827; XM_047415766; NM_001347830; |
| | NM_006206; XM_006714041 |
| PDGFRB | NM_001355016; NM_002609; NM_001355017; NR_149150 |
| PDPK1 | XM_047434199; XM_011522523; XM_047434198; NM_002613; |
| | NM_031268; XM_047434201; XM_047434202; NM_001261816; |
| | XM_011522521; XM_024450296; XM_047434200 |
| PDPN | XM_047434471; NM_001006625; NM_198389; NM_001385053; |
| | NM_001006624; XM_006710295; NM_006474; NM_013317; |
| | XM_024451404 |
| PEBP1 | NM_002567 |
| PER1 | NM_002616 |
| PFKFB3 | XM_047425346; NM_001282630; XM_005252464; XM_017016329; |
| | NM_004566; NM_001363545; XM_047425341; XM_011519493; |
| | XM_047425345; XM_047425349; NR_136554; NM_001145443; |
| | XM_047425343; XM_047425344; NM_001323016; NM_001323017; |
| | XM_047425342; XM_047425347; NM_001314063 |
| PGLYRP2 | NM_052890; NM_001363546 |
| PGR | XM_011542869; NM_001271161; NR_073142; XM_006718858; |
| | NM_000926; NM_001202474; NM_001271162; NR_073141; NR_073143 |
| PIAS3 | NM_006099 |
| PIK3AP1 | XM_005269499; XM_047424566; NM_152309; XM_011539248 |
| PIK3CA | NM_006218; XM_006713658 |
| PIK3CB | XM_011512895; XM_047448307; NM_001256045; XM_017006619; |
| | NM_006219; XM_047448311; XM_047448312; XM_047448314; |
| | XM_047448315; XM_047448309; XM_006713659; XM_047448308; |
| | XM_047448310; XM_047448313 |
| PIK3CD | XM_024447663; XM_047422552; XM_047422561; XM_047422568; |
| | XM_047422573; XM_047422574; XM_047422575; XM_047422577; |
| | XM_024447664; XM_047422553; XM_047422564; XM_047422566; |
| | NM_005026; XM_047422567; XM_047422569; NM_001350234; |
| | XM_047422554; XM_047422555; XM_047422589; XM_006710689; |
| | XM_047422550; XM_047422557; XM_006710687; XM_047422558; |
| | XM_047422559; XM_047422563; XM_047422565; XM_047422580; |
| | XM_047422551; XM_047422556; XM_047422562; XM_047422570; |
| | XM_047422571; NM_001350235; XM_047422560; XM_047422572; |
| | XM_047422576; XM_047422578 |
| PIK3CG | XM_017012328; XM_005250443; XM_047420479; NM_001282426; |
| | XM_011516317; XM_047420481; XM_047420480; NM_001282427; |
| | XM_011516316; NM_002649 |
| PIK3IP1 | NM_001135911; NM_052880 |
| PIK3R1 | XM_017009585; XM_047417315; NM_181504; NM_181524; |
| | XM_047417316; NM_001242466; NM_181523; XM_005248542; |
| | XM_047417317 |
| PIK3R2 | NR_073517; NM_005027; NR_162071 |
| PIK3R3 | NM_001114172; NM_001328651; NM_001303429; NM_001328653; |
| | NM_001328649; NR_137329; NM_001328648; NM_001328652; |
| | NM_001328650; NM_001328654; NM_001303428; NM_003629 |
| PIK3R5 | NM_001388398; NM_001388400; NM_001388396; NM_014308; |
| | XM_047435711; NM_001142633; NM_001251853; XM_047435710; |
| | NM_001251852; NM_001388397; XM_047435709; NM_001251851; |
| | NM_001251855; NM_001388399 |
| PIM1 | NM_002648; NM_001243186 |
| PIM3 | NM_001001852 |
| PINX1 | NM_001284356; NM_017884 |
| PIPOX | NM_016518 |
| PKP1 | NM_000299; NM_001005337 |
| PKP2 | NM_001005242; NM_004572 |
| PLA2G7 | XM_047419360; NM_001168357; XM_005249408; NM_005084; |
| | XM_047419359 |
| PLCB2 | XM_047432672; XM_047432683; XM_017022317; XM_047432676; |
| | XM_047432679; NM_004573; XR_007064458; XM_017022314; |
| | XM_047432670; NM_001284297; XM_047432678; XM_047432681; |
| | NM_001284298; NM_001284299; XM_047432669; XM_047432671; |
| | XM_047432673; XM_047432674; XM_047432677; XM_047432682; |
| | XM_047432689; XM_017022319; XM_047432675; XM_047432684; |
| | XM_047432686; XM_047432667; XM_047432668; XM_047432680; |
| | XM_047432687; XM_047432685; XM_047432688 |
| PLCG2 | NM_002661 |
| PLEK | NM_002664; XM_047444772 |
| PLG | NM_000301; NM_001168338 |
| PLIN1 | NM_002666; XM_005254934; NM_001145311 |
| PLK1 | NM_005030 |
| PLOD2 | XM_017006625; NM_000935; XM_047448320; NM_182943; |
| | XM_047448319 |
| PLXNC1 | XM_047428050; XM_011537730; NR_037687; NM_005761; |
| | XM_006719186; XM_011537731 |
| PMAIP1 | NM_021127; NM_001382616; NM_001382615; NM_001382617; |
| | NM_001382618; NM_001382623 |
| PML | NM_033238; NM_033240; NM_033249; NM_033244; NM_033247; |
| | NM_033250; NM_002675; NM_033239; NM_033246 |
| PMS1 | XM_017004344; NM_001128143; NM_001321045; NM_001321044; |
| | XM_006712596; XM_017004347; NM_000534; NM_001289408; |
| | NM_001321049; NM_001321051; NR_110332; XM_017004348; |
| | XM_017004350; XM_024452967; XM_047444777; NM_001321046; |
| | XM_047444775; XM_047444776; NM_001321047; XM_024452966; |
| | XM_047444778; NM_001128144; NM_001289409; NM_001321048 |
| PMS2 | XM_024446800; NM_001322004; NM_001322006; NM_001322013; |
| | NM_001322007; NM_001322010; NM_001322014; XM_047420483; |
| | NM_001322003; NM_001322008; XM_047420485; XM_047420486; |
| | NM_001322005; NM_001322009; NM_001322015; XM_047420484; |
| | XM_047420482; NM_001322011; NM_001322012; NM_000535; NR_136154 |
| PNKD | XM_017003771; XM_017003772; NM_015488; NM_001077399; |
| | NM_022572 |
| PNPLA2 | NM_020376 |
| POLB | XM_005273538; XM_017013583; XM_005273537; XM_005273540; |
| | XM_017013584; XM_047421901; XM_047421900; XM_005273536; |
| | XM_005273539; NM_002690; XM_005273535; XR_428311 |
| POLD1 | XM_005259008; XM_047438946; NM_001256849; XM_011527038; |
| | XM_047438948; XM_047438949; XM_047438950; NM_002691; |
| | XR_935835; NM_001308632; XM_017026882; XM_047438947; NR_046402 |
| POLD2 | XM_047420498; XM_047420501; NM_006230; NM_001256879; |
| | XM_024446802; XM_047420497; XM_047420500; NM_001127218; |
| | XM_047420499 |
| POLD3 | XM_011544734; NM_001363597; NM_006591; NR_046409; NR_046410; |
| | XM_005273716; XM_047426295 |
| POLD4 | NM_001256870; NR_046412; NM_021173; NR_046411; NR_046413 |
| POLE | XM_011534797; XM_011534799; NM_006231; XM_047429018; |
| | XR_941395; XM_011534795; XM_011534802 |
| POLE2 | XM_047431483; XM_047431484; NM_001197330; NM_001348384; |
| | XR_007064015; NM_001348385; NM_002692; NM_001197331; |
| | XR_007064016 |
| POLE3 | NM_001278255; NM_017443; NR_027261 |
| POLE4 | NM_019896 |
| POLG | NM_001126131; NM_002693 |
| POLH | NM_001291970; NM_006502; XM_047418900; NM_001291969 |
| POLI | NM_001351612; NM_001351615; NM_001351620; NM_001351621; |
| | NM_007195; NM_001351617; NM_001351611; NM_001351618; |
| | XM_011525797; NM_001351613; NM_001351632; NR_147257; |
| | NM_001351616; NM_001351610; NM_001351614; XM_024451081; |
| | XM_005258192; NM_001351619 |
| POLK | NM_001387111; NM_001395900; NM_016218; NM_001387112; |
| | NM_001395894; NR_170560; NM_001387110; NR_170559; |
| | NM_001395893; NM_001395899; NM_001395901; NM_001345922; |
| | NM_001395897; NR_144315; NM_001345921; NM_001387113; |
| | NM_001395902 |
| POLL | XM_011539664; XM_017016088; XM_047425089; XM_011539650; |
| | XM_011539656; XM_024447943; XM_047425087; XM_047425092; |
| | XM_047425095; XM_011539657; XM_024447942; XM_047425094; |
| | NR_033406; XM_011539662; XM_017016090; XM_024447945; |
| | XM_047425090; NM_001174085; XM_006717775; XM_017016085; |
| | XM_017016091; XM_047425093; NM_001174084; XM_047425086; |
| | XM_047425097; XM_047425098; NM_013274; XM_006717777; |
| | XM_047425088; XM_047425091; XM_047425096; NM_001308382; |
| | XM_011539651; XM_011539654; XM_011539655; XM_017016084 |
| POLM | NR_156113; NR_156112; NM_001284330; NM_001284331; |
| | NM_001362683; NM_013284; NR_104299 |
| POLN | NM_181808 |
| POLQ | NM_199420; NM_006596 |
| PON1 | NM_000446 |
| POU2AF1 | XM_006718860; XM_017017932; XM_006718859; XM_005271593; |
| | XM_047427137; NM_006235 |
| PPARG | NM_001354669; NM_001354670; NM_001374263; NM_001330615; |
| | NM_001374262; NM_005037; NM_001374261; NM_138711; NM_138712; |
| | NM_001374264; NM_001374266; NM_001354668; NM_015869; |
| | NM_001354666; NM_001354667; NM_001374265 |
| PPM1D | NM_003620; XR_934577; XR_007065507 |
| PPP1R13B | XM_017021117; XM_047431170; XR_001750206; XM_047431171; |
| | NM_015316; XM_011536593; XR_007063997; XR_001750205; |
| | XM_017021116; XM_017021119; XM_005267487; XM_047431172; |
| | XR_001750204 |
| PPP1R16B | NM_001172735; NM_015568; XM_011528768; XM_047440086 |
| PPP1R9B | NM_032595 |
| PPP2R1A | NM_001363656; NM_014225; NR_033500 |
| PPP3CC | XM_047421941; XM_047421942; NM_001243975; NM_005605; |
| | XR_007060744; NM_001243974 |
| PRC1 | XM_011522187; XM_047433307; NM_003981; XM_017022712; |
| | XM_011522189; XM_011522191; XM_047433299; XM_047433301; |
| | XM_047433306; XM_047433308; NM_199413; XM_011522190; |
| | XM_017022713; XM_017022714; XM_047433297; XM_047433304; |
| | XM_047433314; XM_017022716; XM_047433313; NM_199414; |
| | XM_005254987; XM_006720759; XM_006720760; XM_011522192; |
| | XM_047433295; XM_047433303; XM_047433305; XM_047433310; |
| | XM_011522188; XM_047433298; XM_047433300; XM_047433309; |
| | XM_047433311; NM_001267580 |
| PRDM1 | XM_047419248; XM_047419247; XM_011536064; XM_017011187; |
| | XM_011536062; XM_047419246; XM_006715550; NM_182907; |
| | NM_001198 |
| PRF1 | NM_005041; NM_001083116 |
| PRG2 | NM_001302926; NM_002728; NM_001243245; NM_001302927 |
| PRKACA | NM_207518; NM_002730; XM_047439070; NM_001304349; |
| | XM_017026948 |
| PRKCA | XM_024450830; XR_007065317; XM_017024836; XR_007065316; |
| | XR_001752558; XM_047436389; XM_017024837; XM_047436388; |
| | NM_002737; XM_024450829; XR_007065315 |
| PRKCB | NM_212535; NM_002738; XM_047434365 |
| PRKCD | XM_047448564; NM_212539; NM_001316327; NM_001354676; |
| | NM_001354680; NM_001354678; NM_006254; NM_001354679; |
| | XR_007095706 |
| PRKCI | XM_047448574; NM_002740; XM_047448575 |
| PRKCQ | XM_005252497; NM_001282645; NM_001282644; NM_001323267; |
| | XM_005252496; NM_001323266; NM_006257; NM_001242413; |
| | NM_001323265 |
| PRKDC | NM_001081640; NM_006904 |
| PRLR | XM_011514068; NM_001204315; NM_001204318; XM_047417390; |
| | NM_001204317; NR_037910; XM_047417388; NM_000949; |
| | NM_001204316; XM_006714484; XM_047417391; NM_001204314; |
| | XM_024446131 |
| PRMT7 | XM_017023304; XM_047434217; XM_047434218; XM_047434222; |
| | XM_047434223; XM_047434235; XM_047434239; XM_011523112; |
| | XM_011523121; XM_047434219; XM_047434225; XM_047434227; |
| | NM_001378022; XR_007064886; XR_007064887; XR_007064888; |
| | XM_011523113; XM_011523115; XM_017023297; XM_047434237; |
| | NM_001378020; NM_001378023; NM_019023; NR_165365; |
| | XR_001751915; XM_047434224; XM_047434226; NM_001184824; |
| | XM_011523116; XM_017023298; XM_017023301; XM_047434228; |
| | XM_047434232; NM_001351143; NM_001378021; NR_165366; |
| | XM_047434229; XM_047434231; NM_001290018; NR_147058; |
| | NR_165368; NR_165369; NR_165373; XR_007064885; XM_017023300; |
| | XM_047434220; XM_047434221; XM_047434233; XM_047434234; |
| | XM_047434238; NM_001351144; NR_147056; XM_017023292; |
| | XM_017023296; XM_017023299; XM_047434230; XM_047434236; |
| | NM_001378018; NR_147057; NR_165367; NR_165370; NR_165371; |
| | NR_165372; XR_002957814 |
| PRODH2 | NM_001195226; NM_001368249; NM_016335; NM_001368250 |
| PRSS33 | NM_001385462; NM_001385463; NM_001385464; NM_152891; NR_169625 |
| PRTN3 | XM_011528136; NM_002777 |
| PSAP | NM_001042466; NM_001042465; NM_002778 |
| PSEN2 | XM_017001836; XM_047425596; XM_047425597; XM_047425601; |
| | NM_000447; NM_012486; XR_001737316; XR_007061979; XR_949150; |
| | XM_005273199; XR_007061980; XM_017001835 |
| PSENEN | NM_001281532; NM_172341; NM_018468 |
| PSMB1 | NM_002793 |
| PSMB5 | NM_001144932; NM_002797; NM_001130725 |
| PTAFR | NM_001164722; NM_000952; NM_001164723; NM_001164721 |
| PTCH1 | NR_149061; NM_001083604; NM_001083605; NM_001083607; |
| | NM_001083606; NM_001354918; NM_001354919; NM_000264; |
| | NM_001083602; NM_001083603 |
| PTGDR | XM_005267891; NM_000953; NM_001281469 |
| PTGS2 | NM_000963 |
| PTK7 | NM_152880; NM_152882; NM_152881; XM_047419157; NM_002821; |
| | NR_072997; NR_072998; NM_152883; NM_001270398; XM_011514766; |
| | XM_011514765 |
| PTPN22 | XM_011541225; NM_001193431; XM_047417632; XM_011541223; |
| | XM_017001006; NM_015967; XM_011541221; XM_011541222; |
| | NM_012411; XM_017001005; XM_047417630; XM_047417631; |
| | NM_001308297 |
| PTPN6 | XM_011520988; NM_002831; XM_047429231; XM_024449106; |
| | NM_080548; XM_047429232; NM_080549 |
| PTPRB | XM_017019724; NM_001206971; NM_001109754; NM_001330204; |
| | XM_006719529; NM_002837; XR_944651; XM_006719528; |
| | XM_011538614; NM_001206972 |
| PTPRC | XM_047426420; NM_001267798; NM_002838; NR_052021; NM_080922; |
| | XM_006711473; XM_006711474; XM_047426417; XM_047426409; |
| | XM_047426381; XM_006711472; XM_047426398; XM_047426415; |
| | NM_080921 |
| PTPRCAP | NM_005608 |
| PTPRD | XM_017014958; XM_017014963; XM_017014968; XM_017014976; |
| | XM_047423645; XM_047423646; XM_047423649; NM_001040712; |
| | NM_001377947; NM_130391; XM_006716827; XM_017014970; |
| | XM_017014971; XM_017014983; XM_047423643; XM_047423644; |
| | XM_047423660; NM_001378058; XM_017014965; XM_017014967; |
| | XM_017014979; XM_047423650; NM_001377958; XM_017014964; |
| | XM_017014974; XM_017014977; XM_017014978; XM_047423648; |
| | XM_047423652; XM_047423655; XM_047423658; NM_001377946; |
| | NM_002839; NM_130392; XM_017014966; XM_047423654; NM_130393; |
| | XM_017014972; XM_017014980; XM_024447625; XM_047423642; |
| | XM_047423647; XM_047423656; XM_017014961; XM_017014969; |
| | XM_017014982; XM_047423641; XM_047423651; XM_047423653; |
| | XM_047423657; XM_047423659; NM_001171025; XM_006716817; |
| | XM_006716823; XM_006716825 |
| PTTG1 | NM_001282382; NM_001282383; NM_004219 |
| PTTG1IP | NM_001286822; NR_104597; NM_004339 |
| PVRIG | NM_024070; NM_001397246; NM_001387134 |
| RAB22A | NM_020673 |
| RAC1 | NM_198235; NM_002933; NM_198232; NM_198234 |
| RAC2 | XM_006724286; NM_002872 |
| RACGAP1 | XM_017019221; XM_017019226; XM_017019227; NM_001126103; |
| | NM_001319999; XM_047428748; NM_001320002; XM_017019222; |
| | XM_017019223; NM_001320005; XM_024448958; NM_001126104; |
| | NM_001320003; XM_047428746; XM_047428750; NM_001320000; |
| | NM_001320006; XM_006719359; XM_011538238; XM_047428747; |
| | XM_047428751; NM_013277; XM_017019224; XM_017019225; |
| | NM_001320004; XM_047428745; XM_047428749; NM_001320001; |
| | NM_001320007 |
| RAD1 | NM_133282; NM_002853; NM_133377; NM_001033673; NR_026591 |
| RAD17 | XM_017009681; XM_047417459; XM_047417462; NM_133344; |
| | XM_047417461; NM_133338; XM_047417456; XM_047417457; |
| | NM_002873; NM_133341; XM_047417458; NM_001278622; NM_133339; |
| | NM_133342; XM_047417460; NM_133340; NM_133343 |
| RAD23B | NM_001244713; NM_001244724; NM_002874 |
| RAD50 | NM_005732; NM_133482 |
| RAD51 | XM_011521859; XM_011521861; NM_002875; XM_011521857; |
| | XM_011521858; NM_001164269; XM_047432925; XM_011521860; |
| | NM_001164270; NM_133487 |
| RAD51C | XM_006722001; NR_103873; XM_006722002; NR_103872; |
| | XM_006722004; XM_011525094; XM_047436505; NM_058216; |
| | NM_002876 |
| RAD51D | NR_037711; NM_001142571; NM_133629; NR_037712; NM_133628; |
| | NM_133630; NM_002878; NM_133627 |
| RAF1 | XM_017006966; XM_047448649; NR_148942; XM_011533974; |
| | NM_001354691; NR_148940; NM_002880; NM_001354690; |
| | NM_001354692; NM_001354695; NM_001354689; NM_001354693; |
| | NM_001354694; NR_148941; XM_047448650; XM_047448651 |
| RALBP1 | NM_006788; XM_047437280; XM_047437284; XM_047437278; |
| | XM_047437281; XM_047437282; XM_047437283; XM_047437279 |
| RALGPS2 | XM_047423755; XM_006711410; XM_047423766; NM_018037; |
| | NR_174383; NM_001400042; NM_001286247; NR_174384; NM_152663; |
| | XM_047423777 |
| RARA | XM_047436508; NM_001024809; NM_001145302; XM_005257552; |
| | NM_001033603; XM_005257553; XM_005257554; XM_047436507; |
| | NM_000964; NM_001145301; XM_011525095; XM_011525096; |
| | XM_047436506 |
| RASA1 | NM_002890; NM_022650 |
| RASAL2 | XM_047434860; XM_005245622; XM_011510166; XM_017002850; |
| | XM_017002854; XM_047434849; XM_047434857; XM_047434859; |
| | NM_170692; XM_017002849; XM_017002853; XM_017002852; |
| | XM_017002855; XM_047434838; XM_011510167; NM_004841; |
| | XM_017002851; XM_047434837; XM_047434839 |
| RASAL3 | XM_047439231; NR_174477; NR_174478; XM_011528187; |
| | NM_001400377; XM_011528186; NM_001400378; NM_001400381; |
| | NM_022904; NM_001348027; NM_001348028; NM_001400379; |
| | NM_001400380 |
| RASGRP1 | XM_047432077; NM_001128602; NM_005739; XM_047432073; |
| | XM_047432076; XM_047432078; XM_047432074; NM_001306086; |
| | XM_047432075 |
| RASGRP3 | XM_047443878; NM_001139488; NM_001349978; XM_047443879; |
| | NM_001349977; NM_001349981; NM_001349976; NM_001349979; |
| | NM_001349980; XM_011532746; XM_047443877; NM_001349975; |
| | NM_170672; XM_011532748; XM_017003761; NM_015376 |
| RASGRP4 | NM_001146205; NM_001146204; XR_935732; NM_001146207; |
| | NM_170604; NM_052949; NM_001146203; NM_170603; NM_001146202; |
| | NM_001146206; NM_170602 |
| RASIP1 | NM_017805 |
| RASSF1 | NM_007182; XM_011533316; NM_170716; NM_001206957; NM_170712; |
| | NM_170713; NM_170714; XM_047447372; NM_170717; NM_170715 |
| RASSF10 | NM_001080521 |
| RASSF5 | NM_182663; NM_031437; NM_182664; NM_182665 |
| RB1 | NM_000321 |
| RBBP8 | XM_006722520; XM_047437728; NM_002894; XM_047437730; |
| | XM_005258325; XM_011526132; XM_047437729; NM_203291; |
| | XM_047437731; XM_047437732; NM_203292; XM_006722519; |
| | XM_047437727; XM_006722521 |
| RBM10 | NM_152856; XM_047442556; NM_001204466; NM_005676; |
| | XM_047442555; XM_047442551; NM_001204467; XM_047442554; |
| | XM_047442552; XM_047442553; XM_005272677; XM_005272678; |
| | XM_005272679; NM_001204468 |
| RBM15 | NM_014092; NM_022768; NM_001201545 |
| RBPJ | XM_047415658; NM_203283; NM_001374400; NM_001379408; |
| | XM_017008171; XM_047415656; NM_001379406; NM_001379409; |
| | NM_001363577; NM_001374401; NM_001374402; NM_005349; |
| | NM_001374403; XM_011513840; XM_047415657; NM_015874; |
| | NM_203284; NM_001379407 |
| RBX1 | NM_014248 |
| RCSD1 | NR_136519; NM_052862; NM_001322923; NM_001322924 |
| RDH16 | NM_001320108; NM_003708 |
| RECQL4 | XM_047422440; XM_047422445; XM_047422446; XM_047422439; |
| | XM_047422448; XM_047422437; XM_047422438; XM_047422443; |
| | XM_047422447; XM_047422442; NM_004260; XM_047422444; |
| | XM_011517384; XM_047422441 |
| RECQL5 | XM_005257818; XM_006722186; XM_047437087; NM_001003715; |
| | NM_004259; XM_011525486; XM_047437089; XM_011525485; |
| | XM_047437086; XM_047437090; XM_005257822; XM_047437091; |
| | XM_047437088; XM_047437085; NM_001003716; XM_011525484; |
| | XM_047437092 |
| RELA | XM_011545206; NM_001404659; NM_001404660; NM_021975; |
| | XM_011545207; NM_001145138; NM_001404662; NM_001404663; |
| | NM_001404657; NM_001243984; XM_047427392; NM_001404658; |
| | NM_001404661; NM_001243985 |
| RELB | XM_005259128; XM_005259127; XM_047439189; NM_006509; |
| | XM_047439190 |
| REPS2 | XM_011545605; XM_047442627; XM_024452479; XM_005274625; |
| | XM_011545603; XM_011545604; XM_047442626; XM_005274626; |
| | XM_011545607; XM_017029955; XM_017029956; NM_001080975; |
| | NM_004726; XM_017029958; XM_011545606; XM_017029957; |
| | XM_047442628 |
| RET | NM_020975; NM_001355216; NM_020630; NM_020629; NM_000323 |
| REV3L | XM_011536028; XM_011536036; XM_047419216; NM_001372078; |
| | XM_047419217; XM_047419218; NM_001286431; XM_011536029; |
| | NM_001286432; XM_047419215; XM_017011155; XM_047419220; |
| | XM_011536030; XM_011536032; XM_047419219; NM_002912 |
| RGN | XM_006724568; XM_017029954; NM_004683; NM_001282848; |
| | NM_152869; NM_001282849; XM_006724567 |
| RHEB | XM_047420685; XM_024446854; XM_011516457; NM_005614 |
| RHOBTB2 | XM_047421607; NM_001160036; XM_047421609; XM_047421608; |
| | NM_001160037; NM_001374791; XM_047421610; XM_047421611; |
| | NM_015178 |
| RHOH | XM_047415675; NM_001278361; NM_001278364; XM_017008189; |
| | NM_001278360; NM_001278363; NM_001278359; NM_001278365; |
| | NM_001278362; XM_047415674; NM_001278369; NM_001278367; |
| | NM_001278368; XM_011513692; NM_001278366; NM_004310 |
| RHOT1 | XM_047436363; NM_001288758; XM_047436353; XM_047436357; |
| | XM_047436359; XM_047436364; XM_047436354; XM_047436356; |
| | NM_001033566; NM_018307; XM_047436362; NM_001033568; |
| | NM_001288754; XM_011524973; XM_047436361; XM_047436355; |
| | XM_047436358; NM_001033567; NM_001288755; NR_110083; |
| | XM_011524969; XM_047436360 |
| RICTOR | XM_006714463; XM_017009311; NM_001285440; NM_152756; |
| | NM_001285439; XM_017009312; XM_011514005; XM_047417068; |
| | XM_047417069; XM_011514006; XM_047417070 |
| RIT1 | NM_001256820; NM_006912; NM_001256821 |
| RLTPR | XM_011522874; XM_047433644; XM_011522875; XM_017022953; |
| | NM_001013838; XM_047433645; NM_001317026 |
| RMI1 | NM_001358293; XM_017015140; NM_001358292; NM_024945; |
| | NM_001358291; NM_001358294 |
| RNF43 | XM_011524956; XM_047436330; NM_017763; XM_047436331; |
| | NM_001305544; XM_017024800; XM_047436332; NM_001305545 |
| ROBO4 | NM_019055; XM_006718861; NM_001301088; XM_011542875 |
| ROR1 | NM_005012; XM_011541526; XM_017001376; XM_017001377; |
| | NM_001083592 |
| ROR2 | XM_005252008; XM_017014762; XM_047423434; XM_047423436; |
| | XM_006717121; XM_047423435; NM_004560; XM_005252009; |
| | XM_047423437; NM_001318204 |
| RORC | XM_006711484; NM_001001523; NM_005060; XM_047427201 |
| ROS1 | XM_011536053; XM_011536055; XM_011536054; XM_011536057; |
| | XM_011536049; XM_011536058; XM_047419232; NM_001378891; |
| | XM_006715548; NM_002944; XM_011536050; XM_017011173; |
| | XM_047419231; XM_011536051; XM_011536056; XM_017011172; |
| | NM_001378902 |
| RP2 | NM_006915 |
| RPN2 | XM_006723852; NM_001135771; NM_001324299; XM_006723851; |
| | NM_001324301; NM_001324302; NM_001324306; NM_002951; |
| | NM_001324303; NM_001324304; NM_001324305 |
| RPS6 | NM_001010 |
| RPS6KA3 | XM_017029719; XM_047442335; XM_011545561; XM_011545556; |
| | XM_011545562; XM_047442333; NM_004586; XM_005274577; |
| | XM_011545557; XM_011545560; XM_017029718; XM_047442332; |
| | XM_047442334; XM_047442336; XM_017029717; XM_005274573; |
| | XM_011545555 |
| RPS6KB1 | NM_001369679; NR_161459; NM_001369673; NR_161455; |
| | NM_001272043; NR_161462; XM_011525102; NM_001272060; |
| | NM_001369674; NM_001369678; NR_161461; NM_001272044; |
| | NM_001369669; NM_001369670; NM_001369671; NM_001369677; |
| | NM_001272042; NM_001369672; NR_161456; NR_161458; NR_161460; |
| | NM_001369675; NM_001369676; NR_161457; NM_003161 |
| RPTOR | NM_001163034; NM_020761 |
| RRAGC | NM_022157; NM_001271851 |
| RRM1 | NM_001033; NM_001330193; NM_001318065; NM_001318064 |
| RRM2 | NM_001034; NR_164157; NR_161344; NM_001165931 |
| RRP1B | NM_015056 |
| RSF1 | NM_016578; XM_005274051; XM_017017923 |
| RSPO1 | XM_006710583; NM_001242909; NM_001242908; NM_001242910; |
| | NM_173640; NM_001038633 |
| RUNX1 | XM_047441010; XM_047441012; XM_047441013; XM_005261069; |
| | XM_047441007; XM_047441016; XM_005261068; XM_011529767; |
| | XM_011529770; XM_047441015; XM_011529766; XM_047441009; |
| | NM_001001890; NM_001122607; XM_047441014; XM_011529768; |
| | XM_047441011; NM_001754 |
| RUNX2 | NM_001278478; NM_001369405; NM_004348; NR_103533; |
| | NM_001024630; NR_103532; NM_001015051 |
| RUNX3 | NM_001031680; NM_004350; XM_011542351; XM_005246024; |
| | XM_047433131; NM_001320672 |
| RYK | NM_002958; NM_001005861; XR_007095716 |
| S1PR5 | NM_001166215; NM_030760 |
| SAMD3 | XM_024446333; XM_024446335; NM_001277185; XM_017010309; |
| | XR_001743174; NM_152552; NM_001258275; XM_017010308; |
| | XM_024446337; XM_047418239; XM_017010305; XM_017010310; |
| | NM_001017373 |
| SAMSN1 | XM_011529684; NM_001256370; NM_001395858; XM_047440942; |
| | NM_001286523; NM_022136; XM_011529685; XM_011529686; |
| | XM_047440941; NM_001256579; NM_001395856; NM_001395857 |
| SASH1 | XM_017010598; XM_017010600; XM_017010605; XM_024446384; |
| | XM_047418497; XM_047418498; NM_015278; XM_017010599; |
| | XM_024446385; NM_001346505; NM_001346506; NM_001346507; |
| | NM_001346508; XM_047418499; XM_047418496; XM_047418500; |
| | NM_001346509 |
| SASH3 | NM_018990; XM_006724763 |
| SATB1 | XM_011533990; NM_001322872; NM_001322874; XM_011533988; |
| | NM_002971; NM_001322873; NM_001131010; NM_001195470; |
| | NM_001322876; NM_001322871; XM_047448683; XM_011533989; |
| | NM_001322875 |
| SATB2 | NM_015265; NM_001172517; NM_001172509; NR_134967; |
| | XM_005246396; XM_047443775 |
| SAV1 | XM_011537057; NM_021818; XM_047431659 |
| SDC1 | NM_001006946; XM_005262620; XM_005262621; NM_002997; |
| | XM_005262622 |
| SEC11C | NM_001307941; NM_033280; XM_011526260 |
| SELE | NM_000450 |
| SELL | NR_029467; NM_000655 |
| SEMA3A | XM_005250110; XM_047419751; NM_006080 |
| SEMA3F | XM_047448701; NM_004186; XM_047448700; NM_001318800; |
| | NM_001318798; XM_005265381; XM_006713289; XM_006713290; |
| | XM_005265382; XM_011533998; XM_011534000; XM_047448699 |
| SERPINA7 | XM_005262180; NM_000354; XM_006724683 |
| SERPINB2 | XM_024451192; NM_002575; NM_001143818 |
| SERPINB5 | NM_002639; XM_006722483 |
| SERPIND1 | NM_000185 |
| SERPINE1 | NM_000602; NM_001386463; NM_001386465; NM_001386457; |
| | NM_001386466; NM_001165413; NM_001386456; NM_001386460; |
| | NM_001386461; NM_001386462; NM_001386459; NM_001386464; |
| | NM_001386458 |
| SERPINF2 | XM_005256701; NM_001165920; XM_047436303; NM_000934; |
| | XM_017024765; NM_001165921 |
| SERPINH1 | XM_047427800; NM_001207014; XM_024448756; XM_011545327; |
| | NM_001235 |
| SF3B1 | XM_047443838; NM_012433; XM_047443841; XM_047443839; |
| | XM_047443840; NM_001005526; NM_001308824 |
| SFRP1 | NM_003012 |
| SFRP2 | NM_003013 |
| SFRP4 | NM_003014 |
| SH2D1A | NM_001114937; NM_002351 |
| SH2D1B | NM_053282 |
| SH3BP2 | NM_001145856; NM_001122681; NM_001145855; NM_003023 |
| SHH | NR_132319; NM_000193; NR_132318; XM_011516480; XM_011516479; |
| | XM_047420718; NM_001310462 |
| SIGLEC1 | NM_001367089; NM_023068 |
| SIGLEC14 | XM_047437991; NM_001098612 |
| SIGLEC5 | NM_001384708; NM_001384709; NM_003830; XM_047446914; |
| | XM_047446915 |
| SIGLEC8 | XM_011526734; NM_014442; NM_001363548 |
| SIGLEC9 | XM_011526732; NM_014441; NM_001198558; XM_047438615; |
| | XM_047438616 |
| SIRPA | XM_011529173; XM_047439919; XM_047439917; XM_024451836; |
| | XM_005260670; XM_047439915; XM_047439916; NM_001330728; |
| | XM_047439918; XM_047439920; NM_001040023; NM_080792; |
| | NM_001040022 |
| SIRPB2 | XM_047440129; XM_047440130; NR_021484; XR_007067453; |
| | XM_005260709; XM_047440127; NM_001122962; XM_047440128; |
| | XR_007067452; XM_005260708; NM_001134836; XR_007067451 |
| SIRT1 | NM_001314049; NM_012238; NM_001142498 |
| SIRT3 | NM_001370314; NM_001370317; NR_163396; NR_163397; XR_007062467; |
| | XM_017017431; NM_001370310; NM_001370318; NM_001370320; |
| | NR_163387; NR_163392; NM_001017524; NM_001370322; NR_163400; |
| | NM_001370319; NR_163388; NR_163398; NR_163401; XM_011519957; |
| | NR_163386; NR_163390; NR_163391; NR_163393; NR_163394; |
| | NM_001370312; NM_001370323; NR_163395; XM_011519956; |
| | XM_047426677; NM_001370316; NM_001370321; NM_001370324; |
| | NM_001370325; NM_012239; NR_163389; NR_163399; NR_163402; |
| | NM_001370315 |
| SIX2 | NM_016932; XM_005264100 |
| SKAP1 | XM_047436975; XM_047436978; XM_047436979; XM_047436980; |
| | XM_005257755; XM_047436976; XM_047436977; NM_001075099; |
| | XM_017025258; XM_047436972; XM_047436974; XM_047436973; |
| | NM_003726 |
| SKP2 | NM_032637; NM_001243120; NM_005983; XM_047417536; XR_001742203 |
| SLA | NM_006748; XM_047422110; NM_001045556; XM_047422108; |
| | NM_001045557; XM_047422109; NM_001282964; XM_047422107; |
| | NM_001282965 |
| SLA2 | NM_175077; NM_032214 |
| SLAMF6 | XM_047443866; NM_001184714; NM_001184715; NM_052931; |
| | NM_001184716; XM_017000216 |
| SLAMF7 | XM_011509828; XM_011509829; NM_001282589; NM_001282590; |
| | NM_001282596; NM_001282591; NM_001282593; NM_001282588; |
| | NM_001282595; XM_047426359; NM_001282592; NM_001282594; |
| | NM_021181 |
| SLC10A1 | NM_003049 |
| SLC16A4 | NM_001319220; NM_004696; XM_006711033; XM_047433990; |
| | XM_005271317; XM_047433986; NM_001201547; NM_001201549; |
| | NM_001201548; NM_001201546; XM_047433967; XM_047433980 |
| SLC22A7 | XM_011514257; NM_153320; XM_024446313; XM_047418105; |
| | XM_017010199; XM_047418102; NM_006672; XM_047418103; |
| | XM_047418104; XM_011514262; XM_017010198; XM_011514256 |
| SLC27A5 | NM_012254; XM_011526364; XM_017026214; NM_001321196 |
| SLC2A2 | XM_011513087; NM_000340; NM_001278659; XM_047448761; |
| | NM_001278658 |
| SLC39A6 | NM_001099406; NM_012319 |
| SLC6A2 | XM_011523295; XM_047434510; XM_047434511; XM_011523299; |
| | XM_011523300; XM_047434512; XM_047434513; NM_001172502; |
| | NM_001043; NM_001172501; XM_006721263; NM_001172504 |
| SLX1A | NM_001014999.3; NM_001015000.2 |
| SMAD6 | NM_005585; XR_931827; NM_001142861; XM_011521561; NR_027654 |
| SMAD7 | NM_005904; NM_001190821; NM_001190822; NM_001190823; |
| | XM_047437509 |
| SMARCA4 | XM_024451667; NM_001128845; NM_001387283; NR_164683; |
| | XM_047439249; NM_001128848; XM_047439243; XM_047439246; |
| | XM_047439247; XM_047439251; XM_006722846; XM_024451661; |
| | XM_047439245; NM_001374457; XM_047439250; NM_001128846; |
| | XM_011528198; XM_024451663; NM_001128847; XM_047439244; |
| | NM_001128844; NM_001128849; NM_003072; XM_024451658; |
| | XM_047439248 |
| SMO | NM_005631; XM_047420759 |
| SMUG1 | XM_011538119; XM_047428641; XM_047428652; XM_047428654; |
| | NM_001243787; NM_001351239; NM_001351240; NM_001351242; |
| | NM_001351245; NM_001351250; NM_001351254; NM_001351261; |
| | XR_007063064; XM_011538116; XM_047428643; XM_047428650; |
| | XM_047428651; NM_001351256; NM_001351262; NM_014311; |
| | XM_047428639; XM_047428645; NM_001351241; NM_001351243; |
| | NM_001351252; XM_011538121; XM_047428635; XM_047428637; |
| | XM_047428640; XM_047428646; XM_047428655; NM_001243789; |
| | NM_001351237; NM_001351258; NM_001351259; XM_047428636; |
| | XM_047428642; XM_047428644; NM_001243790; NM_001351244; |
| | NM_001351248; NM_001351253; XM_006719319; XM_047428632; |
| | XM_047428649; XM_047428653; NM_001243788; NM_001351249; |
| | NM_001351251; NM_001351255; NM_001351260; XM_011538112; |
| | XM_047428633; XM_047428634; XM_047428638; XM_047428648; |
| | NM_001243791; NM_001351257; XM_011538118; NM_001351238; |
| | NM_001351246; NM_001351247 |
| SMURF1 | XM_047420635; XM_047420637; NM_020429; XM_017012457; |
| | NM_181349; NM_001199847; XM_047420636 |
| SNAI2 | NM_003068 |
| SNCA | XM_011532204; NM_001146054; NM_000345; NM_001375287; |
| | XM_011532206; NM_007308; XM_047416097; NR_164675; |
| | XM_011532207; NM_001375288; NM_001375290; NR_164676; |
| | XM_011532203; XM_011532205; NR_164674; NM_001146055; |
| | NM_001375286; NM_001375285 |
| SNX20 | NM_001144972; NM_153337; NM_182854 |
| SOD2 | NM_001322817; NM_001322820; NM_001322815; NM_001322814; |
| | NM_000636; NM_001322816; NM_001024465; NM_001024466; |
| | NM_001322819 |
| SOS1 | XM_047445586; XM_011533064; XM_047445584; XM_047445585; |
| | XM_047445582; NM_001382395; NM_005633; XM_047445581; |
| | XM_047445583; NM_001382394 |
| SOST | NM_025237 |
| SOX11 | NM_003108 |
| SOX18 | NM_018419 |
| SOX30 | NM_001308165; NM_178424; NM_007017 |
| SOX9 | NM_000346 |
| SP140 | XM_017003249; XM_047443078; XM_011510515; XM_011510516; |
| | XM_011510517; XM_017003250; XM_017003253; XM_047443073; |
| | XM_047443076; XM_047443077; NM_001278452; NM_001278453; |
| | XM_011510520; XM_017003245; XM_017003246; XM_017003252; |
| | XM_047443074; XM_005246253; XM_005246255; XM_011510518; |
| | XM_017003247; XM_005246252; XM_005246256; XM_017003248; |
| | XM_047443079; XM_047443080; NM_001278451; XM_017003242; |
| | XM_005246254; XM_006712223; XM_017003240; XM_017003243; |
| | XM_047443072; XM_047443081; NM_007237; XM_011510519; |
| | XM_017003239; NM_001005176 |
| SPARC | NM_001309443; NM_001309444; NM_003118 |
| SPEN | NM_015001 |
| SPHK1 | NM_021972; NM_001355139; NM_001142602; NM_182965; |
| | NM_001142601 |
| SPI1 | XM_017018173; NM_003120; XM_047427487; NM_001080547 |
| SPIB | NM_001243999; NM_001243998; NM_001244000; NM_003121 |
| SPN | NM_001030288; NM_003123 |
| SPOCK1 | NM_004598 |
| SPP1 | NM_001251829; NM_001040060; NM_001251830; NM_000582; |
| | NM_001040058 |
| SPP2 | NM_006944; XM_011511699; XM_005246102; XM_011511698; |
| | XM_011511700 |
| SPRY2 | NM_001318537; NM_005842; NM_001318536; NM_001318538 |
| SQLE | NM_003129; XM_011517246 |
| SSBP2 | XM_047417058; NM_001400348; NM_001400358; NR_174545; |
| | NR_174546; NR_174563; XM_047417059; XM_047417066; |
| | NM_001345886; NM_001400343; NM_001400345; NM_001400346; |
| | NM_001400366; NR_174529; NR_174531; NR_174536; NR_174548; |
| | NR_174558; XM_017009308; XM_047417065; NM_001400356; |
| | NM_001400368; NM_001400369; NM_001400375; NM_012446; |
| | NR_174525; NR_174528; NR_174535; NR_174537; NR_174544; |
| | NR_174547; NR_174561; NM_001400347; NM_001400349; |
| | NM_001400351; NM_001400352; NM_001400364; NM_001400371; |
| | NR_174538; NR_174540; NR_174552; XM_047417061; NM_001256733; |
| | NM_001256736; NM_001394351; NM_001400344; NM_001400359; |
| | NM_001400360; NM_001400363; NM_001400367; NM_001400374; |
| | NR_174530; NR_174539; NR_174543; NR_174553; NR_174555; |
| | NR_174560; XM_047417062; NM_001256732; NM_001400340; |
| | NM_001400350; NM_001400355; XM_047417060; NM_001256734; |
| | NM_001256735; NM_001394350; NM_001394352; NM_001400341; |
| | NM_001400342; NM_001400353; NM_001400357; NM_001400361; |
| | NM_001400362; NR_174526; NR_174541; NR_174542; NR_174550; |
| | NR_174554; NR_174562; XM_017009309; XM_047417064; |
| | NM_001400354; NR_174527; NR_174532; NR_174533; NR_174534; |
| | NR_174549; NR_174551; NR_174556; NR_174557; NR_174559 |
| SSR4 | XM_047442390; XM_047442389; NM_001204527; NM_006280; |
| | NR_037927; NM_001204526 |
| STAB1 | XM_047447774; XM_006713065; XM_005264974; XM_047447777; |
| | NM_015136; XM_005264973; XM_047447775; XM_047447776 |
| STAT1 | NM_001384890; NM_139266; NM_001384881; NM_007315; |
| | NM_001384891; XM_006712718; NM_001384882; NM_001384889; |
| | NM_001384883; NM_001384885; NM_001384880; NM_001384884; |
| | NM_001384888; NM_001384886; NM_001384887 |
| STAT2 | XM_047429470; XM_047429471; XR_007063122; XM_047429472; |
| | NM_001385111; NM_001385115; NM_005419; NM_001385110; |
| | NM_001385113; XM_011538699; XM_011538698; XM_047429469; |
| | NM_001385114; XM_011538697; XR_245953; XM_047429468; NM_198332 |
| STAT4 | XM_047445601; XM_047445609; XM_047445602; XM_047445604; |
| | NM_003151; XM_006712719; XM_047445606; XM_047445605; |
| | XM_047445607; NM_001243835; XM_047445603; XM_047445608; |
| | XM_047445600 |
| STAT5A | NM_001288719; XM_047436591; XM_047436590; NM_001288720; |
| | NM_003152; XM_047436589; NM_001288718; XM_047436588; |
| | XM_005257624 |
| STAT5B | XM_005257626; XM_047436593; XM_024450898; XM_024450897; |
| | NM_012448; XM_017024977 |
| STAT6 | NM_001178078; NM_001178080; NM_001178081; XM_047429475; |
| | NM_001178079; XM_047429476; XM_047429473; XM_047429477; |
| | NM_003153; XM_047429474; NR_033659 |
| STC2 | NM_003714 |
| STK11 | NM_000455 |
| STK3 | XM_017013756; XM_047422133; XR_007060752; NM_001256312; |
| | XM_011517248; XM_047422137; NM_006281; XR_007060753; |
| | XM_011517257; XM_017013757; XM_017013761; XM_047422132; |
| | XM_047422135; NM_001256313; XR_007060755; XM_011517252; |
| | XM_047422134; XM_047422136; XR_007060754; XM_011517251; |
| | XM_017013758 |
| STMN1 | NM_203399; NM_203401; NM_152497; NM_005563; NM_001145454 |
| STUB1 | NM_001293197; NM_005861 |
| STX11 | XM_047419437; XM_011536213; XM_011536217; XM_047419436; |
| | XM_011536214; XM_047419438; NM_003764; XM_047419440; |
| | XM_011536218; XM_047419439; XM_047419441 |
| SUFU | XM_011539864; XM_047425335; XM_047425339; NM_016169; |
| | XM_011539863; XM_047425337; NM_001178133; XM_011539858; |
| | XM_047425336; XM_011539860; XM_011539861; XM_047425338 |
| SULT2A1 | NM_003167 |
| SUSD6 | NM_014734 |
| SYK | XM_047423811; XM_005252147; XM_047423809; NM_001174168; |
| | XM_011518946; XM_047423810; NM_001174167; NM_001135052; |
| | NM_003177 |
| SYNE1 | XM_006715416; XM_006715417; XM_006715420; XM_017010611; |
| | XM_017010614; XM_017010617; XM_047418508; XM_006715414; |
| | XM_017010608; XM_017010615; NM_033071; XM_006715410; |
| | XM_047418503; XM_047418505; XM_047418513; NM_001347702; |
| | XM_006715411; XM_006715425; XM_047418504; NM_001134379; |
| | NM_015293; XM_006715412; XM_006715422; XM_006715424; |
| | XM_011535642; XM_017010613; XM_017010616; XM_017010618; |
| | XM_017010619; XM_047418502; XM_047418507; XM_047418509; |
| | NM_182961; NM_133650; XM_006715407; XM_006715421; |
| | XM_011535644; XM_047418501; XM_047418512; NM_001099267; |
| | XM_006715408; XM_017010609; XM_017010612; XM_047418506; |
| | XM_047418510; XM_047418511; XM_006715409; XM_006715413; |
| | XM_006715415; XM_006715423; XM_011535641; XM_011535643; |
| | XM_011535645; XM_017010610; NM_001347701 |
| TACSTD2 | NM_002353 |
| TAF1 | XM_047442392; NM_001286074; XM_047442400; XM_047442396; |
| | XM_047442403; NR_104389; NR_104392; XM_047442391; |
| | XM_047442399; XM_047442404; XM_047442405; NR_104391; |
| | NR_104393; NR_104388; NR_104394; XM_024452430; XM_047442393; |
| | XM_047442406; NM_004606; NR_104387; NR_104390; NR_104395; |
| | NR_104396; XM_005262300; XM_047442394; XM_047442397; |
| | XM_047442398; XM_047442401; NM_138923; XM_047442395; |
| | XM_047442402 |
| TAGAP | NM_001278733; NM_138810; NM_054114; NM_152133 |
| TAGLN | NM_001001522; NM_003186 |
| TAOK2 | XM_011545983; XM_011545984; XM_011545986; NM_004783; |
| | XM_047434922; NM_016151; NM_001252043; XM_011545985; |
| | XM_047434918; XM_047434920; XM_047434921; XM_011545982; |
| | XM_047434917; XM_047434919 |
| TAOK3 | XM_017019410; NM_001346487; NM_001346494; NM_016281; |
| | XM_047428945; XM_047428947; NM_001346489; NM_001346490; |
| | XM_006719445; XM_017019409; XM_047428952; NM_001346488; |
| | NM_001346492; NM_001346495; XM_047428941; XM_047428948; |
| | XM_047428951; XM_047428953; NM_001346491; NM_001346493; |
| | XM_017019408; XM_047428942; XM_047428944; NM_001346497; |
| | XM_005253898; XM_011538437; XM_024449010; XM_047428943; |
| | XM_047428946; XM_047428950; XM_047428954; NM_001346496 |
| TARDBP | NM_007375; XR_007058559; XR_007058563; XR_007058564; |
| | XR_007058558; XR_007058560; XR_007058562; XR_007058561 |
| TAT | NM_000353 |
| TBC1D10C | XM_011545002; NM_001369495; XM_047426913; NM_001369492; |
| | NM_001256508; NM_001369494; NM_198517; XM_006718539; |
| | XM_047426910; NM_001369498; XM_006718538; XM_047426911; |
| | XM_047426914; NM_001369496; NR_046266; XM_047426909; |
| | NM_001369497 |
| TBX21 | NM_013351 |
| TBX3 | NM_005996; NM_016569 |
| TCF21 | NM_003206; NM_198392 |
| TCF7 | XM_006714679; XM_006714686; XM_006714685; XM_047417637; |
| | XM_047417638; XM_047417639; XM_011543606; XM_006714684; |
| | XM_047417636; XM_047417640; XM_047417643; NM_001134852; |
| | XM_006714682; NM_201634; XM_006714678; XM_047417634; |
| | XM_047417642; NM_001346425; NM_213648; XM_011543604; |
| | XM_047417641; NM_001366502; NM_003202; XM_047417633; |
| | XM_047417635; NM_001134851; NM_001346450; NM_201632; NR_033449 |
| TCF7L1 | XM_006712109; NM_031283 |
| TDG | XM_047429489; XM_047429488; XM_047429487; NM_001363612; |
| | NM_003211; XM_047429486; NM_001008411 |
| TEAD1 | NM_021961 |
| TEAD2 | XM_011527404; XM_047439523; XM_011527403; XM_047439531; |
| | XM_047439535; XM_047439537; XM_047439540; NM_001256658; |
| | NM_003598; XM_047439538; XM_047439541; XM_005259334; |
| | XM_011527402; XM_047439525; XM_047439530; NM_001256662; |
| | XM_047439526; XM_047439529; XM_047439533; XM_047439534; |
| | XM_047439536; XM_047439542; XM_047439543; NM_001256660; |
| | XM_006723428; XM_011527400; XM_047439520; XM_047439522; |
| | XM_047439524; XM_047439527; XM_011527401; XM_011527405; |
| | XM_011527406; XM_047439532; XM_047439539; XM_047439544; |
| | NM_001256659; NM_001256661; XM_006723429; XM_011527399; |
| | XM_047439521; XM_047439528 |
| TEAD3 | NM_001395214; NM_003214 |
| TEAD4 | NM_201443; NM_003213; NM_201441 |
| TEK | NM_001375475; NM_000459; NM_001290077; NM_001290078; |
| | NM_001375476 |
| TERT | NR_149162; NM_198255; NM_198253; NR_149163; NM_001193376; |
| | NM_198254 |
| TES | NM_015641; NM_152829 |
| TESPA1 | XM_006719715; XM_047429930; NM_001136030; NR_147068; |
| | XR_007063147; XM_011539035; NR_147064; NR_147065; NR_147072; |
| | NR_147073; XM_017020262; XM_047429929; NM_001261844; |
| | NM_001351152; NR_147066; NR_147071; XM_017020263; |
| | NM_001351149; NR_147069; XM_011539037; NM_001098815; |
| | NM_001351151; NM_014796; NR_147067; NM_001351150; |
| | NM_001351154; NM_001351155; NR_147062; NR_147063; NR_147070; |
| | XM_047429931; NM_001351148; NM_001351153; XR_007063146 |
| TF | NM_001063; NM_001354703; NM_001354704 |
| TFEB | XM_006715212; NM_001271943; NM_001271945; NM_001167827; |
| | XM_047419361; NM_007162; NM_001271944; XM_005249411 |
| TFR2 | NM_003227; NM_001206855 |
| TGFB1 | NM_000660; XM_011527242 |
| TGFB2 | NM_003238; NR_138149; NR_138148; NM_001135599 |
| TGFB3 | NM_001329938; NM_003239; NM_001329939 |
| TGFBR2 | NM_003242; XM_011534045; XM_011534043; NM_001024847; |
| | XM_047448787 |
| TGM3 | NM_003245 |
| THBD | NM_000361 |
| THBS2 | NM_003247; NM_001381940; NM_001381941; NM_001381939; |
| | NR_167745; NM_001381942; NR_167744 |
| THEMIS | XM_047418763; XM_047418766; XM_047418767; NM_001164687; |
| | XM_047418764; NM_001318531; NM_001394521; XM_047418765; |
| | NM_001164685; NM_001394520; NM_001394522; NM_001010923 |
| THY1 | NM_006288; NM_001311162; NR_164077; NM_001311160; NM_001372050 |
| TIAM1 | NM_001353685; NM_001353686; NM_001353688; NM_001353692; |
| | NM_001353689; NM_001353687; NM_001353690; NM_001353691; |
| | NM_003253; NM_001353694; NM_001353693; XM_005261040; |
| | XM_047440969; NM_001353684 |
| TIE1 | XM_047429354; XM_005271163; NM_001253357; XM_017002207; |
| | XM_047429343; NM_005424 |
| TIGIT | XM_047447672; XM_047447671; NM_173799 |
| TIMP1 | NM_003254 |
| TIMP2 | NM_003255 |
| TJP1 | XM_011521972; XM_047432982; XM_047432984; NM_001330239; |
| | XM_005254619; XM_017022523; XM_017022524; XM_047432988; |
| | XM_047432990; NM_001301025; NM_001355014; XM_017022525; |
| | XM_047432991; NM_003257; XM_017022522; XM_017022526; |
| | XM_047432983; XM_047432986; XM_047432989; XM_047432981; |
| | XM_047432985; NM_001301026; XM_017022521; XM_017022527; |
| | NM_001355013; XM_005254620; XM_047432987; NM_001355012; |
| | NM_001355015; NM_175610 |
| TJP2 | XM_011519206; NM_001369871; NM_001369872; XM_011519208; |
| | XM_011519209; NM_001369870; NM_004817; XM_047424090; |
| | XM_011519207; XM_047424094; NM_001369874; NM_001170630; |
| | XM_047424092; NM_001369875; XM_047424091; NM_001170415; |
| | NM_001170416; XM_047424095; NM_001170414; NM_001369873; |
| | NM_201629 |
| TJP3 | XM_047438611; NM_001267560; NM_001267561; NM_014428 |
| TLE1 | XM_005252151; XM_005252153; XM_011518951; NM_001303103; |
| | NM_001303104; XM_005252154; XM_005252163; XM_006717260; |
| | XM_047423814; XM_047423816; XM_006717263; XM_047423813; |
| | XM_047423818; XM_005252152; XM_047423815; XM_047423817; |
| | XM_006717258; XM_005252156; XM_005252162; XM_006717259; |
| | XM_006717261; XM_006717262; NM_005077 |
| TLE2 | NM_001144761; XM_006722864; NM_001144762; NM_001300846; |
| | XM_011528230; NM_003260 |
| TLE4 | XM_011518952; NM_001282753; XM_011518956; XM_011518969; |
| | XM_011518970; XM_017015068; XM_047423820; XM_047423823; |
| | XM_047423824; NM_001351546; XM_011518955; XM_011518957; |
| | XM_011518962; XM_011518965; XM_011518966; XM_047423821; |
| | XM_011518972; NM_001351542; NM_001351543; NM_001351547; |
| | NM_001351564; NM_007005; XM_011518953; XM_011518967; |
| | NM_001282748; NM_001282749; NM_001282760; NM_001351541; |
| | XM_006717264; XM_006717268; XM_011518961; XM_017015067; |
| | XM_017015074; XM_047423819; XM_047423822; NM_001351550; |
| | NR_104239; XM_011518958; XM_011518959; XM_011518963; |
| | XM_011518964; NM_001351552; NM_001351556; NM_001351563; |
| | XM_011518954; XM_011518960; XM_011518968; XM_017015083; |
| | NM_001351558; NM_001351560; NM_001351562 |
| TLN1 | NM_006289 |
| TLR2 | XM_017008575; NM_001318791; NM_001318796; NM_001318795; |
| | XM_017008573; XM_047416111; XM_047416113; NM_001318793; |
| | XM_047416114; NM_001318787; NM_003264; XM_011532216; |
| | XM_047416112; NM_001318790; XM_011532215; NM_001318789 |
| TMC6 | XM_047435250; XM_047435270; XM_047435271; XM_047435274; |
| | NM_001321185; NM_007267; NR_168289; XM_011524257; |
| | XM_047435252; XM_047435265; NM_001374594; NR_168288; |
| | XM_024450556; XM_047435256; XM_047435263; XM_047435278; |
| | NM_001374593; NR_168290; XM_011524256; XM_047435261; |
| | XM_047435268; XM_047435269; XM_047435277; NM_001127198; |
| | NM_001375354; NR_168291; XM_047435251; XM_047435253; |
| | XM_047435258; XM_047435272; XM_047435273; XM_047435255; |
| | XM_047435257; XM_047435266; XM_047435275; NM_001375353; |
| | XM_047435254; XM_047435259; XM_047435276; NM_001374596; |
| | XM_047435260; XM_047435264; XM_047435267 |
| TMC8 | XM_024450618; XM_024450620; XM_047435479; XM_047435494; |
| | XM_047435488; XR_007065273; XM_024450623; XM_047435492; |
| | XM_017024244; XM_024450619; XM_024450624; XM_047435482; |
| | XM_047435489; XR_002957973; XR_007065271; XM_024450622; |
| | XM_047435484; XM_047435485; XM_047435487; XM_047435491; |
| | XM_047435493; XR_007065274; XM_024450621; XR_007065276; |
| | XM_024450617; XM_024450626; XM_024450627; XM_047435478; |
| | XM_047435480; XM_047435481; XM_047435486; XM_047435490; |
| | XR_007065272; XM_024450625; NM_152468; XR_007065275 |
| TMED7- | NM_001164468; NM_001164469 |
| TICAM2 | |
| TNC | XM_005251975; XM_006717096; XM_011518628; XM_017014681; |
| | XM_0474233U; XM_047423321; XM_047423323; XM_047423328; |
| | XM_011518626; XM_047423312; XM_047423313; XM_047423317; |
| | XM_047423318; XM_005251973; XM_006717098; XM_047423322; |
| | XM_047423324; XM_047423327; XM_005251972; XM_006717097; |
| | XM_011518629; XM_047423309; XM_047423314; XM_047423325; |
| | XM_017014680; XM_047423315; XM_047423329; XM_011518625; |
| | XM_017014679; XM_047423310; XM_047423320; XM_047423330; |
| | XM_047423331; XM_005251974; XM_006717101; XM_047423316; |
| | XM_047423319; XM_047423326; XM_047423332; XM_017014678; |
| | XM_024447530; NM_002160 |
| TNFAIP3 | XM_024446533; XM_047419285; XM_011536095; XM_024446532; |
| | XM_047419282; XM_047419283; NM_006290; XM_011536096; |
| | NM_001270507; XM_005267119; XM_047419284; NM_001270508 |
| TNFAIP6 | XM_047445635; NM_007115 |
| TNFRSF10A | NM_003844 |
| TNFRSF10B | NM_003842; NR_027140; NM_147187 |
| TNFRSF10D | NM_003840 |
| TNFRSF11A | NM_001270950; XM_011526244; XM_017026066; NM_001270949; |
| | NM_001278268; XM_011526245; XM_017026064; NM_001270951; |
| | NM_003839; XM_017026065 |
| TNFRSF1A | NM_001346091; NM_001065; NM_001346092; NR_144351 |
| TNFRSF4 | XM_011542074; NM_003327; XR_007063145; XM_011542077; |
| | XM_011542075; XM_011542076 |
| TNFRSF8 | NM_001243; NM_152942; XM_011542441; XM_047434799; |
| | XM_011542443; XM_047434793; NM_001281430 |
| TNFSF10 | NR_033994; NM_001190943; NM_003810; NM_001190942 |
| TNFSF13 | NM_001198622; NM_172088; NR_073490; NM_001198624; NM_003808; |
| | NM_172087; NM_001198623 |
| TNFSF13B | NM_006573; XM_047430055; NM_001145645 |
| TNFSF18 | NM_005092 |
| TNFSF4 | XM_047429908; NM_003326; XM_047429896; NM_001297562; |
| | XM_017002228; XM_047429902; XM_011509964 |
| TNFSF8 | NM_001252290; NM_001244 |
| TNIP1 | XM_047416622; XM_047416623; XM_047416624; XM_047416627; |
| | NM_001252391; NM_001252393; XM_005268355; XM_006714752; |
| | XM_047416615; XM_047416620; XM_047416626; NM_001258455; |
| | NM_001258456; XM_047416617; XM_047416625; NM_001364487; |
| | XM_047416616; XM_047416618; NM_001252392; NM_001258454; |
| | NM_001364486; NM_001252385; XM_047416621; NM_001252386; |
| | XM_047416619; NM_001252390; NM_006058 |
| TNIP3 | NM_001244764; XM_017008625; NM_001128843; XM_047416181; |
| | XM_047416182; NM_024873; XM_011532256; XM_011532257 |
| TNS4 | XM_017025237; XM_047436949; XM_005257744; NM_032865; |
| | XM_017025236; XM_047436950 |
| TONSL | XM_011517050; XM_011517048; XM_011517049; NM_013432 |
| TOP1 | NM_003286 |
| TOP2A | XM_005257632; XM_011525165; NM_001067 |
| TOPBP1 | XM_047447356; NM_001363889; XM_047447355; XM_047447357; |
| | XM_017005636; XM_047447358; NM_007027; XM_011512357 |
| TOX3 | NM_001080430; XM_047433909; NM_001146188; XM_005255892; |
| | XM_011523002 |
| TP53 | NM_000546; NM_001126112; NM_001276695; NM_001126115; |
| | NM_001126116; NM_001126118; NM_001276697; NM_001276698; |
| | NM_001276760; NM_001276761; NM_001126114; NM_001276696; |
| | NM_001126113; NM_001126117; NM_001276699 |
| TP53BP1 | XM_047432994; XM_047432999; XM_047432997; NM_001141979; |
| | XR_007064488; XM_047432995; XM_047432998; XM_047432996; |
| | NM_001355001; NM_005657; NM_001141980 |
| TP63 | NM_001114978; NM_001329144; NM_001329146; NM_001329964; |
| | NM_001329145; NM_003722; NM_001114979; NM_001114982; |
| | NM_001329149; NM_001114980; NM_001114981; NM_001329150; |
| | NM_001329148 |
| TPBG | NM_001166392; NM_001376922; NM_006670 |
| TPSAB1 | NM_003294 |
| TRAC | NG_001332.3 |
| TRAF2 | XM_011518977; NM_021138; XM_011518976; XM_047423828; |
| | XM_047423829 |
| TRAF3 | XM_011537117; XM_017021618; NM_145726; XM_047431739; |
| | XM_047431741; NM_003300; NM_145725; XM_047431742; |
| | NM_001385143; NM_001199427; XM_011537116; XM_017021617; |
| | XM_017021619; XM_017021620; XM_011537118; XM_047431738; |
| | XM_047431740; NM_001385142 |
| TRAF3IP3 | NM_025228; NR_109871; XM_047430963; NM_001287754; |
| | NM_001320143; XM_005273279; XM_047430964; XM_011510018; |
| | XM_017002400; XM_011510019; NM_001320144; XM_047430976 |
| TRAF6 | NM_145803; NM_004620 |
| TRAT1 | NM_016388; NM_001317747 |
| TRIM37 | XM_005257385; XM_017024669; XM_047436106; XM_047436113; |
| | NM_001320988; NM_001320990; NM_001353082; XM_017024662; |
| | XM_047436109; XM_047436112; XM_047436120; XM_047436124; |
| | NM_001320987; NM_001353084; NM_001353086; NR_148346; |
| | XM_017024670; XM_047436110; XM_047436119; NM_001005207; |
| | NM_015294; XM_011524831; XM_047436123; NM_001353083; |
| | NM_001353085; XM_011524833; XM_047436114; XM_047436117; |
| | XM_047436121; XM_047436122; XM_047436125; XM_047436126; |
| | NM_001320989; XM_017024667; XM_047436107; XM_047436108; |
| | XM_047436115; XM_011524834; XM_011524836; XM_017024663; |
| | XM_011524832; XM_017024665; XM_017024673; XM_047436111; |
| | XM_047436116; XM_047436118; NR_148347 |
| TRIP10 | NM_001288962; XM_005259683; NM_004240; NR_110231; |
| | XM_006722940; NM_001288963 |
| TRPM1 | NM_001252024; NM_001252030; NM_001252020; NM_002420 |
| TSC1 | XM_017015096; NM_000368; XM_006717271; XM_017015098; |
| | XM_011518979; XM_047423836; NM_001162426; XM_017015097; |
| | XM_047423830; XM_047423835; XM_005272211; NM_001162427; |
| | NM_001362177; XM_047423834; XM_047423831; XM_047423832; |
| | XM_047423837; NM_001008567; XM_047423833 |
| TSC2 | XM_047434556; NM_021056; NM_001318831; XM_047434555; |
| | XM_011522637; NM_001077183; NM_001318832; NM_001363528; |
| | XM_011522639; XM_017023615; XM_047434557; NM_001318827; |
| | NM_001370405; XM_011522636; XM_011522640; NM_000548; |
| | NM_001370404; NM_021055; XM_011522638; NM_001114382; |
| | NM_001318829 |
| TUBB3 | NM_006086; NM_001197181 |
| TWIST1 | NR_149001; NM_000474 |
| TXNDC11 | NM_015914; XM_047434192; NM_001324024; NR_136671; NR_136673; |
| | NR_136674; XM_047434191; NM_001324022; NM_001324025; |
| | NM_001303447; NR_136672; XM_011522515 |
| TXNDC5 | NM_030810; NM_022085; NM_001145549 |
| TXNRD1 | NM_001261446; NM_182742; NM_182743; NM_003330; NM_182729; |
| | NM_001093771; NM_001261445 |
| TYK2 | XM_011528246; XM_011528247; XM_047439304; NM_001385201; |
| | NM_001385204; XM_011528245; NM_003331; NM_001385197; |
| | NM_001385206; XM_011528249; NM_001385207; XM_047439305; |
| | NM_001385198; NM_001385200; NM_001385202; XM_047439306; |
| | XM_047439307; NM_001385199; NM_001385203; NM_001385205 |
| TYMP | NM_001257988; NM_001113755; NM_001257989; NM_001953; |
| | NM_001113756 |
| TYMS | NM_001354867; NM_001354868; XM_024451242; NM_001071 |
| TYRO3 | NM_001330264; XM_017022543; NM_006293 |
| TYROBP | NM_001173515; NM_003332; NR_033390; NM_001173514; NM_198125 |
| U2AF1 | XM_017028468; XM_024452129; XM_024452130; NM_001025204; |
| | NM_001025203; XM_024452131; XM_011529743; NM_006758 |
| UBASH3A | XM_047440833; XM_047440834; XM_047440835; XM_047440838; |
| | XM_047440836; XM_047440840; NM_001001895; NM_001243467; |
| | NM_018961; XM_047440837; XM_047440839; XM_011529609; |
| | XM_047440831; XM_047440832; XR_007067789 |
| UBE2C | NM_001281742; NM_001281741; NM_181802; NM_181803; NR_104036; |
| | NR_104037; NM_007019; NM_181800; NM_181801; NM_181799 |
| UBE2V2 | NM_003350; XM_017013808; XM_011517583 |
| UBR5 | XM_047421847; NM_001282873; XM_047421849; XM_005250962; |
| | XM_047421848; XM_024447178; XM_024447179; NM_015902 |
| UGCG | NM_003358; XM_047423844; XM_017015107 |
| UGP2 | XM_024453120; NM_001377526; NM_001377527; NM_001001521; |
| | NM_001377525; NM_001377528; NM_001377524; NM_001377529; |
| | NM_006759 |
| UGT1A4 | NM_007120 |
| UGT2B4 | NM_001297616; NM_021139; NM_001297615 |
| UHRF1 | XM_047438707; XM_011527942; XM_047438709; NM_001048201; |
| | NM_001290051; NM_001290052; NM_001290050; XM_047438708; |
| | NM_013282 |
| ULK1 | XM_011538799; XM_011538798; NM_003565; XR_007063134 |
| UNC5B | NM_170744; NM_001244889 |
| UNG | NM_003362; NM_080911 |
| UPB1 | NM_016327; XM_047441404; XR_001755249; XM_011530223; |
| | XM_047441405 |
| USP22 | XM_005256575; XM_047435703; NM_015276 |
| UVSSA | XM_017008490; XM_017008494; XM_047416025; NM_020894; |
| | XM_017008492; XM_017008493; XM_017008497; XR_001741302; |
| | XM_017008499; XR_001741304; XR_007057948; XM_017008498; |
| | XM_017008500; NM_001317934; XR_001741303; XM_017008495; |
| | XM_017008496; XM_024454162; XM_047416026; XR_007057949; |
| | XM_047416027; XM_047416028; NM_001317935 |
| VANGL1 | NM_138959; NM_001172412; NM_001172411 |
| VAV1 | XM_005259642; NM_005428; NM_001258206; NM_001258207 |
| VAV3 | XM_017000053; NM_001079874; NM_006113; XM_017000054; |
| | XM_047430439; XM_005270360; XM_017000055; XM_047430543; |
| | XM_005270361; XM_017000056; XM_024450319; XM_047430476; |
| | XM_047430555; XR_007063680 |
| VCAM1 | NM_080682; NM_001078; NM_001199834 |
| VCAN | NM_001126336; NM_001164098; NM_004385; NM_001164097 |
| VCL | NM_003373; NM_014000 |
| VEGFA | NM_001171625; NM_003376; NM_001033756; NM_001171624; |
| | NM_001171626; NM_001171630; NM_001025366; NM_001317010; |
| | NM_001025368; NM_001025370; NM_001171623; NM_001171622; |
| | NM_001171628; NM_001171629; NM_001204385; NM_001025367; |
| | NM_001025369; NM_001171627; NM_001204384; NM_001287044 |
| VEGFB | NM_003377; NM_001243733 |
| VEGFC | NM_005429 |
| VHL | NM_198156; NM_000551; NM_001354723 |
| VIM | NM_003380 |
| VMP1 | NM_001329396; NM_001329398; NM_001329394; NM_001329395; |
| | NM_001329397; NM_001329401; NM_030938; NM_001329399; |
| | NM_001329400; NM_001329402 |
| VSIG4 | NM_007268; NM_001184830; NM_001184831; NM_001100431; |
| | NM_001257403 |
| VWF | NM_000552; XM_047429501 |
| WAS | XM_011543977; XM_047442434; XM_047442432; XM_017029786; |
| | XM_047442433; NM_000377 |
| WHSC1 | XM_047416137; XM_047416144; NM_001042424; NM_133335; |
| | NM_014919; XM_005248002; XM_005248005; XM_047416141; |
| | NM_133330; XM_047416139; XM_011513560; XM_047416138; |
| | NM_133333; XM_005248001; XM_047416142; NM_133332; NM_133336; |
| | XM_047416143; NM_007331; NM_133331; NM_133334 |
| WIFI | NM_007191 |
| WIPI1 | NR_135471; NM_017983; XM_017024808; NM_001320772; NR_135470 |
| WNT10A | XM_011511930; XM_011511929; NM_025216 |
| WNT2 | NM_003391; NR_024047 |
| WNT2B | NM_004185; NM_001291880; NM_024494 |
| WNT3 | NM_030753 |
| WNT3A | NM_033131 |
| WNT5B | NM_030775; NM_032642 |
| WNT6 | NM_006522 |
| WNT7A | XM_011534091; NM_004625; XM_047448863 |
| WNT7B | XM_011530366; NM_058238 |
| WNT9A | NM_003395; XM_011544271 |
| WNT9B | XM_011525178; NM_003396; NM_001320458 |
| WRN | XM_011544639; XR_949472; NM_000553; XR_949471; XR_949470; |
| | XM_011544640 |
| WT1 | NM_000378; NR_160306; NM_001367854; NM_001198551; |
| | NM_001198552; NM_024424; NM_024426; NM_024425 |
| WWC1 | XM_011534487; XM_011534489; XM_047417020; XM_047417021; |
| | NM_015238; XM_005265850; XM_011534485; XM_011534486; |
| | XM_047417017; XM_047417018; XM_005265853; XM_011534488; |
| | XM_011534490; XM_011534491; XM_017009276; XM_047417019; |
| | NM_001161662; NM_001161661 |
| WWTR1 | XM_047447930; NM_001168278; XM_047447933; XM_047447932; |
| | NM_001168280; NM_001348362; XM_017006122; XM_047447931; |
| | NM_015472 |
| XAB2 | NM_020196 |
| XAF1 | XM_011523948; XM_011523949; NM_001353136; NM_001353140; |
| | XR_007065309; NM_001353137; NM_001353138; NM_199139; |
| | XM_047436318; NM_017523; XR_007065310; XR_934053; |
| | XM_047436316; NM_001353134; NM_001353135; NM_001353139; |
| | NR_046398; XR_007065308; XM_047436317 |
| XBP1 | NM_001394000; NM_005080; NM_001079539; NM_001393999 |
| XCR1 | NM_001024644; NM_005283; NR_170111; NM_001381860 |
| XIAP | NR_037916; NM_001204401; NR_165803; NM_001167; NM_001378592; |
| | NM_001378591; NM_001378590 |
| XPA | NR_149093; XM_006717278; NM_001354975; NR_149092; NM_000380; |
| | NR_027302; NR_149094; NR_149091 |
| XPC | NM_001354729; NM_001145769; NM_001354730; NM_004628; |
| | XM_047448864; XM_047448865; NM_001354726; NM_001354727; |
| | NR_148950; NR_148951 |
| XRCC1 | NM_006297 |
| XRCC3 | XM_005268046; NM_001371231; XM_047431767; XM_047431768; |
| | NM_001100119; NM_001371229; XM_047431766; NM_001371232; |
| | NM_001100118; NM_005432 |
| XRCC4 | XM_047417695; XM_047417696; XM_047417698; XM_017009828; |
| | NM_003401; NM_022550; XM_011543626; XM_017009827; |
| | NM_001318012; NM_022406; XM_047417694; NM_001318013; |
| | XM_047417697 |
| XRCC5 | NM_021141 |
| XRCC6 | NM_001469; NM_001288976; NM_001288977; NM_001288978; |
| | XM_047441304 |
| YAP1 | XM_005271380; XM_005271381; NM_001130145; NM_001282099; |
| | NM_006106; NM_001282097; XM_005271378; XM_005271383; |
| | XM_011542555; NM_001282098; NM_001195045; NM_001282100; |
| | NM_001195044; NM_001282101 |
| ZAP70 | NM_001378594; NM_207519; XR_007081582; NM_001079; |
| | XM_047445775; XM_047445774; XM_047445776; XR_007081583 |
| ZBED2 | NM_024508 |
| ZBTB16 | XM_047427563; NM_001354751; NM_001354752; NM_006006; |
| | XM_005271658; NM_001018011; NM_001354750 |
| ZBTB7A | NM_015898; NM_001317990; XM_005259570; NM_020224; |
| | XM_005259571 |
| ZEB2 | NM_001171653; NM_014795; NR_033258 |
| ZFYVE9 | XM_011542437; XM_047434682; NM_007324; XM_047434674; |
| | NM_007323; NM_004799 |
| ZMYND11 | NM_001202465; NM_001370103; NM_001370110; NM_001370115; |
| | NM_001370101; NM_001370104; NM_001370108; NM_001370122; |
| | NM_001202468; NM_001330057; NM_001370098; NM_001370099; |
| | NM_001370106; XM_017015594; NM_001370102; NM_001370119; |
| | NM_001370107; NM_001370109; NM_001370114; NM_001370117; |
| | NM_001370118; NM_006624; NM_001202466; NM_001202467; |
| | NM_001370100; NM_001370113; NM_001370121; NM_001370123; |
| | NM_212479; NM_001370097; NM_001370111; NM_001370120; |
| | NM_001370124; NM_001202464; NM_001370105; NM_001370112; |
| | NM_001370116; NR_163254 |
| ZNF101 | XM_047439722; XM_047439723; NM_033204; NM_001300949; |
| | XM_024451785 |
| ZNRF3 | NM_001206998; NM_032173 |

### EQUIVALENTS

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure. The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for mapping RNA expression levels for genes expressed in a biological sample and obtained from a subject using a first protocol to RNA expression levels as would have been determined through a second protocol if the second protocol were used to process the biological sample instead of the first protocol, the method comprising:
using at least one computer hardware processor to perform:
(A) obtaining first RNA expression data for a set of genes expressed in the biological sample obtained from the subject, the first RNA expression data indicative of first RNA expression levels of genes in the set of genes, the first RNA expression data previously determined by processing the biological sample using the first protocol; and
(B) mapping the first RNA expression levels of genes in the set of genes to second RNA expression levels of genes in the set of genes, the second RNA expression levels indicating RNA expression levels as would have been determined through the second protocol, the second protocol being different from the first protocol, if the second protocol were used to process the biological sample instead of the first protocol, the mapping comprising:
for a first gene in the set of genes:
obtaining, from among the first RNA expression levels, a first set of RNA expression levels including a first RNA expression level for the first gene and zero, one, or multiple first RNA expression levels for zero, one, or multiple other genes in the set of genes associated with the first gene;
obtaining a first transformation for estimating an RNA expression level for the first gene as would have been determined according to the second protocol from RNA expression levels of one or more genes as determined through the first protocol; and
determining, for inclusion in the second RNA expression levels, a second RNA expression level for the first gene by applying the first transformation to the first set of RNA expression levels.

2. A method for identifying a subject as a member of a cohort, the method comprising:
using at least one computer hardware processor to perform:
(A) obtaining first RNA expression data for a set of genes expressed in a biological sample obtained from the subject, the first RNA expression data indicative of first RNA expression levels of genes in the set of genes, the first RNA expression data previously determined by processing the biological sample using a first protocol;
(B) mapping the first RNA expression levels of genes in the set of genes to second RNA expression levels of genes in the set of genes, the second RNA expression levels indicating RNA expression levels as would have been determined through a second protocol different from the first protocol if the second protocol were used to process the biological sample instead of the first protocol, the mapping comprising:
for a first gene in the set of genes:
obtaining, from among the first RNA expression levels, a first set of RNA expression levels including a first RNA expression level for the first gene and zero, one, or multiple first RNA expression levels for zero, one, or multiple other genes, in the set of genes, which are associated with the first gene;
obtaining a first transformation for estimating, from RNA expression levels of one or more genes as determined through the first protocol, an RNA expression level for the first gene as would have been determined according to the second protocol; and
determining, for inclusion in the second RNA expression levels, a second RNA expression level for the first gene by applying the first transformation to the first set of RNA expression levels; and
(C) identifying a cohort, from among a plurality of cohorts, with which to associate the subject using the second RNA expression levels.

3. The method of claim 1, wherein the set of genes comprises a second gene and a second set of genes associated with the second gene;
wherein the mapping comprises:
obtaining, from among the first RNA expression levels, a second set of RNA expression levels including a first RNA expression level for the second gene and RNA expression levels for genes in the second set of genes associated with the second gene;
obtaining a second transformation for estimating, from RNA expression levels of one or more genes as determined through the first protocol, an RNA expression level for the second gene as would have been determined according to the second protocol, wherein the second transformation is different than the first transformation; and
determining, for inclusion in the second RNA expression levels a second RNA expression level for the second gene by applying the second transformation to the second set of RNA expression levels.

4. The method of claim 1, wherein the set of genes comprises one or more additional genes, and a further set of genes associated with the one or more additional genes;
wherein the mapping comprises:
obtaining, from among the first RNA expression levels, a set of RNA expression levels including RNA expression levels for each of at least some of the one or more additional genes and RNA expression levels for at least some of the genes of the further set of genes associated with the one or more additional genes;
obtaining respective transformations for estimating RNA expression levels for each of the one or more additional genes as would have been determined according to the second protocol; and
determining, for inclusion in the second RNA expression levels second RNA expression levels for each of the at least some of the additional genes of the subset by applying the second transformation to the first set of RNA expression levels.

5. The method of claim 1, comprising, prior to the mapping:
determining, for each gene of at least a subset of the set of genes, a respective transformation for estimating the RNA expression level for each gene of the subset as would have been determined according to the second protocol from RNA expression levels of one or more genes of the subset as determined through the first protocol.

6. The method of claim 1, wherein the transformation is a linear transformation, and wherein determining the first transformation is performed using a regularized linear regression technique using training data.

7. The method of claim 6, wherein the training data comprises a plurality of paired values of RNA expression levels for each at least some of the set of genes, wherein each pair of values in the plurality of paired values comprises an RNA expression level as determined through applying the first protocol to a particular biological sample and another RNA expression level as determined through applying the second protocol to the particular biological sample.

8. The method of claim 1, wherein the obtaining the first set of RNA expression levels consists of:
obtaining a first RNA expression level for the first gene and zero other RNA expression levels.

9. The method of claim 1, wherein the obtaining the first set of RNA expression levels comprises:
identifying one or multiple other genes associated with the first gene.

10. The method of claim 9, wherein the identifying is performed using Pearson correlation.

11. The method of claim 1, wherein the multiple other genes in the set of genes comprises between 2 and 100 genes associated with the first gene.

12. The method of claim 1, wherein the biological sample comprises a blood sample or tissue sample.

13. The method of claim 12, wherein the tissue sample comprises tumor tissue.

14. The method of claim 1, wherein the subject is a mammal, optionally wherein the subject is a human.

15. The method of claim 1, wherein the first expression data and the second expression data each comprise normalized RNA expression levels.

16. The method of claim 1, wherein the normalized RNA expression levels are normalized to transcripts per million (TPM) units.

17. The method of claim 1, wherein the first protocol comprises preserving the biological sample by a formalin-fixation and paraffin-embedding (FFPE) technique.

18. The method of claim 17, wherein the first protocol further comprises performing exome capture (EC) RNA sequencing on the FFPE preserved biological sample.

19. The method of claim 1, wherein the second protocol comprises preserving the biological sample by a freshly frozen (FF) technique.

20. The method of claim 19, wherein the second protocol comprises performing poly-A RNA sequencing on the FF preserved biological sample.

21. The method of claim 1 further comprising generating the first RNA expression data by applying the first protocol to the biological sample.

22. The method of claim 2, wherein the identifying the cohort comprises:
associating the second RNA expression levels to RNA expression levels of a particular cohort of the plurality of cohorts; and
identifying the subject as a member of the particular cohort to which the second RNA expression levels are associated.

23. The method of claim 1, further comprising selecting a cancer therapeutic for the subject using the second RNA expression levels.

24. The method of claim 23, wherein the selecting a cancer therapeutic comprises:
determining a plurality of gene group RNA expression levels using the second RNA expression levels, the plurality of gene group RNA expression levels comprising a gene group RNA expression level for each gene group in a set of gene groups, wherein the set of gene groups comprises at least one gene group associated with cancer malignancy, and at least one gene group associated with cancer microenvironment; and
selecting a cancer therapeutic using the determined gene group RNA expression levels.

25. The method of claim 23 further comprising administering the selected cancer therapeutic to the subject.

26. A system, comprising:
at least one computer hardware processor; and
at least one computer-readable storage medium storing processor-executable instructions that, when executed by the at least one computer hardware processor, cause the at least one computer hardware processor to perform a method for mapping RNA expression levels for genes expressed in a biological sample and obtained from a subject using a first protocol to RNA expression levels as would have been determined through a second protocol if the second protocol were used to process the biological sample instead of the first protocol, the method comprising:
using at least one computer hardware processor to perform:
(A) obtaining first RNA expression data for a set of genes expressed in the biological sample obtained from the subject, the first RNA expression data indicative of first RNA expression levels of genes in the set of genes, the first RNA expression data previously determined by processing the biological sample using the first protocol; and
(B) mapping the first RNA expression levels of genes in the set of genes to second RNA expression levels of genes in the set of genes, the second RNA expression levels indicating RNA expression levels as would have been determined through the second protocol, the second protocol being different from the first protocol, if the second protocol were used to process the biological sample instead of the first protocol, the mapping comprising:
for a first gene in the set of genes:
obtaining, from among the first RNA expression levels, a first set of RNA expression levels including a first RNA expression level for the first gene and zero, one, or multiple first RNA expression levels for zero, one, or multiple other genes in the set of genes associated with the first gene;
obtaining a first transformation for estimating an RNA expression level for the first gene as would have been determined according to the second protocol from RNA expression levels of one or more genes as determined through the first protocol; and
determining, for inclusion in the second RNA expression levels, a second RNA expression level for the first gene by applying the first transformation to the first set of RNA expression levels.

27. At least one computer-readable storage medium storing processor-executable instructions that, when executed by at least one computer hardware processor, cause the at least one computer hardware processor to perform a method for mapping RNA expression levels for genes expressed in a biological sample and obtained from a subject using a first protocol to RNA expression levels as would have been determined through a second protocol if the second protocol were used to process the biological sample instead of the first protocol, the method comprising:
using at least one computer hardware processor to perform:
(A) obtaining first RNA expression data for a set of genes expressed in the biological sample obtained from the subject, the first RNA expression data indicative of first RNA expression levels of genes in the set of genes, the first RNA expression data previously determined by processing the biological sample using the first protocol; and
(B) mapping the first RNA expression levels of genes in the set of genes to second RNA expression levels of genes in the set of genes, the second RNA expression levels indicating RNA expression levels as would have been determined through the second protocol, the second protocol being different from the first protocol, if the second protocol were used to process the biological sample instead of the first protocol, the mapping comprising:
for a first gene in the set of genes:
obtaining, from among the first RNA expression levels, a first set of RNA expression levels including a first RNA expression level for the first gene and zero, one, or multiple first RNA expression levels for zero, one, or multiple other genes in the set of genes associated with the first gene;
obtaining a first transformation for estimating an RNA expression level for the first gene as would have been determined according to the second protocol from RNA expression levels of one or more genes as determined through the first protocol; and
determining, for inclusion in the second RNA expression levels, a second RNA expression level for the first gene by applying the first transformation to the first set of RNA expression levels.
